(19) United States
(12) Patent Application Publication
(10) Pub. No.: US 2023/0050965 A1
(43) Pub. Date: Feb. 16, 2023

(54) SSTR5 ANTAGONISTS

(71) Applicant: Kallyope, Inc., New York, NY (US)

(72) Inventors: Iyassu SEBHAT, New York, NY (US); Shuwen HE, New York, NY (US)

(21) Appl. No.: 17/782,370

(22) PCT Filed: Dec. 2, 2020
(86) PCT No.: PCT/US2020/062891

§ 371 Date: Jun. 3, 2022

Related U.S. Application Data
(60) Provisional application No. 62/943,099, filed on Dec. 3, 2019.

(51) Int. Cl. A61K 31/675; C07F 9/6561; C07F 5/02; C07D 498/10; C07D 471/10; C07F 9/6509; C07D 295/205; A61K 31/69; A61K 31/438; A61K 31/495; A61K 45/06

(52) U.S. Cl. CPC A61K 31/675; C07F 9/6561; C07F 5/025; C07D 498/10; C07D 471/10; C07F 9/650952; C07D 295/205; A61K 31/69; A61K 31/438; A61K 31/495; A61K 45/06

(57) ABSTRACT

This disclosure is directed, at least in part, to SSTR5 antagonists useful for the treatment of conditions or disorders involving the gut-brain axis. In some embodiments, the SSTR5 antagonists are gut-restricted compounds. In some embodiments, the condition or disorder is a metabolic disorder, such as diabetes, obesity, nonalcoholic steatohepatitis (NASH), or a nutritional disorder such as short bowel syndrome.

### RELATED APPLICATIONS

This application claims the benefit of U.S. Provisional Patent Application No. 62/943,099 filed on Dec. 3, 2019, which is incorporated herein by reference in its entirety.

### BRIEF SUMMARY OF THE INVENTION

Disclosed herein, in certain embodiments, are somatostatin receptor 5 (SSTR5) antagonists useful for the treatment of conditions or disorders involving the gut-brain axis. In some embodiments, the SSTR5 antagonists are gut-restricted or selectively modulate SSTR5 located in the gut. In some embodiments, the condition is selected from the group consisting of: central nervous system (CNS) disorders including mood disorders, anxiety, depression, affective disorders, schizophrenia, malaise, cognition disorders, addiction, autism, epilepsy, neurodegenerative disorders, Alzheimer's disease, and Parkinson's disease, Lewy Body dementia, episodic cluster headache, migraine, pain; metabolic conditions including diabetes and its complications such as chronic kidney disease/diabetic nephropathy, diabetic retinopathy, diabetic neuropathy, and cardiovascular disease, metabolic syndrome, obesity, dyslipidemia, and nonalcoholic steatohepatitis (NASH); eating and nutritional disorders including hyperphagia, cachexia, anorexia nervosa, short bowel syndrome, intestinal failure, intestinal insufficiency and other eating disorders; inflammatory disorders and autoimmune diseases such as inflammatory bowel disease, ulcerative colitis, Crohn's disease, psoriasis, and celiac disease; necrotizing enterocolitis; gastrointestinal injury resulting from toxic insults such as radiation or chemotherapy; diseases/disorders of gastrointestinal barrier dysfunction including environmental enteric dysfunction, spontaneous bacterial peritonitis; functional gastrointestinal disorders such as irritable bowel syndrome, functional dyspepsia, functional abdominal bloating/distension, functional diarrhea, functional constipation, and opioid-induced constipation; gastroparesis; nausea and vomiting; disorders related to microbiome dysbiosis, and other conditions involving the gut-brain axis.

Disclosed herein, in certain embodiments, is a compound of Formula (I):

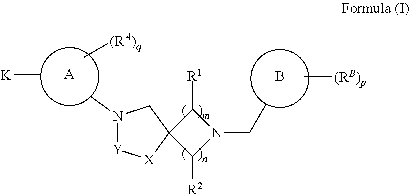

or a pharmaceutically acceptable salt, solvate, stereoisomer, or prodrug thereof, wherein:
X is —O—, —NR³—, or —C(R⁴)₂—;
Y is —C(═O)—, or —S(═O)₂—;
Ring A is aryl, heteroaryl, cycloalkyl, or heterocycloalkyl;
Ring B is aryl or heteroaryl;
K is —(CH₂)ⱼ-G;
   G is —P(═O)(OH)₂, —P(═O)(OH)(R^{D}), —P(═O)(OH)(H), —P(═O)(OH)(OR^{D}), —B(OH)₂, —B(OR^{D})(OH), —NHC(═O)H, —NHC(═O)(R^{D}), —NHS(═O)₂(R^{D}) —NHC(═O)NHS(═O)₂(R^{D}), —N(R^{D})C(═O)NHS(═O)₂(R^{D}), —C(═O)NHS(═O)₂(R^{D}), —S(═O)₂NHC(═O)(R^{D}), —NHC(═O)NH₂, —NHC(═O)NH(R^{D}), —NHC(═NH)NH₂, —NHC(═NH)NH(R^{D}), —NHC(═NH)N(R^{D})₂, —N(R^{D})C(═NH)NH₂, —N(R^{D})C(═NH)NH(R^{D}), —N(R^{D})C(═NH)N(R^{D})₂, —NHC(═N(R^{D}))NH₂, —NHC(═N(R^{D}))NH(R^{D}), —NHC(═N(R^{D}))N(R^{D})₂, N(R^{D})C(═N(R^{D}))NH₂, —N(R^{D})C(═N(R^{D}))NH(R^{D}), —N(R^{D})C(═N(R^{D}))N(R^{D})₂, —NHC(═NH)NHC(═NH)NH₂, —N(R^{D})C(═NH)NHC(═NH)NH₂,

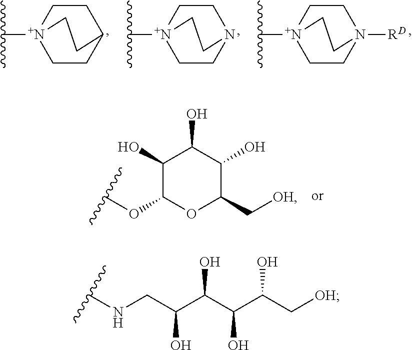

j is 0-4;
each R¹ and R² is independently hydrogen, C₁₋₆ alkyl, or C₁₋₆ fluoroalkyl;
or one R¹ and one R² are taken together to form a ring; R³ is hydrogen, C₁₋₆ alkyl, C₁₋₆ fluoroalkyl, or C₃₋₆ cycloalkyl;
each R⁴ is independently hydrogen, C₁₋₆ alkyl, C₁₋₆ fluoroalkyl, or C₃₋₆ cycloalkyl;
each R^{D} is independently C₁₋₆ alkyl or C₃₋₆ cycloalkyl; wherein the alkyl and cycloalkyl are unsubstituted or substituted by 1-3 halogen or —OH groups;
each R^{A} is independently halogen, —OH, —O—(C₁-C₆ alkyl), C₁-C₆ alkyl, C₃-C₆ cycloalkyl, 3- to 8-membered heterocycloalkyl, wherein each alkyl, cycloalkyl, and heterocycloalkyl is unsubstituted or substituted with 1, 2, or 3 substituents selected from halogen, —CN, —OH, —O—(C₁-C₆ alkyl), C₁-C₆ alkyl, C₁-C₆ fluoroalkyl, C₁-C₆ hydroxyalkyl, —O—(C₁-C₆ fluoroalkyl), C₃-C₆ cycloalkyl, and 3- to 6-membered heterocycloalkyl;
each R^{B} is independently halogen, C₁-C₆ alkyl, C₃-C₆ cycloalkyl, C₃-C₆ cycloalkenyl, 3- to 8-membered heterocycloalkyl, 3- to 8-membered heterocycloalkenyl, aryl, heteroaryl, —CN, —OR⁹, —OCH₂R⁹, —CO₂R⁹, —CH₂CO₂R⁹, —OC(═O)R⁹, —C(═O)N(R⁹)₂, —N(R⁹)₂, —NR⁹C(═O)R⁹, —NR⁹C(═O)OR¹⁰, —OC(═O)NR⁹, —NR⁹C(═O)N(R⁹)₂, —C(R⁹)═N—OR⁹, —SR⁹, —S(═O)R¹⁰, —S(═O)₂R¹⁰, —S(═O)₂N(R⁹)₂, —P(═O)(OR⁹)₂, —P(═O)(OR⁹)R¹⁰ or —P(═O)(R¹⁰)₂, wherein each alkyl, aryl, and heteroaryl is unsubstituted or substituted with 1, 2, or 3 substituents selected from halogen, —CN, —OH, —O—(C₁-C₆ alkyl), —CO₂—(C₁-C₆ alkyl), C₁-C₆ alkyl, C₁-C₆ fluoroalkyl, C₁-C₆ hydroxyalkyl, —O—(C₁-C₆ fluoroalkyl), C₃-C₆ cycloalkyl, and 3- to 6-membered heterocycloalkyl; and wherein each cycloalkyl, cycloalkenyl, heterocycloalkyl, and heterocycloalkenyl is unsubstituted or substituted with 1, 2, or 3 substituents selected from halogen, —CN, —OH, ═O, —O—(C₁-C₆ alkyl), C₁-C₆ alkyl, C₁-C₆ fluoroalkyl, C₁-C₆ hydroxyalkyl, —O—(C₁-C₆ fluoroalkyl), C₃-C₆ cycloalkyl, and 3- to 6-membered heterocycloalkyl;
each R⁹ is independently selected from hydrogen, C₁-C₆ alkyl, C₁-C₆ fluoroalkyl, C₃-C₆cycloalkyl, 3- to 8-membered heterocycloalkyl, phenyl, and monocyclic heteroaryl, wherein each alkyl, fluoroalkyl, cycloalkyl, heterocycloalkyl, phenyl, and heteroaryl is unsubstituted or substituted with 1, 2, or 3 substituents selected from halogen, —CN, —OH, —O—(C₁-C₆ alkyl), —NH₂, —NH(C₁-C₆ alkyl), —N(C₁-C₆ alkyl)₂, C₁-C₆ alkyl, C₁-C₆ fluoroalkyl, C₁-C₆ hydroxyalkyl, —O—(C₁-C₆ fluoroalkyl), C₃-C₆ cycloalkyl, 3- to 6-membered heterocycloalkyl, and

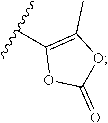 [SMILES: *c1oc(=O)oc1C]

or two R⁹ on the same N atom are taken together with the N atom to which they are attached to form a N-containing heterocycle, which is unsubstituted or substituted with 1, 2, or 3 substituents selected from halogen, —CN, —OH, —O—(C₁-C₆ alkyl), —NH₂, —NH(C₁-C₆ alkyl), —N(C₁-C₆ alkyl)₂, C₁-C₆ alkyl, C₁-C₆ fluoroalkyl, C₁-C₆ hydroxyalkyl, —O—(C₁-C₆ fluoroalkyl), C₃-C₆ cycloalkyl, and 3- to 6-membered heterocycloalkyl;
each R¹⁰ is independently selected from C₁-C₆ alkyl, C₁-C₆ fluoroalkyl, C₃-C₆ cycloalkyl, 3- to 8-membered heterocycloalkyl, phenyl, and monocyclic heteroaryl, wherein each alkyl, fluoroalkyl, cycloalkyl, heterocycloalkyl, phenyl, and heteroaryl is unsubstituted or substituted with 1, 2, or 3 substituents selected from halogen, —CN, —OH, —O—(C₁-C₆ alkyl), —NH₂, —NH(C₁-C₆ alkyl), —N(C₁-C₆ alkyl)₂, C₁-C₆ alkyl, C₁-C₆ fluoroalkyl, C₁-C₆ hydroxyalkyl, —O—(C₁-C₆ fluoroalkyl), C₃-C₆ cycloalkyl, 3- to 6-membered heterocycloalkyl, and

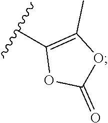 [SMILES: *c1oc(=O)oc1C]

m is 1 or 2; L
n is 1 or 2;
p is 0-4; and
q is 0-4.

Also disclosed herein, in certain embodiments, is a compound of Formula (II):

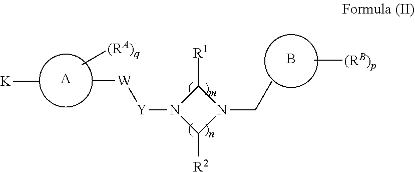

or a pharmaceutically acceptable salt, solvate, stereoisomer, or prodrug thereof, wherein:
W is a bond, —O—, —NR³—, or —C(R⁴)₂—;
Y is —C(═O)—, or —S(═O)₂—;
Ring A is aryl, heteroaryl, cycloalkyl, or heterocycloalkyl;
Ring B is aryl or heteroaryl;
K is —(CH₂)ⱼ-G;
   G is —P(═O)(OH)₂, —P(═O)(OH)(R^{D}), —P(═O)(OH)(H), —P(═O)(OH)(OR^{D}), —B(OH)₂, —B(OR^{D})(OH), —NHC(═O)H, —NHC(═O)(R^{D}), —NHS(═O)₂(R^{D}), —NHC(═O)NHS(═O)₂(R^{D}), —N(R^{D})C(═O)NHS(═O)₂(R^{D}), —C(═O)NHS(═O)₂(R^{D}), —S(═O)₂NHC(═O)(R^{D}), —NHC(═O)NH₂, —NHC(═O)NH(R^{D}), —NHC(═NH)NH₂, —NHC(═NH)NH(R^{D}), —NHC(═NH)N(R^{D})₂, —N(R^{D})C(═NH)NH₂, —N(R^{D})C(═NH)NH(R^{D}), —N(R^{D})C(═NH)N(R^{D})₂, —NHC(═N(R^{D}))NIH2, —NHC(═N(R^{D}))NH(R^{D}), —NHC(═N(R^{D}))N(R^{D})₂, N(R^{D})C(═N(R^{D}))NH₂, —N(R^{D})C(═N(R^{D}))NH(R^{D}), —N(R^{D})C(═N(R^{D}))N(R^{D})₂, —NHC(═NH)NHC(═NH)NH₂, —N(R^{D})C(═NH)NHC(═NH)NH₂,

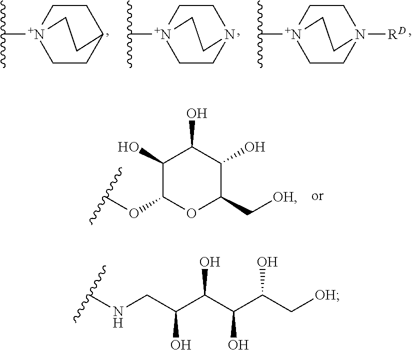

j is 0-4;
wherein when K is —B(OH)₂, W is —O—, —NR³—, or —C(R⁴)₂—;
each R¹ and R² is independently hydrogen, C₁₋₆ alkyl, or C₁₋₆ fluoroalkyl;
or one R¹ and one R² are taken together to form a ring;
R³ is hydrogen, C₁₋₆ alkyl, C₁₋₆ fluoroalkyl, or C₃₋₆ cycloalkyl;
each R⁴ is independently hydrogen, C₁₋₆ alkyl, C₁₋₆ fluoroalkyl, or C₃₋₆ cycloalkyl;
each R^{D} is independently C₁₋₆ alkyl or C₃₋₆ cycloalkyl; wherein the alkyl and cycloalkyl are unsubstituted or substituted by 1-3 halogen or —OH groups;
each R^{A} is independently halogen, —OH, —O—(C₁-C₆ alkyl), C₁-C₆ alkyl, C₃-C₆ cycloalkyl, 3- to 8-membered heterocycloalkyl, wherein each alkyl, cycloalkyl, and heterocycloalkyl is unsubstituted or substituted with 1, 2, or 3 substituents selected from halogen, —CN, —OH, —O—(C₁-C₆ alkyl), C₁-C₆ alkyl, C₁-C₆ fluoroalkyl, C₁-C₆ hydroxyalkyl, —O—(C₁-C₆ fluoroalkyl), C₃-C₆ cycloalkyl, and 3- to 6-membered heterocycloalkyl;
each R^{B} is independently halogen, C₁-C₆ alkyl, C₃-C₆ cycloalkyl, C₃-C₆ cycloalkenyl, 3- to 8-membered heterocycloalkyl, 3- to 8-membered heterocycloalkenyl, aryl, heteroaryl, —CN, —OR⁹, —OCH₂R⁹, —CO₂R⁹, —CH₂CO₂R⁹, —OC(═O)R⁹, —C(═O)N(R⁹)₂, —N(R⁹)₂, —NR⁹C(═O)R⁹, —NR⁹C(═O)OR¹⁰, —OC(═O)NR⁹, —NR⁹C(═O)N(R⁹)₂, —C(R⁹)═N—OR⁹, —SR⁹, —S(═O)R¹⁰, —S(═O)₂R¹⁰, —S(═O)₂N(R⁹)₂, —P(═O)(OR⁹)₂, —P(═O)(OR⁹)R¹⁰ or —P(═O)(R¹⁰)₂, wherein each alkyl, aryl, and heteroaryl is unsubstituted or substituted with 1, 2, or 3 substituents selected from halogen, —CN, —OH, —O—(C₁-C₆ alkyl), —CO₂—(C₁-C₆ alkyl), C₁-C₆ alkyl, C₁-C₆ fluoroalkyl, C₁-C₆ hydroxyalkyl, —O—(C₁-C₆ fluoroalkyl), C₃-C₆ cycloalkyl, and 3- to 6-membered heterocycloalkyl; and wherein each cycloalkyl, cycloalkenyl, heterocycloalkyl, and heterocycloalkenyl is unsubstituted or substituted with 1, 2, or 3 substituents selected from halogen, —CN, —OH, ═O, —O—(C₁-C₆ alkyl), C₁-C₆ alkyl, C₁-C₆ fluoroalkyl, C₁-C₆ hydroxyalkyl, —O—(C₁-C₆ fluoroalkyl), C₃-C₆ cycloalkyl, and 3- to 6-membered heterocycloalkyl;
each R⁹ is independently selected from hydrogen, C₁-C₆ alkyl, C₁-C₆ fluoroalkyl, C₃-C₆ cycloalkyl, 3- to 8-membered heterocycloalkyl, phenyl, benzyl, and monocyclic heteroaryl, wherein each alkyl, fluoroalkyl, cycloalkyl, heterocycloalkyl, phenyl, benzyl, and heteroaryl is unsubstituted or substituted with 1, 2, or 3 substituents selected from halogen, —CN, —OH, —O—(C₁-C₆ alkyl)₂, —NH₂, —NH(C₁-C₆ alkyl), —N(C₁-C₆ alkyl)₂, C₁-C₆ alkyl, C₁-C₆ fluoroalkyl, C₁-C₆ hydroxyalkyl, —O—(C₁-C₆ fluoroalkyl), C₃-C₆ cycloalkyl, 3- to 6-membered heterocycloalkyl, and

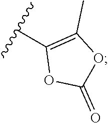 [SMILES: *c1oc(=O)oc1C]

or two R⁹ on the same N atom are taken together with the N atom to which they are attached to form a N-containing heterocycle, which is unsubstituted or substituted with 1, 2, or 3 substituents selected from halogen, —CN, —OH, —O—(C₁-C₆ alkyl), —NH₂, —NH(C₁-C₆ alkyl), —N(C₁-C₆ alkyl)₂, C₁-C₆ alkyl, C₁-C₆ fluoroalkyl, C₁-C₆ hydroxyalkyl, —O—(C₁-C₆ fluoroalkyl), C₃-C₆ cycloalkyl, and 3- to 6-membered heterocycloalkyl;
each R¹⁰ is independently selected from C₁-C₆ alkyl, C₁-C₆ fluoroalkyl, C₃-C₆ cycloalkyl, 3- to 8-membered heterocycloalkyl, phenyl, benzyl, and monocyclic heteroaryl, wherein each alkyl, fluoroalkyl, cycloalkyl, heterocycloalkyl, phenyl, benzyl, and heteroaryl is unsubstituted or substituted with 1, 2, or 3 substituents selected from halogen, —CN, —OH, —O—(C₁-C₆ alkyl), —NH₂, —NH(C₁-C₆ alkyl), —N(C₁-C₆ alkyl)₂, C₁-C₆ alkyl, C₁-C₆ fluoroalkyl, C₁-C₆ hydroxyalkyl, —O—(C₁-C₆ fluoroalkyl), C₃-C₆ cycloalkyl, 3- to 6-membered heterocycloalkyl, and

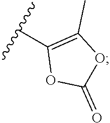 [SMILES: *c1oc(=O)oc1C]

m is 1 or 2;
n is 1 or 2;
p is 1-4; and
q is 0-4.

Disclosed herein, in certain embodiments, are pharmaceutical compositions comprising a compound disclosed herein, or a pharmaceutically acceptable salt, solvate, stereoisomer, or prodrug thereof, and at least one pharmaceutically acceptable excipient.

Disclosed herein, in certain embodiments, are methods of treating a condition or disorder involving the gut-brain axis in a subject in need thereof, the method comprising administering to the subject a therapeutically effective amount of a compound disclosed herein, or a pharmaceutically acceptable salt, solvate, stereoisomer, or prodrug thereof. In some embodiments, the condition or disorder is associated with SSTR5 activity. In some embodiments, the condition or disorder is a metabolic disorder. In some embodiments, the condition or disorder is type 2 diabetes, hyperglycemia, metabolic syndrome, obesity, hypercholesterolemia, nonalcoholic steatohepatitis, or hypertension. In some embodiments, the condition or disorder is a nutritional disorder. In some embodiments, the condition or disorder is short bowel syndrome, intestinal failure, or intestinal insufficiency.

In some embodiments, the condition or disorder is gastrointestinal injury resulting from toxic insults such as radiation or chemotherapy.

In some embodiments, disclosed herein are methods of augmenting weight loss or preventing weight gain or weight regain, the method comprising administering to the subject a therapeutically effective amount of a compound disclosed herein, or a pharmaceutically acceptable salt, solvate, stereoisomer, or prodrug thereof. In some embodiments, the subject has had bariatric surgery.

In some embodiments, the compound disclosed herein is gut-restricted. In some embodiments, the compound disclosed herein has low systemic exposure.

In some embodiments, the methods disclosed herein further comprise administering one or more additional therapeutic agents to the subject. In some embodiments, the one or more additional therapeutic agents are selected from a TGR5 agonist, a GPR40 agonist, a GPR119 agonist, a CCK1 agonist, a PDE4 inhibitor, a DPP-4 inhibitor, a GLP-1 receptor agonist, metformin, or a combination thereof. In some embodiments, the TGR5 agonist, GPR40 agonist, GPR119 agonist, or CCK1 agonist is gut-restricted.

Also disclosed herein, in certain embodiments, is the use of a compound disclosed herein, or a pharmaceutically acceptable salt, solvate, stereoisomer, or prodrug thereof, for the preparation of a medicament for the treatment of a condition or disorder involving the gut-brain axis in a subject in need thereof.

Also disclosed herein, in certain embodiments, are methods of treating a condition or disorder involving the gut-brain axis in a subject in need thereof, the method comprising administering to the subject a therapeutically effective amount of a gut-restricted SSTR5 modulator.

Also disclosed herein, in certain embodiments, is the use of a gut-restricted SSTR5 modulator for the preparation of a medicament for the treatment of a condition or disorder involving the gut-brain axis in a subject in need thereof.

### DETAILED DESCRIPTION OF THE INVENTION

This disclosure is directed, at least in part, to SSTR5 antagonists useful for the treatment of conditions or disorders involving the gut-brain axis. In some embodiments, the SSTR5 antagonists are gut-restricted compounds.

### Definitions

As used herein and in the appended claims, the singular forms “a,” “an,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulas, all combinations and subcombinations of ranges and specific embodiments therein are intended to be included.

The term “about” when referring to a number or a numerical range means that the number or numerical range referred to is an approximation within experimental variability (or within statistical experimental error), and thus the number or numerical range, in some instances, will vary between 1% and 15% of the stated number or numerical range.

The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

As used in the specification and appended claims, unless specified to the contrary, the following terms have the meaning indicated below:

As used herein, C₁-Cₓ includes C₁-C₂, C₁-C₃ . . . C₁-Cₓ. By way of example only, a group designated as “C₁-C₄” indicates that there are one to four carbon atoms in the moiety, i.e., groups containing 1 carbon atom, 2 carbon atoms, 3 carbon atoms or 4 carbon atoms. Thus, by way of example only, “C₁-C₄ alkyl” indicates that there are one to four carbon atoms in the alkyl group, i.e., the alkyl group is selected from among methyl, ethyl, propyl, iso-propyl, n-butyl, isobutyl, sec-butyl, and t-butyl.

“Alkyl” refers to an optionally substituted straight-chain, or optionally substituted branched-chain saturated hydrocarbon monoradical having from one to about ten carbon atoms, or more preferably, from one to six carbon atoms, wherein an sp³-hybridized carbon of the alkyl residue is attached to the rest of the molecule by a single bond. Examples include, but are not limited to, methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, sec-butyl, t-butyl, n-pentyl, isopentyl, neopentyl, tert-amyl and hexyl, and longer alkyl groups, such as heptyl, octyl, and the like. Whenever it appears herein, a numerical range such as “C₁-C₆ alkyl” means that the alkyl group consists of 1 carbon atom, 2 carbon atoms, 3 carbon atoms, 4 carbon atoms, 5 carbon atoms or 6 carbon atoms, although the present definition also covers the occurrence of the term “alkyl” where no numerical range is designated. In some embodiments, the alkyl is a C₁-C₁₀ alkyl, a C₁-C₉ alkyl, a C₁-C₈ alkyl, a C₁-C₇ alkyl, a C₁-C₆ alkyl, a C₁-C₅ alkyl, a C₁-C₄ alkyl, a C₁-C₃ alkyl, a C₁-C₂ alkyl, or a C₁ alkyl. Unless stated otherwise specifically in the specification, an alkyl group is optionally substituted as described below by one or more of the following substituents: halo, cyano, nitro, oxo, thioxo, imino, oximo, trimethylsilanyl, —OR^{a}, —SR^{a}, —OC(O)R^{a}, —OC(O)—OR, —N(R^{a})₂, —N⁺(R^{a})₃, —C(O)R^{a}, —C(O)OR^{a}, —C(O)N(R^{a})₂, —N(R^{a})C(O)OR^{f}, —OC(O)—N(R^{a})₂, —N(R^{a})C(O)R^{a}, —N(R^{a})S(O)ₜR^{f} (where t is 1 or 2), —S(O)ₜOR^{a} (where t is 1 or 2), —S(O)ₜR (where t is 1 or 2) and —S(O)ₜN(R^{a})₂ (where t is 1 or 2) where each R^{a} is independently hydrogen, alkyl, haloalkyl, cycloalkyl, aryl, aralkyl, heterocycloalkyl, heteroaryl or heteroarylalkyl, and each R^{f} is independently alkyl, haloalkyl, cycloalkyl, aryl, aralkyl, heterocycloalkyl, heteroaryl or heteroarylalkyl.

“Alkenyl” refers to an optionally substituted straight-chain, or optionally substituted branched-chain hydrocarbon monoradical having one or more carbon-carbon double-bonds and having from two to about ten carbon atoms, more preferably two to about six carbon atoms, wherein an sp²-hybridized carbon or an sp³-hybridized carbon of the alkenyl residue is attached to the rest of the molecule by a single bond. The group may be in either the cis or trans conformation about the double bond(s), and should be understood to include both isomers. Examples include, but are not limited to ethenyl (—CH═CH₂), 1-propenyl (—CH₂CH═CH₂), isopropenyl (—C(CH₃)═CH₂), butenyl, 1,3-butadienyl and the like. Whenever it appears herein, a numerical range such as “C₂-C₆ alkenyl” means that the alkenyl group may consist of 2 carbon atoms, 3 carbon atoms, 4 carbon atoms, 5 carbon atoms or 6 carbon atoms, although the present definition also covers the occurrence of the term “alkenyl” where no numerical range is designated. In some embodiments, the alkenyl is a C₂-C₁₀ alkenyl, a C₂-C₉ alkenyl, a C₂-C₈ alkenyl, a C₂-C₇ alkenyl, a C₂-C₆ alkenyl, a C₂-C₅ alkenyl, a C₂-C₄ alkenyl, a C₂-C₃ alkenyl, or a C₂ alkenyl. Unless stated otherwise specifically in the specification, an alkenyl group is optionally substituted as described below, for example, with oxo, halogen, amino, nitrile, nitro, hydroxyl, haloalkyl, alkoxy, aryl, cycloalkyl, heterocycloalkyl, heteroaryl, and the like. Unless stated otherwise specifically in the specification, an alkenyl group is optionally substituted as described below by one or more of the following substituents: halo, cyano, nitro, oxo, thioxo, imino, oximo, trimethylsilanyl, —OR^{a}, —SR^{a}, —OC(O)—R^{f}, —OC(O)—OR^{f}, —N(R^{a})₂, —N+(R^{a})₃, —C(O)R^{a}, —C(O)OR^{a}, —C(O)N(R^{a})₂, —N(R^{a})C(O)OR, —OC(O)—N(R^{a})₂, —N(R^{a})C(O)R^{f}, —N(R^{a})S(O)ₜR^{f} (where t is 1 or 2), —S(O)ₜOR^{a} (where t is 1 or 2), —S(O)ₜR (where t is 1 or 2) and —S(O)ₜN(R^{a})₂ (where t is 1 or 2) where each R^{a} is independently hydrogen, alkyl, haloalkyl, cycloalkyl, aryl, aralkyl, heterocycloalkyl, heteroaryl or heteroarylalkyl, and each R^{f} is independently alkyl, haloalkyl, cycloalkyl, aryl, aralkyl, heterocycloalkyl, heteroaryl or heteroarylalkyl.

“Alkynyl” refers to an optionally substituted straight-chain or optionally substituted branched-chain hydrocarbon monoradical having one or more carbon-carbon triple-bonds and having from two to about ten carbon atoms, more preferably from two to about six carbon atoms, wherein an sp-hybridized carbon or an sp³-hybridized carbon of the alkynyl residue is attached to the rest of the molecule by a single bond. Examples include, but are not limited to ethynyl, 2-propynyl, 2-butynyl, 1,3-butadiynyl and the like. Whenever it appears herein, a numerical range such as “C₂-C₆ alkynyl” means that the alkynyl group may consist of 2 carbon atoms, 3 carbon atoms, 4 carbon atoms, 5 carbon atoms or 6 carbon atoms, although the present definition also covers the occurrence of the term “alkynyl” where no numerical range is designated. In some embodiments, the alkynyl is a C₂-C₁₀ alkynyl, a C₂-C₉ alkynyl, a C₂-C₈ alkynyl, a C₂-C₇ alkynyl, a C₂-C₆ alkynyl, a C₂-C₅ alkynyl, a C₂-C₄ alkynyl, a C₂-C₃ alkynyl, or a C₂ alkynyl. Unless stated otherwise specifically in the specification, an alkynyl group is optionally substituted as described below by one or more of the following substituents: halo, cyano, nitro, oxo, thioxo, imino, oximo, trimethylsilanyl, —OR^{a}, —SR^{a}, —OC(O)R^{a}, —OC(O)—OR^{f}, —N(R^{a})₂, —N⁺(R^{a})₃, —C(O)R^{a}, —C(O)OR^{a}, —C(O)N(R^{a})₂, —N(R^{a})C(O)OR, —OC(O)—N(R^{a})₂, —N(R^{a})C(O)R^{f}, —N(R^{a})S(O)ₜR^{f} (where t is 1 or 2), —S(O)ₜOR^{a} (where t is 1 or 2), —S(O)ₜR (where t is 1 or 2) and —S(O)ₜN(R^{a})₂ (where t is 1 or 2) where each R^{a} is independently hydrogen, alkyl, haloalkyl, cycloalkyl, aryl, aralkyl, heterocycloalkyl, heteroaryl or heteroarylalkyl, and each R^{f} is independently alkyl, haloalkyl, cycloalkyl, aryl, aralkyl, heterocycloalkyl, heteroaryl or heteroarylalkyl.

“Alkylene” or “alkylene chain” refers to a straight or branched divalent hydrocarbon chain linking the rest of the molecule to a radical group, consisting solely of carbon and hydrogen, containing no unsaturation and having from one to twelve carbon atoms, for example, methylene, ethylene, propylene, n-butylene, and the like. The alkylene chain is attached to the rest of the molecule through a single bond and to the radical group through a single bond. The points of attachment of the alkylene chain to the rest of the molecule and to the radical group are through one carbon in the alkylene chain or through any two carbons within the chain. Unless stated otherwise specifically in the specification, an alkylene group is optionally substituted as described below by one or more of the following substituents: halo, cyano, nitro, oxo, thioxo, imino, oximo, trimethylsilanyl, —OR^{a}, —SR^{a}, —OC(O)R^{a}, —OC(O)—OR^{f}, —N(R^{a})₂, —N⁺(R^{a})₃, —C(O)R^{a}, —C(O)OR^{a}, —C(O)N(R^{a})₂, —N(R^{a})C(O)OR^{f}, —OC(O)—N(R^{a})₂, —N(R^{a})C(O)R^{f}, —N(R^{a})S(O)ₜR^{f} (where t is 1 or 2), —S(O)ₜOR^{a} (where t is 1 or 2), —S(O)ₜR (where t is 1 or 2) and —S(O)ₜN(R^{a})₂ (where t is 1 or 2) where each R^{a} is independently hydrogen, alkyl, haloalkyl, cycloalkyl, aryl, aralkyl, heterocycloalkyl, heteroaryl or heteroarylalkyl, and each R^{f} is independently alkyl, haloalkyl, cycloalkyl, aryl, aralkyl, heterocycloalkyl, heteroaryl or heteroarylalkyl.

“Alkenylene” or “alkenylene chain” refers to a straight or branched divalent hydrocarbon chain linking the rest of the molecule to a radical group, consisting solely of carbon and hydrogen, containing at least one carbon-carbon double bond, and having from two to twelve carbon atoms. The alkenylene chain is attached to the rest of the molecule through a single bond and to the radical group through a single bond. Unless stated otherwise specifically in the specification, an alkenylene group is optionally substituted as described below by one or more of the following substituents: halo, cyano, nitro, oxo, thioxo, imino, oximo, trimethylsilanyl, —OR^{a}, —SR^{a}, —OC(O)—R^{f}, —OC(O)—OR^{f}, —N(R^{a})₂, —N⁺(R^{a})₃, —C(O)R^{a}, —C(O)OR^{a}, —C(O)N(R^{a})₂, —N(R^{a})C(O)OR^{f}, —OC(O)—N(R^{a})₂, —N(R^{a})C(O)R^{f}, —N(R^{a})S(O)ₜR^{f} (where t is 1 or 2), —S(O)ₜOR^{a} (where t is 1 or 2), —S(O)ₜR^{f} (where t is 1 or 2) and —S(O)ₜN(R^{a})₂ (where t is 1 or 2) where each R^{a} is independently hydrogen, alkyl, haloalkyl, cycloalkyl, aryl, aralkyl, heterocycloalkyl, heteroaryl or heteroarylalkyl, and each R^{f} is independently alkyl, haloalkyl, cycloalkyl, aryl, aralkyl, heterocycloalkyl, heteroaryl or heteroarylalkyl.

“Alkynylene” or “alkynylene chain” refers to a straight or branched divalent hydrocarbon chain linking the rest of the molecule to a radical group, consisting solely of carbon and hydrogen, containing at least one carbon-carbon triple bond, and having from two to twelve carbon atoms. The alkynylene chain is attached to the rest of the molecule through a single bond and to the radical group through a single bond. Unless stated otherwise specifically in the specification, an alkynylene group is optionally substituted as described below by one or more of the following substituents: halo, cyano, nitro, oxo, thioxo, imino, oximo, trimethylsilanyl, —OR^{a}, —SR^{a}, —OC(O)R^{a}, —OC(O)—OR^{f}, —N(R^{a})₂, —N⁺(R^{a})₃, —C(O)R^{a}, —C(O)OR^{a}, —C(O)N(R^{a})₂, —N(R^{a})C(O)OR^{f}, —OC(O)—N(R^{a})₂, —N(R^{a})C(O)R^{f}, —N(R^{a})S(O)ₜR^{f} (where t is 1 or 2), —S(O)ₜOR^{a} (where t is 1 or 2), —S(O)ₜR (where t is 1 or 2) and —S(O)ₜN(R^{a})₂ (where t is 1 or 2) where each R^{a} is independently hydrogen, alkyl, haloalkyl, cycloalkyl, aryl, aralkyl, heterocycloalkyl, heteroaryl or heteroarylalkyl, and each R^{f} is independently alkyl, haloalkyl, cycloalkyl, aryl, aralkyl, heterocycloalkyl, heteroaryl or heteroarylalkyl.

“Alkoxy” or “alkoxyl” refers to a radical bonded through an oxygen atom of the formula —O-alkyl, where alkyl is an alkyl chain as defined above.

“Aryl” refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon from 6 to 18 carbon atoms, where at least one of the rings in the ring system is fully unsaturated, i.e., it contains a cyclic, delocalized (4n+2) π-electron system in accordance with the Hückel theory. The ring system from which aryl groups are derived include, but are not limited to, groups such as benzene, fluorene, indane, indene, tetralin and naphthalene. In some embodiments, the aryl is a C₆-C₁₀ aryl. In some embodiments, the aryl is a phenyl. Unless stated otherwise specifically in the specification, the term “aryl” or the prefix “ar-” (such as in “aralkyl”) is meant to include aryl radicals optionally substituted as described below by one or more substituents independently selected from alkyl, alkenyl, alkynyl, halo, haloalkyl, cyano, nitro, aryl, aralkyl, aralkenyl, aralkynyl, cycloalkyl, heterocycloalkyl, heteroaryl, heteroarylalkyl, —R^{b}—OR^{a}, —R^{b}—SR^{a}, —R^{b}—OC(O)—R^{a}, —R^{b}—OC(O)—OR^{f}, —R^{b}—OC(O)—N(R^{a})₂, —R^{b}—N(R^{a})₂, —R^{b}—N⁺(R^{a})₃, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR, —R^{b}—N(R^{a})C(O)R^{a}, —R^{b}—N(R^{a})S(O)ₜR^{f} (where t is 1 or 2), —R^{b}—S(O)ₜOR^{a} (where t is 1 or 2), —R^{b}—S(O)ₜR^{f} (where t is 1 or 2) and —R^{b}—S(O)ₜN(R^{a})₂ (where t is 1 or 2), where each R^{a} is independently hydrogen, alkyl, haloalkyl, cycloalkyl, cycloalkylalkyl, aryl (optionally substituted with one or more halo groups), aralkyl, heterocycloalkyl, heteroaryl or heteroarylalkyl, R^{f} is independently alkyl, haloalkyl, cycloalkyl, cycloalkylalkyl, aryl (optionally substituted with one or more halo groups), aralkyl, heterocycloalkyl, heteroaryl or heteroarylalkyl, each R^{b} is independently a direct bond or a straight or branched alkylene or alkenylene chain, and R^{c} is a straight or branched alkylene or alkenylene chain.

An “arylene” refers to a divalent radical derived from an “aryl” group as described above linking the rest of the molecule to a radical group. The arylene is attached to the rest of the molecule through a single bond and to the radical group through a single bond. In some embodiments, the arylene is a phenylene. Unless stated otherwise specifically in the specification, an arylene group is optionally substituted as described above for an aryl group.

“Cycloalkyl” refers to a stable, partially or fully saturated, monocyclic or polycyclic carbocyclic ring, which may include fused (when fused with an aryl or a heteroaryl ring, the cycloalkyl is bonded through a non-aromatic ring atom) or bridged ring systems. Representative cycloalkyls include, but are not limited to, cycloalkyls having from three to fifteen carbon atoms (C₃-C₁₅ cycloalkyl), from three to ten carbon atoms (C₃-C₁₀ cycloalkyl), from three to eight carbon atoms (C₃-C₅ cycloalkyl), from three to six carbon atoms (C₃-C₆ cycloalkyl), from three to five carbon atoms (C₃-C₅ cycloalkyl), or three to four carbon atoms (C₃-C₄ cycloalkyl). In some embodiments, the cycloalkyl is a 3- to 6-membered cycloalkyl. In some embodiments, the cycloalkyl is a 5- to 6-membered cycloalkyl. Monocyclic cycloalkyls include, for example, cyclopropyl, cyclobutyl, cyclopentyl, cyclohexyl, cycloheptyl, and cyclooctyl. Polycyclic cycloalkyls or carbocycles include, for example, adamantyl, norbornyl, decalinyl, bicyclo[1.1.1]pentyl, bicyclo[3.3.0]octane, bicyclo[4.3.0]nonane, cis-decalin, trans-decalin, bicyclo[2.1.1]hexane, bicyclo[2.2.1]heptane, bicyclo[2.2.2]octane, bicyclo[3.2.2]nonane, and bicyclo[3.3.2]decane, 7,7-dimethyl-bicyclo[2.2.1]heptanyl, and the like. Unless otherwise stated specifically in the specification, the term “cycloalkyl” is meant to include cycloalkyl radicals optionally substituted as described below by one or more substituents independently selected from alkyl, alkenyl, alkynyl, halo, haloalkyl, cyano, nitro, aryl, aralkyl, aralkenyl, aralkynyl, cycloalkyl, heterocycloalkyl, heteroaryl, heteroarylalkyl, —R^{b}—OR^{a}, —R^{b}—SR^{a}, —R^{b}—OC(O)—R^{a}, —R^{b}—OC(O)—OR^{f}, —R^{b}—OC(O)—N(R^{a})₂, —R^{b}—N(R^{a})₂, —R^{b}—N⁺(R^{a})₃, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{f}, —R^{b}—N(R^{a})C(O)R^{a}, —R^{b}—N(R^{a})S(O)ₜR (where t is 1 or 2), —R^{b}—S(O)ₜOR^{a} (where t is 1 or 2), —R^{b}—S(O)ₜR^{f} (where t is 1 or 2) and —R^{b}—S(O)ₜN(R^{a})₂ (where t is 1 or 2), where each R^{a} is independently hydrogen, alkyl, haloalkyl, cycloalkyl, cycloalkylalkyl, aryl (optionally substituted with one or more halo groups), aralkyl, heterocycloalkyl, heteroaryl or heteroarylalkyl, R is independently alkyl, haloalkyl, cycloalkyl, cycloalkylalkyl, aryl (optionally substituted with one or more halo groups), aralkyl, heterocycloalkyl, heteroaryl or heteroarylalkyl, each R^{b} is independently a direct bond or a straight or branched alkylene or alkenylene chain, and R^{c} is a straight or branched alkylene or alkenylene chain.

A “cycloalkylene” refers to a divalent radical derived from a “cycloalkyl” group as described above linking the rest of the molecule to a radical group. The cycloalkylene is attached to the rest of the molecule through a single bond and to the radical group through a single bond. Unless stated otherwise specifically in the specification, a cycloalkylene group is optionally substituted as described above for a cycloalkyl group.

“Halo” or “halogen” refers to bromo, chloro, fluoro or iodo. In some embodiments, halogen is fluoro or chloro. In some embodiments, halogen is fluoro.

“Haloalkyl” refers to an alkyl radical, as defined above, that is substituted by one or more hydroxy radicals, e.g., trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like.

“Fluoroalkyl” refers to an alkyl radical, as defined above, that is substituted by one or more fluoro radicals, as defined above, for example, trifluoromethyl, difluoromethyl, fluoromethyl, 2,2,2-trifluoroethyl, 1-fluoromethyl-2-fluoroethyl, and the like.

“Haloalkoxy” or “haloalkoxyl” refers to an alkoxyl radical, as defined above, that is substituted by one or more halo radicals, as defined above.

“Fluoroalkoxy” or “fluoroalkoxyl” refers to an alkoxy radical, as defined above, that is substituted by one or more fluoro radicals, as defined above, for example, trifluoromethoxy, difluoromethoxy, fluoromethoxy, and the like.

“Hydroxyalkyl” refers to an alkyl radical, as defined above, that is substituted by one or more halo radicals, as defined above, e.g., hydroxymethyl, 1-hydroxyethyl, 2-hydroxyethyl, 2-hydroxypropyl, 3-hydroxypropyl, 1,2-dihydroxyethyl, 2,3-dihydroxypropyl, 2,3,4,5,6-pentahydroxyhexyl, and the like.

“Heterocycloalkyl” refers to a stable 3- to 24-membered partially or fully saturated ring radical comprising 2 to 23 carbon atoms and from one to 8 heteroatoms selected from the group consisting of nitrogen, oxygen, and sulfur. Unless stated otherwise specifically in the specification, the heterocycloalkyl radical may be a monocyclic, bicyclic, tricyclic or tetracyclic ring system, which may include fused (when fused with an aryl or a heteroaryl ring, the heterocycloalkyl is bonded through a non-aromatic ring atom) or bridged ring systems; and the nitrogen, carbon or sulfur atoms in the heterocycloalkyl radical may be optionally oxidized; the nitrogen atom may be optionally quaternized. In some embodiments, the heterocycloalkyl is a 3- to 8-membered heterocycloalkyl. In some embodiments, the heterocycloalkyl is a 3- to 6-membered heterocycloalkyl. In some embodiments, the heterocycloalkyl is a 5- to 6-membered heterocycloalkyl. Examples of such heterocycloalkyl radicals include, but are not limited to, aziridinyl, azetidinyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, 1,3-dihydroisobenzofuran-1-yl, 3-oxo-1,3-dihydroisobenzofuran-1-yl, methyl-2-oxo-1,3-dioxol-4-yl, and 2-oxo-1,3-dioxol-4-yl. The term heterocycloalkyl also includes all ring forms of the carbohydrates, including but not limited to the monosaccharides, the disaccharides and the oligosaccharides. More preferably, heterocycloalkyls have from 2 to 10 carbons in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e., skeletal atoms of the heterocycloalkyl ring). Unless stated otherwise specifically in the specification, the term “heterocycloalkyl” is meant to include heterocycloalkyl radicals as defined above that are optionally substituted by one or more substituents selected from alkyl, alkenyl, alkynyl, halo, fluoroalkyl, oxo, thioxo, cyano, nitro, aryl, aralkyl, aralkenyl, aralkynyl, cycloalkyl, heterocycloalkyl, heteroaryl, heteroarylalkyl, —R^{b}—OR^{a}, —R^{b}—SR^{a}, —R^{b}—OC(O)—R^{a}, —R^{b}—OC(O)—OR, —R^{b}—OC(O)—N(R^{a})₂, —R^{b}—N(R^{a})₂, —R^{b}—N⁺(R^{a})₃, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR, —R^{b}—N(R^{a})C(O)R^{a}, —R^{b}—N(R^{a})S(O)ₜR^{f} (where t is 1 or 2), —R^{b}—S(O)ₜOR^{a} (where t is 1 or 2), —R^{b}—S(O)ₜR (where t is 1 or 2) and —R^{b}—S(O)ₜN(R^{a})₂ (where t is 1 or 2), where each R^{a} is independently hydrogen, alkyl, haloalkyl, cycloalkyl, cycloalkylalkyl, aryl (optionally substituted with one or more halo groups), aralkyl, heterocycloalkyl, heteroaryl or heteroarylalkyl, R is independently alkyl, haloalkyl, cycloalkyl, cycloalkylalkyl, aryl (optionally substituted with one or more halo groups), aralkyl, heterocycloalkyl, heteroaryl or heteroarylalkyl, each R^{b} is independently a direct bond or a straight or branched alkylene or alkenylene chain, and R^{c} is a straight or branched alkylene or alkenylene chain.

“N-heterocycloalkyl” refers to a heterocycloalkyl radical as defined above containing at least one nitrogen and where the point of attachment of the heterocycloalkyl radical to the rest of the molecule is through a nitrogen atom in the heterocycloalkyl radical. An N-heterocycloalkyl radical is optionally substituted as described above for heterocycloalkyl radicals.

“C-heterocycloalkyl” refers to a heterocycloalkyl radical as defined above and where the point of attachment of the heterocycloalkyl radical to the rest of the molecule is through a carbon atom in the heterocycloalkyl radical. A C-heterocycloalkyl radical is optionally substituted as described above for heterocycloalkyl radicals.

A “heterocycloalkylene” refers to a divalent radical derived from a “heterocycloalkyl” group as described above linking the rest of the molecule to a radical group. The heterocycloalkylene is attached to the rest of the molecule through a single bond and to the radical group through a single bond. Unless stated otherwise specifically in the specification, a heterocycloalkylene group is optionally substituted as described above for a heterocycloalkyl group.

“Heteroaryl” refers to a radical derived from a 5- to 18-membered aromatic ring radical that comprises one to seventeen carbon atoms and from one to six heteroatoms selected from nitrogen, oxygen and sulfur. As used herein, the heteroaryl radical is a monocyclic, bicyclic, tricyclic or tetracyclic ring system, wherein at least one of the rings in the ring system is fully unsaturated, i.e., it contains a cyclic, delocalized (4n+2) π-electron system in accordance with the Hückel theory. In some embodiments, the heteroaryl is a 5- to 10-membered heteroaryl. In some embodiments, the heteroaryl is a monocyclic heteroaryl, or a monocyclic 5- or 6-membered heteroaryl. In some embodiments, the heteroaryl is a 6,5-fused bicyclic heteroaryl. The heteroatom(s) in the heteroaryl radical is optionally oxidized. One or more nitrogen atoms, if present, are optionally quaternized. The heteroaryl is attached to the rest of the molecule through any atom of the ring(s). Unless stated otherwise specifically in the specification, the term “heteroaryl” is meant to include heteroaryl radicals as defined above that are optionally substituted by one or more substituents selected from alkyl, alkenyl, alkynyl, halo, haloalkyl, oxo, thioxo, cyano, nitro, aryl, aralkyl, aralkenyl, aralkynyl, cycloalkyl, heterocycloalkyl, heteroaryl, heteroarylalkyl, —R^{b}—OR^{a}, —R^{b}—SR^{a}, —R^{b}—OC(O)—R^{a}, —R^{b}—OC(O)—OR^{f}, —R^{b}—OC(O)—N(R^{a})₂, —R^{b}—N(R^{a})₂, —R^{b}—N⁺(R^{a})₃, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{f}, —R^{b}—N(R^{a})C(O)R^{a}, —R^{b}—N(R^{a})S(O)ₜR^{f} (where t is 1 or 2), —R^{b}—S(O)ₜOR^{a} (where t is 1 or 2), —R^{b}—S(O)ₜR^{f} (where t is 1 or 2) and —R^{b}—S(O)ₜN(R^{a})₂ (where t is 1 or 2), where each R^{a} is independently hydrogen, alkyl, haloalkyl, cycloalkyl, cycloalkylalkyl, aryl (optionally substituted with one or more halo groups), aralkyl, heterocycloalkyl, heteroaryl or heteroarylalkyl, R is independently alkyl, haloalkyl, cycloalkyl, cycloalkylalkyl, aryl (optionally substituted with one or more halo groups), aralkyl, heterocycloalkyl, heteroaryl or heteroarylalkyl, each R^{b} is independently a direct bond or a straight or branched alkylene or alkenylene chain, and R^{c} is a straight or branched alkylene or alkenylene chain.

A “heteroarylene” refers to a divalent radical derived from a “heteroaryl” group as described above linking the rest of the molecule to a radical group. The heteroarylene is attached to the rest of the molecule through a single bond and to the radical group through a single bond. Unless stated otherwise specifically in the specification, a heteroarylene group is optionally substituted as described above for a heteroaryl group.

The term “optional” or “optionally” means that the subsequently described event or circumstance may or may not occur, and that the description includes instances where said event or circumstance occurs and instances in which it does not. For example, “optionally substituted alkyl” means either “alkyl” or “substituted alkyl” as defined above. Further, an optionally substituted group may be unsubstituted (e.g., —CH₂CH₃), fully substituted (e.g., —CF₂CF₃), mono-substituted (e.g., —CH₂CH₂F) or substituted at a level anywhere in-between fully substituted and mono-substituted (e.g., —CH₂CHF₂, —CH₂CF₃, —CF₂CH₃, —CFHCHF₂, etc.). It will be understood by those skilled in the art with respect to any group containing one or more substituents that such groups are not intended to introduce any substitution or substitution patterns (e.g., substituted alkyl includes optionally substituted cycloalkyl groups, which in turn are defined as including optionally substituted alkyl groups, potentially ad infinitum) that are sterically impractical and/or synthetically non-feasible.

The term “modulate” or “modulating” or “modulation” refers to an increase or decrease in the amount, quality, or effect of a particular activity, function or molecule. By way of illustration and not limitation, agonists, partial agonists, inverse agonists, antagonists, and allosteric modulators of a G protein-coupled receptor are modulators of the receptor.

The term “agonism” as used herein refers to the activation of a receptor or enzyme by a modulator, or agonist, to produce a biological response.

The term “agonist” as used herein refers to a modulator that binds to a receptor or target enzyme and activates the receptor or enzyme to produce a biological response. By way of example, “GPR119 agonist” can be used to refer to a compound that exhibits an EC₅₀ with respect to GPR119 activity of no more than about 100 μM, as measured in the as measured in the inositol phosphate accumulation assay. In some embodiments, the term “agonist” includes full agonists or partial agonists.

The term “full agonist” refers to a modulator that binds to and activates a receptor or target enzyme with the maximum response that an agonist can elicit at the receptor or enzyme.

The term “partial agonist” refers to a modulator that binds to and activates a receptor or target enzyme, but has partial efficacy, that is, less than the maximal response, at the receptor or enzyme relative to a full agonist.

The term “positive allosteric modulator” refers to a modulator that binds to a site distinct from the orthosteric binding site and enhances or amplifies the effect of an agonist.

The term “antagonism” as used herein refers to the inactivation of a receptor or target enzyme by a modulator, or antagonist. Antagonism of a receptor, for example, is when a molecule binds to the receptor or target enzyme and does not allow activity to occur.

The term “antagonist” or “neutral antagonist” as used herein refers to a modulator that binds to a receptor or target enzyme and blocks a biological response. By way of example, “SSTR5 antagonist” can be used to refer to a compound that exhibits an IC₅₀ with respect to SSTR5 activity of no more than about 100 μM, as measured in the as measured in the inositol phosphate accumulation assay. An antagonist has no activity in the absence of an agonist or inverse agonist but can block the activity of either, causing no change in the biological response.

The term “inverse agonist” refers to a modulator that binds to the same receptor or target enzyme as an agonist but induces a pharmacological response opposite to that agonist, i.e., a decrease in biological response.

The term “negative allosteric modulator” refers to a modulator that binds to a site distinct from the orthosteric binding site and reduces or dampens the effect of an agonist.

As used herein, “EC₅₀” is intended to refer to the concentration of a substance (e.g., a compound or a drug) that is required for 50% activation or enhancement of a biological process. In some instances, EC₅₀ refers to the concentration of agonist that provokes a response halfway between the baseline and maximum response in an in vitro assay. In some embodiments as used herein, EC₅₀ refers to the concentration of an agonist (e.g., a GPR119 agonist) that is required for 50% activation of a receptor or target enzyme (e.g., GPR119).

As used herein, “IC₅₀” is intended to refer to the concentration of a substance (e.g., a compound or a drug) that is required for 50% inhibition of a biological process. For example, IC₅₀ refers to the half maximal (50%) inhibitory concentration (IC) of a substance as determined in a suitable assay. In some instances, an IC₅₀ is determined in an in vitro assay system. In some embodiments as used herein, IC₅₀ refers to the concentration of a modulator (e.g., an SSTR5 antagonist) that is required for 50% inhibition of a receptor or a target enzyme (e.g., SSTR5).

The terms “subject,” “individual,” and “patient” are used interchangeably. These terms encompass mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like.

The term “gut-restricted” as used herein refers to a compound, e.g., an SSTR5 antagonist, that is predominantly active in the gastrointestinal system. In some embodiments, the biological activity of the gut-restricted compound, e.g., a gut-restricted SSTR5 antagonist, is restricted to the gastrointestinal system. In some embodiments, gastrointestinal concentration of a gut-restricted modulator, e.g., a gut-restricted SSTR5 antagonist, is higher than the IC₅₀ value or the EC₅₀ value of the gut-restricted modulator against its receptor or target enzyme, e.g., SSTR5, while the plasma levels of said gut-restricted modulator, e.g., gut-restricted SSTR5 antagonist, are lower than the IC₅₀ value or the EC₅₀ value of the gut-restricted modulator against its receptor or target enzyme, e.g., SSTR5. In some embodiments, the gut-restricted compound, e.g., a gut-restricted SSTR5 antagonist, is non-systemic. In some embodiments, the gut-restricted compound, e.g., a gut-restricted SSTR5 antagonist, is a non-absorbed compound. In other embodiments, the gut-restricted compound, e.g., a gut-restricted SSTR5 antagonist, is absorbed, but is rapidly metabolized to metabolites that are significantly less active than the modulator itself toward the target receptor or enzyme, i.e., a “soft drug.” In other embodiments, the gut-restricted compound, e.g., a gut-restricted SSTR5 antagonist, is minimally absorbed and rapidly metabolized to metabolites that are significantly less active than the modulator itself toward the target receptor or enzyme.

In some embodiments, the gut-restricted modulator, e.g., a gut-restricted SSTR5 antagonist, is non-systemic but is instead localized to the gastrointestinal system. For example, the modulator, e.g., a gut-restricted SSTR5 antagonist, may be present in high levels in the gut, but low levels in serum. In some embodiments, the systemic exposure of a gut-restricted modulator, e.g., a gut-restricted SSTR5 antagonist, is, for example, less than 100, less than 50, less than 20, less than 10, or less than 5 nM, bound or unbound, in blood serum. In some embodiments, the intestinal exposure of a gut-restricted modulator, e.g., a gut-restricted SSTR5 antagonist, is, for example, greater than 1000, 5000, 10000, 50000, 100000, or 500000 nM. In some embodiments, a modulator, e.g., a SSTR5 antagonist, is gut-restricted due to poor absorption of the modulator itself, or because of absorption of the modulator which is rapidly metabolized in serum resulting in low systemic circulation, or due to both poor absorption and rapid metabolism in the serum. In some embodiments, a modulator, e.g., a SSTR5 antagonist, is covalently bonded to a kinetophore, optionally through a linker, which changes the pharmacokinetic profile of the modulator.

In particular embodiments, the gut-restricted SSTR5 antagonist is a soft drug. The term “soft drug” as used herein refers to a compound that is biologically active but is rapidly metabolized to metabolites that are significantly less active than the compound itself toward the target receptor. In some embodiments, the gut-restricted SSTR5 antagonist is a soft drug that is rapidly metabolized in the blood to significantly less active metabolites. In some embodiments, the gut-restricted SSTR5 antagonist is a soft drug that is rapidly metabolized in the liver to significantly less active metabolites. In some embodiments, the gut-restricted SSTR5 antagonist is a soft drug that is rapidly metabolized in the blood and the liver to significantly less active metabolites. In some embodiments, the gut-restricted SSTR5 antagonist is a soft drug that has low systemic exposure. In some embodiments, the biological activity of the metabolite(s) is/are 10-fold, 20-fold, 50-fold, 100-fold, 500-fold, or 1000-fold lower than the biological activity of the soft drug gut-restricted SSTR5 antagonist.

The term “kinetophore” as used herein refers to a structural unit tethered to a small molecule modulator, e.g., an SSTR5 antagonist, optionally through a linker, which makes the whole molecule larger and increases the polar surface area while maintaining biological activity of the small molecule modulator. The kinetophore influences the pharmacokinetic properties, for example solubility, absorption, distribution, rate of elimination, and the like, of the small molecule modulator, e.g., an SSTR5 antagonist, and has minimal changes to the binding to or association with a receptor or target enzyme. The defining feature of a kinetophore is not its interaction with the target, for example a receptor, but rather its effect on specific physiochemical characteristics of the modulator to which it is attached, e.g., an SSTR5 antagonist. In some instances, kinetophores are used to restrict a modulator, e.g., an SSTR5 antagonist, to the gut.

The term “linked” as used herein refers to a covalent linkage between a modulator, e.g., an SSTR5 antagonist, and a kinetophore. The linkage can be through a covalent bond, or through a “linker.” As used herein, “linker” refers to one or more bifunctional molecules which can be used to covalently bond to the modulator, e.g., an SSTR5 antagonist, and kinetophore. In some embodiments, the linker is attached to any part of the modulator, e.g., an SSTR5 antagonist, so long as the point of attachment does not interfere with the binding of the modulator to its receptor or target enzyme. In some embodiments, the linker is non-cleavable. In some embodiments, the linker is cleavable. In some embodiments, the linker is cleavable in the gut. In some embodiments, cleaving the linker releases the biologically active modulator, e.g., an SSTR5 antagonist, in the gut.

The term “gastrointestinal system” (GI system) or “gastrointestinal tract” (GI tract) as used herein, refers to the organs and systems involved in the process of digestion. The gastrointestinal tract includes the esophagus, stomach, small intestine, which includes the duodenum, jejunum, and ileum, and large intestine, which includes the cecum, colon, and rectum. In some embodiments herein, the GI system refers to the “gut,” meaning the stomach, small intestines, and large intestines or to the small and large intestines, including, for example, the duodenum, jejunum, and/or colon.

Gut-Brain Axis

The gut-brain axis refers to the bidirectional biochemical signaling that connects the gastrointestinal tract (GI tract) with the central nervous system (CNS) through the peripheral nervous system (PNS) and endocrine, immune, and metabolic pathways.

In some instances, the gut-brain axis comprises the GI tract; the PNS including the dorsal root ganglia (DRG) and the sympathetic and parasympathetic arms of the autonomic nervous system including the enteric nervous system and the vagus nerve; the CNS; and the neuroendocrine and neuroimmune systems including the hypothalamic-pituitary-adrenal axis (HPA axis). The gut-brain axis is important for maintaining homeostasis of the body and is regulated and modulates physiology through the central and peripheral nervous systems and endocrine, immune, and metabolic pathways.

The gut-brain axis modulates several important aspects of physiology and behavior. Modulation by the gut-brain axis occurs via hormonal and neural circuits. Key components of these hormonal and neural circuits of the gut-brain axis include highly specialized, secretory intestinal cells that release hormones (enteroendocrine cells or EECs), the autonomic nervous system (including the vagus nerve and enteric nervous system), and the central nervous system. These systems work together in a highly coordinated fashion to modulate physiology and behavior.

Defects in the gut-brain axis are linked to a number of diseases, including those of high unmet need. Diseases and conditions affected by the gut-brain axis, include central nervous system (CNS) disorders including mood disorders, anxiety, depression, affective disorders, schizophrenia, malaise, cognition disorders, addiction, autism, epilepsy, neurodegenerative disorders, Alzheimer's disease, and Parkinson's disease, Lewy Body dementia, episodic cluster headache, migraine, pain; metabolic conditions including diabetes and its complications such as chronic kidney disease/diabetic nephropathy, diabetic retinopathy, diabetic neuropathy, and cardiovascular disease, metabolic syndrome, obesity, dyslipidemia, and nonalcoholic steatohepatitis (NASH); eating and nutritional disorders including hyperphagia, cachexia, anorexia nervosa, short bowel syndrome, intestinal failure, intestinal insufficiency and other eating disorders; inflammatory disorders and autoimmune diseases such as inflammatory bowel disease, ulcerative colitis, Crohn's disease, psoriasis, and celiac disease; necrotizing enterocolitis; gastrointestinal injury resulting from toxic insults such as radiation or chemotherapy; necrotizing enterocolitis; diseases/disorders of gastrointestinal barrier dysfunction including environmental enteric dysfunction, spontaneous bacterial peritonitis; functional gastrointestinal disorders such as irritable bowel syndrome, functional dyspepsia, functional abdominal bloating/distension, functional diarrhea, functional constipation, and opioid-induced constipation; gastroparesis; nausea and vomiting; disorders related to microbiome dysbiosis, and other conditions involving the gut-brain axis.

SSTR5 in the Gut-Brain Axis

Somatostatin acts at many sites to inhibit the release of many hormones and other secretory proteins. Somatostatin is predominantly expressed in two forms, SST-14 in gastric and pancreatic delta cells and neurons and SST-28 in intestinal muscosal cells. In some instances, the biological effects of somatostatin are mediated by a family of G protein-coupled receptors that are expressed in a tissue-specific manner. SSTR5 is a member of the superfamily of receptors and is expressed on p cells of pancreatic islets, GI epithelium and enteroendocrine cells, and cardiac tissue. In some instances, somatostatin binding to SSTR5 inhibits the release of GLP-1, GLP-2, GIP, PYY, or other hormones in enteroendocrine cells. SSTR5 antagonists may be useful in the treatment of metabolic disorders such as diabetes and obesity, and other diseases involving the gut-brain axis.

In some instances, inhibiting SSTR5 activity results in an elevated level of GLP-1, GLP-2, GIP, PYY, and other hormones in enteroendocrine cells. In some instances, modulators of SSTR5, for example, SSTR5 antagonists, facilitate the release of GLP-1, GLP-2, GIP, PYY, and other hormones in enteroendocrine cells by blocking the activity of somatostatin. In some instances, modulators of SSTR5, for example, SSTR5 antagonists, lead to increased cAMP levels by blocking the activity of somatostatin. In some instances, SSTR5 activity, upon binding of somatostatin, inhibits intracellular cAMP production and GLP-1, GLP-2, GIP, PYY, and other hormone secretion. In some instances, inhibiting SSTR5 activity results in elevated intracellular cAMP levels and elevated GLP-1, GIP, PYY, or other hormone secretion. In some instances, inhibiting SSTR5 activity results in elevated intracellular cAMP levels and elevated GLP-1 secretion.

Described herein is a method of treating a condition or disorder involving the gut-brain axis in an individual in need thereof, the method comprising administering to the individual a SSTR5 receptor antagonist. In other embodiments, the method comprises administering to the individual a SSTR5 inverse agonist.

In some embodiments, the condition or disorder involving the gut-brain axis is selected from the group consisting of: central nervous system (CNS) disorders including mood disorders, anxiety, depression, affective disorders, schizophrenia, malaise, cognition disorders, addiction, autism, epilepsy, neurodegenerative disorders, Alzheimer's disease, and Parkinson's disease, Lewy Body dementia, episodic cluster headache, migraine, pain; metabolic conditions including diabetes and its complications such as chronic kidney disease/diabetic nephropathy, diabetic retinopathy, diabetic neuropathy, and cardiovascular disease, metabolic syndrome, obesity, dyslipidemia, and nonalcoholic steatohepatitis (NASH); eating and nutritional disorders including hyperphagia, cachexia, anorexia nervosa, short bowel syndrome, intestinal failure, intestinal insufficiency and other eating disorders; inflammatory disorders and autoimmune diseases such as inflammatory bowel disease, ulcerative colitis, Crohn's disease, psoriasis, and celiac disease; necrotizing enterocolitis; gastrointestinal injury resulting from toxic insults such as radiation or chemotherapy; necrotizing enterocolitis; diseases/disorders of gastrointestinal barrier dysfunction including environmental enteric dysfunction, spontaneous bacterial peritonitis; functional gastrointestinal disorders such as irritable bowel syndrome, functional dyspepsia, functional abdominal bloating/distension, functional diarrhea, functional constipation, and opioid-induced constipation; gastroparesis; nausea and vomiting; disorders related to microbiome dysbiosis, other conditions involving the gut-brain axis. In some embodiments, the condition is a metabolic disorder. In some embodiments, the metabolic disorder is type 2 diabetes, hyperglycemia, metabolic syndrome, obesity, hypercholesterolemia, nonalcoholic steatohepatitis, or hypertension. In some embodiments, the metabolic disorder is diabetes. In other embodiments, the metabolic disorder is obesity. In other embodiments, the metabolic disorder is nonalcoholic steatohepatitis. In some embodiments, the condition involving the gut-brain axis is a nutritional disorder. In some embodiments, the nutritional disorder is short bowel syndrome, intestinal failure, or intestinal insufficiency. In some embodiments, the nutritional disorder is short bowel syndrome. In some embodiments, the condition involving the gut-brain axis is gastrointestinal injury. In some embodiments, the condition involving the gut-brain axis is gastrointestinal injury resulting from toxic insults such as radiation or chemotherapy. In some embodiments, the condition involving the gut-brain axis is weight loss or preventing weight gain or weight regain. In some embodiments, the condition involving the gut-brain axis is weight loss or preventing weight gain or weight regain post-bariatric surgery. In some embodiments, the condition involving the gut-brain axis is weight loss or preventing weight gain or weight regain, wherein the subject has had bariatric surgery.

Gut-Restricted Antagonists

In some instances, differentiation of systemic effects of an SSTR5 antagonist from beneficial, gut-driven effects would be critical for the development of an SSTR5 antagonist for the treatment of disease.

In some embodiments, the SSTR5 antagonist is gut-restricted. In some embodiments, the SSTR5 antagonist is designed to be substantially non-permeable or substantially non-bioavailable in the blood stream. In some embodiments, the SSTR5 antagonist is designed to inhibit SSTR5 activity in the gut and is substantially non-systemic. In some embodiments, the SSTR5 antagonist has low systemic exposure.

In some embodiments, a gut-restricted SSTR5 antagonist has low oral bioavailability. In some embodiments, a gut-restricted SSTR5 antagonist has <10% oral bioavailability, <8% oral bioavailability, <5% oral bioavailability, <3% oral bioavailability, or <2% oral bioavailability.

In some embodiments, the unbound plasma levels of a gut-restricted SSTR5 antagonist are lower than the IC₅₀ value of the SSTR5 antagonist against SSTR5. In some embodiments, the unbound plasma levels of a gut-restricted SSTR5 antagonist are significantly lower than the IC₅₀ value of the gut-restricted SSTR5 antagonist against SSTR5. In some embodiments, the unbound plasma levels of the SSTR5 antagonist are 2-fold, 10-fold, 20-fold, 30-fold, 40-fold, 50-fold, or 100-fold lower than the IC₅₀ value of the gut-restricted SSTR5 antagonist against SSTR5.

In some embodiments, a gut-restricted SSTR5 antagonist has low systemic exposure. In some embodiments, the systemic exposure of a gut-restricted SSTR5 antagonist is, for example, less than 500, less than 200, less than 100, less than 50, less than 20, less than 10, or less than 5 nM, bound or unbound, in blood serum. In some embodiments, the systemic exposure of a gut-restricted SSTR5 antagonist is, for example, less than 500, less than 200, less than 100, less than 50, less than 20, less than 10, or less than 5 ng/mL, bound or unbound, in blood serum.

In some embodiments, a gut-restricted SSTR5 antagonist has low permeability. In some embodiments, a gut-restricted SSTR5 antagonist has low intestinal permeability. In some embodiments, the permeability of a gut-restricted SSTR5 antagonist is, for example, less than 5.0×10⁻⁶ cm/s, less than 2.0×10⁻⁶ cm/s, less than 1.5×10⁻⁶ cm/s, less than 1.0×10⁻⁶ cm/s, less than 0.75×10⁻⁶ cm/s, less than 0.50×10⁻⁶ cm/s, less than 0.25×10⁻⁶ cm/s, less than 0.10×10⁻⁶ cm/s, or less than 0.05×10⁻⁶ cm/s.

In some embodiments, a gut-restricted SSTR5 antagonist has low absorption. In some embodiments, the absorption of a gut-restricted SSTR5 antagonist is less than less than 20%, or less than 10%, less than 5%, or less than 1%.

In some embodiments, a gut-restricted SSTR5 antagonist has high plasma clearance. In some embodiments, a gut-restricted SSTR5 antagonist is undetectable in plasma in less than 8 hours, less than 6 hours, less than 4 hours, less than 3 hours, less than 120 min, less than 90 min, less than 60 min, less than 45 min, less than 30 min, or less than 15 min.

In some embodiments of the methods described herein, the SSTR5 antagonist is gut-restricted. In some embodiments, the SSTR5 antagonist is covalently bonded to a kinetophore. In some embodiments, the SSTR5 antagonist is covalently bonded to a kinetophore through a linker. In some embodiments, the SSTR5 antagonist is a soft drug.

In other embodiments, the methods described herein comprise administering an SSTR5 inverse agonist. In some embodiments, the SSTR5 inverse agonist is gut-restricted. In some embodiments, the SSTR5 inverse agonist is covalently bonded to a kinetophore. In some embodiments, the SSTR5 inverse agonist is covalently bonded to a kinetophore through a linker. In some embodiments, the SSTR5 inverse agonist is a soft drug.

Compounds

Disclosed herein, in certain embodiments, is a compound of Formula (I):

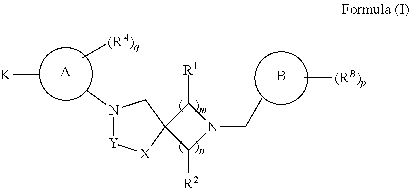

or a pharmaceutically acceptable salt, solvate, stereoisomer, or prodrug thereof, wherein:
X is —O—, —NR³—, or —C(R⁴)₂—;
Y is —C(═O)—, or —S(═O)₂—;
Ring A is aryl, heteroaryl, cycloalkyl, or heterocycloalkyl;
Ring B is aryl or heteroaryl;
K is —(CH₂)ⱼ-G;
   G is —P(═O)(OH)₂, —P(═O)(OH)(R^{D}), —P(═O)(OH)(H), —P(═O)(OH)(OR^{D}), —B(OH)₂, —B(OR^{D})(OH), —NHC(═O)H, —NHC(═O)(R^{D}), —NHS(═O)₂(R^{D}), —NHC(═O)NHS(═O)₂(R^{D}), —N(R^{D})C(═O)NHS(═O)₂(R^{D}), —C(═O)NHS(═O)₂(R^{D}), —S(═O)₂NHC(═O)(R^{D}), —NHC(═O)NH₂, —NHC(═O)NH(R^{D}), —NHC(═NH)NH₂, —NHC(═NH)NH(R^{D}), —NHC(═NH)N(R^{D})₂, —N(R^{D})C(═NH)NH₂, —N(R^{D})C(═NH)NH(R^{D}), —N(R^{D})C(═NH)N(R^{D})₂, —NHC(═N(R^{D}))NH₂, —NHC(═N(R^{D}))NH(R^{D}), —NHC(═N(R^{D}))N(R^{D})₂, N(R^{D})C(═N(R^{D}))NH₂, —N(R^{D})C(═N(R^{D}))NH(R^{D}), —N(R^{D})C(═N(R^{D}))N(R^{D})₂, —NHC(═NH)NHC(═NH)NH₂, —N(R^{D})C(═NH)NHC(═NH)NH²,

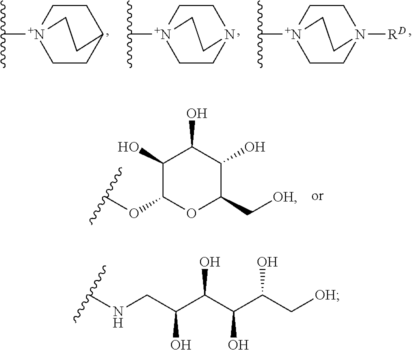

j is 0-4;
each R¹ and R² is independently hydrogen, C₁₋₆ alkyl, or C₁₋₆ fluoroalkyl;
or one R¹ and one R² are taken together to form a ring; R³ is hydrogen, C₁₋₆ alkyl, C₁₋₆ fluoroalkyl, or C₃₋₆ cycloalkyl;
each R⁴ is independently hydrogen, C₁₋₆ alkyl, C₁₋₆ fluoroalkyl, or C₃₋₆ cycloalkyl; each R^{D} is independently C₁₋₆ alkyl or C₃₋₆ cycloalkyl; wherein the alkyl and cycloalkyl are unsubstituted or substituted by 1-3 halogen or —OH groups; each R^{A} is independently halogen, —OH, —O—(C₁-C₆ alkyl), C₁-C₆ alkyl, C₃-C₆ cycloalkyl, 3- to 8-membered heterocycloalkyl, wherein each alkyl, cycloalkyl, and heterocycloalkyl is unsubstituted or substituted with 1, 2, or 3 substituents selected from halogen, —CN, —OH, —O—(C₁-C₆ alkyl), C₁-C₆ alkyl, C₁-C₆ fluoroalkyl, C₁-C₆ hydroxyalkyl, —O—(C₁-C₆ fluoroalkyl), C₃-C₆ cycloalkyl, and 3- to 6-membered heterocycloalkyl;
each R^{B} is independently halogen, C₁-C₆ alkyl, C₃-C₆ cycloalkyl, C₃-C₆ cycloalkenyl, 3- to 8-membered heterocycloalkyl, 3- to 8-membered heterocycloalkenyl, aryl, heteroaryl, —CN, —OR⁹, —OCH₂R⁹, —CO₂R⁹, —CH₂CO₂R⁹, —OC(═O)R⁹, —C(═O)N(R⁹)₂, —N(R⁹)₂, —NR⁹C(═O)R⁹, —NR⁹C(═O)OR¹⁰, —OC(═O)NR⁹, —NR⁹C(═O)N(R⁹)₂, —C(R⁹)═N—OR⁹, —SR⁹, —S(═O)R¹⁰, —S(═O)₂R¹⁰, —S(═O)₂N(R⁹)₂, —P(═O)(OR⁹)₂, —P(═O)(OR⁹)R¹⁰ or —P(═O)(R¹⁰)₂, wherein each alkyl, aryl, and heteroaryl is unsubstituted or substituted with 1, 2, or 3 substituents selected from halogen, —CN, —OH, —O—(C₁-C₆ alkyl), —CO₂—(C₁-C₆ alkyl), C₁-C₆ alkyl, C₁-C₆ fluoroalkyl, C₁-C₆ hydroxyalkyl, —O—(C₁-C₆ fluoroalkyl), C₃-C₆ cycloalkyl, and 3- to 6-membered heterocycloalkyl; and wherein each cycloalkyl, cycloalkenyl, heterocycloalkyl, and heterocycloalkenyl is unsubstituted or substituted with 1, 2, or 3 substituents selected from halogen, —CN, —OH, ═O, —O—(C₁-C₆ alkyl), C₁-C₆ alkyl, C₁-C₆ fluoroalkyl, C₁-C₆ hydroxyalkyl, —O—(C₁-C₆ fluoroalkyl), C₃-C₆ cycloalkyl, and 3- to 6-membered heterocycloalkyl;
each R⁹ is independently selected from hydrogen, C₁-C₆ alkyl, C₁-C₆ fluoroalkyl, C₃-C₆ cycloalkyl, 3- to 8-membered heterocycloalkyl, phenyl, and monocyclic heteroaryl, wherein each alkyl, fluoroalkyl, cycloalkyl, heterocycloalkyl, phenyl, and heteroaryl is unsubstituted or substituted with 1, 2, or 3 substituents selected from halogen, —CN, —OH, —O—(C₁-C₆ alkyl), —NH₂, —NH(C₁-C₆ alkyl), —N(C₁-C₆ alkyl)₂, C₁-C₆ alkyl, C₁-C₆ fluoroalkyl, C₁-C₆ hydroxyalkyl, —O—(C₁-C₆ fluoroalkyl), C₃-C₆ cycloalkyl, 3- to 6-membered heterocycloalkyl, and

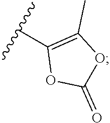 [SMILES: *c1oc(=O)oc1C]

or two R⁹ on the same N atom are taken together with the N atom to which they are attached to form a N-containing heterocycle, which is unsubstituted or substituted with 1, 2, or 3 substituents selected from halogen, —CN, —OH, —O—(C₁-C₆ alkyl), —NH₂, —NH(C₁-C₆ alkyl), —N(C₁-C₆ alkyl)₂, C₁-C₆ alkyl, C₁-C₆ fluoroalkyl, C₁-C₆ hydroxyalkyl, —O—(C₁-C₆ fluoroalkyl), C₃-C₆ cycloalkyl, and 3- to 6-membered heterocycloalkyl;
each R¹⁰ is independently selected from C₁-C₆ alkyl, C₁-C₆ fluoroalkyl, C₃-C₆ cycloalkyl, 3- to 8-membered heterocycloalkyl, phenyl, and monocyclic heteroaryl, wherein each alkyl, fluoroalkyl, cycloalkyl, heterocycloalkyl, phenyl, and heteroaryl is unsubstituted or substituted with 1, 2, or 3 substituents selected from halogen, —CN, —OH, —O—(C₁-C₆ alkyl), —NH₂, —NH(C₁-C₆ alkyl), —N(C₁-C₆ alkyl)₂, C₁-C₆ alkyl, C₁-C₆ fluoroalkyl, C₁-C₆ hydroxyalkyl, —O—(C₁-C₆ fluoroalkyl), C₃-C₆ cycloalkyl, 3- to 6-membered heterocycloalkyl, and

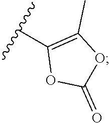 [SMILES: *c1oc(=O)oc1C]

m is 1 or 2;
n is 1 or 2;
p is 0-4; and
q is 0-4.

In some embodiments, G is —P(═O)(OH)₂, —P(═O)(OH)(R^{D}), —P(═O)(OH)(H), —P(═O)(OH)(OR^{D}), —B(OH)₂, —B(OR^{D})(OH), —NHC(═O)H, —NHC(═O)(R^{D}), —NHS(═O)₂(R^{D}), —NHC(═O)NHS(═O)₂(R^{D}), —N(R^{D})C(═O)NHS(═O)₂(R^{D}), —C(═O)NHS(═O)₂(R^{D}), —S(═O)₂NHC(═O)(R^{D}), —NHC(═O)NH₂, —NHC(═O)NH(R^{D}), —NHC(═NH)NH₂, —NHC(═NH)NH(R^{D}), —NHC(═NH)N(R^{D})₂, —N(R^{D})C(═NH)NH₂, —N(R^{D})C(═NH)NH(R^{D}), —N(R^{D})C(═NH)N(R^{D})₂, —NHC(═N(R^{D}))NH₂, —NHC(═N(R^{D}))NH(R^{D}), —NHC(═N(R^{D}))N(R^{D})₂, —N(R^{D})C(═N(R^{D}))NH₂, —N(R^{D})C(═N(R^{D}))NH(R^{D}), —N(R^{D})C(═N(R^{D}))N(R^{D})₂, —NHC(═NH)NHC(═NH)NH₂, —N(R^{D})C(═NH)NHC(═NH)NH₂,

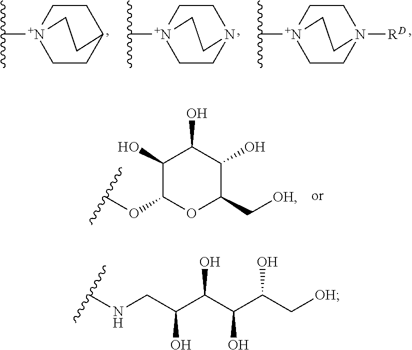

In some embodiments, each R¹ and R² is independently hydrogen, C₁₋₆ alkyl, or C₁₋₆ fluoroalkyl. In some embodiments, each R¹ and R² is independently hydrogen or C₁₋₆ alkyl. In some embodiments, each R¹ and R² is independently —H, —CH₃, —CH₂CH₃, —CH₂CH₂CH₃, —CH(CH₃)₂, —CH₂CH₂CH₂CH₃, —CH₂CH(CH₃)₂, —CH(CH₃)(CH₂CH₃), —C(CH₃)₃, —CH₂F, —CHF₂, —CF₃, —CH₂CH₂F, —CH₂CHF₂, or —CH₂CF₃. In some embodiments, each R¹ and R² is independently —H, —CH₃, —CH₂CH₃, or —CH₂CH₂CH₃. In some embodiments, each R¹ and R² is —H.

In some embodiments, one R¹ and one R² are taken together to form a ring. In some embodiments, one R¹ and one R² are taken together to form a 3- to 6-membered heterocycloalkyl ring.

In some embodiments, m is 1. In some embodiments, m is 2. In some embodiments, n is 1. In some embodiments, n is 2. In some embodiments, m is 1 and n is 1. In some embodiments, m is 1 and n is 2. In some embodiments, m is 2 and n is 1. In some embodiments, m is 2 and n is 2.

In some embodiments, Ring B is phenyl, naphthyl, monocyclic 6-membered heteroaryl, monocyclic 5-membered heteroaryl, or bicyclic heteroaryl.

In some embodiments, Ring B is phenyl or monocyclic heteroaryl. In some embodiments, Ring B is phenyl, monocyclic 6-membered heteroaryl, or monocyclic 5-membered heteroaryl. In some embodiments, Ring B is phenyl, pyridinyl, pyrimidinyl, pyrazinyl, pyridazinyl, triazinyl, furanyl, thienyl, pyrrolyl, oxazolyl, thiazolyl, imidazolyl, pyrazolyl, triazolyl, tetrazolyl, isoxazolyl, isothiazolyl, oxadiazolyl, or thiadiazolyl.

In some embodiments, Ring B is phenyl or 6-membered heteroaryl. In some embodiments, Ring B is phenyl, pyridinyl, pyrimidinyl, pyrazinyl, or pyridazinyl.

In some embodiments, Ring B is phenyl, or pyridinyl.

In some embodiments, Ring B is

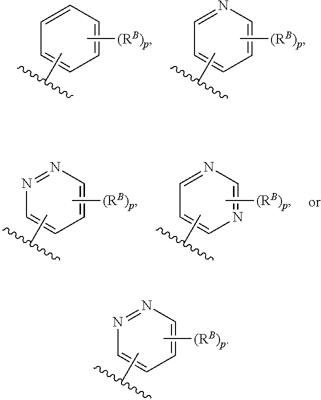

In some embodiments, Ring B is

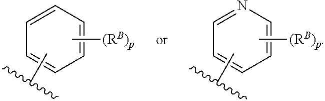

In some embodiments, Ring B is

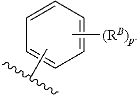

In some embodiments Ring B is

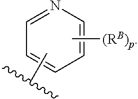

In some embodiments, Ring B is

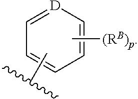

where D is CH or N.

In some embodiments, Ring B is phenyl or 6-membered heteroaryl; each R¹ and R² is independently hydrogen or C₁₋₆ alkyl; m is 2; and n is 2.

In some embodiments, the compound of Formula (I) has the structure of Formula (Ia), or a pharmaceutically acceptable salt, solvate, stereoisomer, or prodrug thereof:

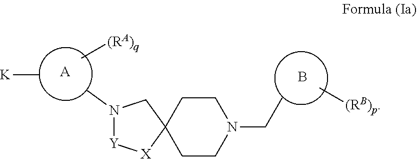

In some embodiments, the compound of Formula (I) has the structure of Formula (Ia-1), or a pharmaceutically acceptable salt, solvate, stereoisomer, or prodrug thereof:

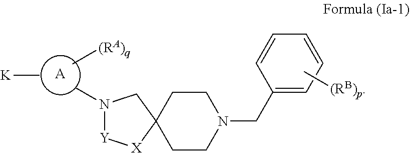

In some embodiments, the compound of Formula (I) has the structure of Formula (Ia-2), or a pharmaceutically acceptable salt, solvate, stereoisomer, or prodrug thereof:

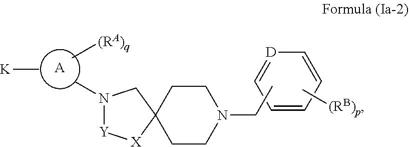

wherein D is CH or N.

In some embodiments, the compound of Formula (I) has the structure of Formula (Ia-3), or a pharmaceutically acceptable salt, solvate, stereoisomer, or prodrug thereof:

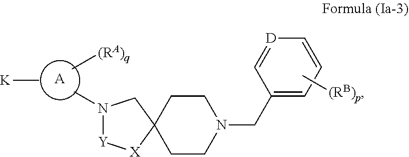

wherein D is CH or N.

In some embodiments, X is —O—. In some embodiments, X is —NR—. In some embodiments, X is —C(R⁴)₂—.

In some embodiments, Y is —C(═O)—. In some embodiments, Y is —S(═O)₂—.

In some embodiments, X is —O—, and Y is —C(═O)—. In some embodiments, X is —NR³—, and Y is —C(═O)—. In some embodiments, X is —C(R⁴)₂—; and Y is —C(═O)—. In some embodiments, X is —O—, and Y is —S(═O)₂—. In some embodiments, X is —NR³—, and Y is —S(═O)₂—. In some embodiments, X is —C(R⁴)₂—; and Y is —S(═O)₂—.

In some embodiments, X is —O—, and Y is —C(═O)—; or X is —NR³—, and Y is —C(═O)—; or X is —C(R⁴)₂—; and Y is —C(═O)—; or X is —O—, and Y is —S(═O)₂—; or X is —NR³—, and Y is —S(═O)₂—; or X is —C(R⁴)₂—; and Y is —S(═O)₂—. In some embodiments, X is —O—, and Y is —C(═O)—; or X is —NR³—, and Y is —C(═O)—; or X is —C(R⁴)₂—; and Y is —C(═O)—; or X is —NR³—, and Y is —S(═O)₂—.

In some embodiments, X is —NR³—, and Y is —C(═O)—; or X is —C(R⁴)₂—; and Y is —C(═O)—; or X is —O—, and Y is —S(═O)₂—; or X is —NR³—, and Y is —S(═O)₂—; or X is —C(R⁴)₂—; and Y is —S(═O)₂—.

In some embodiments, the compound of Formula (I) has the structure of Formula (Ib), Formula (Ic), Formula (Id), or Formula (Ie), or a pharmaceutically acceptable salt, solvate, stereoisomer, or prodrug thereof:

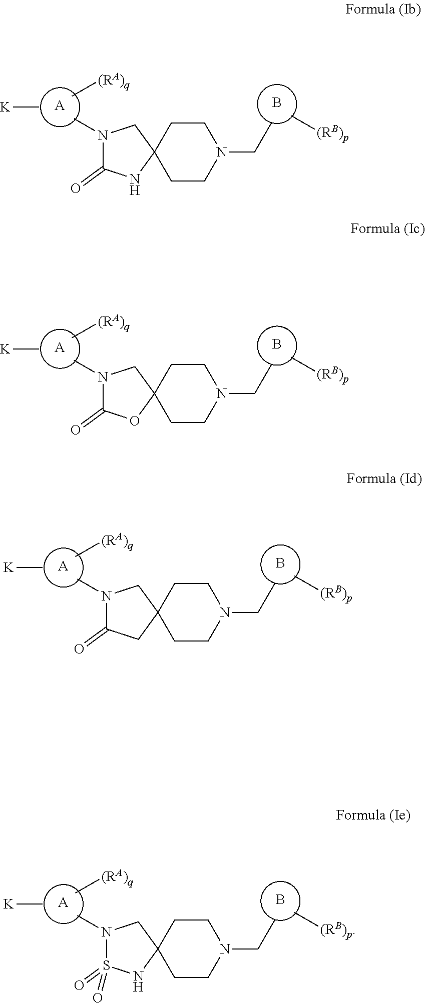

In some embodiments, the compound of Formula (I) has the structure of Formula (Ib). In some embodiments, the compound of Formula (I) has the structure of Formula (Ic). In some embodiments, the compound of Formula (I) has the structure of Formula (Id). In some embodiments, the compound of Formula (I) has the structure of Formula (Ie).

In some embodiments, the compound of Formula (I) has the structure of Formula (Ib), or a pharmaceutically acceptable salt, solvate, stereoisomer, or prodrug thereof:

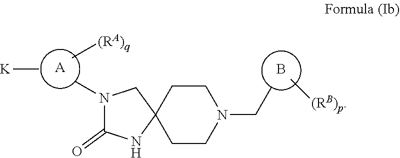

In some embodiments, the compound of Formula (I) has the structure of Formula (Ib-1), (Ib-2), or (Ib-3), or a pharmaceutically acceptable salt, solvate, stereoisomer, or prodrug thereof:

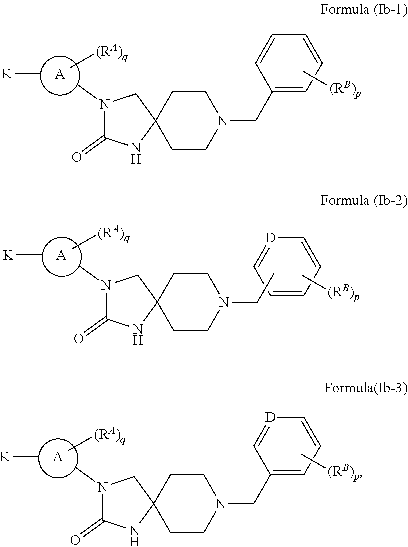

wherein D is CH or N.

In some embodiments, the compound of Formula (I) has the structure of Formula (Ic), or a pharmaceutically acceptable salt, solvate, stereoisomer, or prodrug thereof:

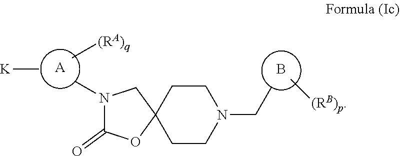

In some embodiments, the compound of Formula (I) has the structure of Formula (Ic-1), (Ic-2), or (Ic-3), or a pharmaceutically acceptable salt, solvate, stereoisomer, or prodrug thereof:

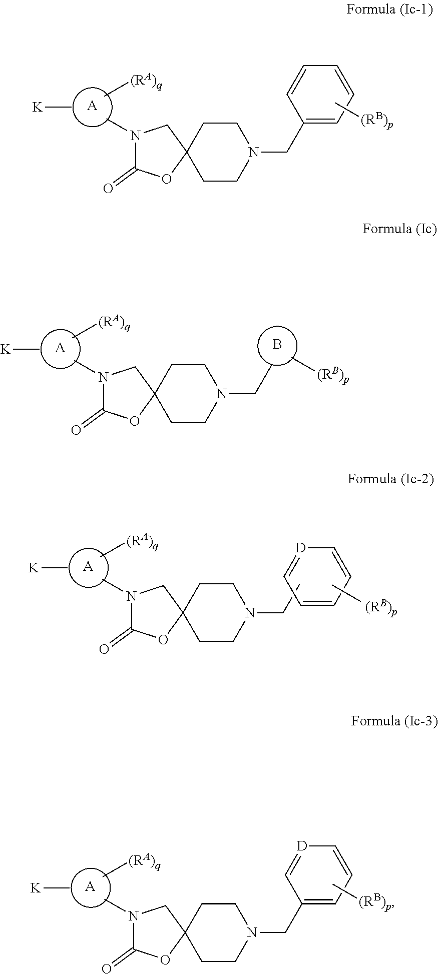

wherein D is CH or N.

In some embodiments, the compound of Formula (I) has the structure of Formula (Ic-1). In some embodiments, the compound of Formula (I) has the structure of Formula (Ic-2). In some embodiments, the compound of Formula (I) has the structure of Formula (Ic-3).

In some embodiments, the compound of Formula (I) has the structure of Formula (Id), or a pharmaceutically acceptable salt, solvate, stereoisomer, or prodrug thereof:

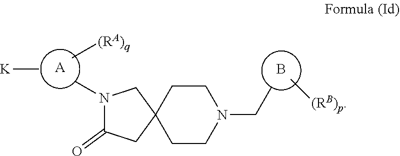

In some embodiments, the compound of Formula (I) has the structure of Formula (Id-1), (Id-2), or (Id-3), or a pharmaceutically acceptable salt, solvate, stereoisomer, or prodrug thereof:

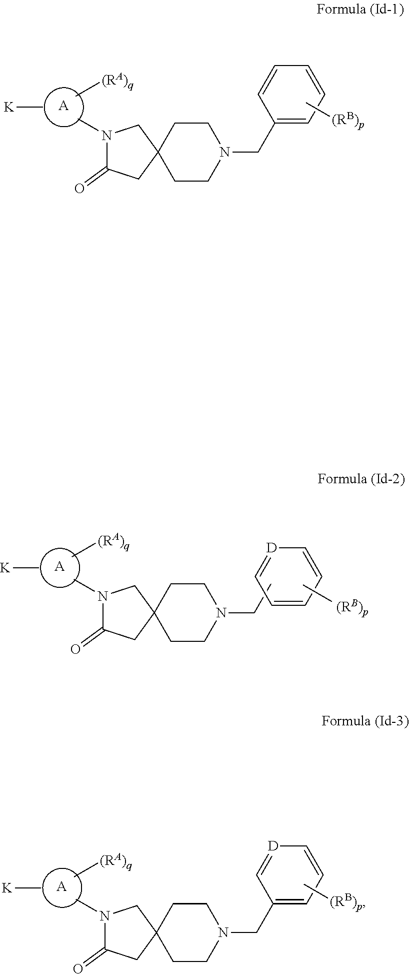

wherein D is CH or N.

In some embodiments, the compound of Formula (I) has the structure of Formula (Id-1). In some embodiments, the compound of Formula (I) has the structure of Formula (Id-2). In some embodiments, the compound of Formula (I) has the structure of Formula (Id-3).

In some embodiments, the compound of Formula (I) has the structure of Formula (Ie), or a pharmaceutically acceptable salt, solvate, stereoisomer, or prodrug thereof:

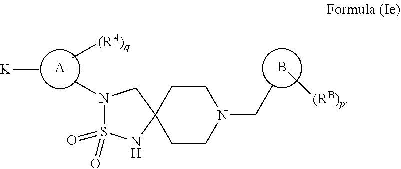

In some embodiments, the compound of Formula (I) has the structure of Formula (le-1), (Ie-2), or (Ie-3), or a pharmaceutically acceptable salt, solvate, stereoisomer, or prodrug thereof:

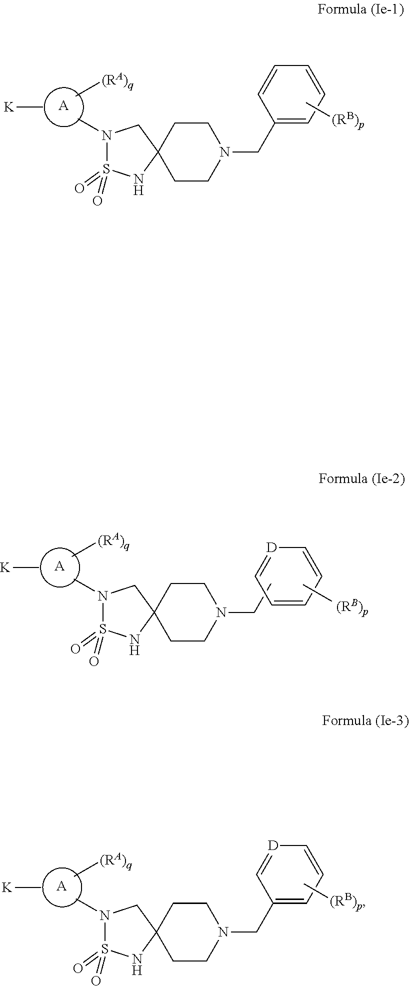

wherein D is CH or N.

In some embodiments, the compound of Formula (I) has the structure of Formula (Ie-1). In some embodiments, the compound of Formula (I) has the structure of Formula (Ie-2). In some embodiments, the compound of Formula (I) has the structure of Formula (Ie-3).

In some embodiments, each R^{B} is independently halogen, C₁-C₆ alkyl, phenyl, C₃-C₆ cycloalkyl, 3- to 6-membered heterocycloalkyl, 3- to 6-membered heterocycloalkenyl, 5-membered heteroaryl, 6-membered heteroaryl, —CN, —OR⁹, —CH₂CO₂R⁹, —CO₂R⁹, —C(═O)N(R⁹)₂, —N(R⁹)₂, —S(═O)₂R¹⁰, —S(═O)₂N(R⁹)₂, or —P(═O)(R₁₀)₂, wherein each alkyl, phenyl, and heteroaryl is unsubstituted or substituted with 1, 2, or 3 substituents selected from halogen, —CN, —OH, —O—(C₁-C₆ alkyl), C₁-C₆ alkyl, C₁-C₆ fluoroalkyl, C₁-C₆ hydroxyalkyl, —O—(C₁-C₆ fluoroalkyl), C₃-C₆ cycloalkyl, and 3- to 6-membered heterocycloalkyl; and wherein each cycloalkyl, heterocycloalkyl, and heterocycloalkenyl is unsubstituted or substituted with 1, 2, or 3 substituents selected from halogen, —CN, —OH, ═O, —O—(C₁-C₆ alkyl), C₁-C₆ alkyl, C₁-C₆ fluoroalkyl, C₁-C₆ hydroxyalkyl, —O—(C₁-C₆ fluoroalkyl), C₃-C₆ cycloalkyl, and 3- to 6-membered heterocycloalkyl. In some embodiments, each R^{B} is independently halogen, C₁-C₆ alkyl, phenyl, C₃-C₆ cycloalkyl, 5-membered heteroaryl, 6-membered heteroaryl, —CN, —OR⁹, —CH₂CO₂R⁹, —CO₂R⁹, —C(═O)N(R⁹)₂, or —S(═O)₂R¹⁰, wherein each alkyl, cycloalkyl, phenyl, and heteroaryl is unsubstituted or substituted with 1, 2, or 3 substituents selected from —F, —Cl, —Br, —CN, —OH, —CH₂OH, —O—(C₁-C₆ alkyl), C₁-C₆ alkyl, C₁-C₆ fluoroalkyl. In some embodiments, each R^{B} is independently phenyl, oxadiazolyl, pyridinyl, —CN, —CH₂CO₂R⁹, —CO₂R⁹, or —S(═O)₂R¹⁰, wherein the phenyl, oxadiazolyl, or pyridinyl is unsubstituted or substituted with 1, 2, or 3 substituents selected from —F, —Cl, —Br, —CN, —OH, —CH₂OH, —O—(C₁-C₆ alkyl), C₁-C₆ alkyl, and C₁-C₆ fluoroalkyl.

In some embodiments, p is 0. In some embodiments, p is 1. In some embodiments, p is 2. In some embodiments, p is 3. In some embodiments, p is 4. In some embodiments, p is 1-4. In some embodiments, p is 2 or 3.

In some embodiments, each R^{B} is independently halogen, C₁-C₆ alkyl, C₃-C₆ cycloalkyl, C₃-C₆ cycloalkenyl, 3- to 8-membered heterocycloalkyl, 3- to 8-membered heterocycloalkenyl, aryl, heteroaryl, —CN, —OR⁹, —OCH₂R⁹, —CO₂R⁹, —CH₂CO₂R⁹, —OC(═O)R⁹, —C(═O)N(R⁹)₂, —N(R⁹)₂, —NR⁹C(═O)R⁹, —NR⁹C(═O)OR¹⁰, —OC(═O)NR⁹, —NR⁹C(═O)N(R⁹)₂, —C(R⁹)═N—OR⁹, —SR⁹, —S(═O)R¹⁰, —S(═O)₂R¹⁰, —S(═O)₂N(R⁹)₂, —P(═O)(OR⁹)₂, —P(═O)(OR⁹)R¹⁰ or —P(═O)(R¹⁰)₂, wherein each alkyl, aryl, and heteroaryl is unsubstituted or substituted with 1, 2, or 3 substituents selected from halogen, —CN, —OH, —O—(C₁-C₆ alkyl), —CO₂—(C₁-C₆ alkyl), C₁-C₆ alkyl, C₁-C₆ fluoroalkyl, C₁-C₆ hydroxyalkyl, —O—(C₁-C₆ fluoroalkyl), C₃-C₆ cycloalkyl, and 3- to 6-membered heterocycloalkyl; and wherein each cycloalkyl, cycloalkenyl, heterocycloalkyl, and heterocycloalkenyl is unsubstituted or substituted with 1, 2, or 3 substituents selected from halogen, —CN, —OH, ═O, —O—(C₁-C₆ alkyl), C₁-C₆ alkyl, C₁-C₆ fluoroalkyl, C₁-C₆ hydroxyalkyl, —O—(C₁-C₆ fluoroalkyl), C₃-C₆ cycloalkyl, and 3- to 6-membered heterocycloalkyl; and p is 1-4.

In some embodiments, the compound of Formula (I) has the structure of Formula (If), or a pharmaceutically acceptable salt, solvate, stereoisomer, or prodrug thereof:

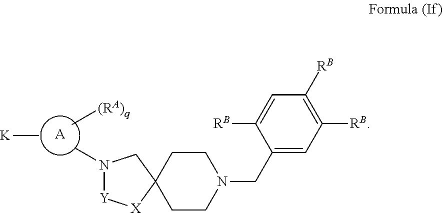

In some embodiments, the compound of Formula (I) has the structure of Formula (Ig), or a pharmaceutically acceptable salt, solvate, stereoisomer, or prodrug thereof:

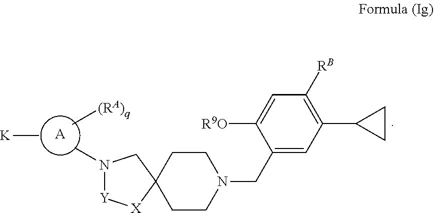

In some embodiments, R^{B} is phenyl, oxadiazolyl, pyridinyl, —CN, —CH₂CO₂R⁹, —CO₂R⁹, or —S(═O)₂R¹⁰, wherein the phenyl, oxadiazolyl, or pyridinyl is unsubstituted or substituted with 1, 2, or 3 substituents selected from —F, —Cl, —Br, —CN, —OH, —CH₂OH, —O—(C₁-C₆ alkyl), C₁-C₆ alkyl, C₁-C₆ fluoroalkyl.

In some embodiments, Ring A is phenyl, naphthyl, monocyclic 6-membered heteroaryl, monocyclic 5-membered heteroaryl, bicyclic heteroaryl, monocyclic C₃-C₈cycloalkyl, bridged C₅-C₁₀ cycloalkyl, spiro C₅-C₁₀ cycloalkyl, monocyclic C₂-C₈ heterocycloalkyl, bridged C₅-C₁₀ heterocycloalkyl, or spiro C₅-C₁₀ heterocycloalkyl.

In some embodiments, Ring A is phenyl, monocyclic heteroaryl, monocyclic cycloalkyl, spirocyclic cycloalkyl, bridged cycloalkyl, monocyclic heterocycloalkyl, spirocyclic heterocycloalkyl, or bridged heterocycloalkyl. In some embodiments, Ring A is phenyl, monocyclic 6-membered heteroaryl, monocyclic 5-membered heteroaryl, monocyclic C₃-C₈cycloalkyl, bridged C₅-C₁₀ cycloalkyl, spiro C₅-C₁₀ cycloalkyl, monocyclic C₂-C₈ heterocycloalkyl, bridged C₅-C₁₀ heterocycloalkyl, or spiro C₅-C₁₀ heterocycloalkyl.

In some embodiments, Ring A is phenyl or heteroaryl. In some embodiments, Ring A is phenyl or monocyclic heteroaryl. In some embodiments, Ring A is phenyl, monocyclic 6-membered heteroaryl, or monocyclic 5-membered heteroaryl. In some embodiments, Ring A is phenyl, pyridinyl, pyrimidinyl, pyrazinyl, pyridazinyl, triazinyl, furanyl, thienyl, pyrrolyl, oxazolyl, thiazolyl, imidazolyl, pyrazolyl, triazolyl, tetrazolyl, isoxazolyl, isothiazolyl, oxadiazolyl, or thiadiazolyl.

In some embodiments, Ring A is phenyl or 6-membered heteroaryl. In some embodiments, Ring A is phenyl, pyridinyl, pyrimidinyl, pyrazinyl, or pyridazinyl.

In some embodiments, Ring A is phenyl, monocyclic C₃-C₆ cycloalkyl, or bridged cycloalkyl. In some embodiments, Ring A is phenyl, monocyclic C₃-C₈ cycloalkyl, or bridged C₅-C₁₀ cycloalkyl. In some embodiments, Ring A is phenyl, cyclopropyl, cyclobutyl, cyclopentyl, cyclohexyl, or bridged C₅-C₁₀cycloalkyl. In some embodiments, Ring A is phenyl, cyclohexyl, or

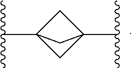 [SMILES: *C12CC(*)(C1)C2]

In some embodiments, Ring A is phenyl. In some embodiments, Ring A is cyclohexyl. In some embodiments, Ring A is

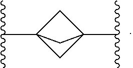 [SMILES: *C12CC(*)(C1)C2]

In some embodiments, Ring A is phenyl, naphthyl, indanyl, indenyl, tetrahyodronaphthyl, cyclopropyl, cyclobutyl, cyclopentyl, cyclopentenyl, cyclohexyl, cyclohexenyl, cycloheptyl, cyclooctyl, spiro[2.2]pentyl, spiro[3.3]heptyl, spiro[3.5]nonyl, spiro[4.4]nonyl, spiro[4.5]decyl, norbornyl, norbornenyl, bicyclo[1.1.1]pentyl, adamantyl, or decalinyl.

In some embodiments, Ring A is monocyclic cycloalkyl, spirocyclic cycloalkyl, bridged cycloalkyl, monocyclic heterocycloalkyl, spirocyclic heterocycloalkyl, or bridged heterocycloalkyl. In some embodiments, Ring A is cyclopropyl, cyclobutyl, cyclopentyl, cyclopentenyl, cyclohexyl, cyclohexenyl, cycloheptyl, cyclooctyl, spiro[2.2]pentyl, spiro[3.3]heptyl, spiro[3.5]nonyl, spiro[4.4]nonyl, spiro[4.5]decyl, norbornyl, norbornenyl, bicyclo[1.1.1]pentyl, adamantyl, or decalinyl. In some embodiments, Ring A is monocyclic C₃-C₆cycloalkyl, or bridged cycloalkyl. In some embodiments, Ring A is cyclopropyl, cyclobutyl, cyclopentyl, cyclohexyl, or bridged C₅-C₁₀cycloalkyl. In some embodiments, Ring A is cyclohexyl or

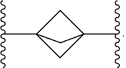 [SMILES: *C12CC(*)(C1)C2]

In some embodiments, Ring A is furanyl, thienyl, pyrrolyl, oxazolyl, thiazolyl, imidazolyl, pyrazolyl, triazolyl, tetrazolyl, isoxazolyl, isothiazolyl, oxadiazolyl, thiadiazolyl, pyridinyl, pyrimidinyl, pyrazinyl, pyridazinyl, triazinyl, quinolinyl, isoquinolinyl, cinnolinyl, phthalazinyl, quinazolinyl, quinoxalinyl, naphthyridinyl, pteridinyl, indolizinyl, azaindolizinyl, indolyl, azaindolyl, indazolyl, azaindazolyl, benzimidazolyl, azabenzimidazolyl, benzotriazolyl, azabenzotriazolyl, benzoxazolyl, azabenzoxazolyl, benzisoxazolyl, azabenzisoxazolyl, benzofuranyl, azabenzofuranyl, benzothienyl, azabenzothienyl, benzothiazolyl, azabenzothiazolyl, or purinyl.

In some embodiments, Ring A is aziridinyl, azetidinyl, oxetanyl, thietanyl, pyrrolidinyl, tetrahydrofuranyl, tetrahydrothienyl, oxazolidinonyl, tetrahydropyranyl, piperidinyl, morpholinyl, thiomorpholinyl, piperazinyl, homopiperidinyl, oxepanyl, thiepanyl, oxazepinyl, diazepinyl, thiazepinyl, azaspiro[3.3]heptanyl, azaspiro[3.4]octanyl, azaspiro[3.4]octanyl, or azaspiro[4.4]nonyl.

In some embodiments, Ring A is phenyl, pyridinyl, pyrimidinyl, pyrazinyl, or pyridazinyl.

In some embodiments, Ring A is an aziridinyl, azetidinyl, oxetanyl, thietanyl, pyrrolidinyl, tetrahydrofuranyl, tetrahydrothienyl, oxazolidinonyl, tetrahydropyranyl, piperidinyl, morpholinyl, thiomorpholinyl, or piperazinyl.

In some embodiments, each R^{A} is independently halogen, —OH, —O—(C₁-C₆ alkyl), C₁-C₆ alkyl, C₃-C₆ cycloalkyl, wherein each alkyl and cycloalkyl is unsubstituted or substituted with 1, 2, or 3 substituents selected from halogen, —CN, —OH, —O—(C₁-C₆ alkyl), C₁-C₆ alkyl, and C₁-C₆ fluoroalkyl. In some embodiments, each R^{A} is independently halogen, —OH, —O—(C₁-C₆ alkyl), or C₁-C₆ alkyl. In some embodiments, each R^{A} is independently —F, —Cl, —Br, —OH, —OCH₃, —OCH₂CH₃, —OCH₂CH₂CH₃, —OCH(CH₃)₂, —CH₃, —CH₂CH₃, —CH₂CH₂CH₃, —CH(CH₃)₂, —CH₂CH₂CH₂CH₃, —CH₂CH(CH₃)₂, —CH(CH₃)(CH₂CH₃), or —C(CH₃)₃. In some embodiments, each R^{A} is independently C₁-C₆ alkyl. In some embodiments, each R^{A} is independently —CH₃, —CH₂CH₃, —CH₂CH₂CH₃, —CH(CH₃)₂, —CH₂CH₂CH₂CH₃, —CH₂CH(CH₃)₂, —CH(CH₃)(CH₂CH₃), or —C(CH₃)₃.

In some embodiments, q is 0. In some embodiments, q is 1-4. In some embodiments, q is 0-2. In some embodiments, q is 0-1. In some embodiments, q is 1. In some embodiments, q is 2. In some embodiments, q is 3. In some embodiments, q is 4.

In some embodiments, Ring A is phenyl, monocyclic heteroaryl, monocyclic cycloalkyl, spirocyclic cycloalkyl, bridged cycloalkyl, monocyclic heterocycloalkyl, spirocyclic heterocycloalkyl, or bridged heterocycloalkyl; each R^{A} is independently halogen, —OH, —O—(C₁-C₆ alkyl), C₁-C₆ alkyl, C₃-C₆ cycloalkyl, wherein each alkyl and cycloalkyl is unsubstituted or substituted with 1, 2, or 3 substituents selected from halogen, —CN, —OH, —O—(C₁-C₆ alkyl), C₁-C₆ alkyl, and C₁-C₆ fluoroalkyl; and q is 0-2.

In some embodiments, Ring A is phenyl, monocyclic C₃-C₆ cycloalkyl, or bridged cycloalkyl; each R^{A} is independently halogen, —OH, —O—(C₁-C₆ alkyl), or C₁-C₆ alkyl; and q is 0-2.

In some embodiments, Ring A is phenyl, cyclohexyl, or

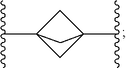 [SMILES: *C12CC(*)(C1)C2]

each R^{A} is independently halogen, —OH, —O—(C₁-C₆ alkyl), or C₁-C₆ alkyl; and q is 0-2.

In some embodiments, Ring A is phenyl; and q is 0.

In some embodiments, when X is —O—, and Y is —C(═O)—, Ring A is phenyl or heteroaryl. In some embodiments, Ring A is phenyl.

In some embodiments, when X is —O—, and Y is —C(═O)—, Ring A is monocyclic cycloalkyl, spirocyclic cycloalkyl, bridged cycloalkyl, monocyclic heterocycloalkyl, spirocyclic heterocycloalkyl, or bridged heterocycloalkyl. In some embodiments, Ring A is monocyclic C₃-C₆ cycloalkyl, or bridged cycloalkyl. In some embodiments, Ring A is cyclohexyl or

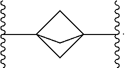 [SMILES: *C12CC(*)(C1)C2]

In some embodiments, each R^{A} is independently halogen, —OH, —O—(C₁-C₆ alkyl), C₁-C₆ alkyl, C₃-C₆ cycloalkyl, wherein each alkyl and cycloalkyl is unsubstituted or substituted with 1, 2, or 3 substituents selected from halogen, —CN, —OH, —O—(C₁-C₆ alkyl), C₁-C₆ alkyl, and C₁-C₆ fluoroalkyl; and q is 0-2. In some embodiments, each R^{A} is independently halogen, —OH, —O—(C₁-C₆ alkyl), or C₁-C₆ alkyl; and q is 0-2. In some embodiments, each R^{A} is independently C₁-C₆ alkyl; and q is 0-2. In some embodiments, q is 0.

In some embodiments, the compound of Formula (I) has the structure of Formula (Ih), or a pharmaceutically acceptable salt, solvate, stereoisomer, or prodrug thereof:

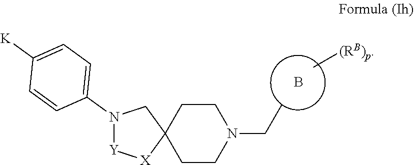

In some embodiments, the compound of Formula (I) has the structure of Formula (Ih-1), (Ih-2), or (Ih-3), or a pharmaceutically acceptable salt, solvate, stereoisomer, or prodrug thereof:

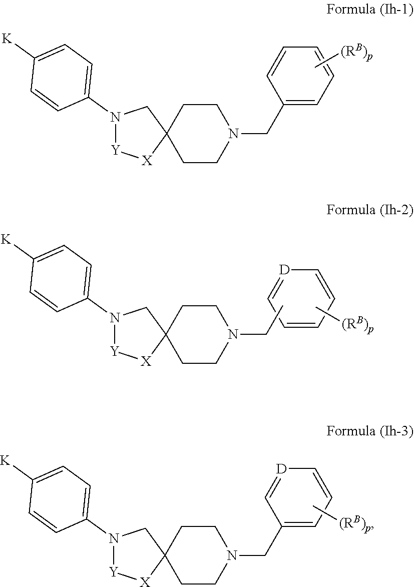

wherein D is CH or N.

In some embodiments, the compound of Formula (I) has the structure of Formula (Ih-1). In some embodiments, the compound of Formula (I) has the structure of Formula (Ih-2). In some embodiments, the compound of Formula (I) has the structure of Formula (Ih-3).

In some embodiments, the compound of Formula (I) has the structure of Formula (Ii), or a pharmaceutically acceptable salt, solvate, stereoisomer, or prodrug thereof:

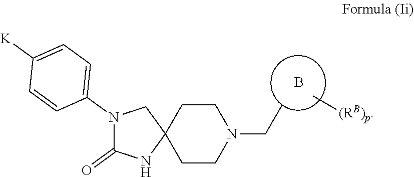

In some embodiments, the compound of Formula (I) has the structure of Formula (Ii-1), (Ii-2), or (Ii-3), or a pharmaceutically acceptable salt, solvate, stereoisomer, or prodrug thereof:

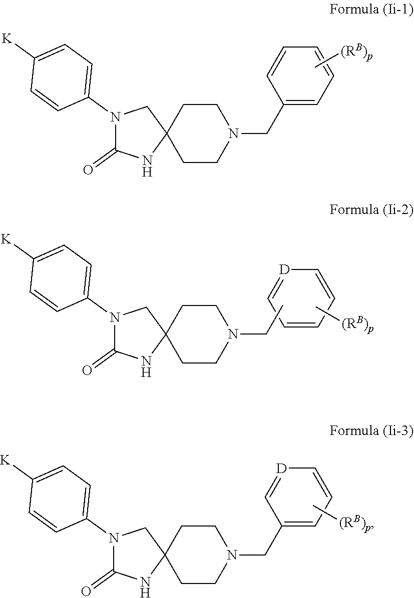

wherein D is CH or N.

In some embodiments, the compound of Formula (I) has the structure of Formula (Ii-1). In some embodiments, the compound of Formula (I) has the structure of Formula (Ii-2). In some embodiments, the compound of Formula (I) has the structure of Formula (Ii-3).

In some embodiments, the compound of Formula (I) has the structure of Formula (Ij), o or a pharmaceutically acceptable salt, solvate, stereoisomer, or prodrug thereof:

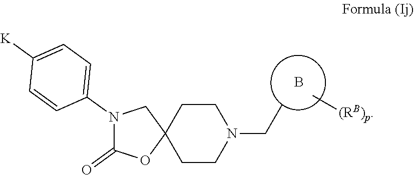

In some embodiments, the compound of Formula (I) has the structure of Formula (Ij-1), (Ij-2), or (Ij-3), or a pharmaceutically acceptable salt, solvate, stereoisomer, or prodrug thereof:

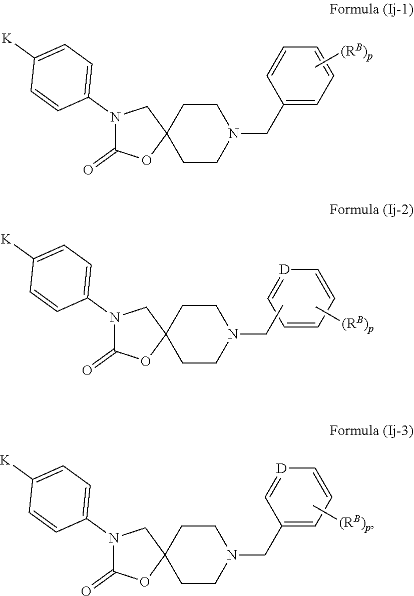

wherein D is CH or N.

In some embodiments, the compound of Formula (I) has the structure of Formula (Ij-1). In some embodiments, the compound of Formula (I) has the structure of Formula (Ij-2). In some embodiments, the compound of Formula (I) has the structure of Formula (Ij-3).

In some embodiments, the compound of Formula (I) has the structure of Formula (Ik), or a pharmaceutically acceptable salt, solvate, stereoisomer, or prodrug thereof:

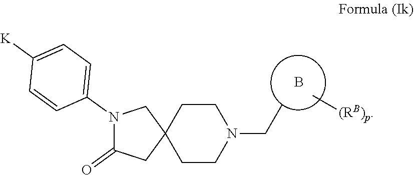

In some embodiments, the compound of Formula (I) has the structure of Formula (Ik-1), (Ik-2), or (Ik-3), or a pharmaceutically acceptable salt, solvate, stereoisomer, or prodrug thereof:

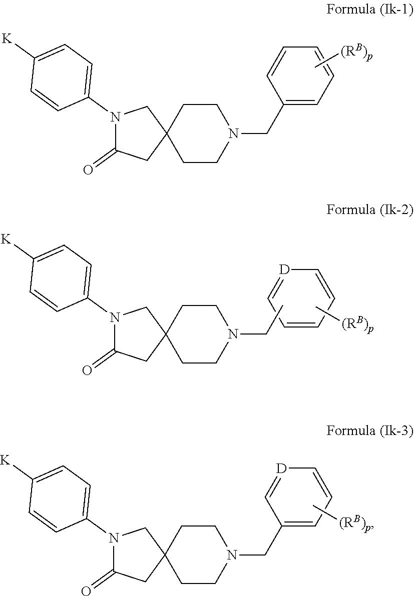

wherein D is CH or N.

In some embodiments, the compound of Formula (I) has the structure of Formula (Il), or a pharmaceutically acceptable salt, solvate, stereoisomer, or prodrug thereof:

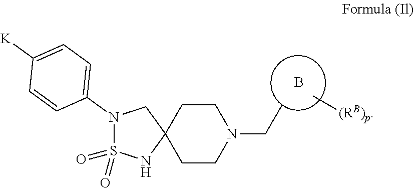

In some embodiments, the compound of Formula (I) has the structure of Formula (Il-1), (Il-2), or (Il-3), or a pharmaceutically acceptable salt, solvate, stereoisomer, or prodrug thereof:

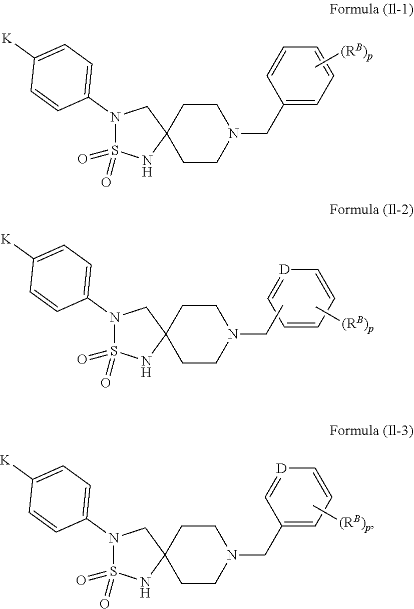

wherein D is CH or N.

In some embodiments, the compound of Formula (I) has the structure of Formula (Il-1). In some embodiments, the compound of Formula (I) has the structure of Formula (Il-2). In some embodiments, the compound of Formula (I) has the structure of Formula (Il-3).

In some embodiments, K is —(CH₂)ⱼ-G. In some embodiments, K is —(CH₂)ⱼ-G and j is 0 or 1.

In some embodiments, j is 0 or 1. In some embodiments, j is 0. In some embodiments, j is 1. In some embodiments, j is 2. In some embodiments, j is 3. In some embodiments, j is 4.

In some embodiments, G is —P(═O)(OH)₂, —P(═O)(OH)(R^{D}), —P(═O)(OH)(OR^{D}), —B(OH)₂, —B(OR^{D})(OH), —NHC(═O)H, —NHC(═O)(R^{D}), —NHS(═O)₂(R^{D}), —NHC(═O)NH₂, —NHC(═O)NH(R^{D}), —N(R^{D})C(═O)NHS(═O)₂(R^{D}), or —C(═O)NHS(═O)₂(R^{D}). In some embodiments, G is —P(═O)(OH)₂, —P(═O)(OH)(R^{D}), —P(═O)(OH)(OR^{D}), —B(OH)₂, —B(OR^{D})(OH), —NHC(═O)H, —NHC(═O)(R^{D}), —NHS(═O)₂(R^{D}), —NHC(═O)NH₂, —NHC(═O)NH(R^{D}), —N(R^{D})C(═O)NHS(═O)₂(R^{D}), or —C(═O)NHS(═O)₂(R^{D}); and R^{D} is alkyl which is unsubstituted or substituted with 1, 2, or 3 —OH groups. In some embodiments, G is —P(═O)(OH)₂, —P(═O)(OH)(R^{D}), —P(═O)(OH)(H), —P(═O)(OH)(OR^{D}), —B(OH)₂, —NHC(═O)H, —NHC(═O)(R^{D}), —NHS(═O)₂(R^{D}), —NHC(═O)NH₂, —NHC(═O)NH(R^{D}), —NHC(═NH)NHC(═NH)NH₂, or

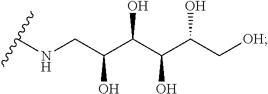 [SMILES: *NC[C@H](O)[C@@H](O)[C@H](O)[C@H](O)CO]

and R^{D} is alkyl which is unsubstituted or substituted with 1, 2, or 3 —OH groups.

In some embodiments, G is —P(═O)(OH)₂, —P(═O)(OH)(R^{D}), or —P(═O)(OH)(OR^{D}). In some embodiments, G is —P(═O)(OH)₂, —P(═O)(OH)(R^{D}), or —P(═O)(OH)(OR^{D}); and R^{D} is C₁₋₆ alkyl. In some embodiments, G is —P(═O)(OH)₂, —P(═O)(OH)(R^{D}), or —P(═O)(OH)(OR^{D}); R^{D} is C₁₋₆ alkyl; and j is 0 or 1. In some embodiments, G is —P(═O)(OH)(H).

In some embodiments, K is —(CH₂)ⱼP(═O)(OH)₂, —(CH₂)ⱼP(═O)(OMe)(OH), —(CH₂)ⱼP(═O)(OEt)(OH), —(CH₂)ⱼP(═O)(Me)(OH), or —(CH₂)ⱼP(═O)(Et)(OH). In some embodiments, K is —(CH₂)ⱼP(═O)(OH)₂, —(CH₂)ⱼP(═O)(OMe)(OH), —(CH₂)ⱼP(═O)(OEt)(OH), —(CH₂)ⱼP(═O)(Me)(OH), or —(CH₂)ⱼP(═O)(Et)(OH); and j is 0 or 1. In some embodiments, K is —P(═O)(OH)₂, —P(═O)(OEt)(OH), or —P(═O)(Me)(OH). In some embodiments, K is —P(═O)(OH)₂, —CH₂P(═O)(OH)₂, —P(═O)(OH)(R^{D}), —P(═O)(OH)(H), —P(═O)(OH)(OR^{D}), —B(OH)₂, —NHC(═O)H, —(CH₂)₄NHC(═O)H, —NHC(═O)(R^{D}), —(CH₂)₄NHC(═O)(R^{D}), —NHS(═O)₂(R^{D}), —(CH₂)₄NHS(═O)₂(R^{D}), —NHC(═O)NH₂, —(CH₂)₄NHC(═O)NH₂, —NHC(═O)NH(R^{D}), —(CH₂)₄NHC(═O)NH(R^{D}), —CH₂NHC(═NH)NHC(═NH)NH₂, —CH₂CH₂NHC(═NH)NHC(═NH)NH₂, or

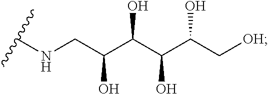 [SMILES: *NC[C@H](O)[C@@H](O)[C@H](O)[C@H](O)CO]

and R^{D} is alkyl which is unsubstituted or substituted with 1, 2, or 3 —OH groups.

In some embodiments, the compound of Formula (I) has the structure of Formula (Ij-a) Formula (Ij-b), or Formula (Ij-c), or a pharmaceutically acceptable salt, solvate, stereoisomer, or prodrug thereof:

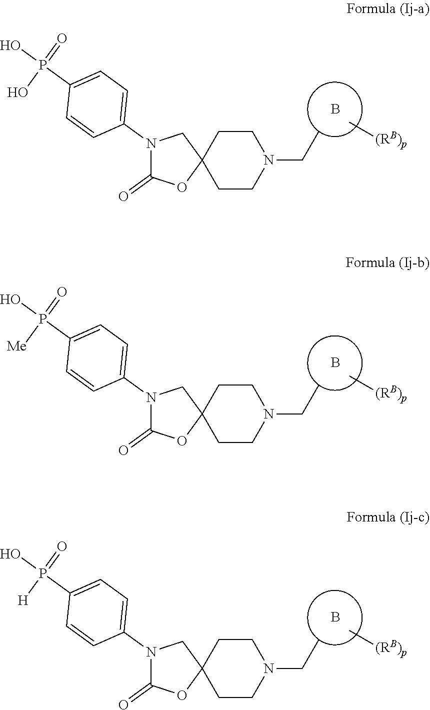

In some embodiments, the compound of Formula (I) has the structure of Formula (Ij-a), or a pharmaceutically acceptable salt, solvate, stereoisomer, or prodrug thereof. In some embodiments, the compound of Formula (I) has the structure of Formula (Ij-b), or a pharmaceutically acceptable salt, solvate, stereoisomer, or prodrug thereof. In some embodiments, the compound of Formula (I) has the structure of Formula (Ij-c), or a pharmaceutically acceptable salt, solvate, stereoisomer, or prodrug thereof. In some embodiments, the compound of Formula (I) has the structure of Formula (Ij-a) or Formula (Ij-b), or a pharmaceutically acceptable salt, solvate, stereoisomer, or prodrug thereof.

In some embodiments, the compound of Formula (I) has the structure of Formula (Im), or a pharmaceutically acceptable salt, solvate, stereoisomer, or prodrug thereof:

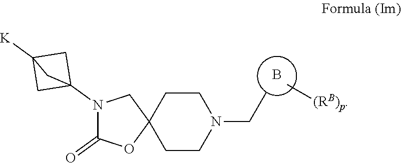

In some embodiments, the compound of Formula (I) has the structure of Formula (Im-1), Formula (Im-2), or Formula (Im-3), or a pharmaceutically acceptable salt, solvate, stereoisomer, or prodrug thereof:

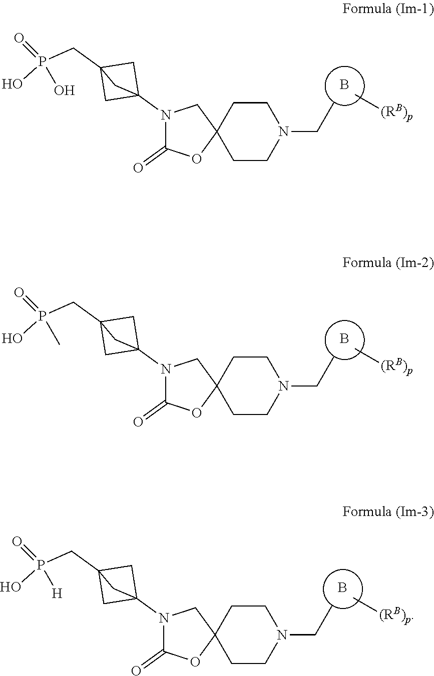

In some embodiments, the compound of Formula (I) has the structure of Formula (In), or a pharmaceutically acceptable salt, solvate, stereoisomer, or prodrug thereof:

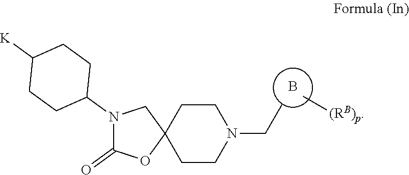

In some embodiments, the compound of Formula (I) has the structure of Formula (In-1), Formula (In-2), Formula (In-3), or Formula (In-4), or a pharmaceutically acceptable salt, solvate, stereoisomer, or prodrug thereof:

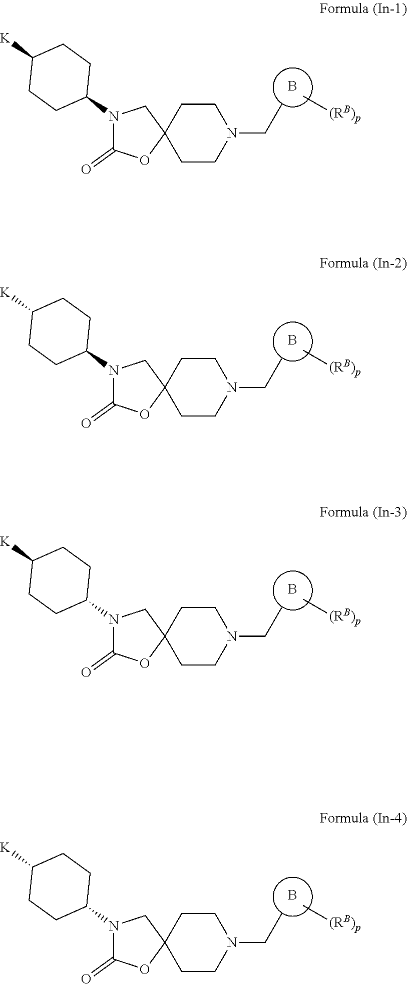

In some embodiments, the compound of Formula (I) has the structure of Formula (In-1). In some embodiments, the compound of Formula (I) has the structure of Formula (In-2). In some embodiments, the compound of Formula (I) has the structure of Formula (In-3). In some embodiments, the compound of Formula (I) has the structure of Formula (In-4).

In some embodiments, Ring B is

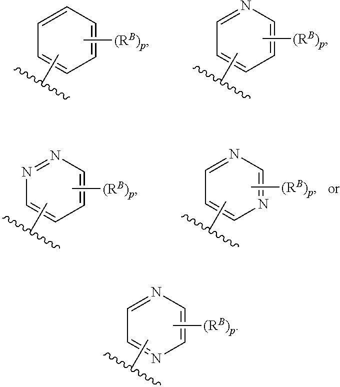

In some embodiments, Ring B is

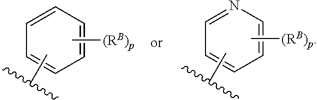

In some embodiments, Ring B is

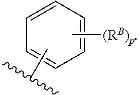

In some embodiments, Ring B is

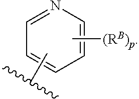

In some embodiments, Ring B is phenyl, or pyridinyl.

In some embodiments, Ring B is

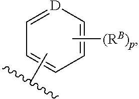

where D is CH or N.

In some embodiments, each R^{B} is independently C₁-C₆ alkyl, C₃-C₆ cycloalkyl, aryl, heteroaryl, —OR⁹, —CO₂R⁹, or —S(═O)₂R¹⁰, wherein each alkyl, aryl, and heteroaryl is unsubstituted or substituted with 1, 2, or 3 substituents selected from halogen, —CN, —OH, —O—(C₁-C₆ alkyl), —CO₂—(C₁-C₆ alkyl), C₁-C₆ alkyl, C₁-C₆ fluoroalkyl, C₁-C₆ hydroxyalkyl, —O—(C₁-C₆ fluoroalkyl), C₃-C₆ cycloalkyl, and 3- to 6-membered heterocycloalkyl; and p is 1-4. In some embodiments, each R^{B} is independently C₁-C₆ alkyl, C₃-C₆ cycloalkyl, aryl, heteroaryl, —OR⁹, —CO₂R⁹, or —S(═O)₂R¹⁰, wherein each alkyl, aryl, and heteroaryl is unsubstituted or substituted with 1 halogen or C₁-C₆ alkyl. In some embodiments, at least one R^{B} is phenyl, pyridinyl, pyrimidinyl, pyridazinyl, or pyrazinyl, unsubstituted or substituted with 1, 2, or 3 halogen. In some embodiments, at least one R^{B} is fluorophenyl, fluoropyridinyl, or fluoropyrimidinyl. In some embodiments, at least one R^{B} is C₁-C₆ alkyl or C₃-C₆ cycloalkyl. In some embodiments, at least one R^{B} is methyl, ethyl, n-propyl, isopropyl, cyclopropyl, n-butyl, tert-butyl, sec-butyl, isobutyl, or cyclobutyl. In some embodiments, at least one R^{B} is ethyl, isopropyl, cyclopropyl, tert-butyl, isobutyl, or cyclobutyl. In some embodiments, at least one R^{B} is isopropyl, cyclopropyl, or cyclobutyl. In some embodiments, at least one R^{B} is —OR⁹. In some embodiments, at least one R^{B} is —S(═O)₂R¹⁰. In some embodiments, at least one R^{B} is —CO₂R⁹. In some embodiments, R⁹ is C₁-C₆ alkyl.

Also disclosed herein, in certain embodiments, is a compound of Formula (II):

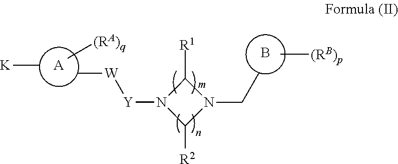

or a pharmaceutically acceptable salt, solvate, stereoisomer, or prodrug thereof, wherein:
W is a bond, —O—, —NR³—, or —C(R⁴)₂—;
Y is —C(═O)—, or —S(═O)₂—;
Ring A is aryl, heteroaryl, cycloalkyl, or heterocycloalkyl;
Ring B is aryl or heteroaryl;
K is —(CH₂)ⱼ-G;
   G is —P(═O)(OH)₂, —P(═O)(OH)(R^{D}), —P(═O)(OH)(H), —P(═O)(OH)(OR^{D}), —B(OH)₂, —B(OR^{D})(OH), —NHC(═O)H, —NHC(═O)(R^{D}), —NHS(═O)₂(R^{D}), —NHC(═O)NHS(═O)₂(R^{D}), —N(R^{D})C(═O)NHS(═O)₂(R^{D}), —C(═O)NHS(═O)₂(R^{D}), —S(═O)₂NHC(═O)(R^{D}), —NHC(═O)NH₂, —NHC(═O)NH(R^{D}), —NHC(═NH)NH₂, —NHC(═NH)NH(R^{D}), —NHC(═NH)N(R^{D})₂, —N(R^{D})C(═NH)NH₂, —N(R^{D})C(═NH)NH(R^{D}), —N(R^{D})C(═NH)N(R^{D})₂, —NHC(═N(R^{D}))NH₂, —NHC(═N(R^{D}))NH(R^{D}), —NHC(═N(R^{D}))N(R^{D})₂, N(R^{D})C(═N(R^{D}))NH₂, —N(R^{D})C(═N(R^{D}))NH(R^{D}), —N(R^{D})C(═N(R^{D}))N(R^{D})₂, —NHC(═NH)NHC(═NH)NH₂, —N(R^{D})C(═NH)NHC(═NH)NH₂,

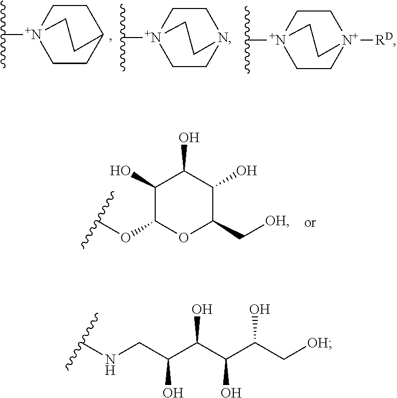

j is 0-4;
each R¹ and R² is independently hydrogen, C₁₋₆ alkyl, or C₁₋₆ fluoroalkyl;
or one R¹ and one R² are taken together to form a ring;
R³ is hydrogen, C₁₋₆ alkyl, C₁₋₆ fluoroalkyl, or C₃₋₆ cycloalkyl;
each R⁴ is independently hydrogen, C₁₋₆ alkyl, C₁₋₆ fluoroalkyl, or C₃₋₆ cycloalkyl;
each R^{D} is independently C₁₋₆ alkyl or C₃₋₆ cycloalkyl; wherein the alkyl and cycloalkyl are unsubstituted or substituted by 1-3 halogen or —OH groups;
each R^{A} is independently halogen, —OH, —O—(C₁-C₆ alkyl), C₁-C₆ alkyl, C₃-C₆ cycloalkyl, 3- to 8-membered heterocycloalkyl, wherein each alkyl, cycloalkyl, and heterocycloalkyl is unsubstituted or substituted with 1, 2, or 3 substituents selected from halogen, —CN, —OH, —O—(C₁-C₆ alkyl), C₁-C₆ alkyl, C₁-C₆ fluoroalkyl, C₁-C₆ hydroxyalkyl, —O—(C₁-C₆ fluoroalkyl), C₃-C₆ cycloalkyl, and 3- to 6-membered heterocycloalkyl;
each R^{B} is independently halogen, C₁-C₆ alkyl, C₃-C₆ cycloalkyl, C₃-C₆ cycloalkenyl, 3- to 8-membered heterocycloalkyl, 3- to 8-membered heterocycloalkenyl, aryl, heteroaryl, —CN, —OR⁹, —OCH₂R⁹, —CO₂R⁹, —CH₂CO₂R⁹, —OC(═O)R⁹, —C(═O)N(R⁹)₂, —N(R⁹)₂, —NR⁹C(═O)R⁹, —NR⁹C(═O)OR¹⁰, —OC(═O)NR⁹, —NR⁹C(═O)N(R⁹)₂, —C(R⁹)═N—OR⁹, —SR⁹, —S(═O)R¹⁰, —S(═O)₂R¹⁰, —S(═O)₂N(R⁹)₂, —P(═O)(OR⁹)₂, —P(═O)(OR⁹)R¹⁰ or —P(═O)(R¹⁰)₂, wherein each alkyl, aryl, and heteroaryl is unsubstituted or substituted with 1, 2, or 3 substituents selected from halogen, —CN, —OH, —O—(C₁-C₆ alkyl), —CO₂—(C₁-C₆ alkyl), C₁-C₆ alkyl, C₁-C₆ fluoroalkyl, C₁-C₆ hydroxyalkyl, —O—(C₁-C₆ fluoroalkyl), C₃-C₆ cycloalkyl, and 3- to 6-membered heterocycloalkyl; and wherein each cycloalkyl, cycloalkenyl, heterocycloalkyl, and heterocycloalkenyl is unsubstituted or substituted with 1, 2, or 3 substituents selected from halogen, —CN, —OH, ═O, —O—(C₁-C₆ alkyl), C₁-C₆ alkyl, C₁-C₆ fluoroalkyl, C₁-C₆ hydroxyalkyl, —O—(C₁-C₆ fluoroalkyl), C₃-C₆ cycloalkyl, and 3- to 6-membered heterocycloalkyl;
each R⁹ is independently selected from hydrogen, C₁-C₆ alkyl, C₁-C₆ fluoroalkyl, C₃-C₆ cycloalkyl, 3- to 8-membered heterocycloalkyl, phenyl, benzyl, and monocyclic heteroaryl, wherein each alkyl, fluoroalkyl, cycloalkyl, heterocycloalkyl, phenyl, benzyl, and heteroaryl is unsubstituted or substituted with 1, 2, or 3 substituents selected from halogen, —CN, —OH, —O—(C₁-C₆ alkyl)₂, —NH₂, —NH(C₁-C₆ alkyl), —N(C₁-C₆ alkyl)₂, C₁-C₆ alkyl, C₁-C₆ fluoroalkyl, C₁-C₆ hydroxyalkyl, —O—(C₁-C₆ fluoroalkyl), C₃-C₆ cycloalkyl, 3- to 6-membered heterocycloalkyl, and

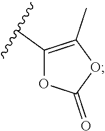 [SMILES: *c1oc(=O)oc1C]

or two R⁹ on the same N atom are taken together with the N atom to which they are attached to form a N-containing heterocycle, which is unsubstituted or substituted with 1, 2, or 3 substituents selected from halogen, —CN, —OH, —O—(C₁-C₆ alkyl), —NH₂, —NH(C₁-C₆ alkyl), —N(C₁-C₆ alkyl)₂, C₁-C₆ alkyl, C₁-C₆ fluoroalkyl, C₁-C₆ hydroxyalkyl, —O—(C₁-C₆ fluoroalkyl), C₃-C₆ cycloalkyl, and 3- to 6-membered heterocycloalkyl;
each R¹⁰ is independently selected from C₁-C₆ alkyl, C₁-C₆ fluoroalkyl, C₃-C₆ cycloalkyl, 3- to 8-membered heterocycloalkyl, phenyl, benzyl, and monocyclic heteroaryl, wherein each alkyl, fluoroalkyl, cycloalkyl, heterocycloalkyl, phenyl, benzyl, and heteroaryl is unsubstituted or substituted with 1, 2, or 3 substituents selected from halogen, —CN, —OH, —O—(C₁-C₆ alkyl), —NH₂, —NH(C₁-C₆ alkyl), —N(C₁-C₆ alkyl)₂, C₁-C₆ alkyl, C₁-C₆ fluoroalkyl, C₁-C₆ hydroxyalkyl, —O—(C₁-C₆ fluoroalkyl), C₃-C₆ cycloalkyl, 3- to 6-membered heterocycloalkyl, and

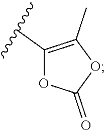 [SMILES: *c1oc(=O)oc1C]

m is 1 or 2;
n is 1 or 2;
p is 1-4; and
q is 0-4.

In some embodiments, when K is —B(OH)₂, W is —O—, —NR³—, or —C(R⁴)₂—.

In some embodiments, G is —P(═O)(OH)₂, —P(═O)(OH)(R^{D}), —P(═O)(OH)(H), —P(═O)(OH)(OR^{D}), —B(OH)₂, —B(OR^{D})(OH), —NHC(═O)H, —NHC(═O)(R^{D}), —NHS(═O)₂(R^{D}), —NHC(═O)NHS(═O)₂(R^{D}), —N(R^{D})C(═O)NHS(═O)₂(R^{D}), —C(═O)NHS(═O)₂(R^{D}), —S(═O)₂NHC(═O)(R^{D}), —NHC(═O)NH₂, —NHC(═O)NH(R^{D}), —NHC(═NH)NH₂, —NHC(═NH)NH(R^{D}), —NHC(═NH)N(R^{D})₂, —N(R^{D})C(═NH)NH₂, —N(R^{D})C(═NH)NH(R^{D}), —N(R^{D})C(═NH)N(R^{D})₂, —NHC(═N(R^{D}))NH₂, —NHC(═N(R^{D}))NH(R^{D}), —NHC(═N(R^{D}))N(R^{D})₂, —N(R^{D})C(═N(R^{D}))NH₂, —N(R^{D})C(═N(R^{D}))NH(R^{D}), —N(R^{D})C(═N(R^{D}))N(R^{D})₂—NHC(═NH)NHC(═NH)NH₂, —N(R^{D})C(═NH)NHC(═NH)NH₂,

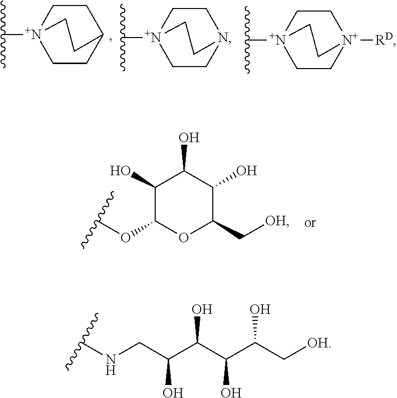

In some embodiments, each R¹ and R² is independently hydrogen, C₁₋₆ alkyl, or C₁₋₆ fluoroalkyl. In some embodiments, each R¹ and R² is independently hydrogen or C₁₋₆ alkyl. In some embodiments, each R¹ and R² is independently —H, —CH₃, —CH₂CH₃, —CH₂CH₂CH₃, —CH(CH₃)₂, —CH₂CH₂CH₂CH₃, —CH₂CH(CH₃)₂, —CH(CH₃)(CH₂CH₃), —C(CH₃)₃, —CH₂F, —CHF₂, —CF₃, —CH₂CH₂F, —CH₂CHF₂, or —CH₂CF₃. In some embodiments, each R¹ and R² is independently —H, —CH₃, —CH₂CH₃, or —CH₂CH₂CH₃. In some embodiments, each R¹ and R² is —H.

In some embodiments, one R¹ and one R² are taken together to form a ring. In some embodiments, one R¹ and one R² are taken together to form a 3- to 6-membered heterocycloalkyl ring.

In some embodiments, m is 1. In some embodiments, m is 2. In some embodiments, n is 1. In some embodiments, n is 2. In some embodiments, m is 1 and n is 1. In some embodiments, m is 1 and n is 2. In some embodiments, m is 2 and n is 1. In some embodiments, m is 2 and n is 2.

In some embodiments, Ring B is phenyl, naphthyl, monocyclic 6-membered heteroaryl, monocyclic 5-membered heteroaryl, or bicyclic heteroaryl.

In some embodiments, Ring B is phenyl or monocyclic heteroaryl. In some embodiments, Ring B is phenyl, monocyclic 6-membered heteroaryl, or monocyclic 5-membered heteroaryl. In some embodiments, Ring B is phenyl, pyridinyl, pyrimidinyl, pyrazinyl, pyridazinyl, triazinyl, furanyl, thienyl, pyrrolyl, oxazolyl, thiazolyl, imidazolyl, pyrazolyl, triazolyl, tetrazolyl, isoxazolyl, isothiazolyl, oxadiazolyl, or thiadiazolyl.

In some embodiments, Ring B is phenyl or 6-membered heteroaryl. In some embodiments, Ring B is phenyl, pyridinyl, pyrimidinyl, pyrazinyl, or pyridazinyl.

In some embodiments, Ring B is phenyl, or pyridinyl.

In some embodiments, Ring B is

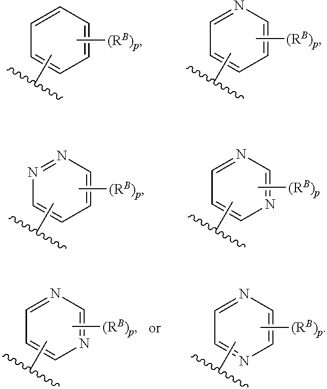

In some embodiments, Ring B is

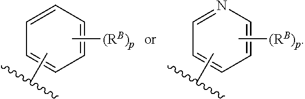

In some embodiments, Ring B is

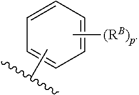

In some embodiments, Ring B is

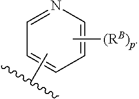

In some embodiments, Ring B is

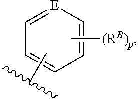

where E is CH or N.

In some embodiments, Ring B is phenyl or 6-membered heteroaryl; each R¹ and R² is independently hydrogen or C₁₋₆ alkyl; m is 2; and n is 2.

In some embodiments, the compound of Formula (II) has the structure of Formula (IIa), or a pharmaceutically acceptable salt, solvate, stereoisomer, or prodrug thereof:

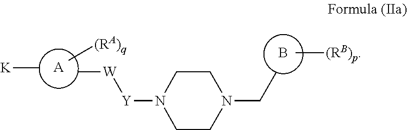

In some embodiments, the compound of Formula (II) has the structure of Formula (IIa-1), or a pharmaceutically acceptable salt, solvate, stereoisomer, or prodrug thereof:

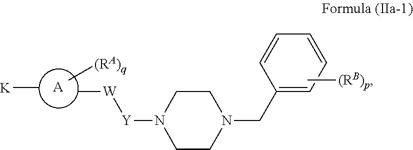

In some embodiments, the compound of Formula (II) has the structure of Formula (IIa-2), or a pharmaceutically acceptable salt, solvate, stereoisomer, or prodrug thereof:

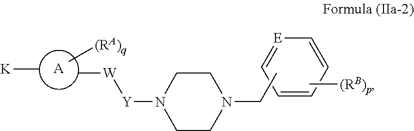

wherein E is CH or N.

In some embodiments, the compound of Formula (II) has the structure of Formula (IIa-3), or a pharmaceutically acceptable salt, solvate, stereoisomer, or prodrug thereof:

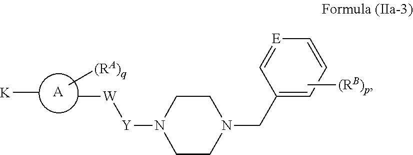

wherein E is CH or N.

In some embodiments, W is —O—. In some embodiments, W is —NR—. In some embodiments, W is —C(R⁴)₂—. In some embodiments, W is a bond.

In some embodiments, Y is —C(═O)—. In some embodiments, Y is —S(═O)₂—.

In some embodiments, W is a bond, and Y is —C(═O)—. In some embodiments, W is —O—, and Y is —C(═O)—. In some embodiments, W is —NR³—, and Y is —C(═O)—. In some embodiments, W is —C(R⁴)₂—; and Y is —C(═O)—. In some embodiments, W is a bond, and Y is —S(═O)₂—. In some embodiments, W is —O—, and Y is —S(═O)₂—. In some embodiments, W is —NR³—, and Y is —S(═O)₂—. In some embodiments, W is —C(R⁴)₂—; and Y is —S(═O)₂—.

In some embodiments, W is a bond, and Y is —C(═O)—; or W is —O—, and Y is —C(═O)—; or W is —NR³—, and Y is —C(═O)—; or W is —C(R⁴)₂—; and Y is —C(═O)—; or W is a bond, and Y is —S(═O)₂—; or W is —O—, and Y is —S(═O)₂—; or W is —NR³—, and Y is —S(═O)₂—; or W is —C(R⁴)₂—; and Y is —S(═O)₂—. In some embodiments, W is —O—, and Y is —C(═O)—; or W is —NR³—, and Y is —C(═O)—; or W is —C(R⁴)₂—; and Y is —C(═O)—; or W is —O—, and Y is —S(═O)₂—; or W is —NR³—, and Y is —S(═O)₂—; or W is —C(R⁴)₂—; and Y is —S(═O)₂—. In some embodiments, W is —O—, and Y is —C(═O)—; or W is —NR³—, and Y is —C(═O)—; or W is —C(R⁴)₂—; and Y is —C(═O)—; or W is a bond, and Y is —C(═O)—.

In some embodiments, the compound of Formula (II) has the structure of Formula (IIb), or a pharmaceutically acceptable salt, solvate, stereoisomer, or prodrug thereof:

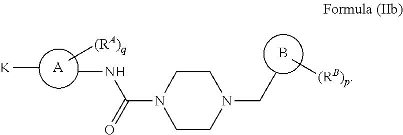

In some embodiments, the compound of Formula (II) has the structure of Formula (IIb-1), (IIb-2), or (IIb-3), or a pharmaceutically acceptable salt, solvate, stereoisomer, or prodrug thereof:

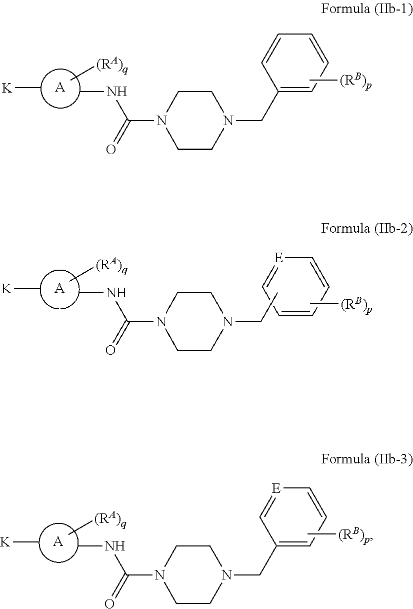

wherein E is CH or N.

In some embodiments, the compound of Formula (II) has the structure of Formula (IIb-1). In some embodiments, the compound of Formula (II) has the structure of Formula (IIb-2). In some embodiments, the compound of Formula (II) has the structure of Formula (IIb-3).

In some embodiments, the compound of Formula (II) has the structure of Formula (IIc), or a pharmaceutically acceptable salt, solvate, stereoisomer, or prodrug thereof:

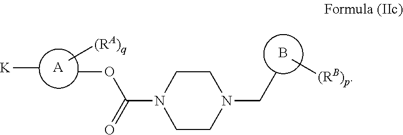

In some embodiments, the compound of Formula (II) has the structure of Formula (IIc-1), (IIc-2), or (IIc-3), or a pharmaceutically acceptable salt, solvate, stereoisomer, or prodrug thereof:

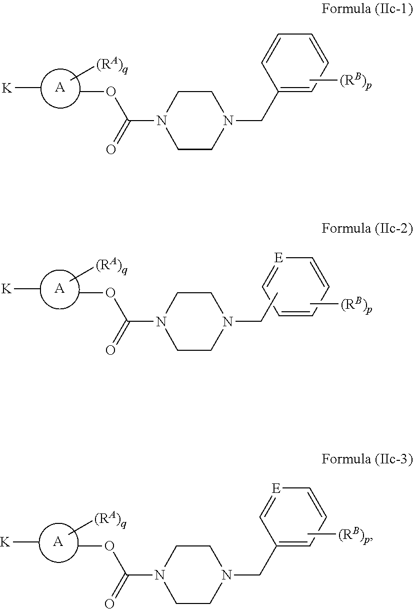

wherein E is CH or N.

In some embodiments, the compound of Formula (II) has the structure of Formula (IIc-1). In some embodiments, the compound of Formula (II) has the structure of Formula (IIc-2). In some embodiments, the compound of Formula (II) has the structure of Formula (IIc-3).

In some embodiments, the compound of Formula (I) has the structure of Formula (Id), or a pharmaceutically acceptable salt, solvate, stereoisomer, or prodrug thereof:

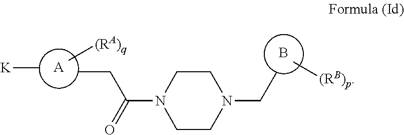

In some embodiments, the compound of Formula (II) has the structure of Formula (IId-1), (IId-2), or (IId-3), or a pharmaceutically acceptable salt, solvate, stereoisomer, or prodrug thereof:

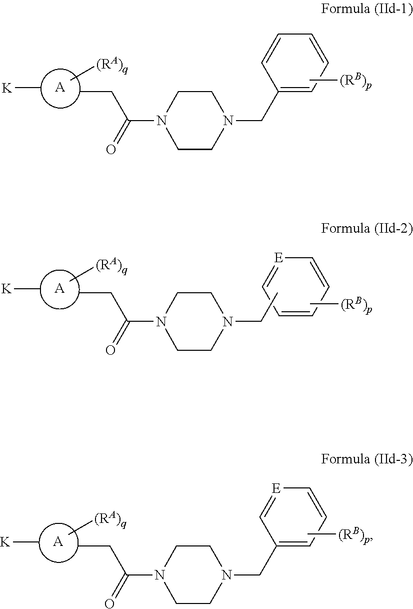

wherein E is CH or N.

In some embodiments, the compound of Formula (II) has the structure of Formula (IId-1). In some embodiments, the compound of Formula (II) has the structure of Formula (IId-2). In some embodiments, the compound of Formula (II) has the structure of Formula (IId-3).

In some embodiments, the compound of Formula (II) has the structure of Formula (IIe), or a pharmaceutically acceptable salt, solvate, stereoisomer, or prodrug thereof:

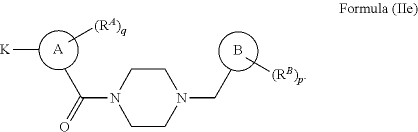

In some embodiments, the compound of Formula (II) has the structure of Formula (IIe-1), (IIe-2), or (IIe-3), or a pharmaceutically acceptable salt, solvate, stereoisomer, or prodrug thereof:

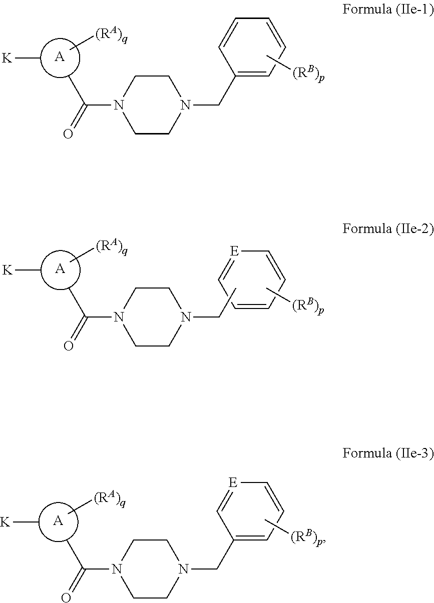

wherein E is CH or N.

In some embodiments, the compound of Formula (II) has the structure of Formula (IIb), Formula (IIc), Formula (IId), or Formula (IIe), or a pharmaceutically acceptable salt, solvate, stereoisomer, or prodrug thereof.

In some embodiments, each R^{B} is independently halogen, C₁-C₆ alkyl, phenyl, C₃-C₆ cycloalkyl, 3- to 6-membered heterocycloalkyl, 3- to 6-membered heterocycloalkenyl, 5-membered heteroaryl, 6-membered heteroaryl, —CN, —OR⁹, —CH₂CO₂R⁹, —CO₂R⁹, —C(═O)N(R⁹)₂, —N(R⁹)₂, —S(═O)₂R¹⁰, —S(═O)₂N(R⁹)₂, or —P(═O)(R¹⁰)₂, wherein each alkyl, phenyl, and heteroaryl is unsubstituted or substituted with 1, 2, or 3 substituents selected from halogen, —CN, —OH, —O—(C₁-C₆ alkyl), C₁-C₆ alkyl, C₁-C₆ fluoroalkyl, C₁-C₆ hydroxyalkyl, —O—(C₁-C₆ fluoroalkyl), C₃-C₆ cycloalkyl, and 3- to 6-membered heterocycloalkyl; and wherein each cycloalkyl, heterocycloalkyl, and heterocycloalkenyl is unsubstituted or substituted with 1, 2, or 3 substituents selected from halogen, —CN, —OH, ═O, —O—(C₁-C₆ alkyl), C₁-C₆ alkyl, C₁-C₆ fluoroalkyl, C₁-C₆ hydroxyalkyl, —O—(C₁-C₆ fluoroalkyl), C₃-C₆ cycloalkyl, and 3- to 6-membered heterocycloalkyl. In some embodiments, each R^{B} is independently halogen, C₁-C₆ alkyl, phenyl, C₃-C₆ cycloalkyl, 5-membered heteroaryl, 6-membered heteroaryl, —CN, —OR⁹, —CH₂CO₂R⁹, —CO₂R⁹, —C(═O)N(R⁹)₂, or —S(═O)₂R¹⁰, wherein each alkyl, cycloalkyl, phenyl, and heteroaryl is unsubstituted or substituted with 1, 2, or 3 substituents selected from —F, —Cl, —Br, —CN, —OH, —CH₂OH, —O—(C₁-C₆ alkyl), C₁-C₆ alkyl, C₁-C₆ fluoroalkyl. In some embodiments, each R^{B} is independently phenyl, oxadiazolyl, pyridinyl, —CN, —CH₂CO₂R⁹, —CO₂R⁹, or —S(═O)₂R¹⁰, wherein the phenyl, oxadiazolyl, or pyridinyl is unsubstituted or substituted with 1, 2, or 3 substituents selected from —F, —Cl, —Br, —CN, —OH, —CH₂OH, —O—(C₁-C₆ alkyl), C₁-C₆ alkyl, C₁-C₆ fluoroalkyl.

In some embodiments, p is 1. In some embodiments, p is 2. In some embodiments, p is 3. In some embodiments, p is 4. In some embodiments, p is 1-3. In some embodiments, p is 2 or 3.

In some embodiments, the compound of Formula (II) has the structure of Formula (IIf), or a pharmaceutically acceptable salt, solvate, stereoisomer, or prodrug thereof:

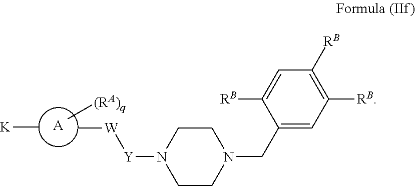

In some embodiments, the compound of Formula (II) has the structure of Formula (IIg), or a pharmaceutically acceptable salt, solvate, stereoisomer, or prodrug thereof:

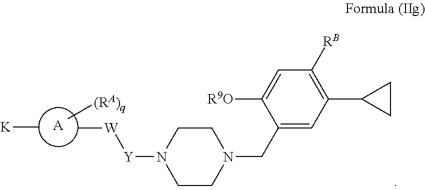

In some embodiments, R^{B} is phenyl, oxadiazolyl, pyridinyl, —CN, —CH₂CO₂R⁹, —CO₂R⁹, or —S(═O)₂R¹⁰, wherein the phenyl, oxadiazolyl, or pyridinyl is unsubstituted or substituted with 1, 2, or 3 substituents selected from —F, —Cl, —Br, —CN, —OH, —CH₂OH, —O—(C₁-C₆ alkyl), C₁-C₆ alkyl, C₁-C₆ fluoroalkyl.

In some embodiments, Ring A is phenyl, naphthyl, monocyclic 6-membered heteroaryl, monocyclic 5-membered heteroaryl, bicyclic heteroaryl, monocyclic C₃-C₈cycloalkyl, bridged C₅-C₁₀cycloalkyl, spiro C₅-C₁₀cycloalkyl, monocyclic C₂-C₈heterocycloalkyl, bridged C₅-C₁₀heterocycloalkyl, or spiro C₅-C₁₀heterocycloalkyl.

In some embodiments, Ring A is phenyl, monocyclic heteroaryl, monocyclic cycloalkyl, spirocyclic cycloalkyl, bridged cycloalkyl, monocyclic heterocycloalkyl, spirocyclic heterocycloalkyl, or bridged heterocycloalkyl. In some embodiments, Ring A is phenyl, monocyclic 6-membered heteroaryl, monocyclic 5-membered heteroaryl, monocyclic C₃-C₈cycloalkyl, bridged C₅-C₁₀cycloalkyl, spiro C₅-C₁₀cycloalkyl, monocyclic C₂-C₈heterocycloalkyl, bridged C₅-C₁₀heterocycloalkyl, or spiro C₅-C₁₀heterocycloalkyl.

In some embodiments, Ring A is phenyl or heteroaryl. In some embodiments, Ring A is phenyl or monocyclic heteroaryl. In some embodiments, Ring A is phenyl, monocyclic 6-membered heteroaryl, or monocyclic 5-membered heteroaryl. In some embodiments, Ring A is phenyl, pyridinyl, pyrimidinyl, pyrazinyl, pyridazinyl, triazinyl, furanyl, thienyl, pyrrolyl, oxazolyl, thiazolyl, imidazolyl, pyrazolyl, triazolyl, tetrazolyl, isoxazolyl, isothiazolyl, oxadiazolyl, or thiadiazolyl.

In some embodiments, Ring A is phenyl or 6-membered heteroaryl. In some embodiments, Ring A is phenyl, pyridinyl, pyrimidinyl, pyrazinyl, or pyridazinyl.

In some embodiments, Ring A is phenyl, monocyclic C₃-C₆ cycloalkyl, or bridged cycloalkyl. In some embodiments, Ring A is phenyl, monocyclic C₃-C₅ cycloalkyl, or bridged C₅-C₁₀ cycloalkyl. In some embodiments, Ring A is phenyl, cyclopropyl, cyclobutyl, cyclopentyl, cyclohexyl, or bridged C₅-C₁₀ cycloalkyl. In some embodiments, Ring A is phenyl, cyclohexyl, or

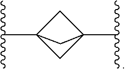 [SMILES: *C12CC(*)(C1)C2]

In some embodiments, Ring A is phenyl. In some embodiments, Ring A is cyclohexyl. In some embodiments, Ring A is

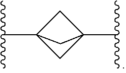 [SMILES: *C12CC(*)(C1)C2]

In some embodiments, Ring A is phenyl, naphthyl, indanyl, indenyl, tetrahyodronaphthyl, cyclopropyl, cyclobutyl, cyclopentyl, cyclopentenyl, cyclohexyl, cyclohexenyl, cycloheptyl, cyclooctyl, spiro[2.2]pentyl, spiro[3.3]heptyl, spiro[3.5]nonyl, spiro[4.4]nonyl, spiro[4.5]decyl, norbornyl, norbornenyl, bicyclo[1.1.1]pentyl, adamantyl, or decalinyl.

In some embodiments, Ring A is monocyclic cycloalkyl, spirocyclic cycloalkyl, bridged cycloalkyl, monocyclic heterocycloalkyl, spirocyclic heterocycloalkyl, or bridged heterocycloalkyl. In some embodiments, Ring A is cyclopropyl, cyclobutyl, cyclopentyl, cyclopentenyl, cyclohexyl, cyclohexenyl, cycloheptyl, cyclooctyl, spiro[2.2]pentyl, spiro[3.3]heptyl, spiro[3.5]nonyl, spiro[4.4]nonyl, spiro[4.5]decyl, norbornyl, norbornenyl, bicyclo[1.1.1]pentyl, adamantyl, or decalinyl. In some embodiments, Ring A is monocyclic C₃-C₆cycloalkyl, or bridged cycloalkyl. In some embodiments, Ring A is cyclopropyl, cyclobutyl, cyclopentyl, cyclohexyl, or bridged C₅-C₁₀cycloalkyl. In some embodiments, Ring A is cyclohexyl or

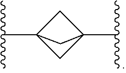 [SMILES: *C12CC(*)(C1)C2]

In some embodiments, Ring A is furanyl, thienyl, pyrrolyl, oxazolyl, thiazolyl, imidazolyl, pyrazolyl, triazolyl, tetrazolyl, isoxazolyl, isothiazolyl, oxadiazolyl, thiadiazolyl, pyridinyl, pyrimidinyl, pyrazinyl, pyridazinyl, triazinyl, quinolinyl, isoquinolinyl, cinnolinyl, phthalazinyl, quinazolinyl, quinoxalinyl, naphthyridinyl, pteridinyl, indolizinyl, azaindolizinyl, indolyl, azaindolyl, indazolyl, azaindazolyl, benzimidazolyl, azabenzimidazolyl, benzotriazolyl, azabenzotriazolyl, benzoxazolyl, azabenzoxazolyl, benzisoxazolyl, azabenzisoxazolyl, benzofuranyl, azabenzofuranyl, benzothienyl, azabenzothienyl, benzothiazolyl, azabenzothiazolyl, or purinyl.

In some embodiments, Ring A is aziridinyl, azetidinyl, oxetanyl, thietanyl, pyrrolidinyl, tetrahydrofuranyl, tetrahydrothienyl, oxazolidinonyl, tetrahydropyranyl, piperidinyl, morpholinyl, thiomorpholinyl, piperazinyl, homopiperidinyl, oxepanyl, thiepanyl, oxazepinyl, diazepinyl, thiazepinyl, azaspiro[3.3]heptanyl, azaspiro[3.4]octanyl, azaspiro[3.4]octanyl, or azaspiro[4.4]nonyl.

In some embodiments, Ring A is phenyl, pyridinyl, pyrimidinyl, pyrazinyl, or pyridazinyl.

In some embodiments, Ring A is an aziridinyl, azetidinyl, oxetanyl, thietanyl, pyrrolidinyl, tetrahydrofuranyl, tetrahydrothienyl, oxazolidinonyl, tetrahydropyranyl, piperidinyl, morpholinyl, thiomorpholinyl, or piperazinyl.

In some embodiments, each R^{A} is independently halogen, —OH, —O—(C₁-C₆ alkyl), C₁-C₆ alkyl, C₃-C₆ cycloalkyl, wherein each alkyl and cycloalkyl is unsubstituted or substituted with 1, 2, or 3 substituents selected from halogen, —CN, —OH, —O—(C₁-C₆ alkyl), C₁-C₆ alkyl, and C₁-C₆ fluoroalkyl. In some embodiments, each R^{A} is independently halogen, —OH, —O—(C₁-C₆ alkyl), or C₁-C₆ alkyl. In some embodiments, each R^{A} is independently —F, —Cl, —Br, —OH, —OCH₃, —OCH₂CH₃, —OCH₂CH₂CH₃, —OCH(CH₃)₂, —CH₃, —CH₂CH₃, —CH₂CH₂CH₃, —CH(CH₃)₂, —CH₂CH₂CH₂CH₃, —CH₂CH(CH₃)₂, —CH(CH₃)(CH₂CH₃), or —C(CH₃)₃. In some embodiments, each R^{A} is independently C₁-C₆ alkyl. In some embodiments, each R^{A} is independently —CH₃, —CH₂CH₃, —CH₂CH₂CH₃, —CH(CH₃)₂, —CH₂CH₂CH₂CH₃, —CH₂CH(CH₃)₂, —CH(CH₃)(CH₂CH₃), or —C(CH₃)₃.

In some embodiments, q is 0. In some embodiments, q is 1-4. In some embodiments, q is 0-2. In some embodiments, q is 0-1. In some embodiments, q is 1. In some embodiments, q is 2. In some embodiments, q is 3. In some embodiments, q is 4.

In some embodiments, Ring A is phenyl, monocyclic heteroaryl, monocyclic cycloalkyl, spirocyclic cycloalkyl, bridged cycloalkyl, monocyclic heterocycloalkyl, spirocyclic heterocycloalkyl, or bridged heterocycloalkyl; each R^{A} is independently halogen, —OH, —O—(C₁-C₆ alkyl), C₁-C₆ alkyl, C₃-C₆ cycloalkyl, wherein each alkyl and cycloalkyl is unsubstituted or substituted with 1, 2, or 3 substituents selected from halogen, —CN, —OH, —O—(C₁-C₆ alkyl), C₁-C₆ alkyl, and C₁-C₆ fluoroalkyl; and q is 0-2.

In some embodiments, Ring A is phenyl, monocyclic C₃-C₆ cycloalkyl, or bridged cycloalkyl; each R^{A} is independently halogen, —OH, —O—(C₁-C₆ alkyl), or C₁-C₆ alkyl; and q is 0-2.

In some embodiments, Ring A is phenyl, cyclohexyl, or

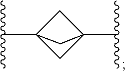 [SMILES: *C12CC(*)(C1)C2]

each R^{A} is independently halogen, —OH, —O—(C₁-C₆ alkyl), or C₁-C₆ alkyl; and q is 0-2.

In some embodiments, Ring A is phenyl; and q is 0.

In some embodiments, the compound of Formula (II) has the structure of Formula (IIh), or a pharmaceutically acceptable salt, solvate, stereoisomer, or prodrug thereof:

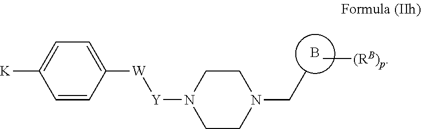

In some embodiments, the compound of Formula (II) has the structure of Formula (IIh-1), (IIh-2), or (IIh-3), or a pharmaceutically acceptable salt, solvate, stereoisomer, or prodrug thereof:

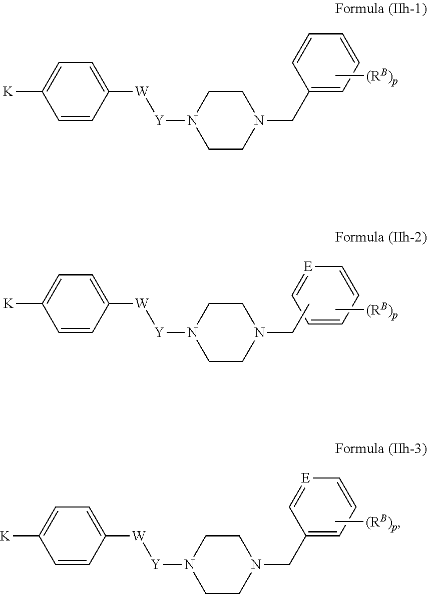

wherein E is CH or N.

In some embodiments, the compound of Formula (II) has the structure of Formula (IIh-1). In some embodiments, the compound of Formula (II) has the structure of Formula (IIh-2). In some embodiments, the compound of Formula (II) has the structure of Formula (IIh-3).

In some embodiments, the compound of Formula (IIi) has the structure of Formula (IIi), or a pharmaceutically acceptable salt, solvate, stereoisomer, or prodrug thereof:

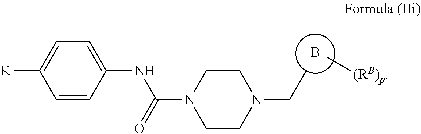

In some embodiments, the compound of Formula (II) has the structure of Formula (IIi-1), (IIi-2), or (IIi-3), or a pharmaceutically acceptable salt, solvate, stereoisomer, or prodrug thereof:

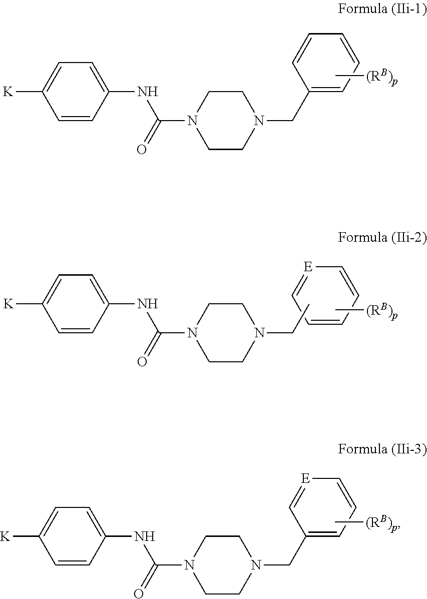

wherein E is CH or N.

In some embodiments, the compound of Formula (II) has the structure of Formula (IIi-1). In some embodiments, the compound of Formula (II) has the structure of Formula (IIi-2). In some embodiments, the compound of Formula (II) has the structure of Formula (IIi-3).

In some embodiments, the compound of Formula (II) has the structure of Formula (IIj), or a pharmaceutically acceptable salt, solvate, stereoisomer, or prodrug thereof:

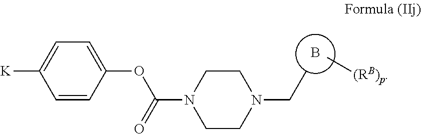

In some embodiments, the compound of Formula (II) has the structure of Formula (IIj-1), (IIj-2), or (IIj-3), or a pharmaceutically acceptable salt, solvate, stereoisomer, or prodrug thereof:

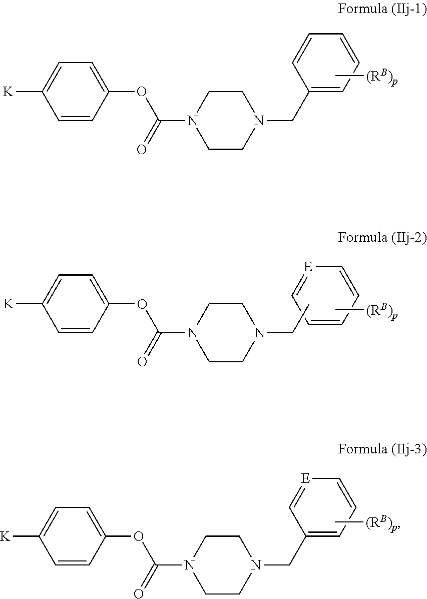

wherein E is CH or N.

In some embodiments, the compound of Formula (II) has the structure of Formula (IIj-1). In some embodiments, the compound of Formula (II) has the structure of Formula (IIj-2). In some embodiments, the compound of Formula (II) has the structure of Formula (IIj-3).

In some embodiments, the compound of Formula (I) has the structure of Formula (Ik), or a pharmaceutically acceptable salt, solvate, stereoisomer, or prodrug thereof:

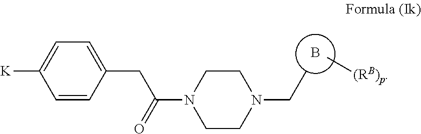

In some embodiments, the compound of Formula (II) has the structure of Formula (Ilk-1), (IIk-2), or (IIk-3), or a pharmaceutically acceptable salt, solvate, stereoisomer, or prodrug thereof:

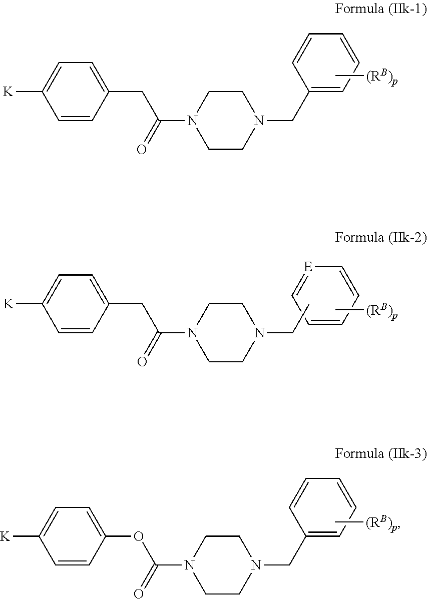

wherein E is CH or N.

In some embodiments, the compound of Formula (II) has the structure of Formula (Ilk-1). In some embodiments, the compound of Formula (II) has the structure of Formula (Ilk-2). In some embodiments, the compound of Formula (II) has the structure of Formula (IIk-3).

In some embodiments, the compound of Formula (II) has the structure of Formula (IIl), or a pharmaceutically acceptable salt, solvate, stereoisomer, or prodrug thereof:

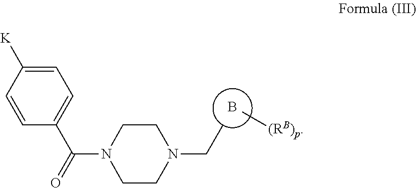

In some embodiments, the compound of Formula (II) has the structure of Formula (IIl-1), (IIl-2), or (IIl-3), or a pharmaceutically acceptable salt, solvate, stereoisomer, or prodrug thereof:

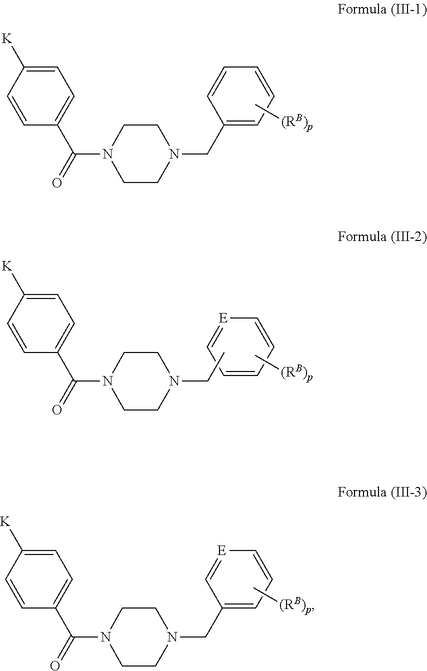

wherein E is CH or N.

In some embodiments, the compound of Formula (II) has the structure of Formula (IIl-1). In some embodiments, the compound of Formula (II) has the structure of Formula (IIl-2). In some embodiments, the compound of Formula (II) has the structure of Formula (IIl-3).

In some embodiments, K is —(CH₂)ⱼ-G. In some embodiments, K is —(CH₂)ⱼ-G and j is 0 or 1.

In some embodiments, j is 0 or 1. In some embodiments, j is 0. In some embodiments, j is 1. In some embodiments, j is 2. In some embodiments, j is 3. In some embodiments, j is 4.

In some embodiments, G is —P(═O)(OH)₂, —P(═O)(OH)(R^{D}), —P(═O)(OH)(OR^{D}), —B(OH)₂, —B(OR^{D})(OH), —NHC(═O)H, —NHC(═O)(R^{D}), —NHS(═O)₂(R^{D}), —NHC(═O)NH₂, —NHC(═O)NH(R^{D}), —N(R^{D})C(═O)NHS(═O)₂(R^{D}), or —C(═O)NHS(═O)₂(R^{D}). In some embodiments, G is —P(═O)(OH)₂, —P(═O)(OH)(R^{D}), —P(═O)(OH)(OR^{D}), —B(OH)₂, —B(OR^{D})(OH), —NHC(═O)H, —NHC(═O)(R^{D}), —NHS(═O)₂(R^{D}), —NHC(═O)NH₂, —NHC(═O)NH(R^{D}), —N(R^{D})C(═O)NHS(═O)₂(R^{D}), or —C(═O)NHS(═O)₂(R^{D}); and R^{D} is alkyl which is unsubstituted or substituted with 1, 2, or 3 —OH groups.

In some embodiments, G is —P(═O)(OH)₂, —P(═O)(OH)(R^{D}), or —P(═O)(OH)(OR^{D}). In some embodiments, G is —P(═O)(OH)₂, —P(═O)(OH)(R^{D}), or —P(═O)(OH)(OR^{D}); and R^{D} is C₁₋₆ alkyl. In some embodiments, G is —P(═O)(OH)₂, —P(═O)(OH)(R^{D}), or —P(═O)(OH)(OR^{D}); R^{D} is C₁₋₆ alkyl; and j is 0 or 1

In some embodiments, K is —(CH₂)ⱼP(═O)(OH)₂, —(CH₂)ⱼP(═O)(OMe)(OH), —(CH₂)ⱼP(═O)(OEt)(OH), —(CH₂)ⱼP(═O)(Me)(OH), or —(CH₂)ⱼP(═O)(Et)(OH). In some embodiments, K is —(CH₂)ⱼP(═O)(OH)₂, —(CH₂)ⱼP(═O)(OMe)(OH), —(CH₂)ⱼP(═O)(OEt)(OH), —(CH₂)ⱼP(═O)(Me)(OH), or —(CH₂)ⱼP(═O)(Et)(OH); and j is 0 or 1. In some embodiments, K is —P(═O)(OH)₂, —P(═O)(OEt)(OH), or —P(═O)(Me)(OH).

In some embodiments, Ring B is

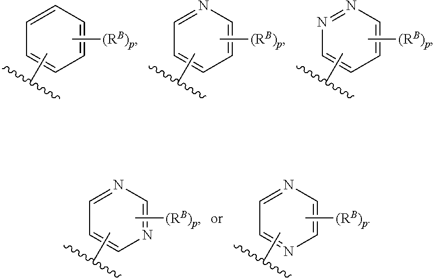

In some embodiments, Ring B is

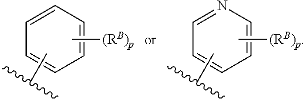

In some embodiments, Ring B is

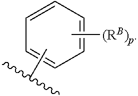

In some embodiments, Ring B is

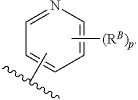

In some embodiments, Ring B is phenyl, or pyridinyl.

In some embodiments, Ring B is

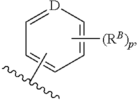

where D is CH or N.

In some embodiments, each R^{B} is independently C₁-C₆ alkyl, C₃-C₆ cycloalkyl, aryl, heteroaryl, —OR⁹, —CO₂R⁹, or —S(═O)₂R¹⁰, wherein each alkyl, aryl, and heteroaryl is unsubstituted or substituted with 1, 2, or 3 substituents selected from halogen, —CN, —OH, —O—(C₁-C₆ alkyl), —CO₂—(C₁-C₆ alkyl), C₁-C₆ alkyl, C₁-C₆ fluoroalkyl, C₁-C₆ hydroxyalkyl, —O—(C₁-C₆ fluoroalkyl), C₃-C₆ cycloalkyl, and 3- to 6-membered heterocycloalkyl; and p is 1-4. In some embodiments, each R^{B} is independently C₁-C₆ alkyl, C₃-C₆ cycloalkyl, aryl, heteroaryl, —OR⁹, —CO₂R⁹, or —S(═O)₂R¹⁰, wherein each alkyl, aryl, and heteroaryl is unsubstituted or substituted with 1 halogen or C₁-C₆ alkyl. In some embodiments, at least one R^{B} is phenyl, pyridinyl, pyrimidinyl, pyridazinyl, or pyrazinyl, unsubstituted or substituted with 1, 2, or 3 halogen. In some embodiments, at least one R^{B} is fluorophenyl, fluoropyridinyl, or fluoropyrimidinyl. In some embodiments, at least one R^{B} is C₁-C₆ alkyl or C₃-C₆ cycloalkyl. In some embodiments, at least one R^{B} is methyl, ethyl, n-propyl, isopropyl, cyclopropyl, n-butyl, tert-butyl, sec-butyl, isobutyl, or cyclobutyl. In some embodiments, at least one R^{B} is ethyl, isopropyl, cyclopropyl, tert-butyl, isobutyl, or cyclobutyl. In some embodiments, at least one R^{B} is isopropyl, cyclopropyl, or cyclobutyl. In some embodiments, at least one R^{B} is —OR⁹. In some embodiments, at least one R^{B} is —S(═O)₂R¹⁰. In some embodiments, at least one R^{B} is —CO₂R⁹. In some embodiments, R⁹ is C₁-C₆ alkyl.

Any combination of the groups described above for the various variables is contemplated herein. Throughout the specification, groups and substituents thereof are chosen by one skilled in the field to provide stable moieties and compounds.

Exemplary compounds of Formulas (I) or Formula (II) include the compounds described in the following tables.

TABLE 1

| Ex. # | Structure | Name |
|---|---|---|
| 1 | 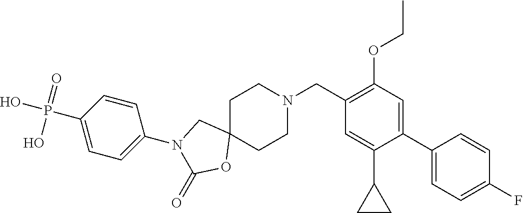 [SMILES: CCOc1cc(-c2ccc(F)cc2)c(C2CC2)cc1CN1CCC2(CC1)CN(c1ccc(P(=O)(O)O)cc1)C(=O)O2] | (4-(8-((2-cyclopropyl-5-ethoxy-4′-fluoro-[1,1′- biphenyl]-4-yl)methyl)-2-oxo-1-oxa-3,8- diazaspiro[4.5]decan-3-yl)phenyl)phosphonic acid |
| 2 | 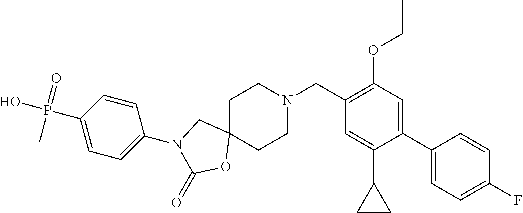 [SMILES: CCOc1cc(-c2ccc(F)cc2)c(C2CC2)cc1CN1CCC2(CC1)CN(c1ccc(P(C)(=O)O)cc1)C(=O)O2] | (4-(8-((2-cyclopropyl-5-ethoxy-4′-fluoro-[1,1′- biphenyl]-4-yl)methyl)-2-oxo-1-oxa-3,8- diazaspiro[4.5]decan-3- yl)phenyl)(methyl)phosphinic acid |
| 3 | 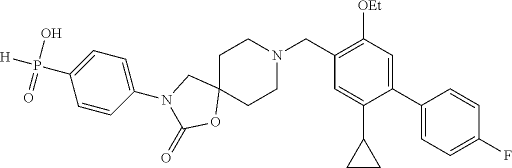 [SMILES: CCOc1cc(-c2ccc(F)cc2)c(C2CC2)cc1CN1CCC2(CC1)CN(c1ccc([PH](=O)O)cc1)C(=O)O2] | (4-(8-((2-cyclopropyl-5-ethoxy-4′-fluoro-[1,1′- biphenyl]-4-yl)methyl)-2-oxo-1-oxa-3,8- diazaspiro[4.5]decan-3-yl)phenyl)phosphinic acid |
| 4 | 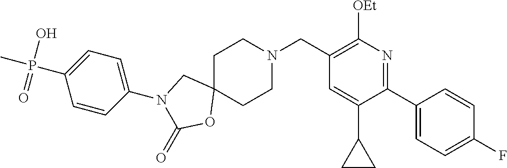 [SMILES: CCOc1nc(-c2ccc(F)cc2)c(C2CC2)cc1CN1CCC2(CC1)CN(c1ccc(P(C)(=O)O)cc1)C(=O)O2] | (4-(8-((5-cyclopropyl-2-ethoxy-6-(4- fluorophenyl)pyridin-3-yl)methyl)-2-oxo-1-oxa-3,8- diazaspiro[4.5]decan-3- yl)phenyl)(methyl)phosphinic acid |
| 5 | 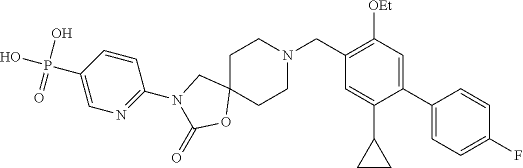 [SMILES: CCOc1cc(-c2ccc(F)cc2)c(C2CC2)cc1CN1CCC2(CC1)CN(c1ccc(P(=O)(O)O)cn1)C(=O)O2] | (6-(8-((2-cyclopropyl-5-ethoxy-4′-fluoro-[1,1′- biphenyl]-4-yl)methyl)-2-oxo-1-oxa-3,8- diazaspiro[4.5]decan-3-yl)pyridin-3-yl)phosphonic acid |
| 6 | 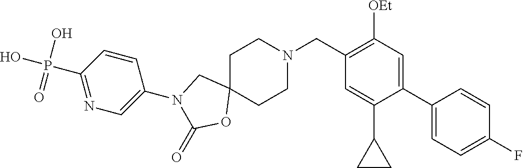 [SMILES: CCOc1cc(-c2ccc(F)cc2)c(C2CC2)cc1CN1CCC2(CC1)CN(c1ccc(P(=O)(O)O)nc1)C(=O)O2] | (5-(8-((2-cyclopropyl-5-ethoxy-4′-fluoro-[1,1′- biphenyl]-4-yl)methyl)-2-oxo-1-oxa-3,8- diazaspiro[4.5]decan-3-yl)pyridin-2-yl)phosphonic acid |
| 7 | 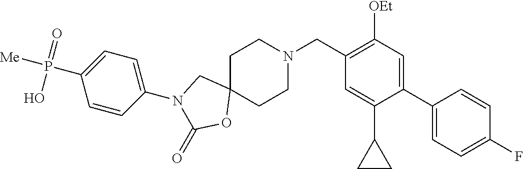 [SMILES: CCOc1cc(-c2ccc(F)cc2)c(C2CC2)cc1CN1CCC2(CC1)CN(c1ccc(P(C)(=O)O)cc1)C(=O)O2] | (4-(8-((2-cyclopropyl-5-ethoxy-4′-fluoro-[1,1′- biphenyl]-4-yl)methyl)-3-oxo-2,8- diazaspiro[4.5]decan-2- yl)phenyl)(methyl)phosphinic acid |
| 8 | 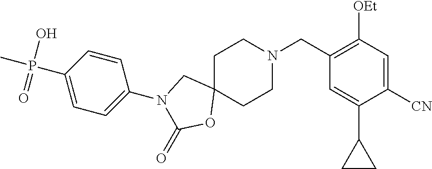 [SMILES: CCOc1cc(C#N)c(C2CC2)cc1CN1CCC2(CC1)CN(c1ccc(P(C)(=O)O)cc1)C(=O)O2] | (4-(8-(4-cyano-5-cyclopropyl-2-ethoxybenzyl)-2- oxo-1-oxa-3,8-diazaspiro[4.5]decan-3- yl)phenyl)(methyl)phosphinic acid |
| 9 | 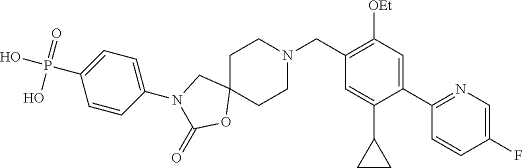 [SMILES: CCOc1cc(-c2ccc(F)cn2)c(C2CC2)cc1CN1CCC2(CC1)CN(c1ccc(P(=O)(O)O)cc1)C(=O)O2] | (4-(8-(5-cyclopropyl-2-ethoxy-4-(5-fluoropyridin-2- yl)benzyl)-2-oxo-1-oxa-3,8-diazaspiro[4.5]decan-3- yl)phenyl)phosphonic acid |
| 10 | 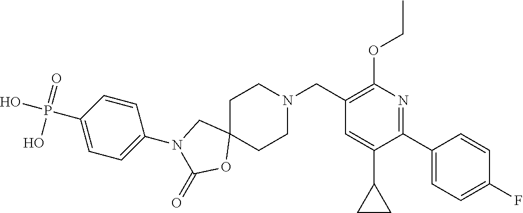 [SMILES: CCOc1nc(-c2ccc(F)cc2)c(C2CC2)cc1CN1CCC2(CC1)CN(c1ccc(P(=O)(O)O)cc1)C(=O)O2] | (4-(8-((5-cyclopropyl-2-ethoxy-6-(4- fluorophenyl)pyridin-3-yl)methyl)-2-oxo-1-oxa-3,8- diazaspiro[4.5]decan-3-yl)phenyl)phosphonic acid |
| 11 | 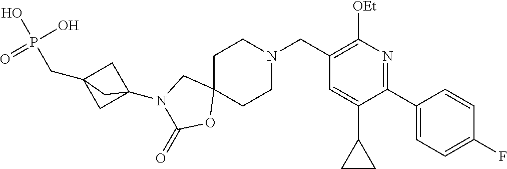 [SMILES: CCOc1nc(-c2ccc(F)cc2)c(C2CC2)cc1CN1CCC2(CC1)CN(C13CC(CP(=O)(O)O)(C1)C3)C(=O)O2] | ((3-(8-((5-cyclopropyl-2-ethoxy-6-(4- fluorophenyl)pyridin-3-yl)methyl)-2-oxo-1-oxa-3,8- diazaspiro[4.5]decan-3-yl)bicyclo[1.1.1]pentan-1- yl)methyl)phosphonic acid |
| 12 | 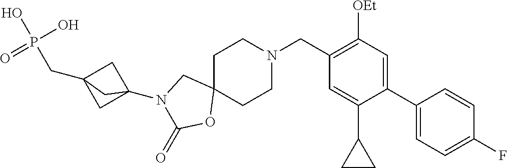 [SMILES: CCOc1cc(-c2ccc(F)cc2)c(C2CC2)cc1CN1CCC2(CC1)CN(C13CC(CP(=O)(O)O)(C1)C3)C(=O)O2] | ((3-(8-((2-cyclopropyl-5-ethoxy-4′-fluoro-[1,1′- biphenyl]-4-yl)methyl)-2-oxo-1-oxa-3,8- diazaspiro[4.5]decan-3-yl)bicyclo[1.1.1]pentan-1- yl)methyl)phosphonic acid |
| 13 | 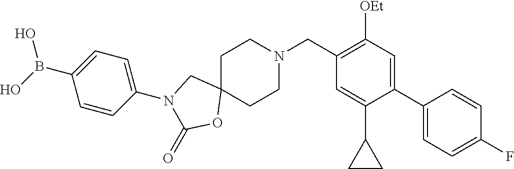 [SMILES: CCOc1cc(-c2ccc(F)cc2)c(C2CC2)cc1CN1CCC2(CC1)CN(c1ccc(B(O)O)cc1)C(=O)O2] | (4-(8-((2-cyclopropyl-5-ethoxy-4′-fluoro-[1,1′- biphenyl]-4-yl)methyl)-2-oxo-1-oxa-3,8- diazaspiro[4.5]decan-3-yl)phenyl)boronic acid |
| 14 | 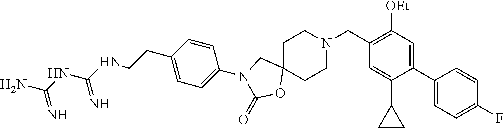 [SMILES: CCOc1cc(-c2ccc(F)cc2)c(C2CC2)cc1CN1CCC2(CC1)CN(c1ccc(CCNC(=N)NC(=N)N)cc1)C(=O)O2] | 1-carbamimidoyl-3-[2-[4-[8-[[5-cyclopropyl-2- ethoxy-4-(4-fluorophenyl)phenyl]methyl]-2-oxo-1- oxa-3,8-diazaspiro[4.5]decan-3- yl]phenyl]ethyl]guanidine |
| 15 | 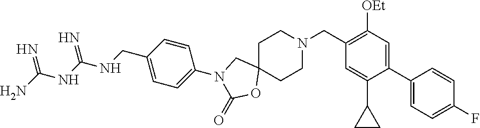 [SMILES: CCOc1cc(-c2ccc(F)cc2)c(C2CC2)cc1CN1CCC2(CC1)CN(c1ccc(CNC(=N)NC(=N)N)cc1)C(=O)O2] | 1-carbamimidoyl-3-[[4-[8-[[5-cyclopropyl-2- ethoxy-4-(4-fluorophenyl)phenyl]methyl]-2-oxo-1- oxa-3,8-diazaspiro[4.5]decan-3- yl]phenyl]methyl]guanidine |
| 16 | 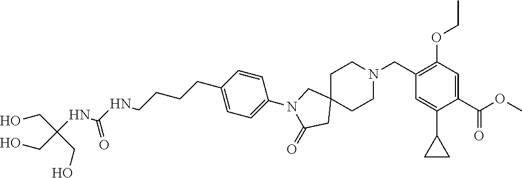 [SMILES: CCOc1cc(C(=O)OC)c(C2CC2)cc1CN1CCC2(CC1)CC(=O)N(c1ccc(CCCCNC(=O)NC(CO)(CO)CO)cc1)C2] | methyl 2-cyclopropyl-4-((2-(4-(4-(3-(1,3- dihydroxy-2-(hydroxymethyl)propan-2- yl)ureido)butyl)phenyl)-3-oxo-2,8- diazaspiro[4.5]decan-8-yl)methyl)-5- ethoxybenzoate |
| 17 | 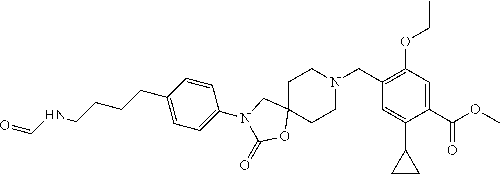 [SMILES: CCOc1cc(C(=O)OC)c(C2CC2)cc1CN1CCC2(CC1)CN(c1ccc(CCCCNC=O)cc1)C(=O)O2] | methyl 2-cyclopropyl-5-ethoxy-4-((2-(4-(4- formamidobutyl)phenyl)-3-oxo-2,8- diazaspiro[4.5]decan-8-yl)methyl)benzoate |
| 18 | 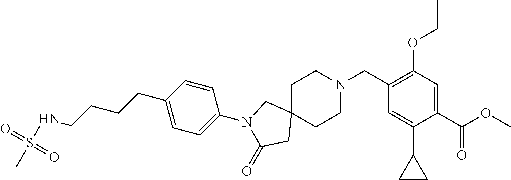 [SMILES: CCOc1cc(C(=O)OC)c(C2CC2)cc1CN1CCC2(CC1)CC(=O)N(c1ccc(CCCCNS(C)(=O)=O)cc1)C2] | methyl 2-cyclopropyl-5-ethoxy-4-((2-(4-(4- (methylsulfonamido)butyl)phenyl)-3-oxo-2,8- diazaspiro[4.5]decan-8-yl)methyl)benzoate |
| 19 | 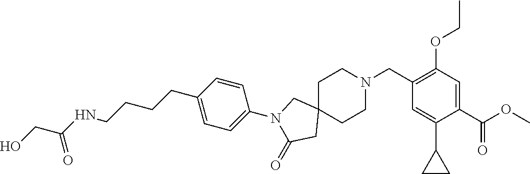 [SMILES: CCOc1cc(C(=O)OC)c(C2CC2)cc1CN1CCC2(CC1)CC(=O)N(c1ccc(CCCCNC(=O)CO)cc1)C2] | methyl 2-cyclopropyl-5-ethoxy-4-((2-(4-(4-(2- hydroxyacetamido)butyl)phenyl)-3-oxo-2,8- diazaspiro[4.5]decan-8-yl)methyl)benzoate |
| 20 | 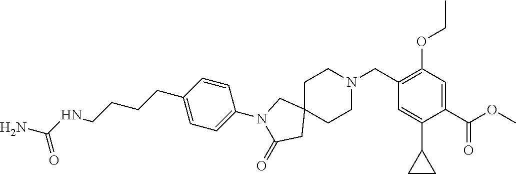 [SMILES: CCOc1cc(C(=O)OC)c(C2CC2)cc1CN1CCC2(CC1)CC(=O)N(c1ccc(CCCCNC(N)=O)cc1)C2] | methyl 2-cyclopropyl-5-ethoxy-4-((3-oxo-2-(4-(4- ureidobutyl)phenyl)-2,8-diazaspiro[4.5]decan-8- yl)methyl)benzoate |
| 21 | 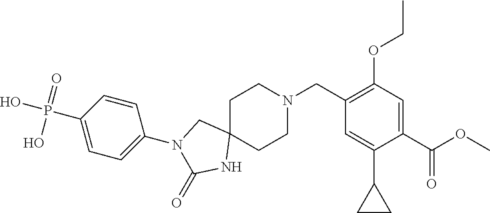 [SMILES: CCOc1cc(C(=O)OC)c(C2CC2)cc1CN1CCC2(CC1)CN(c1ccc(P(=O)(O)O)cc1)C(=O)N2] | (4-(8-(5-cyclopropyl-2-ethoxy-4- (methoxycarbonyl)benzyl)-2-oxo-1,3,8- triazaspiro[4.5]decan-3-yl)phenyl)phosphonic acid |
| 22 | 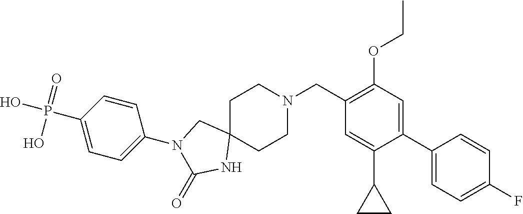 [SMILES: CCOc1cc(-c2ccc(F)cc2)c(C2CC2)cc1CN1CCC2(CC1)CN(c1ccc(P(=O)(O)O)cc1)C(=O)N2] | (4-(8-((2-cyclopropyl-5-ethoxy-4′-fluoro-[1,1′- biphenyl]-4-yl)methyl)-2-oxo-1,3,8- triazaspiro[4.5]decan-3-yl)phenyl)phosphonic acid |
| 23 | 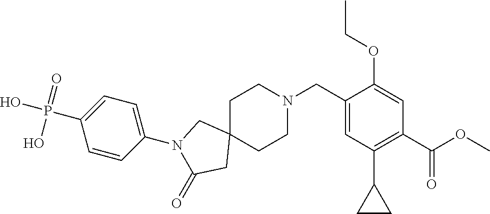 [SMILES: CCOc1cc(C(=O)OC)c(C2CC2)cc1CN1CCC2(CC1)CC(=O)N(c1ccc(P(=O)(O)O)cc1)C2] | (4-(8-(5-cyclopropyl-2-ethoxy-4- (methoxycarbonyl)benzyl)-3-oxo-2,8- diazaspiro[4.5]decan-2-yl)phenyl)phosphonic acid |
| 24 | 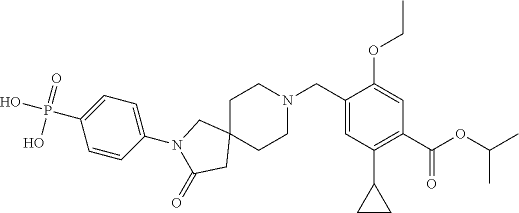 [SMILES: CCOc1cc(C(=O)OC(C)C)c(C2CC2)cc1CN1CCC2(CC1)CC(=O)N(c1ccc(P(=O)(O)O)cc1)C2] | (4-(8-(5-cyclopropyl-2-ethoxy-4- (isopropoxycarbonyl)benzyl)-3-oxo-2,8- diazaspiro[4.5]decan-2-yl)phenyl)phosphonic acid |
| 25 | 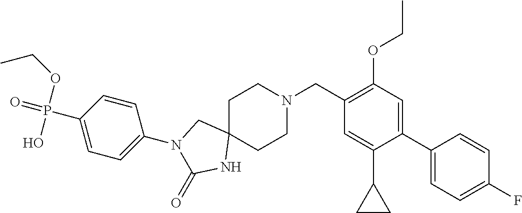 [SMILES: CCOc1cc(-c2ccc(F)cc2)c(C2CC2)cc1CN1CCC2(CC1)CN(c1ccc(P(=O)(O)OCC)cc1)C(=O)N2] | ethyl hydrogen (4-(8-((2-cyclopropyl-5-ethoxy-4′- fluoro-[1,1′-biphenyl]-4-yl)methyl)-2-oxo-1,3,8- triazaspiro[4.5]decan-3-yl)phenyl)phosphonate |
| 26 | 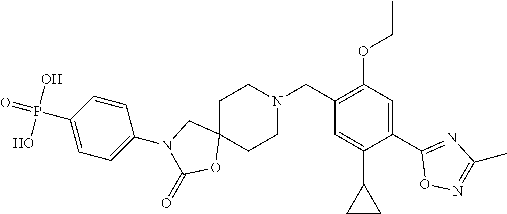 [SMILES: CCOc1cc(-c2nc(C)no2)c(C2CC2)cc1CN1CCC2(CC1)CN(c1ccc(P(=O)(O)O)cc1)C(=O)O2] | (4-(8-(5-cyclopropyl-2-ethoxy-4-(3-methyl-1,2,4- oxadiazol-5-yl)benzyl)-2-oxo-1-oxa-3,8- diazaspiro[4.5]decan-3-yl)phenyl)phosphonic acid |
| 27 | 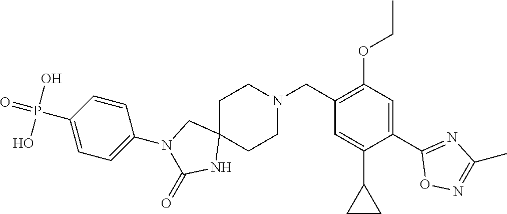 [SMILES: CCOc1cc(-c2nc(C)no2)c(C2CC2)cc1CN1CCC2(CC1)CN(c1ccc(P(=O)(O)O)cc1)C(=O)N2] | (4-(8-(5-cyclopropyl-2-ethoxy-4-(3-methyl-1,2,4- oxadiazol-5-yl)benzyl)-2-oxo-1,3,8- triazaspiro[4.5]decan-3-yl)phenyl)phosphonic acid |
| 28 | 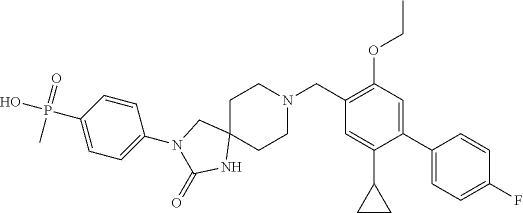 [SMILES: CCOc1cc(-c2ccc(F)cc2)c(C2CC2)cc1CN1CCC2(CC1)CN(c1ccc(P(C)(=O)O)cc1)C(=O)N2] | (4-(8-((2-cyclopropyl-5-ethoxy-4′-fluoro-[1,1′- biphenyl]-4-yl)methyl)-2-oxo-1,3,8- triazaspiro[4.5]decan-3- yl)phenyl)(methyl)phosphinic acid |
| 29 | 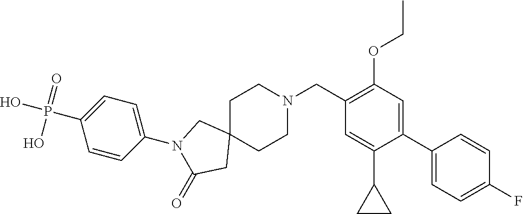 [SMILES: CCOc1cc(-c2ccc(F)cc2)c(C2CC2)cc1CN1CCC2(CC1)CC(=O)N(c1ccc(P(=O)(O)O)cc1)C2] | (4-(8-((2-cyclopropyl-5-ethoxy-4′-fluoro-[1,1′- biphenyl]-4-yl)methyl)-3-oxo-2,8- diazaspiro[4.5]decan-2-yl)phenyl)phosphonic acid |
| 30 | 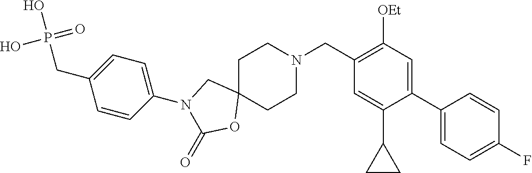 [SMILES: CCOc1cc(-c2ccc(F)cc2)c(C2CC2)cc1CN1CCC2(CC1)CN(c1ccc(CP(=O)(O)O)cc1)C(=O)O2] | (4-(8-((2-cyclopropyl-5-ethoxy-4'-fluoro-[1,1'- biphenyl]-4-yl)methyl)-2-oxo-1-oxa-3,8- diazaspiro[4.5]decan-3-yl)benzyl)phosphonic acid |
| 31 | 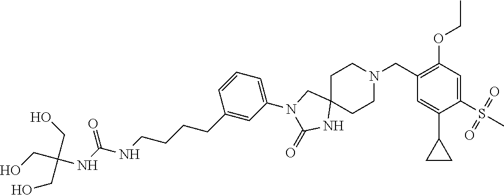 [SMILES: CCOc1cc(S(C)(=O)=O)c(C2CC2)cc1CN1CCC2(CC1)CN(c1cccc(CCCCNC(=O)NC(CO)(CO)CO)c1)C(=O)N2] | 1-(4-(3-(8-(5-cyclopropyl-2-ethoxy-4- (methylsulfonyl)benzyl)-2-oxo-1,3,8- triazaspiro[4.5]decan-3-yl)phenyl)butyl)-3-(1,3- dihydroxy-2-(hydroxymethyl)propan-2-yl)urea |
| 32 | 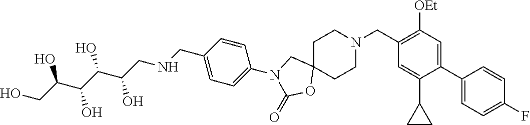 [SMILES: CCOc1cc(-c2ccc(F)cc2)c(C2CC2)cc1CN1CCC2(CC1)CN(c1ccc(CNC[C@H](O)[C@@H](O)[C@H](O)[C@H](O)CO)cc1)C(=O)O2] | 8-((2-cyclopropyl-5-ethoxy-4'-fluoro-[1,1'- biphenyl]-4-yl)methyl)-3-(4-((((2S,3R,4R,5R)- 2,3,4,5,6- pentahydroxyhexyl)amino)methyl)phenyl)-1-oxa- 3,8-diazaspiro [4.5]decan-2-one |
| 33 | 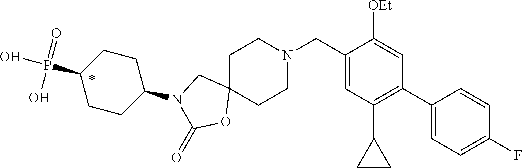 [SMILES: CCOc1cc(-c2ccc(F)cc2)c(C2CC2)cc1CN1CCC2(CC1)CN([C@H]1CC[C@@H](P(=O)(O)O)CC1)C(=O)O2] | ((cis)-4-(8-((2-cyclopropyl-5-ethoxy-4'-fluoro-[1,1'- biphenyl]-4-yl)methyl)-2-oxo-1-oxa-3,8- diazaspiro[4.5]decan-3-yl)cyclohexyl)phosphonic acid |
| 34 | 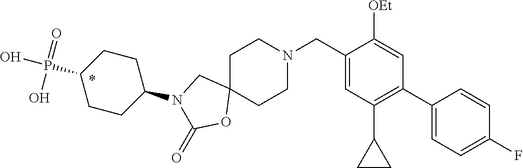 [SMILES: CCOc1cc(-c2ccc(F)cc2)c(C2CC2)cc1CN1CCC2(CC1)CN([C@H]1CC[C@H](P(=O)(O)O)CC1)C(=O)O2] | ((trans)-4-(8-((2-cyclopropyl-5-ethoxy-4'-fluoro- [1,1'-biphenyl]-4-yl)methyl)-2-oxo-1-oxa-3,8- diazaspiro[4.5]decan-3-yl)cyclohexyl)phosphonic acid |
| 35 | 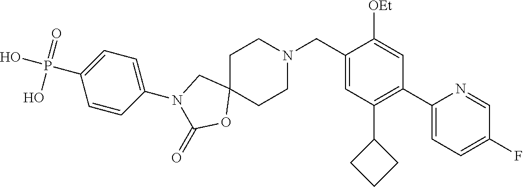 [SMILES: CCOc1cc(-c2ccc(F)cn2)c(C2CCC2)cc1CN1CCC2(CC1)CN(c1ccc(P(=O)(O)O)cc1)C(=O)O2] | (4-(8-(5-cyclobulyl-2-ethoxy-4-(5-fluoropyridin-2- yl)benzyl)-2-oxo-1-oxa-3,8-diazaspiro[4.5]decan-3- yl)phenyl)phosphonic acid |
| 36 | 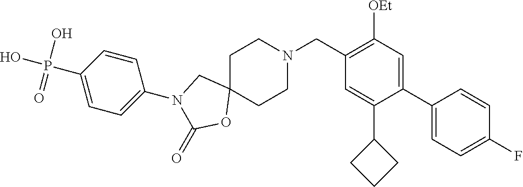 [SMILES: CCOc1cc(-c2ccc(F)cc2)c(C2CCC2)cc1CN1CCC2(CC1)CN(c1ccc(P(=O)(O)O)cc1)C(=O)O2] | (4-(8-((2-cyclobutyl-5-ethoxy-4'-fluoro-[1,1'- biphenyl]-4-yl)methyl)-2-oxo-1-oxa-3,8- diazaspiro[4.5]decan-3-yl)phenyl)phosphonic acid |
| 37 | 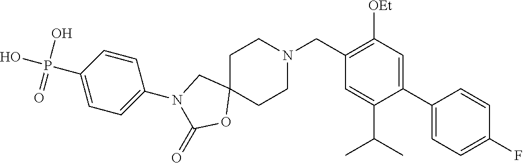 [SMILES: CCOc1cc(-c2ccc(F)cc2)c(C(C)C)cc1CN1CCC2(CC1)CN(c1ccc(P(=O)(O)O)cc1)C(=O)O2] | (4-(8-((5-ethoxy-4'-fluoro-2-isopropyl-[1,1'- biphenyl]-4-yl)methyl)-2-oxo-1-oxa-3,8- diazaspiro[4.5]decan-3-yl)phenyl)phosphonic acid |

* absolute stereochemistry not confirmed

In some embodiments, compounds of Table 1 are provided as pharmaceutically acceptable salts.

TABLE 2

| Ex.# | Structure | Name |
|---|---|---|
| 38 | 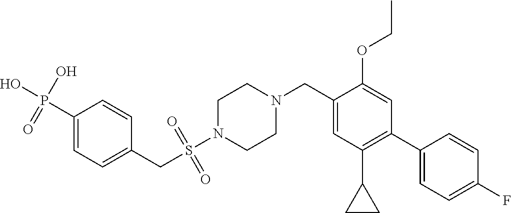 [SMILES: CCOc1cc(-c2ccc(F)cc2)c(C2CC2)cc1CN1CCN(S(=O)(=O)Cc2ccc(P(=O)(O)O)cc2)CC1] | (4-(((4-((2-cyclopropyl-5-ethoxy-4′-fluoro-[1,1′- biphenyl]-4-yl)methyl)piperazin-1- yl)sulfonyl)methyl)phenyl)phosphonic acid |
| 39 | 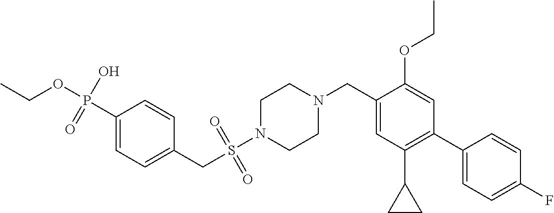 [SMILES: CCOc1cc(-c2ccc(F)cc2)c(C2CC2)cc1CN1CCN(S(=O)(=O)Cc2ccc(P(=O)(O)OCC)cc2)CC1] | ethyl hydrogen (4-(((4-((2-cyclopropyl-5-ethoxy-4′- fluoro-[1,1′-biphenyl]-4-yl)methyl)piperazin-1- yl)sulfonyl)methyl)phenyl)phosphonate |
| 40 | 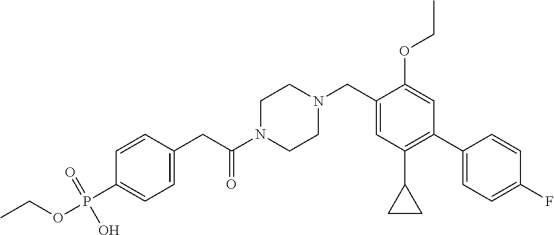 [SMILES: CCOc1cc(-c2ccc(F)cc2)c(C2CC2)cc1CN1CCN(C(=O)Cc2ccc(P(=O)(O)OCC)cc2)CC1] | ethyl hydrogen (4-(2-(4-((2-cyclopropyl-5-ethoxy- 4′-fluoro-[1,1′-biphenyl]-4-yl)methyl)piperazin-1- yl)-2-oxoethyl)phenyl)phosphonate |
| 41 | 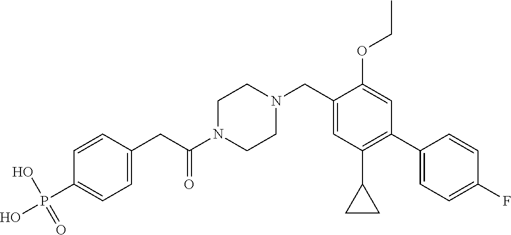 [SMILES: CCOc1cc(-c2ccc(F)cc2)c(C2CC2)cc1CN1CCN(C(=O)Cc2ccc(P(=O)(O)O)cc2)CC1] | (4-(2-(4-((2-cyclopropyl-5-ethoxy-4′-fluoro-[1,1′- biphenyl]-4-yl)methyl)piperazin-1-yl)-2- oxoethyl)phenyl)phosphonic acid |
| 42 | 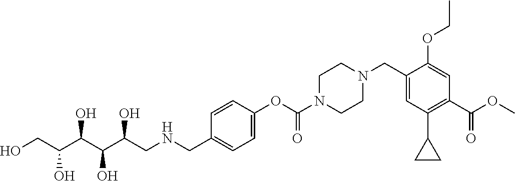 [SMILES: CCOc1cc(C(=O)OC)c(C2CC2)cc1CN1CCN(C(=O)Oc2ccc(CNC[C@H](O)[C@@H](O)[C@H](O)[C@H](O)CO)cc2)CC1] | 4-((((2S,3R,4R,5R)-2,3,4,5,6- pentahydroxyhexyl)amino)methyl)phenyl 4-(5- cyclopropyl-2-ethoxy-4- (methoxycarbonyl)benzyl)piperazine-1-carboxylate |

In some embodiments, compounds of Table 2 are provided as pharmaceutically acceptable salts.

Further Forms of Compounds

Furthermore, in some embodiments, the compounds described herein exist as “geometric isomers.” In some embodiments, the compounds described herein possess one or more double bonds. The compounds presented herein include all cis, trans, syn, anti, entgegen (E), and zusammen (Z) isomers as well as the corresponding mixtures thereof. In some situations, compounds exist as tautomers.

A “tautomer” refers to a molecule wherein a proton shift from one atom of a molecule to another atom of the same molecule is possible. In certain embodiments, the compounds presented herein exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

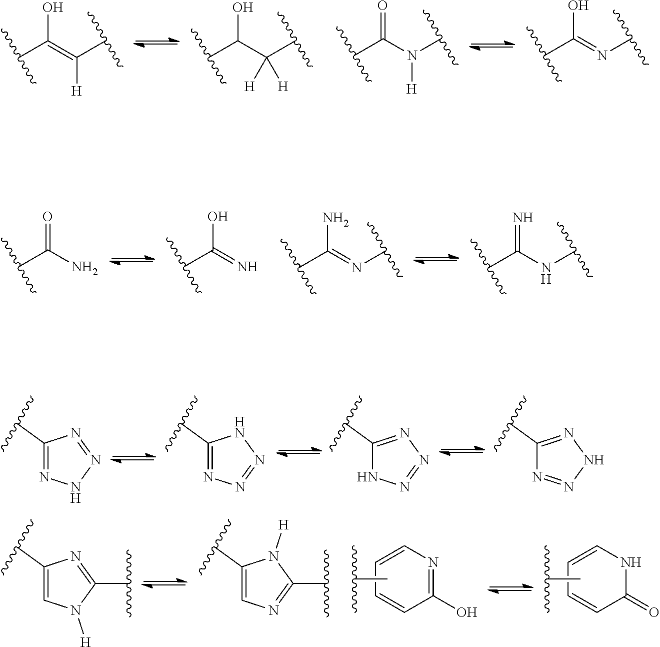

In some situations, the compounds described herein possess one or more chiral centers and each center exists in the (R)-configuration or (S)-configuration. The compounds described herein include all diastereomeric, enantiomeric, and epimeric forms as well as the corresponding mixtures thereof. In additional embodiments of the compounds and methods provided herein, mixtures of enantiomers and/or diastereoisomers, resulting from a single preparative step, combination, or interconversion are useful for the applications described herein. In some embodiments, the compounds described herein are prepared as optically pure enantiomers by chiral chromatographic resolution of the racemic mixture. In some embodiments, the compounds described herein are prepared as their individual stereoisomers by reacting a racemic mixture of the compound with an optically active resolving agent to form a pair of diastereoisomeric compounds, separating the diastereomers and recovering the optically pure enantiomers. In some embodiments, dissociable complexes are preferred (e.g., crystalline diastereomeric salts). In some embodiments, the diastereomers have distinct physical properties (e.g., melting points, boiling points, solubilities, reactivity, etc.) and are separated by taking advantage of these dissimilarities. In some embodiments, the diastereomers are separated by chiral chromatography, or preferably, by separation/resolution techniques based upon differences in solubility. In some embodiments, the optically pure enantiomer is then recovered, along with the resolving agent, by any practical means that would not result in racemization.

The term “positional isomer” refers to structural isomers around a central ring, such as ortho-, meta-, and para-isomers around a benzene ring.

The methods and formulations described herein include the use of N-oxides (if appropriate), crystalline forms (also known as polymorphs), or pharmaceutically acceptable salts of compounds described herein, as well as active metabolites of these compounds having the same type of activity.

“Pharmaceutically acceptable salt” includes both acid and base addition salts. A pharmaceutically acceptable salt of any one of the compounds described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997). Acid addition salts of basic compounds are prepared by contacting the free base forms with a sufficient amount of the desired acid to produce the salt.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. In some embodiments, pharmaceutically acceptable base addition salts are formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

“Prodrug” is meant to indicate a compound that is, in some embodiments, converted under physiological conditions or by solvolysis to an active compound described herein. Thus, the term prodrug refers to a precursor of an active compound that is pharmaceutically acceptable. A prodrug is typically inactive when administered to a subject, but is converted in vivo to an active compound, for example, by hydrolysis. The prodrug compound often offers advantages of solubility, tissue compatibility or delayed release in a mammalian organism (see, e.g., Bundgard, H., Design of Prodrugs (1985), pp. 7-9, 21-24 (Elsevier, Amsterdam).

A discussion of prodrugs is provided in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987.

The term “prodrug” is also meant to include any covalently bonded carriers, which release the active compound in vivo when such prodrug is administered to a mammalian subject. Prodrugs of an active compound, as described herein, are prepared by modifying functional groups present in the active compound in such a way that the modifications are cleaved, either in routine manipulation or in vivo, to the parent active compound. Prodrugs include compounds wherein a hydroxy, amino, carboxy, or mercapto group is bonded to any group that, when the prodrug of the active compound is administered to a mammalian subject, cleaves to form a free hydroxy, free amino, free carboxy, or free mercapto group, respectively. Examples of prodrugs include, but are not limited to, acetate, formate and benzoate derivatives of alcohol or amine functional groups in the active compounds and the like.

“Pharmaceutically acceptable solvate” refers to a composition of matter that is the solvent addition form. In some embodiments, solvates contain either stoichiometric or non-stoichiometric amounts of a solvent, and are formed during the process of making with pharmaceutically acceptable solvents such as water, ethanol, and the like. “Hydrates” are formed when the solvent is water, or “alcoholates” are formed when the solvent is alcohol. Solvates of compounds described herein are conveniently prepared or formed during the processes described herein. The compounds provided herein optionally exist in either unsolvated as well as solvated forms.

The compounds disclosed herein, in some embodiments, are used in different enriched isotopic forms, e.g., enriched in the content of ²H, ³H, ¹¹C, ¹³C and/or ¹⁴C. In some embodiments, the compound is deuterated in at least one position. Such deuterated forms can be made by the procedure described in U.S. Pat. Nos. 5,846,514 and 6,334,997. As described in U.S. Pat. Nos. 5,846,514 and 6,334,997, deuteration can improve the metabolic stability and or efficacy, thus increasing the duration of action of drugs.

Unless otherwise stated, structures depicted herein are intended to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of the present disclosure.

The compounds of the present disclosure optionally contain unnatural proportions of atomic isotopes at one or more atoms that constitute such compounds. For example, the compounds may be labeled with isotopes, such as for example, deuterium (²H), tritium (H), iodine-125 (¹²⁵I) or carbon-14 (¹⁴C). Isotopic substitution with ²H, ³H, ¹¹C, ¹³C, ¹⁴C, ¹⁵C, ¹²N, ¹³N, ¹⁵N, ¹⁶N, ¹⁷O, ¹⁸O, ¹⁴F, ¹⁵F, ¹⁶F, ¹⁷F, ¹⁸F, ³³S, ³⁴S, ³⁵S, ³⁶S, ³⁵Cl, ³⁷Cl, ⁷⁹Br, ⁸¹Br, ¹²⁵I are all contemplated. All isotopic variations of the compounds of the present invention, whether radioactive or not, are encompassed within the scope of the present invention.

In certain embodiments, the compounds disclosed herein have some or all of the ¹H atoms replaced with ²H atoms. The methods of synthesis for deuterium-containing compounds are known in the art. In some embodiments deuterium substituted compounds are synthesized using various methods such as described in: Dean, Dennis C.; Editor. Recent Advances in the Synthesis and Applications of Radiolabeled Compounds for Drug Discovery and Development. [In: Curr., Pharm. Des., 2000; 6(10)] 2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64(1-2), 9-32.

In some embodiments, the compounds described herein are labeled by other means, including, but not limited to, the use of chromophores or fluorescent moieties, bioluminescent labels, or chemiluminescent labels.

In certain embodiments, the compounds described herein, or a pharmaceutically acceptable salt, solvate, stereoisomer, or prodrug thereof, as described herein are substantially pure, in that it contains less than about 5%, or less than about 1%, or less than about 0.1%, of other organic small molecules, such as contaminating intermediates or by-products that are created, for example, in one or more of the steps of a synthesis method.

Preparation of the Compounds

Compounds described herein are synthesized using standard synthetic techniques or using methods known in the art in combination with methods described herein.

Unless otherwise indicated, conventional methods of mass spectroscopy, NMR, HPLC, protein chemistry, biochemistry, recombinant DNA techniques and pharmacology are employed.

Compounds are prepared using standard organic chemistry techniques such as those described in, for example, March's Advanced Organic Chemistry, 6th Edition, John Wiley and Sons, Inc. Alternative reaction conditions for the synthetic transformations described herein may be employed such as variation of solvent, reaction temperature, reaction time, as well as different chemical reagents and other reaction conditions.

In some embodiments, compounds described herein are prepared as described as outlined in the Examples.

Pharmaceutical Compositions

In some embodiments, disclosed herein is a pharmaceutical composition comprising an SSTR5 antagonist described herein, or a pharmaceutically acceptable salt, solvate, stereoisomer, or prodrug thereof, and a pharmaceutically acceptable excipient. In some embodiments, the SSTR5 antagonist is combined with a pharmaceutically suitable (or acceptable) carrier (also referred to herein as a pharmaceutically suitable (or acceptable) excipient, physiologically suitable (or acceptable) excipient, or physiologically suitable (or acceptable) carrier) selected on the basis of a chosen route of administration, e.g., oral administration, and standard pharmaceutical practice as described, for example, in Remington: The Science and Practice of Pharmacy (Gennaro, 21^{st} Ed. Mack Pub. Co., Easton, Pa. (2005)).

Accordingly, provided herein is a pharmaceutical composition comprising a compound described herein, or a pharmaceutically acceptable salt, solvate, stereoisomer, or prodrug thereof, together with a pharmaceutically acceptable excipient.

Examples of suitable aqueous and non-aqueous carriers which are employed in the pharmaceutical compositions include water, ethanol, polyols (such as glycerol, propylene glycol, polyethylene glycol, and the like), and suitable mixtures thereof, vegetable oils, such as olive oil, and injectable organic esters, such as ethyl oleate and cyclodextrins. Proper fluidity is maintained, for example, by the use of coating materials, such as lecithin, by the maintenance of the required particle size in the case of dispersions, and by the use of surfactants.

Combination Therapies

In certain embodiments, it is appropriate to administer at least one compound described herein, or a pharmaceutically acceptable salt, solvate, stereoisomer, or prodrug thereof, in combination with one or more other therapeutic agents. In some embodiments, a compound described herein, or a pharmaceutically acceptable salt, solvate, stereoisomer, or prodrug thereof, is administered in combination with a TGR5 agonist, a GPR40 agonist, a GPR119 agonist, a CCK1 agonist, a PDE4 inhibitor, a DPP-4 inhibitor, a GLP-1 receptor agonist, metformin, or combinations thereof. In certain embodiments, the pharmaceutical composition further comprises one or more anti-diabetic agents. In certain embodiments, the pharmaceutical composition further comprises one or more anti-obesity agents. In certain embodiments, the pharmaceutical composition further comprises one or more agents to treat nutritional disorders.

Examples of a TGR5 agonist to be used in combination with a compound described herein, or a pharmaceutically acceptable salt, solvate, stereoisomer, or prodrug thereof, include: INT-777, XL-475, SRX-1374, RDX-8940, RDX-98940, SB-756050, and those disclosed in WO-2008091540, WO-2010059853, WO-2011071565, WO-2018005801, WO-2010014739, WO-2018005794, WO-2016054208, WO-2015160772, WO-2013096771, WO-2008067222, WO-2008067219, WO-2009026241, WO-2010016846, WO-2012082947, WO-2012149236, WO-2008097976, WO-2016205475, WO-2015183794, WO-2013054338, WO-2010059859, WO-2010014836, WO-2016086115, WO-2017147159, WO-2017147174, WO-2017106818, WO-2016161003, WO-2014100025, WO-2014100021, WO-2016073767, WO-2016130809, WO-2018226724, WO-2018237350, WO-2010093845, WO-2017147137, WO-2015181275, WO-2017027396, WO-2018222701, WO-2018064441, WO-2017053826, WO-2014066819, WO-2017079062, WO-2014200349, WO-2017180577, WO-2014085474.

Examples of a GPR40 agonist to be used in combination with a compound described herein, or a pharmaceutically acceptable salt, solvate, stereoisomer, or prodrug thereof, include: fasiglifam, MR-1704, SCO-267, SHR-0534, HXP-0057-SS, LY-2922470, P-11187, JTT-851, ASP-4178, AMG-837, ID-11014A, HD-C₇₁₅, CNX-011-67, JNJ-076, TU-5113, HD-6277, MK-8666, LY-2881835, CPL-207-280, ZYDG-2, and those described in U.S. Ser. No. 07/750,048, WO-2005051890, WO-2005095338, WO-2006011615, WO-2006083612, WO-2006083781, WO-2007088857, WO-2007123225, WO-2007136572, WO-2008054674, WO-2008054675, WO-2008063768, WO-2009039942, WO-2009039943, WO-2009054390, WO-2009054423, WO-2009054468, WO-2009054479, WO-2009058237, WO-2010085522, WO-2010085525, WO-2010085528, WO-2010091176, WO-2010123016, WO-2010123017, WO-2010143733, WO-2011046851, WO-2011052756, WO-2011066183, WO-2011078371, WO-2011161030, WO-2012004269, WO-2012004270, WO-2012010413, WO-2012011125, WO-2012046869, WO-2012072691, WO-2012111849, WO-2012147518, WO-2013025424, WO-2013057743, WO-2013104257, WO-2013122028, WO-2013122029, WO-2013128378, WO-2013144097, WO-2013154163, WO-2013164292, WO-2013178575, WO-2014019186, WO-2014073904, WO-2014082918, WO-2014086712, WO-2014122067, WO-2014130608, WO-2014146604, WO-2014169817, WO-2014170842, WO-2014187343, WO-2015000412, WO-2015010655, WO-2015020184, WO-2015024448, WO-2015024526, WO-2015028960, WO-2015032328, WO-2015044073, WO-2015051496, WO-2015062486, WO-2015073342, WO-2015078802, WO-2015084692, WO-2015088868, WO-2015089809, WO-2015097713, WO-2015105779, WO-2015105786, WO-2015119899, WO-2015176267, WO-201600771, WO-2016019587, WO-2016022446, WO-2016022448, WO-2016022742, WO-2016032120, WO-2016057731, WO-2017025368, WO-2017027309, WO-2017027310, WO-2017027312, WO-2017042121, WO-2017172505, WO-2017180571, WO-2018077699, WO-2018081047, WO-2018095877, WO-2018106518, WO-2018111012, WO-2018118670, WO-2018138026, WO-2018138027, WO-2018138028, WO-2018138029, WO-2018138030, WO-2018146008, WO-2018172727, WO-2018181847, WO-2018182050, WO-2018219204, WO-2019099315, and WO-2019134984.

Examples of a GPR119 agonist to be used in combination with a compound described herein, or a pharmaceutically acceptable salt, solvate, stereoisomer, or prodrug thereof, include: DS-8500a, HD-2355, LC34AD3, PSN-491, HM-47000, PSN-821, MBX-2982, GSK-1292263, APD597, DA-1241, and those described in WO-2009141238, WO-2010008739, WO-2011008663, WO-2010013849, WO-2012046792, WO-2012117996, WO-2010128414, WO-2011025006, WO-2012046249, WO-2009106565, WO-2011147951, WO-2011127106, WO-2012025811, WO-2011138427, WO-2011140161, WO-2011061679, WO-2017175066, WO-2017175068, WO-2015080446, WO-2013173198, US-20120053180, WO-2011044001, WO-2010009183, WO-2012037393, WO-2009105715, WO-2013074388, WO-2013066869, WO-2009117421, WO-201008851, WO-2012077655, WO-2009106561, WO-2008109702, WO-2011140160, WO-2009126535, WO-2009105717, WO-2013122821, WO-2010006191, WO-2009012275, WO-2010048149, WO-2009105722, WO-2012103806, WO-2008025798, WO-2008097428, WO-2011146335, WO-2012080476, WO-2017106112, WO-2012145361, WO-2012098217, WO-2008137435, WO-2008137436, WO-2009143049, WO-2014074668, WO-2014052619, WO-2013055910, WO-2012170702, WO-2012145604, WO-2012145603, WO-2011030139, WO-2018153849, WO-2017222713, WO-2015150565, WO-2015150563, WO-2015150564, WO-2014056938, WO-2007120689, WO-2016068453, WO-2007120702, WO-2013167514, WO-2011113947, WO-2007003962, WO-2011153435, WO-2018026890, WO-2011163090, WO-2011041154, WO-2008083238, WO-2008070692, WO-2011150067, and WO-2009123992.

Examples of a CCK1 agonist to be used in combination with a compound described herein, or a pharmaceutically acceptable salt, solvate, stereoisomer, or prodrug thereof, include: A-70874, A-71378, A-71623, A-74498, CE-326597, GI-248573, GSKI-181771X, NN-9056, PD-149164, PD-134308, PD-135158, PD-170292, PF-04756956, SR-146131, SSR-125180, and those described in EP-00697403, US-20060177438, WO-2000068209, WO-2000177108, WO-2000234743, WO-2000244150, WO-2009119733, WO-2009314066, WO-2009316982, WO-2009424151, WO-2009528391, WO-2009528399, WO-2009528419, WO-2009611691, WO-2009611940, WO-2009851686, WO-2009915525, WO-2005035793, WO-2005116034, WO-2007120655, WO-2007120688, WO-2008091631, WO-2010067233, WO-2012070554, and WO-2017005765.

Examples of a PDE4 inhibitor to be used in combination with a compound described herein, or a pharmaceutically acceptable salt, solvate, stereoisomer, or prodrug thereof, include: apremilast, cilomilast, crisaborole, diazepam, luteolin, piclamilast, and roflumilast.

Examples of a DPP-4 inhibitor to be used in combination with a compound described herein, or a pharmaceutically acceptable salt, solvate, stereoisomer, or prodrug thereof, include: sitagliptin, vildagliptin, saxagliptin, linagliptin, gemigliptin, teneligliptin, alogliptin, trelagliptin, omarigliptin, evogliptin, gosogliptin, and dutogliptin.

Examples of a GLP-1 receptor agonist to be used in combination with a compound described herein, or a pharmaceutically acceptable salt, solvate, stereoisomer, or prodrug thereof, include: albiglutide, dulaglutide, exenatide, extended-release exenatide, liraglutide, lixisenatide, and semaglutide.

Examples of anti-diabetic agents to be used in combination with a compound described herein, or a pharmaceutically acceptable salt, solvate, stereoisomer, or prodrug thereof, include: GLP-1 receptor agonists such as exenatide, liraglutide, taspoglutide, lixisenatide, albiglutide, dulaglutide, semaglutide, OWL833 and ORMD 0901; SGLT2 inhibitors such as dapagliflozin, canagliflozin, empagliflozin, ertugliflozin, ipragliflozin, luseogliflozin, remogliflozin, sergliflozin, sotagliflozin, and tofogliflozin; biguinides such as metformin; insulin and insulin analogs.

Examples of anti-obesity agents to be used in combination with a compound described herein, or a pharmaceutically acceptable salt, solvate, stereoisomer, or prodrug thereof, include: GLP-1 receptor agonists such as liraglutide, semaglutide; SGLT1/2 inhibitors such as LIK066, pramlintide and other amylin analogs such as AM-833, AC2307, and BI 473494; PYY analogs such as NN-9747, NN-9748, AC-162352, AC-163954, GT-001, GT-002, GT-003, and RHS-08; GIP receptor agonists such as APD-668 and APD-597; GLP-1/GIP co-agonists such as tirzepatide (LY329176), BHM-089, LBT-6030, CT-868, SCO-094, NNC-0090-2746, RG-7685, NN-9709, and SAR-438335; GLP-1/glucagon co-agonist such as cotadutide (MEDI0382), BI 456906, TT-401, G-49, H&D-001A, ZP-2929, and HM-12525A; GLP-1/GIP/glucagon triple agonist such as SAR-441255, HM-15211, and NN-9423; GLP-1/secretin co-agonists such as GUB06-046; leptin analogs such as metreleptin; GDF15 modulators such as those described in WO2012138919, WO2015017710, WO2015198199, WO-2017147742 and WO-2018071493; FGF21 receptor modulators such as NN9499, NGM386, NGM313, BFKB8488A (RG7992), AKR-001, LLF-580, CVX-343, LY-2405319, BI089-100, and BMS-986036; MC4 agonists such as setmelanotide; MetAP2 inhibitors such as ZGN-1061; ghrelin receptor modulators such as HM04 and AZP-531; ghrelin O-acyltransferase inhibitors such as T-3525770 (RM-852) and GLWL-01; and oxytocin analogs such as carbetocin.

Examples of agents for nutritional disorders to be used in combination with a compound described herein, or a pharmaceutically acceptable salt, solvate, stereoisomer, or prodrug thereof, include: GLP-2 receptor agonists such as tedaglutide, glepaglutide (ZP1848), elsiglutide (ZP1846), apraglutide (FE 203799), HM-15912, NB-1002, GX-G8, PE-0503, SAN-134, and those described in WO-2011050174, WO-2012028602, WO-2013164484, WO-2019040399, WO-2018142363, WO-2019090209, WO-2006117565, WO-2019086559, WO-2017002786, WO-2010042145, WO-2008056155, WO-2007067828, WO-2018229252, WO-2013040093, WO-2002066511, WO-2005067368, WO-2009739031, WO-2009632414, and WO2008028117; and GLP-1/GLP-2 receptor co-agonists such as ZP-GG-72 and those described in WO-2018104561, WO-2018104558, WO-2018103868, WO-2018104560, WO-2018104559, WO-2018009778, WO-2016066818, and WO-2014096440.

In one embodiment, the therapeutic effectiveness of one of the compounds described herein is enhanced by administration of an adjuvant (i.e., by itself the adjuvant has minimal therapeutic benefit, but in combination with another therapeutic agent, the overall therapeutic benefit to the patient is enhanced). Or, in some embodiments, the benefit experienced by a patient is increased by administering one of the compounds described herein with another agent (which also includes a therapeutic regimen) that also has therapeutic benefit.

In one specific embodiment, a compound described herein, or a pharmaceutically acceptable salt, solvate, stereoisomer, or prodrug thereof, is co-administered with one or more additional therapeutic agents, wherein the compound described herein, or a pharmaceutically acceptable salt, solvate, stereoisomer, or prodrug thereof, and the additional therapeutic agent(s) modulate different aspects of the disease, disorder or condition being treated, thereby providing a greater overall benefit than administration of either therapeutic agent alone. In some embodiments, the additional therapeutic agent(s) is a TGR5 agonist, a GPR40 agonist, a GPR119 agonist, a CCK1 agonist, a PDE4 inhibitor, a DPP-4 inhibitor, a GLP-1 receptor agonist, metformin, or combinations thereof. In some embodiments, the additional therapeutic agent is an anti-diabetic agent. In some embodiments, the additional therapeutic agent is an anti-obesity agent. In some embodiments, the additional therapeutic agent is an agent to treat nutritional disorders.

In combination therapies, the multiple therapeutic agents (one of which is one of the compounds described herein) are administered in any order or even simultaneously. If administration is simultaneous, the multiple therapeutic agents are, by way of example only, provided in a single, unified form, or in multiple forms (e.g., as a single pill or as two separate pills).

The compounds described herein, or pharmaceutically acceptable salts, solvates, stereoisomers, or prodrugs thereof, as well as combination therapies, are administered before, during or after the occurrence of a disease or condition, and the timing of administering the composition containing a compound varies. Thus, in one embodiment, the compounds described herein are used as a prophylactic and are administered continuously to subjects with a propensity to develop conditions or diseases in order to prevent the occurrence of the disease or condition. In another embodiment, the compounds and compositions are administered to a subject during or as soon as possible after the onset of the symptoms. In specific embodiments, a compound described herein is administered as soon as is practicable after the onset of a disease or condition is detected or suspected, and for a length of time necessary for the treatment of the disease.

In some embodiments, a compound described herein, or a pharmaceutically acceptable salt thereof, is administered in combination with anti-inflammatory agent, anti-cancer agent, immunosuppressive agent, steroid, non-steroidal anti-inflammatory agent, antihistamine, analgesic, hormone blocking therapy, radiation therapy, monoclonal antibodies, or combinations thereof.

### EXAMPLES

### List of Abbreviations

As used above, and throughout the description of the invention, the following abbreviations, unless otherwise indicated, shall be understood to have the following meanings:
ACN or MeCN acetonitrile
Boc or BOC tert-butyloxycarbonyl
Bn benzyl
BnBr benzyl bromide
Cbz carboxybenzyl
CbzCl benzyl chloroformate
CDI 1,1′-Carbonyldiimidazole
Cy cyclohexyl
DCC N,N′-dicyclohexylcarbodiimide
DCM dichloromethane (CH₂Cl₂)
DIBAL-H diisobutylaluminium hydride
DIPEA or DIEA diisopropylethylamine
DMA dimethylacetamide
DMAP 4-dimethylaminopyridine
DME 1,2-dimethoxyethane
DMEDA 1,2-dimethylethylenediamine
DMEM Dulbecco's Modified Eagle Medium
DMF dimethylformamide
DMFDMA dimethylformamide dimethylacetal
DMSO dimethylsulfoxide
DPPF 1,1′-Bis(diphenylphosphino)ferrocene
EDCI 1-ethyl-3-(3-dimethylaminopropyl)carbodiimide
eq equivalent(s)
Et ethyl
EtI ethyl iodide
EtOH ethanol
EtOAc or EA ethyl acetate
FA formic acid
FBS fetal bovine serum
h, hr(s)hour(s)
HATU 1-[bis(dimethylamino)methylene]-1H-1,2,3-triazolo[4,5-b]pyridinium 3-oxid hexafluorophosphate
HPLC high performance liquid chromatography
HTRF homogeneous time resolved fluorescence
i-pr or ipr isopropyl
iPrMgCl isopropylmagnesium chloride
i-PrOHiso-propanol
LCMS liquid chromatography-mass spectrometry
LDA lithium diisopropylamide
Me methyl
MeOH methanol
MS mass spectroscopy
Ms methanesulfonyl (mesyl)
MsC1 methanesulfonyl chloride (mesyl chloride)
NBS N-bromosuccinimide
NiCl₂.dtbbpy [4,4′-bis(1,1-dimethylethyl)-2,2′-bipyridine] nickel (II) dichloride
NMR nuclear magnetic resonance
PCy₃ tricyclohexylphosphine
Pd(dba)₂ bis(dibenzylideneacetone)palladium(O)
Pd(dppf)Cl₂ [1,1′-Bis(diphenylphosphino)ferrocene]dichloropalladium(II)
PE petroleum ether
PMB p-methoxybenzyl
psi pounds per square inch
Py pyridine
Rt or RT room temperature
SPhos 2-dicyclohexylphosphino-2′,6′-dimethoxybiphenyl
SPhos-Pd-G2 chloro(2-dicyclohexylphosphino-2′,6′-dimethoxy-1,1′-biphenyl)[2-(2′-amino-1,1′-biphenyl)]palladium(II)
t-Bu tert-butyl
t-Bu₃P—Pd-G2 chloro[(tri-tert-butylphosphine)-2-(2-aminobiphenyl)]palladium(II)
TEA triethylamine
Tf trifluoromethylsulfonyl (triflyl)
TFA trifluoroacetic acid
THE tetrahydrofuran
TLC thin layer chromatography
TMSBr bromotrimethylsilane
Tol or tol toluene
TR-FRET time-resolved Forster resonance energy transfer
Ts toluenesulfonyl (tosyl)
TsOH p-toluenesulfonic acid
TTMSS tris(trimethylsilyl)silane
XPhos 2-dicyclohexylphosphino-2′,4′,6′-triisopropylbiphenyl
XPhos-Pd-G2 chloro(2-dicyclohexylphosphino-2′,4′,6′-triisopropyl-1,1′-biphenyl)[2-(2′-amino-1,1′-biphenyl)]palladium(II)

### I. Chemical Synthesis

Unless otherwise noted, reagents and solvents were used as received from commercial suppliers. Anhydrous solvents and oven-dried glassware were used for synthetic transformations sensitive to moisture and/or oxygen. Yields were not optimized. Reaction times are approximate and were not optimized. Column chromatography and thin layer chromatography (TLC) were performed on silica gel unless otherwise noted.

### Example 1: (4-(8-((2-cyclopropyl-5-ethoxy-4′-fluoro-[1,1′-biphenyl]-4-yl)methyl)-2-oxo-1-oxa-3,8-diazaspiro[4.5]decan-3-yl)phenyl)phosphonic acid (Compound 1)

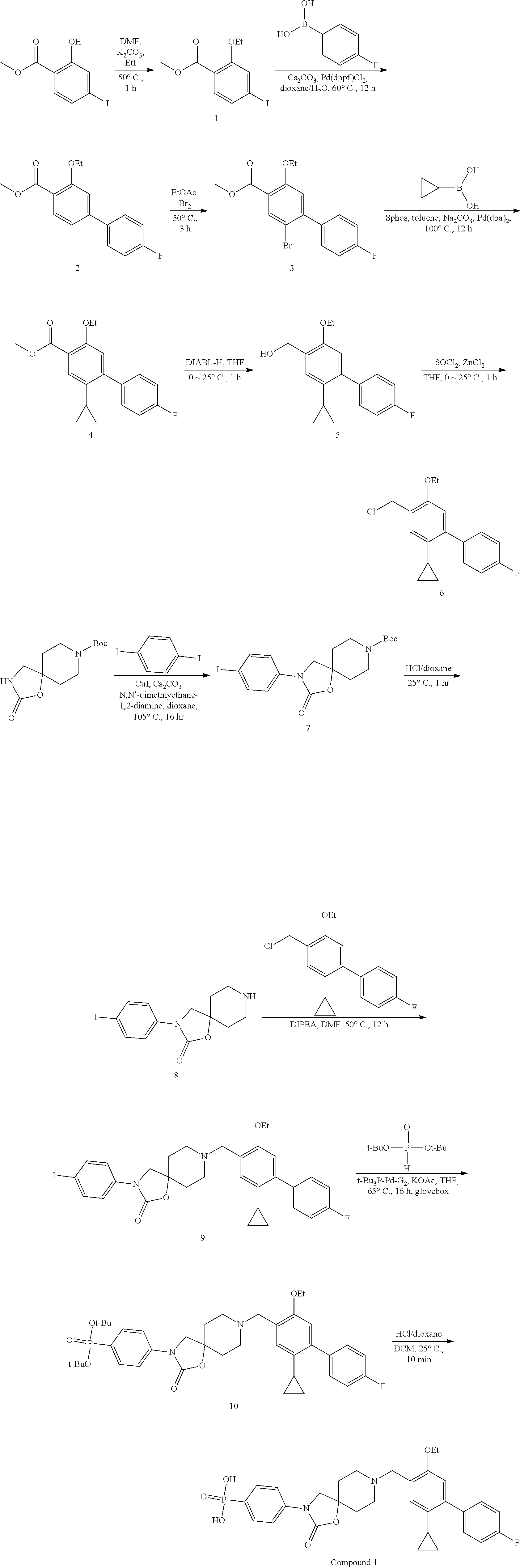

Step 1: methyl 2-ethoxy-4-iodo-benzoate (1): To a solution of methyl 2-hydroxy-4-iodo-benzoate (13 g, 47 mmol, 1 eq) in DMF (130 mL) was added K₂CO₃ (13 g, 94 mmol, 2 eq) and EtI (14.6 g, 94 mmol, 7.5 mL, 2 eq). The mixture was stirred at 50° C. for 1 hour. The reaction mixture was poured into H₂O (50 mL) and extracted with EA (50 mL×3). The combined organic layer was washed with water (50 mL×2) and brine (50 mL×2), dried over Na₂SO₄, filtered and concentrated in vacuo to give 1 (14.2 g, crude) as a yellow oil.

Step 2: methyl 2-ethoxy-4-(4-fluorophenyl)benzoate (2): To a solution of 1 (7.5 g, 25 mmol, 1 eq), (4-fluorophenyl)boronic acid (3.8 g, 27 mmol, 1.1 eq), Cs₂CO₃ (16 g, 49 mmol, 2 eq) and Pd(dppf)Cl₂ (896 mg, 1.2 mmol, 0.05 eq) was added H₂O (20 mL) and dioxane (60 mL). Then the mixture was stirred at 60° C. for 12 hours. The reaction mixture was diluted with H₂O (100 mL) and extracted with EA (90 mL×2). The combined organic layers were washed with saturated brine (50 mL×2), dried over Na₂SO₄, filtered and concentrated under reduced pressure to give a residue. The residue was purified by column chromatography (SiO₂, petroleum ether/ethyl acetate=100/1 to 10/1) to give 2 (6.7 g, 99% yield) as a yellow solid.

Step 3: methyl 5-bromo-2-ethoxy-4-(4-fluorophenyl)benzoate (3): To a solution of 2 (7.2 g, 26 mmol, 1 eq) in EtOAc (72 mL) was added Br₂ (5.0 g, 32 mmol, 1.6 mL, 1.2 eq). Then the mixture was stirred at 50° C. for 3 hours. The reaction mixture was diluted with H₂O (120 mL) and extracted with EA (75 mL×2). The combined organic layers were washed with saturated brine (50 mL×2), dried over Na₂SO₄, filtered and concentrated under reduced pressure to give a residue. The residue was purified by column chromatography (SiO₂, petroleum ether/ethyl acetate, 50/1 to 5/1) to give 3 (5.5 g, 59% yield) as a white solid. ¹H NMR (400 MHz, CD₃OD) δ 8.07 (s, 1H), 7.35-7.42 (m, 2H), 7.10-7.17 (m, 2H), 6.90 (s, 1H), 4.11 (q, J=7.2 Hz, 2H), 3.91 (s, 3H), 1.47 (t, J=6.8 Hz, 3H).

Step 4: methyl 5-cyclopropyl-2-ethoxy-4-(4-fluorophenyl)benzoate (4): To a solution of 3 (5.5 g, 16 mmol, 1 eq), cyclopropylboronic acid (3.3 g, 39 mmol, 2.5 eq) and Na₂CO₃ (4.1 g, 39 mmol, 2.5 eq) in toluene (16 mL) was added SPhos (959 mg, 2.3 mmol, 0.15 eq) and Pd(dba)₂ (269 mg, 467 μmol, 0.03 eq) at N₂. Then the mixture was stirred at 100° C. for 12 hours. The reaction mixture was diluted with H₂O (120 mL) and extracted with EA (75 mL×2). The combined organic layers were washed with saturated brine (50 mL×2), dried over Na₂SO₄, filtered and concentrated under reduced pressure and purified by column chromatography (SiO₂, petroleum ether/ethyl acetate, 50/1 to 5/1) to give 4 (4.8 g, 98% yield) as a yellow solid.

Step 5: [5-cyclopropyl-2-ethoxy-4-(4-fluorophenyl)phenyl]methanol (5): To a solution of 4 (4.8 g, 15 mmol, 1 eq) in THE (50 mL) was added DIBAL-H (1 M, 46 mL, 3 eq) at 0° C. Then the mixture was stirred at 25° C. for 1 hour. The reaction mixture was quenched by addition H₂O (70 mL) at 0° C., and then added 1N aqueous HCl (60 mL) and extracted with EA (50 mL×2). The combined organic layers were washed with saturated brine (40 mL×2), dried over Na₂SO₄, filtered and concentrated under reduced pressure to give 5 (5 g, crude) as a white solid.

Step 6: 1-(chloromethyl)-5-cyclopropyl-2-ethoxy-4-(4-fluorophenyl)benzene (6): To a solution of 5 (5 g, 17 mmol, 1 eq) in THE (50 mL) was added SOCl₂ (3.1 g, 26 mmol, 1.9 mL, 1.5 eq) and ZnCl₂ (238 mg, 1.8 mmol, 82 μL, 0.1 eq) at 0° C. Then the mixture was stirred at 25° C. for 1 hour. The reaction mixture was diluted with H₂O (100 mL) and extracted with EA (90 mL×2). The combined organic layers were washed with saturated brine (50 mL×2), dried over Na₂SO₄, filtered and concentrated under reduced pressure to give 6 (4.8 g, 90% yield) as a yellow oil.

Step 7: tert-butyl 3-(4-iodophenyl)-2-oxo-1-oxa-3,8-diazaspiro[4.5]decane-8-carboxylate (7): To a solution of 1,4-diiodobenzene (19 g, 59 mmol, 1.5 eq), tert-butyl 2-oxo-1-oxa-3,8-diazaspiro[4.5]decane-8-carboxylate (10 g, 39 mmol, 1 eq), CuI (7.4 g, 39 mmol, 1 eq), Cs₂CO₃ (51 g, 156 mmol, 4 eq) and N,N′-dimethylethane-1,2-diamine (3.4 g, 39 mmol, 4.2 mL, 1 eq) in dioxane (50 mL). The mixture was stirred at 105° C. for 16 hours. The mixture was diluted with EA (150 mL) and then filtered. The residue was dissolved in water (300 mL) and NH₃.H₂O (60 mL) and extracted with EA (70 mL×3). The combined organic layers were washed with saturated brine (100 mL), dried over Na₂SO₄, filtered and concentrated under reduced pressure to give a residue. The residue was purified by flash silica gel chromatography (ISCO®; 120 g SepaFlash® Silica Flash Column, eluent of 0-20% ethyl acetate/petroleum ether gradient) to give 7 (10.8 g, 57% yield) as a white solid. LCMS: (ES⁺) m/z (M+H)⁺=403.1., ¹H NMR (400 MHz, CDCl₃) δ 7.70-7.63 (m, 2H), 7.31 (d, J=8.8 Hz, 2H), 3.90 (br d, J=13.2 Hz, 2H), 3.72 (s, 2H), 3.40-3.25 (m, 2H), 1.98 (br d, J=13.2 Hz, 2H), 1.83-1.71 (m, 2H), 1.48 (s, 9H).

Step 8: 3-(4-iodophenyl)-1-oxa-3,8-diazaspiro[4.5]decan-2-one (8): A mixture of 7 (10.5 g, 23 mmol, 1 eq) in HCl/dioxane (4 M, 172 mL, 30 eq) was stirred at 25° C. for 1 hour. The reaction mixture was concentrated under reduced pressure to give 8 (9.1 g, crude, HCl salt) as a white solid. LCMS: (ES⁺) m/z (M+H)⁺=359.2.

Step 9: 8-((2-cyclopropyl-5-ethoxy-4′-fluoro-[1,1′-biphenyl]-4-yl)methyl)-3-(4-iodophenyl)-1-oxa-3,8-diazaspiro[4.5]decan-2-one (9): To a solution of 8 (9.1 g, 23 mmol, 1 eq, HCl salt) and 6 (7.0 g, 23 mmol, 1 eq) in DMF (145 mL) was added DIEA (8.9 g, 69 mmol, 12 mL, 3 eq). The mixture was stirred at 50° C. for 12 hours. The reaction mixture was poured into H₂O (120 mL) and extracted with EA (100 mL×3). The combined organic layer was washed with water (100 mL×2) and brine (100 mL×2), dried over Na₂SO₄, filtered and concentrated in vacuo. The residue was purified by flash silica gel chromatography (ISCO®; 120 g SepaFlash® Silica Flash Column, eluent of 0-25% ethyl acetate/petroleum ether) to give 9 (12 g, 65% yield) as a yellow solid. LCMS: (ES⁺) m/z (M+H)⁺=627.0. ¹H NMR (400 MHz, CD₃OD) δ 7.59 (br d, J=7.6 Hz, 2H), 7.38-7.23 (m, 4H), 7.04 (br d, J=7.6 Hz, 2H), 6.86 (br s, 1H), 6.64 (br s, 1H), 3.95 (br d, J=6.4 Hz, 2H), 3.69-3.52 (m, 4H), 2.61 (br s, 4H), 2.03-1.83 (m, 4H), 1.70 (br s, 1H), 1.33 (br s, 3H), 0.70 (br d, J=6.4 Hz, 2H), 0.53 (br s, 2H).

Step 10: di-tert-butyl (4-(8-((2-cyclopropyl-5-ethoxy-4′-fluoro-[1,1′-biphenyl]-4-yl)methyl)-2-oxo-1-oxa-3,8-diazaspiro[4.5]decan-3-yl)phenyl)phosphonate (10): To a mixture of 9 (12 g, 19 mmol, 1 eq), 2-tert-butoxyphosphonoyloxy-2-methyl-propane (19 g, 96 mmol, 5 eq), and KOAc (5.6 g, 57 mmol, 3 eq) in THE (140 mL) in a glove box was added tBu₃P—Pd-G2 (785 mg, 1.5 mmol, 0.08 eq). Then the mixture was stirred at 65° C. for 16 hours. The mixture was poured into 60 mL of H₂O and extracted with EA (40 mL×2). The combined organic layer was washed with water (60 mL×2) and brine (60 mL×2), dried over Na₂SO₄, filtered and concentrated in vacuo. The residue was purified by column chromatography (Al₂O₃, petroleum ether/ethyl acetate, 3/1 to 1/1) to give 10 (9.5 g, 70% yield) as a yellow oil. LCMS: (ES+) m/z (M+H)⁺=693.1. ¹H NMR (400 MHz, CDCl₃) δ 7.79 (dd, J=12.8, 8.8 Hz, 2H), 7.61 (dd, J=8.8, 3.2 Hz, 2H), 7.48-7.38 (m, 2H), 7.10 (t, J=8.8 Hz, 2H), 6.93 (s, 1H), 6.71 (s, 1H), 4.02 (q, J=6.8 Hz, 2H), 3.78 (s, 2H), 3.63 (s, 2H), 2.68 (br s, 4H), 2.16-2.01 (m, 4H), 1.77 (tt, J=8.4, 5.2 Hz, 1H), 1.49-1.43 (m, 18H), 1.40 (t, J=6.8 Hz, 3H), 0.82-0.73 (m, 2H), 0.63-0.55 (m, 2H).

Step 11: (4-(8-((2-cyclopropyl-5-ethoxy-4′-fluoro-[1,1′-biphenyl]-4-yl)methyl)-2-oxo-1-oxa-3,8-diazaspiro[4.5]decan-3-yl)phenyl)phosphonic acid (Compound 1): To a solution of 10 (9.5 g, 14 mmol, 1 eq) in DCM (50 mL) was added HCl/dioxane (4 M, 69 mL, 20 eq). The mixture was stirred at 25° C. for 10 minutes. The reaction mixture was concentrated under reduced pressure. The mixture was purified by HPLC (column: Waters Xbridge BEH C18 250×50 mm×10 μm; mobile phase: A: water (0.05% ammonium hydroxide (30% solution of ammonia in water) v/v), B: ACN; B %: 10%-40%, gradient over 20 min) to give Compound 65 (3.97 g, 50% yield, 98.38% purity, ammonium salt) as a yellow solid. LCMS: (ES⁺) m/z (M+H)⁺=581.1. ¹H NMR (400 MHz, CD₃OD) δ 7.83 (br dd, J₁=11.2 Hz, J₂=8.4 Hz, 2H), 7.60 (br dd, J₁=8.8 Hz, J₂=2.4 Hz, 2H), 7.51-7.39 (m, 2H), 7.17 (br t, J=8.8 Hz, 2H), 7.00 (s, 1H), 6.79 (s, 1H), 4.07 (q, J=6.8 Hz, 2H), 3.93 (s, 2H), 3.71 (s, 2H), 2.89-2.57 (m, 4H), 2.13-1.94 (m, 4H), 1.85-1.71 (m, 1H), 1.43 (br t, J=6.8 Hz, 3H), 0.86-0.70 (m, 2H), 0.66-0.52 (m, 2H).

### Example 2: sodium (4-(8-((2-cyclopropyl-5-ethoxy-4′-fluoro-[1,1′-biphenyl]-4-yl)methyl)-2-oxo-1-oxa-3,8-diazaspiro[4.5]decan-3-yl)phenyl)(methyl)phosphinate (Compound 2)

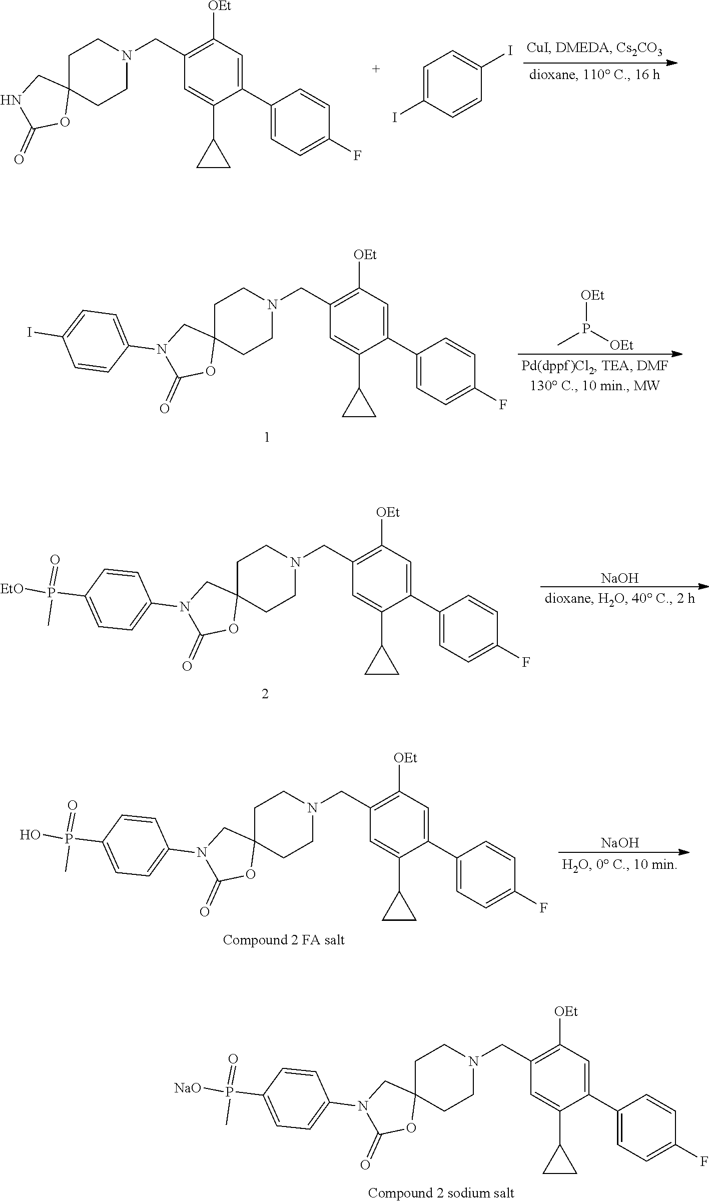

Step 1: 8-((2-cyclopropyl-5-ethoxy-4′-fluoro-[1,1′-biphenyl]-4-yl)methyl)-3-(4-iodophenyl)-1-oxa-3,8-diazaspiro[4.5]decan-2-one (1): To a mixture of 8-((2-cyclopropyl-5-ethoxy-4′-fluoro-[1,1′-biphenyl]-4-yl)methyl)-1-oxa-3,8-diazaspiro[4.5]decan-2-one (100 mg, 236 μmol, 1 eq) and 1,4-diiodobenzene (155 mg, 471 μmol, 2 eq) in dioxane (3 mL) was added CuI (45 mg, 236 μmol, 1 eq), DMEDA (21 mg, 236 μmol, 25 μL, 1 eq) and Cs₂CO₃ (307 mg, 942 μmol, 4 eq). The resulting reaction mixture was stirred at 110° C. for 16 hours. The reaction mixture was concentrated. Water (10 mL) was added to the residue, and the crude product was taken up with EtOAc (10 mL×2). The combined organic layer was concentrated and purified by prep-TLC (SiO₂, EtOAc, R^{f}=0.75) to afford 1 (130 mg, 88% yield) as a white solid. LCMS: (ES⁺) m/z (M+H)⁺=627.2.

Step 2: ethyl (4-(8-((2-cyclopropyl-5-ethoxy-4′-fluoro-[1,1′-biphenyl]-4-yl)methyl)-2-oxo-1-oxa-3,8-diazaspiro[4.5]decan-3-yl)phenyl)(methyl)phosphinate (2): To a mixture of 1 (110 mg, 176 μmol, 1 eq) and diethoxy(methyl)phosphane (48 mg, 351 μmol, 2 eq) in DMF (2 mL) was added TEA (36 mg, 351 μmol, 49 μL, 2 eq) and Pd(dppf)Cl₂ (13 mg, 18 μmol, 0.1 eq). The resulting reaction mixture was stirred under N₂ under microwave at 130° C. for 10 minutes. Water (10 mL) and EtOAc (15 mL) were added to the reaction mixture. The organic layer was separated and washed with brine (10 mL). The organic layer was concentrated to give a crude product which was purified by prep-TLC (SiO₂, EtOAc:MeOH, 10:1, R^{f}=0.5) to give 2 (100 mg, 94% yield) as a yellow oil. LCMS: (ES⁺) m/z (M+H)⁺=607.3. ¹H NMR (400 MHz, CDCl₃) δ 7.79 (dd, J=8.8, 11.2 Hz, 2H), 7.72-7.64 (m, 2H), 7.40 (dd, J=5.6, 8.4 Hz, 2H), 7.10 (t, J=8.8 Hz, 2H), 6.92 (s, 1H), 6.70 (s, 1H), 4.16-3.97 (m, 4H), 3.87-3.73 (m, 3H), 3.63 (s, 2H), 2.68 (br s, 4H), 2.06 (br s, 1H), 1.98-1.86 (m, 2H), 1.81-1.72 (m, 1H), 1.66 (s, 3H), 1.39 (t, J=6.8 Hz, 3H), 1.30-1.20 (m, 4H), 0.82-0.72 (m, 2H), 0.58 (q, J=5.2 Hz, 2H).

Step 3: (4-(8-((2-cyclopropyl-5-ethoxy-4′-fluoro-[1,1′-biphenyl]-4-yl)methyl)-2-oxo-1-oxa-3,8-diazaspiro[4.5]decan-3-yl)phenyl)(methyl)phosphinic acid (Compound 2 FA salt): To a solution of 2 (50 mg, 82 μmol, 1 eq) in dioxane (2 mL) and H₂O (1 mL) was added NaOH (26 mg, 659 μmol, 8 eq). The resulting reaction mixture was stirred at 40° C. for 2 hours. The pH was adjusted to 6 with aqueous HCl (1 M). The solution was concentrated to give a crude product, which was purified by prep-HPLC (column: Phenomenex Luna C18 200×40 mm×10 μm; mobile phase: A: water (0.2% FA, v/v), B: ACN; B %: 20%-50%, gradient over 10 min) to afford Compound 2 FA salt (45.5 mg, 86% yield, 96.94% purity, FA) as a white solid. LCMS: (ES+) m/z (M+H)⁺=579.2. ¹H NMR (400 MHz, CD₃OD) δ 7.81 (br t, J=9.6 Hz, 2H), 7.61 (br d, J=7.6 Hz, 2H), 7.46 (dd, J=5.2, 8.4 Hz, 2H), 7.24-7.14 (m, 3H), 6.93 (s, 1H), 4.38 (s, 2H), 4.17 (q, J=7.2 Hz, 2H), 3.85 (br s, 2H), 3.48 (br s, 2H), 3.41-3.32 (m, 2H), 2.27-2.18 (m, 2H), 2.08 (br s, 2H), 1.80 (br d, J=6.0 Hz, 1H), 1.52-1.36 (m, 6H), 0.84 (br d, J=8.8 Hz, 2H), 0.70 (br d, J=4.0 Hz, 2H).

Step 4: sodium (4-(8-((2-cyclopropyl-5-ethoxy-4′-fluoro-[1,1′-biphenyl]-4-yl)methyl)-2-oxo-1-oxa-3,8-diazaspiro[4.5]decan-3-yl)phenyl)(methyl)phosphinate (Compound 2 sodium salt): Compound 2 FA salt (45.5 mg, 73 μmol, 1 eq) was dissolved in H₂O (2 mL). Then NaOH (5.8 mg, 146 μmol, 2 eq) in H₂O (1 mL) was added to the suspension at 0° C. drop-wise. The suspension was stirred at 0° C. for 10 min until a clear solution was obtained. The solution was lyophilized to give Compound 2 sodium salt (45.40 mg, 99% yield, 98.91% purity, Na salt) as a white solid. LCMS: (ES⁺) m/z (M+H)⁺=579.2. ¹H NMR (400 MHz, CD₃OD) δ 7.81 (dd, J=8.8, 10.8 Hz, 2H), 7.68-7.61 (m, 2H), 7.46 (dd, J=5.6, 8.4 Hz, 2H), 7.17 (t, J=8.8 Hz, 2H), 7.00 (s, 1H), 6.78 (s, 1H), 4.06 (q, J=6.8 Hz, 2H), 3.93 (s, 2H), 3.68 (s, 2H), 2.83-2.60 (m, 4H), 2.12-1.92 (m, 4H), 1.85-1.73 (m, 1H), 1.47-1.34 (m, 6H), 0.84-0.75 (m, 2H), 0.65-0.57 (m, 2H).

### Example 3: (4-(8-((2-cyclopropyl-5-ethoxy-4′-fluoro-[1,1′-biphenyl]-4-yl)methyl)-2-oxo-1-oxa-3,8-diazaspiro[4.5]decan-3-yl)phenyl)phosphinic acid, ammonia salt (Compound 3)

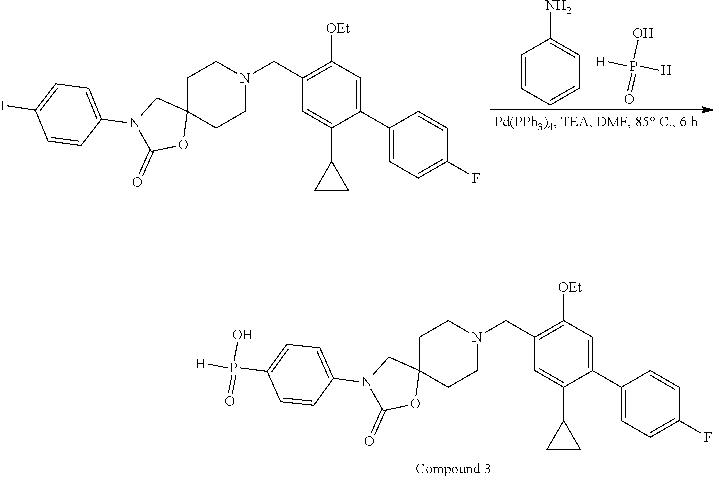

Step 1: (4-(8-((2-cyclopropyl-5-ethoxy-4′-fluoro-[1,1′-biphenyl]-4-yl)methyl)-2-oxo-1-oxa-3,8-diazaspiro[4.5]decan-3-yl)phenyl)phosphinic acid, ammonia salt (Compound 3): A solution of 8-((2-cyclopropyl-5-ethoxy-4′-fluoro-[1,1′-biphenyl]-4-yl)methyl)-3-(4-iodophenyl)-1-oxa-3,8-diazaspiro[4.5]decan-2-one (1.5 g, 2.39 mmol, 1 eq), aniline; phosphenous acid (0.56 g, 3.5 mmol, 1.5 eq), TEA (0.73 g, 7.1 mmol, 1 mL, 3 eq) and Pd(PPh₃)₄ (55 mg, 48 μmol, 0.02 eq) in DMF (13 mL) was stirred at 85° C. for 6 hours. The reaction mixture was filtered and concentrated under reduced pressure to give a residue. The residue was purified by prep-HPLC (column: Waters Xbridge C18 150×50 mm×10 μm; mobile phase: [A: water (0.05% ammonia hydroxide v/v), B: ACN]; B %: 20%-50%, 11.5 min) to give Compound 3 (228.46 mg, 16% yield, 97.1% purity, ammonium salt) as a white solid. LCMS: (ES⁺) m/z (M+H)⁺=564.9. ¹H NMR (400 MHz, CD₃OD) δ ppm 7.78-7.63 (m, 4H), 7.50-7.41 (m, 2H), 7.21-7.11 (m, 3H), 6.88 (d, J=7.6 Hz, 1H), 4.34-3.94 (m, 4H), 3.94 (br s, 2H), 3.29-3.13 (m, 3H), 2.28-2.05 (m, 4H), 1.85-1.73 (m, 1H), 1.45 (t, J=6.8 Hz, 3H), 1.31 (t, J=7.2 Hz, 1H), 0.84-0.78 (m, 2H), 0.67-0.64 (m, 2H).

The following compounds were prepared according to the procedures described above using the appropriate intermediates.

| Cpd | Characterization Data |
|---|---|
| 4 | LCMS: (ES⁺) m/z (M + H)⁺ = 580.3. ¹H NMR (400 MHz, MeOD-d4) δ 7.83-7.72 (m, |
| | 4H), 7.61 (dd, J = 2.0, 8.4 Hz, 2H), 7.51 (s, 1H), 7.18 (t, J = 8.8 Hz, 2H), 4.44 (q, J = |
| | 7.2 Hz, 2H), 3.94 (br s, 2H), 3.87 (s, 2H), 3.16-2.86 (m, 4H), 2.16-1.91 (m, 5H), 1.43- |
| | 1.35 (m, 6H), 0.96-0.88 (m, 2H), 0.70-0.61 (m, 2H). |
| 5 | LCMS: (ES+) m/z (M + H)⁺ = 596.2. ¹H NMR (400 MHz, CD₃OD) δ 8.69 (br d, J = 6.8 Hz, 1H), |
| | 8.17-8.05 (m, 2H), 7.49-7.40 (m, 2H), 7.16 (br t, J = 8.8 Hz, 2H), 7.03 (s, 1H), 6.81 (s, 1H), |
| | 4.17-4.01 (m, 4H), 3.97-3.76 (m, 2H), 3.06-2.73 (m, 4H), 2.17-1.98 (m, 4H), 1.83-1.72 (m, |
| | 1H), 1.43 (br t, J = 6.8 Hz, 3H), 0.78 (br d, J = 1.6 Hz, 2H), 0.62 (br d, J = 4.0 Hz, 2H). |
| 6 | LCMS: (ES+) m/z (M + H)⁺ = 582.1. ¹H NMR (400 MHz, CD₃OD) δ 8.74-8.65 (m, 1H), 8.16- |
| | 8.06 (m, 2H), 7.48-7.41 (m, 2H), 7.16 (t, J = 8.8 Hz, 2H), 7.07 (s, 1H), 6.84 (s, 1H), 4.14-4.06 |
| | (m, 4H), 4.00 (s, 2H), 3.18-2.94 (m, 4H), 2.19-2.04 (m, 4H), 1.78 (m, 1H), 1.43 (t, J = 6.8 Hz, |
| | 3H), 0.82-0.75 (m, 2H), 0.66-0.60 (m, 2H). |
| 7 | LCMS: (ES⁺) m/z (M + H)⁺ = 577.1 ¹H NMR (400 MHz, CD₃OD) δ 7.81 (dd, J = 10.4, |
| | 8.8 Hz, 2 H), 7.65 (dd, J = 8.4, 1.6 Hz, 2 H), 7.45 (dd, J = 8.4, 5.6 Hz, 2 H), 7.28-7.13 |
| | (m, 3 H), 6.87 (s, 1 H), 4.25-4.08 (m, 4 H), 3.56 (br s, 2 H), 3.31-3.19 (m, 2 H), 3.13- |
| | 2.90 (m, 2 H), 2.57 (s, 2 H), 1.94-1.74 (m, 5 H), 1.47-1.37 (m, 6 H), 0.87-0.67 (m, |
| | 4 H). |
| 8 | LCMS: (ES+) m/z (M + H)⁺ = 510.2. ¹H NMR (400 MHz, CD₃OD) δ 7.83-7.73 (m, |
| | 2H), 7.64-7.57 (m, 2H), 7.28 (s, 1H), 7.20 (s, 1H), 4.13 (q, J = 7.2 Hz, 2H), 3.97 (s, |
| | 2H), 3.85 (s, 2H), 3.11-2.88 (m, 4H), 2.25-2.15 (m, 1H), 2.13-2.04 (m, 2H), 2.04- |
| | 1.93 (m, 2H), 1.45 (t, J = 6.8 Hz, 3H), 1.39 (d, J = 14.0 Hz, 3H), 1.14-1.08 (m, 2H), 0.85- |
| | 0.79 (m, 2H). |

### Example 4: (4-(8-(5-cyclopropyl-2-ethoxy-4-(5-fluoropyridin-2-yl)benzyl)-2-oxo-1-oxa-3,8-diazaspiro[4.5]decan-3-yl)phenyl)phosphonic acid (Compound 9)

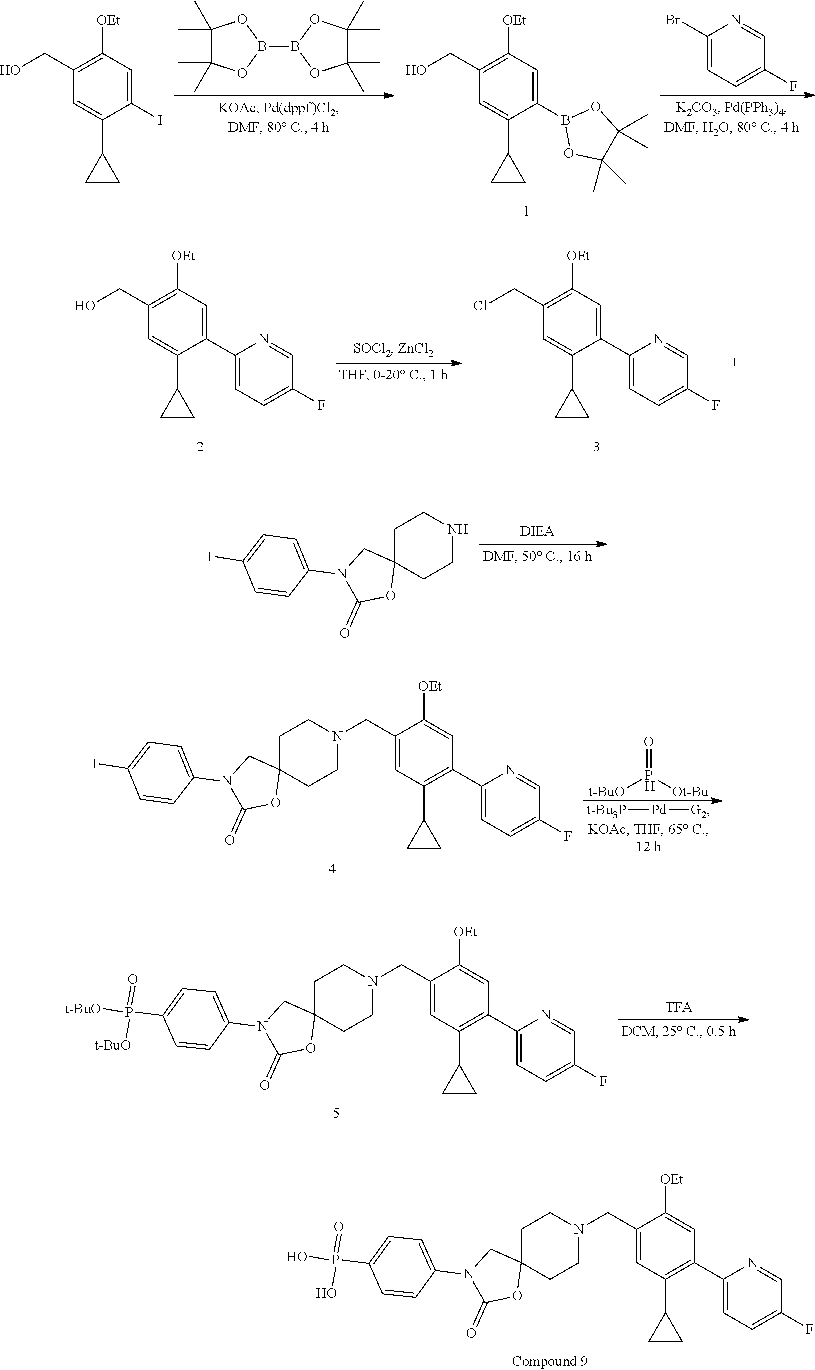

Step 1: (5-cyclopropyl-2-ethoxy-4-(4,4,5,5-tetramethyl-1,3,2-dioxaborolan-2-yl)phenyl)methanol (1): To a solution of (5-cyclopropyl-2-ethoxy-4-iodo-phenyl)methanol (3.5 g, 11 mmol, 1 eq) and 4,4,4′,4′,5,5,5′,5′-octamethyl-2,2′-bi(1,3,2-dioxaborolane) (4.2 g, 17 mmol, 1.5 eq), KOAc (3.2 g, 33 mmol, 3 eq) in DMF (60 mL) was added Pd(dppf)Cl₂ (1.2 g, 1.6 mmol, 0.15 eq) under N₂, and the mixture was stirred at 80° C. for 4 hours to give 1 (3.5 g, crude) in 60 mL DMF. The solution was used in the next step.

Step 2: (5-cyclopropyl-2-ethoxy-4-(5-fluoropyridin-2-yl)phenyl)methanol (2): To a solution of 1 (3.5 g, 11 mmol, 1 eq) in 60 mL of DMF and 2-bromo-5-fluoro-pyridine (3.9 g, 22 mmol, 2 eq) and K₂CO₃ (3.0 g, 22 mmol, 2 eq) in DMF (30 mL) and H₂O (10 mL) was added Pd(PPh₃)₄ (1.3 g, 1.1 mmol, 0.1 eq) under N₂, and the mixture was stirred at 80° C. for 4 hours. The residue was poured H₂O (30 ml) and then extracted with EA (40 mL×3). The combined organic layer was washed with water (30 mL×3) and brine (30 mL×3), dried over Na₂SO₄, filtered and concentrated in vacuo. The residue was purified by column chromatography (SiO₂, petroleum ether:ethyl acetate, 4:1) to give 2 (1.4 g, 42% yield) as a yellow oil. LCMS: (ES⁺) m/z (M+H)⁺=287.9.

Step 3: 2-(4-(chloromethyl)-2-cyclopropyl-5-ethoxyphenyl)-5-fluoropyridine (3): To a solution of 2 (1.4 g, 4.9 mmol, 1 eq) and ZnCl₂ (66 mg, 0.49 mmol, 0.1 eq) in THE (20 mL) was added SOCl₂ (0.87 g, 7.3 mmol, 1.5 eq) at 0° C., and the mixture was stirred at 20° C. for 1 hour. The residue was poured into H₂O (30 mL) and extracted with EA (40 mL×3). The combined organic layer was washed with water (30 mL×3) and brine (30 mL×3), dried over Na₂SO₄, filtered and concentrated in vacuo. The residue was purified by column chromatography (SiO₂, petroleum ether:ethyl acetate, 20:1) to give 3 (0.9 g, 55% yield) as a yellow oil. LCMS: (ES⁺) m/z (M+H)⁺=305.8. ¹H NMR (400 MHz, CDCl₃) δ 8.57 (d, J=2.8 Hz, 1H), 7.60-7.57 (m, 1H), 7.49-7.45 (m, 1H), 7.06 (s, 1H), 6.96 (s, 1H), 4.67 (s, 2H), 4.11 (q, J=6.8 Hz, 2H), 1.92-1.89 (m, 1H), 1.44 (t, J=6.8 Hz, 3H), 0.80-0.78 (m, 2H), 0.60-0.58 (m, 2H).

Step 4: 8-(5-cyclopropyl-2-ethoxy-4-(5-fluoropyridin-2-yl)benzyl)-3-(4-iodophenyl)-1-oxa-3,8-diazaspiro[4.5]decan-2-one (4): To a solution of 3-(4-iodophenyl)-1-oxa-3,8-diazaspiro[4.5]decan-2-one (0.2 g, 0.51 mmol, 1 eq, HCl salt) and 3 (0.15 g, 0.51 mmol, 1 eq) in DMF (8 mL) was added DIEA (0.20 g, 1.5 mmol, 3 eq), and the mixture was stirred at 50° C. for 12 hours. The reaction mixture was diluted with H₂O (20 mL) and extracted with ethyl acetate (20 mL×3). The combined organic layers were washed with brine (20 mL×3), dried over Na₂SO₄, filtered and concentrated under reduced pressure to give a residue. The residue was purified by column chromatography (SiO₂, petroleum ether/ethyl acetate, 1:1) to give 4 (0.20 g, 52% yield, 82% purity) as a yellow oil. LCMS: (ES⁺) m/z (M+H)⁺=627.9.

Step 5: di-tert-butyl (4-(8-(5-cyclopropyl-2-ethoxy-4-(5-fluoropyridin-2-yl)benzyl)-2-oxo-1-oxa-3,8-diazaspiro[4.5]decan-3-yl)phenyl)phosphonate (5): A mixture of 4 (0.2 g, 0.32 mmol, 1 eq), 2-tert-butoxyphosphonoyloxy-2-methyl-propane (0.31 g, 1.6 mmol, 5 eq), KOAc (94 mg, 0.96 mmol, 3 eq), and tBu₃P—Pd-G₂ (15 mg, 29 mmol, 0.10 eq) in THF (3 mL) was degassed and purged with N₂ 3 times, and then the mixture was stirred at 65° C. for 12 hours under N₂ atmosphere. The reaction mixture was diluted with H₂O (20 mL) and extracted with ethyl acetate (20 mL×3). The combined organic layers were washed with brine (20 mL×3), dried over Na₂SO₄, filtered and concentrated under reduced pressure to give 5 (0.30 g, crude) as a yellow oil. LCMS: (ES⁺) m/z (M+H)⁺=694.1.

Step 6: (4-(8-(5-cyclopropyl-2-ethoxy-4-(5-fluoropyridin-2-yl)benzyl)-2-oxo-1-oxa-3,8-diazaspiro[4.5]decan-3-yl)phenyl)phosphonic acid (Compound 9): To a solution of 5 (0.15 g, 0.22 mmol, 1 eq) in DCM (2 mL) was added TFA (0.62 g, 5.4 mmol, 25 eq), and the mixture was stirred at 25° C. for 0.5 hour. The mixture was concentrated in vacuo. The residue was purified by prep-HPLC (column: Xtimate C18 150×40 mm×10 μm; mobile phase: A: water (0.05% ammonium hydroxide v/v), B: ACN; B %: 5%-35%, gradient over 10 min) to give Compound 9 (23 mg, 48% yield, 99.9% purity, ammonium salt) as a white solid. LCMS: (ES⁺) m/z (M+H)⁺=582.1. ¹H NMR (400 MHz, CD₃OD) δ 8.56 (d, J=2.4 Hz, 1H), 7.85-7.80 (m, 2H), 7.74-7.72 (m, 2H), 7.57 (dd, J₁=8.4 Hz, J₂=2.4 Hz, 2H), 7.26 (s, 1H), 7.06 (s, 1H), 4.23 (s, 2H), 4.16 (q, J=6.8 Hz, 2H), 3.85 (s, 2H), 3.35 (s, 2H), 3.16 (t, J=10.0 Hz, 2H), 2.18-2.10 (m, 4H), 1.94-1.88 (m, 1H), 1.46 (t, J=6.8 Hz, 3H), 0.83-0.79 (m, 2H), 0.65-0.61 (m, 2H).

### Example 5: [4-[8-[[5-cyclopropyl-2-ethoxy-6-(4-fluorophenyl)-3-pyridyl]methyl]-2-oxo-1-oxa-3,8-diazaspiro[4.5]decan-3-yl]phenyl]phosphonic acid (Compound 10)

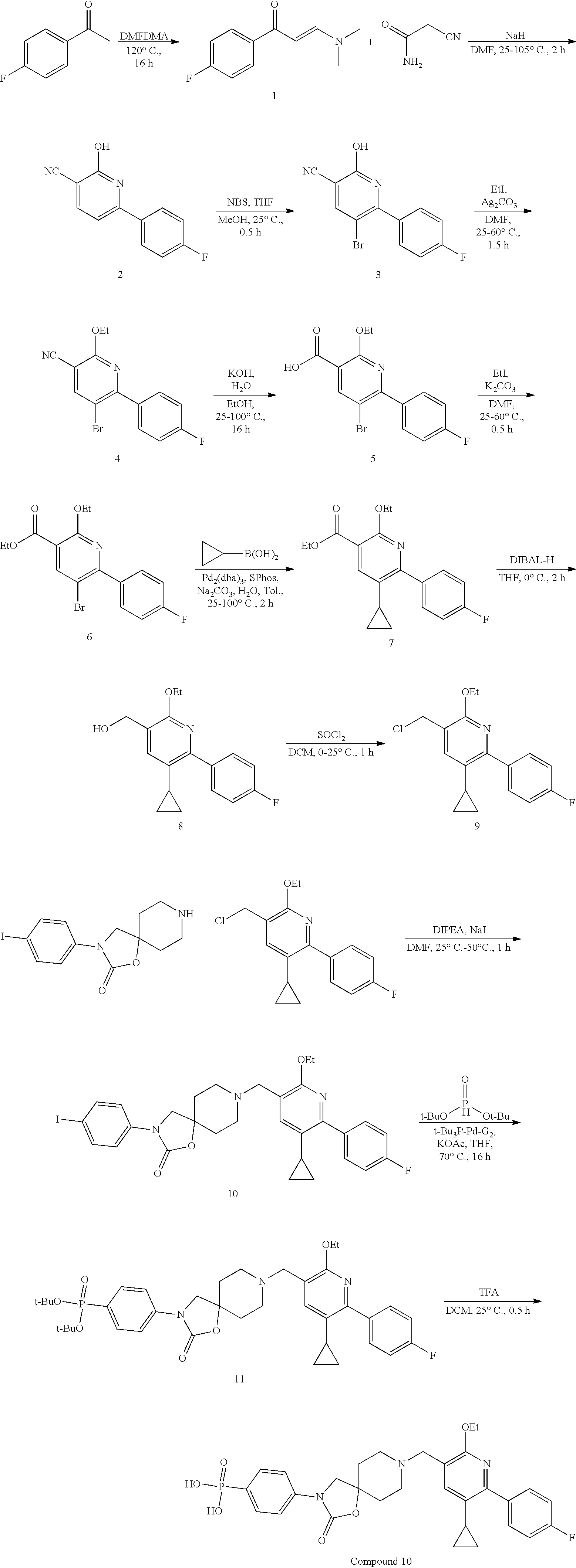

Step 1: (E)-3-(dimethylamino)-1-(4-fluorophenyl)prop-2-en-1-one (1): The mixture of 1-(4-fluorophenyl)ethanone (5.0 g, 36 mmol, 4.4 mL, 1.0 eq) and DMFDMA (26 g, 0.22 mol, 29 mL, 6.0 eq) was heated to 120° C. and stirred for 16 hours. The reaction was concentrated under reduced pressure to remove DMFDMA and then triturated with MTBE (20 mL) for 10 minutes and filtered, and the cake was washed with MTBE (5 mL) to give 1 (6.3 g, 90% yield) as a light yellow solid.

Step 2: 6-(4-fluorophenyl)-2-hydroxy-pyridine-3-carbonitrile (2): To a mixture of NaH (3.8 g, 96 mmol, 60% purity, 3.2 eq) in DMF (60 mL) was added 1 (5.8 g, 30 mmol, 1.0 eq) and 2-cyanoacetamide (2.8 g, 33 mmol, 1.1 eq) at 25° C. under N₂ protection. The mixture was degassed and purged with N₂ 3 times, then heated to 105° C. and stirred for 2 hours. The reaction mixture was poured into cooled saturated NH₄Cl solution (800 mL) and stirred for 10 minutes, then filtered and triturated with MTBE (20 mL) at 25° C. for 30 minutes to give 2 (6.0 g, 93% yield) as a yellow solid.

Step 3: 5-bromo-6-(4-fluorophenyl)-2-hydroxy-pyridine-3-carbonitrile(3): 2 (6.0 g, 28 mmol, 1.0 eq) was concentrated with THF (3.0 mL×3) in vacuo to remove water, and then to the mixture of 2 (6 g, 28 mmol, 1.0 eq) in THF (5.0 mL) and MeOH (5.0 mL) was added NBS (5.5 g, 31 mmol, 1.1 eq) at 25° C. The reaction mixture was stirred for 0.5 hour. The reaction mixture was concentrated under reduced pressure to remove solvent. Ethyl acetate (30 mL) and water (30 mL) were added to the mixture, stirred for 10 minutes and separated. The organic phase was washed with brine (30 mL), dried with anhydrous Na₂SO₄, filtered and concentrated in vacuum. The residue was triturated with MTBE (30 mL) to give 3 (5.8 g, 71% yield) as a yellow solid.

Step 4: 5-bromo-2-ethoxy-6-(4-fluorophenyl)pyridine-3-carbonitrile (4): To a mixture of 3 (5.8 g, 20 mmol, 1.0 eq) in DMF (100 mL) was added Ag₂CO₃ (8.2 g, 30 mmol, 1.3 mL, 1.5 eq) and EtI (3.7 g, 24 mmol, 1.9 mL, 1.2 eq) at 25° C. The mixture was stirred for 0.5 hour at 25° C. and then heated to 60° C. and stirred for 1 h. The mixture was filtered and then ethyl acetate (300 mL) and water (200 mL) were added to the mixture, stirred for 10 minutes and separated. The aqueous phase was extracted with ethyl acetate (100 mL), and the combined organic phase was washed with brine (100 mL), dried with anhydrous Na₂SO₄, filtered and concentrated. The residue was purified by column chromatography (SiO₂, Petroleum ether: Ethyl acetate=20:1 to 1:5) to give 4 (3.6 g, 57% yield) as a white solid.

Step 5: 5-bromo-2-ethoxy-6-(4-fluorophenyl)pyridine-3-carboxylic acid (5): To a mixture of 4 (3.6 g, 11 mmol, 1.0 eq) in H₂O (10 mL) and EtOH (100 mL) was added KOH (6.3 g, 0.11 mol, 10 eq) at 25° C. The mixture was heated to 100° C. and stirred for 16 hours. The reaction mixture was cooled to 0° C. and adjusted pH to 2-3 with 6 M aqueous HCl, and then diluted with water (50 mL) and extracted with ethyl acetate (100 mL×2). The combined organic layers were washed with brine (100 mL), dried with anhydrous Na₂SO₄, filtered and concentrated under reduced pressure. The residue was triturated with MTBE (20 mL) to give 5 (3.8 g, 11 mmol, 99% yield) as a light yellow solid.

Step 6: ethyl 5-bromo-2-ethoxy-6-(4-fluorophenyl)pyridine-3-carboxylate (6): To a mixture of 5 (2.4 g, 7.0 mmol, 1.0 eq) in DMF (60 mL) was added EtI (1.7 g, 10 mmol, 0.85 mL, 1.5 eq) and K₂CO₃ (1.9 g, 14 mmol, 2.0 eq) at 25° C., and then the mixture was heated to 60° C. and stirred for 0.5 hour. Water (300 mL) was added to the mixture and extracted with ethyl acetate (200 mL×2), the combined organic phase was washed with brine (100 mL), dried with anhydrous Na₂SO₄, filtered and concentrated under reduced pressure. The residue was purified by column chromatography (SiO₂, petroleum ether:ethyl acetate, 50:1 to 20:1) to give 6 (3.5 g, crude) as a white solid.

Step 7: ethyl-cyclopropyl-2-ethoxy-6-(4-fluorophenyl)pyridine-3-carboxylate (7): To a mixture of 6 (3.4 g, 9.2 mmol, 1.0 eq), cyclopropylboronic acid (2.4 g, 28 mmol, 3.0 eq), Na₂CO₃ (2.9 g, 28 mmol, 3.0 eq) and S-Phos (0.57 g, 1.4 mmol, 0.15 eq) in H₂O (17 mL) and toluene (28 mL) was added Pd₂(dba)₃ (0.59 g, 0.65 mmol, 0.07 eq) at 25° C. The reaction mixture was degassed and replaced with nitrogen 3 times, then heated to 100° C. and stirred for 2 hours. The reaction mixture was cooled to 25° C. and poured into water (100 mL). The mixture was extracted with ethyl acetate (50 mL×2). The combined organic phase was washed with brine (50 mL), dried with anhydrous Na₂SO₄, filtered and concentrated to give residue. The residue was purified by column chromatography (SiO₂, petroleum ether:ethyl acetate, 50:1 to 20:1) to give 7 (3.5 g, crude) as a colorless liquid.

Step 8: [5-cyclopropyl-2-ethoxy-6-(4-fluorophenyl)-3-pyridyl]methanol (8): To a mixture of 7 (1.0 g, 3.0 mmol, 1.0 eq) in THE (20 mL) was added dropwise DIBAL-H (1 M, 11 mL, 3.5 eq) at 0° C. under N₂ protection. The reaction mixture was stirred at 0° C. for 2 hours. The reaction mixture was quenched by seignette salt solution (20 mL) at 0° C. The reaction mixture was extracted with ethyl acetate (30 mL×2). The combined organic phase was washed with brine (20 mL), dried with anhydrous Na₂SO₄, filtered and concentrated to give residue. The residue was purified by column chromatography (SiO₂, petroleum ether:ethyl acetate, 30:1 to 6:1) to give 8 (0.81 g, 93% yield) as a colorless liquid.

Step 9: 3-(chloromethyl)-5-cyclopropyl-2-ethoxy-6-(4-fluorophenyl)pyridine (9): To a mixture of 8 (0.77 g, 2.7 mmol, 1.0 eq) in DCM (15 mL) was added dropwise SOCl₂ (0.35 g, 2.9 mmol, 0.21 mL, 1.1 eq) at 0° C. under N₂ protection. Then the reaction mixture was heated to 25° C. and stirred for 1 h. Water (20 mL) was added to the reaction mixture, and the mixture was stirred at 25° C. for 15 minutes, then separated. The aqueous phase was extracted with DCM (20 mL). The combined organic phase was washed with brine (20 mL), dried with anhydrous Na₂SO₄, filtered and concentrated to give 9 (0.85 g, crude) as a light yellow solid.

Step 10: 8-[[5-cyclopropyl-2-ethoxy-6-(4-fluorophenyl)-3-pyridyl]methyl]-3-(4-iodophenyl)-1-oxa-3,8-diazaspiro[4.5]decan-2-one (10): To a mixture of 3-(4-iodophenyl)-1-oxa-3,8-diazaspiro[4.5]decan-2-one (0.37 g, 1.0 mmol, 1.0 eq) and 9 (0.47 g, 1.5 mmol, 1.5 eq) in DMF (5 mL) was added DIEA (0.66 g, 5.1 mmol, 0.89 mL, 5.0 eq) and NaI (31 mg, 0.20 mmol, 0.20 eq) at 25° C., and then the mixture was heated to 50° C. and stirred for 1 hour. Water (20 mL) was added to the reaction mixture, and the mixture was extracted with ethyl acetate (30 mL×2). The combined organic phase was washed with brine (20 mL), dried with anhydrous Na₂SO₄, filtered and concentrated under reduced pressure. The residue was purified by prep-TLC (petroleum ether:ethyl acetate, 1:1) to give 10 (0.20 g, 32% yield) as a white solid.

Step 11: 8-[[5-cyclopropyl-2-ethoxy-6-(4-fluorophenyl)-3-pyridyl]methyl]-3-(4-ditert-butoxyphosphorylphenyl)-1-oxa-3,8-diazaspiro[4.5]decan-2-one(11): To a mixture of 10 (0.14 g, 0.22 mmol, 1.0 eq), 2-tert-butoxyphosphonoyloxy-2-methyl-propane (0.22 g, 1.1 mmol, 5.0 eq), and KOAc (66 mg, 0.67 mmol, 3.0 eq) in THE (2 mL) in a glove box was added tBu₃P—Pd-G2 (46 mg, 89 μmol, 0.40 eq). Then the reaction mixture was heated to 70° C. and stirred for 16 hours. The reaction mixture was filtered, and the filtered solution was purified by prep-TLC (petroleum ether:ethyl acetate, 0:1) to give 11 (68 mg, 44% yield) as a colorless solid.

Step 12: [4-[8-[[5-cyclopropyl-2-ethoxy-6-(4-fluorophenyl)-3-pyridyl]methyl]-2-oxo-1-oxa-3,8-diazaspiro[4.5]decan-3-yl]phenyl]phosphonic acid (Compound 10): To a mixture of 8-[[5-cyclopropyl-2-ethoxy-6-(4-fluorophenyl)-3-pyridyl]methyl]-3-(4-ditert-butoxyphosphorylphenyl)-1-oxa-3,8-diazaspiro[4.5]decan-2-one (68 mg, 98 μmol, 1 eq) in DCM (3 mL) was added TFA (1.23 g, 11 mmol, 0.80 mL, 110 eq) at 25° C., then the mixture was stirred for 0.5 hour at 25° C. The reaction mixture was concentrated under reduced pressure. The residue was purified by prep-HPLC (column: Welch Xtimate C18 150×25 mm×5 μm; mobile phase: A: water (0.2% FA, v/v), B: ACN; B %: 20%-40%, gradient over 10 min). The product was dissolved in water (4 mL) and ammonium hydroxide (30% NH₃ in water, 1 mL) and lyophilized to give Compound 10 (27 mg, 42% yield) as a white solid. LCMS: (ES⁺) m/z (M+H)⁺=582.1. ¹H NMR (400 MHz, DMSO-d6) δ 7.75 (dd, J=8.4, 5.6 Hz, 2H), 7.67-7.57 (m, 2H), 7.56-7.47 (m, 2H), 7.36 (s, 1H), 7.27 (t, J=8.8 Hz, 2H), 4.37-4.27 (m, 2H), 3.88 (s, 2H), 3.51-3.49 (m, 2H), 2.62-2.53 (m, 4H), 1.99-1.83 (m, 5H), 1.31 (t, J=7.2 Hz, 3H), 0.91-0.82 (m, 2H), 0.60-0.51 (m, 2H).

### Example 6: ((3-(8-((5-cyclopropyl-2-ethoxy-6-(4-fluorophenyl)pyridin-3-yl)methyl)-2-oxo-1-oxa-3,8-diazaspiro[4.5]decan-3-yl)bicyclo[1.1.1]pentan-1-yl)methyl)phosphonic acid (Compound 11)

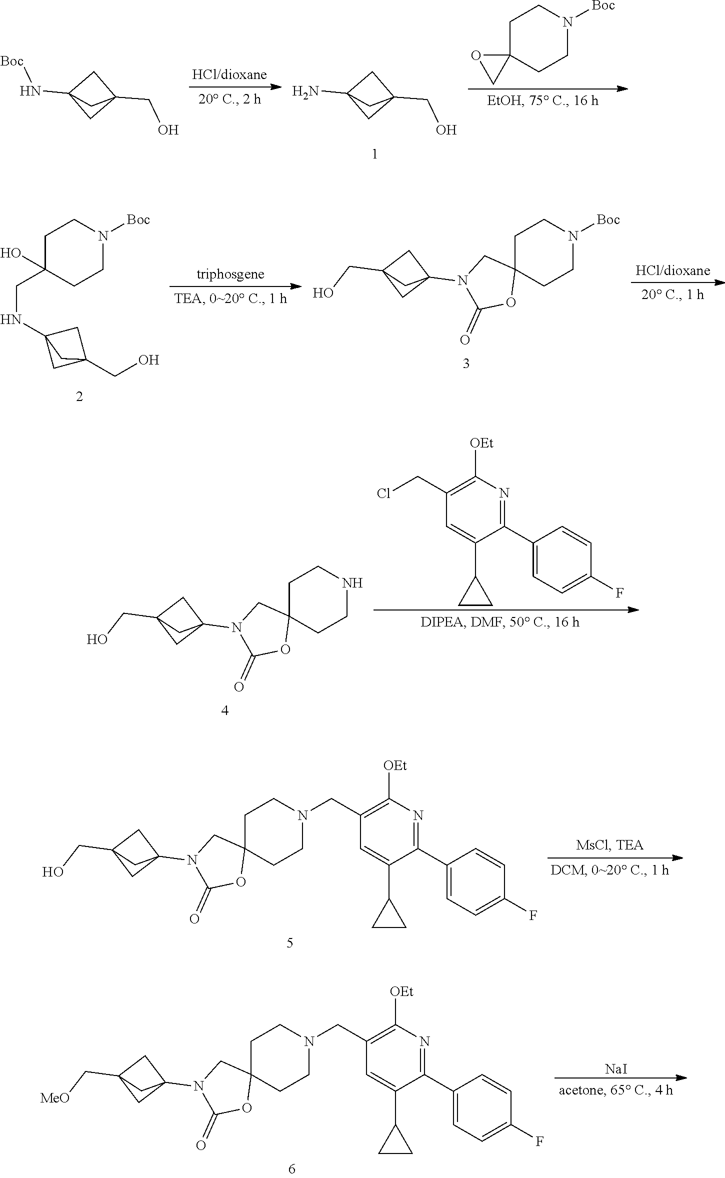

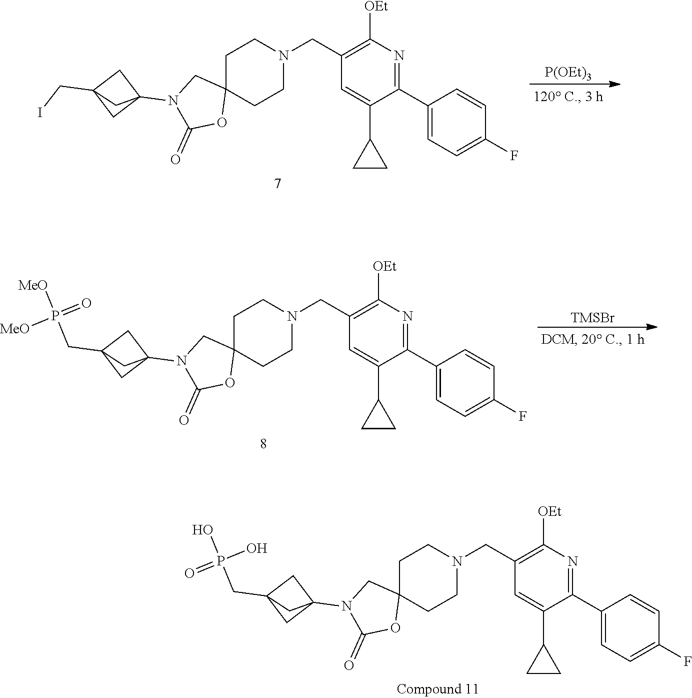 [SMILES: CCOc1nc(-c2ccc(F)cc2)c(C2CC2)cc1CN1CCC2(CC1)CN(C13CC(CI)(C1)C3)C(=O)O2 ; "TMSBr" ; CCOc1nc(-c2ccc(F)cc2)c(C2CC2)cc1CN1CCC2(CC1)CN(C13CC(CP(=O)(OC)OC)(C1)C3)C(=O)O2 ; "P(OEt)3" ; CCOc1nc(-c2ccc(F)cc2)c(C2CC2)cc1CN1CCC2(CC1)CN(C13CC(CP(=O)(O)O)(C1)C3)C(=O)O2]

Step 1: (3-aminobicyclo[1.1.1]pentan-1-yl)methanol (1): To a solution of tert-butyl N-[1-(hydroxymethyl)-3-bicyclo[1.1.1]pentanyl]carbamate (0.9 g, 4.2 mmol, 1 eq) in HCl/dioxane (4 M, 15 mL, 14.22 eq) was stirred at 20° C. for 2 hours. After completion, the reaction mixture was concentrated under reduced pressure to remove solvent. MeOH (20 mL) was added, and the mixture was basified to pH 9 by basic resin. The mixture was filtered through a Celite pad, and the filtrate was concentrated to give product 1 (600 mg, crude) as a yellow oil. ¹H NMR (400 MHz, DMSO-d6) δ 6.62-5.33 (m, 1H), 4.74-4.24 (m, 1H), 3.43 (s, 2H), 1.68 (s, 6H).

Step 2: tert-butyl 4-hydroxy-4-(((3-(hydroxymethyl)bicyclo[1.1.1]pentan-1-yl)amino)methyl)piperidine-1-carboxylate (2): A solution of 1 (150 mg, 1.3 mmol, 1 eq) and tert-butyl 1-oxa-6-azaspiro[2.5]octane-6-carboxylate (283 mg, 1.3 mmol, 1 eq) in EtOH (8 mL) was stirred at 75° C. for 16 hours. After completion, the reaction mixture was concentrated under reduced pressure to remove solvent. The residue was purified by prep-TLC (SiO₂, Ethyl acetate:Methanol=5:1, R^{f}=0.3) to afford product 2 (250 mg, 58% yield) as a yellow oil. ¹H NMR (400 MHz, CDCl₃-d) δ 3.85 (br s, 2H), 3.71 (s, 2H), 3.16 (br t, J=11.6 Hz, 2H), 2.53 (s, 2H), 1.71 (s, 6H), 1.54-1.36 (m, 14H).

Step 3: tert-butyl 3-(3-(hydroxymethyl)bicyclo[1.1.1]pentan-1-yl)-2-oxo-1-oxa-3,8-diazaspiro[4.5]decane-8-carboxylate (3): To a solution of 2 (80 mg, 245 μmol, 1 eq) in DCM (5 mL) was added TEA (124 mg, 1.2 mmol, 0.17 mL, 5 eq). The mixture was cooled to 0° C. To this mixture was added a solution of triphosgene (73 mg, 245 μmol, 1 eq) in DCM (1 mL). The mixture was stirred at 20° C. for 1 hour. After completion, the mixture was quenched by H₂O (10 mL) and extracted with DCM (10 mL×2). The combined organic phase was dried with anhydrous Na₂SO₄, filtered and concentrated in vacuum. The residue was purified by prep-TLC (SiO₂, Petroleum ether: Ethyl acetate=0:1, R^{f}=0.4) to give 3 (50 mg, 58% yield) as a yellow solid. ¹H NMR (400 MHz, CDCl₃-d) δ 3.82 (br s, 2H), 3.75 (br s, 2H), 3.35-3.25 (m, 4H), 2.10-1.98 (m, 6H), 1.90 (br d, J=13.2 Hz, 2H), 1.72-1.61 (m, 2H), 1.47 (s, 9H).

Step 4: 3-(3-(hydroxymethyl)bicyclo[1.1.1]pentan-1-yl)-1-oxa-3,8-diazaspiro[4.5]decan-2-one (4): A solution of 3 (0.20 g, 0.57 mmol, 1.0 eq) in HCl/dioxane (4.0 M, 4.0 mL, 28 eq) was stirred at 20° C. for 1 hour. The reaction mixture was concentrated under reduced pressure to remove solvent. The crude product 4 (0.16 g, 98% yield, HCl salt) as a white solid was used in the next step directly without purification. ¹H NMR (400 MHz, DMSO-d6) δ 3.47 (s, 2H), 3.40 (s, 3H), 3.16 (br s, 2H), 3.06 (br s, 2H), 2.01-1.92 (m, 5H), 1.86 (s, 6H).

Step 5: 8-((5-cyclopropyl-2-ethoxy-6-(4-fluorophenyl)pyridin-3-yl)methyl)-3-(3-(hydroxymethyl)bicyclo[1.1.1]pentan-1-yl)-1-oxa-3,8-diazaspiro[4.5]decan-2-one (5): To a solution of 4 (0.16 g, 0.55 mmol, 1.0 eq, HCl salt) and 3-(chloromethyl)-5-cyclopropyl-2-ethoxy-6-(4-fluorophenyl)pyridine (0.17 g, 0.55 mmol, 1.0 eq) in DMF (3.0 mL) was added DIEA (0.57 g, 4.4 mmol, 0.77 mL, 8.0 eq) and NaI (17 mg, 0.11 mmol, 0.20 eq). The mixture was stirred at 50° C. for 16 hours. The mixture was quenched with H₂O (30 mL) and extracted with ethyl acetate (30 mL×3). The combined organic phase was dried with anhydrous Na₂SO₄, filtered and concentrated in vacuum. The residue was purified by prep-TLC (SiO₂, Ethyl acetate:Methanol=5:1, R^{f}=0.41) to give 5 (0.23 g, 78% yield) as a white solid. ¹H NMR (400 MHz, CDCl₃-d) δ 7.78-7.70 (m, 2H), 7.27-7.25 (m, 1H), 7.16-7.08 (m, 2H), 4.39 (q, J=7.2 Hz, 2H), 3.74 (s, 2H), 3.56 (s, 2H), 3.27 (s, 2H), 2.61 (br s, 4H), 2.04 (s, 6H), 2.00-1.90 (m, 2H), 1.85-1.74 (m, 3H), 1.36 (t, J=7.2 Hz, 3H), 0.92-0.82 (m, 2H), 0.63-0.53 (m, 2H).

Step 6: (3-(8-((5-cyclopropyl-2-ethoxy-6-(4-fluorophenyl)pyridin-3-yl)methyl)-2-oxo-1-oxa-3,8-diazaspiro[4.5]decan-3-yl)bicyclo[1.1.1]pentan-1-yl)methyl methanesulfonate (6): To a solution of 5 (0.17 g, 0.33 mmol, 1.0 eq) in DCM (8.0 mL) was added TEA (66 mg, 0.65 mmol, 91 μL, 2.0 eq). Then MsCl (45 mg, 0.39 mmol, 30 μL, 1.2 eq) was added at 0° C. The mixture was stirred at 20° C. for 1 hour. The mixture was quenched by addition of saturated aqueous NaHCO₃ (20 mL) and extracted with DCM (20 mL×2). The combined organic phase was dried with anhydrous Na₂SO₄, filtered and concentrated in vacuum. The crude product 6 (0.19 g, 97% yield) as a yellow oil was used in the next step directly without purification. ¹H NMR (400 MHz, CDCl₃-d) δ 7.79-7.70 (m, 2H), 7.26 (s, 1H), 7.16-7.09 (m, 2H), 4.39 (q, J=7.2 Hz, 2H), 4.34 (s, 2H), 3.56 (s, 2H), 3.27 (s, 2H), 3.03 (s, 3H), 2.61 (br s, 4H), 2.15 (s, 6H), 2.02-1.91 (m, 3H), 1.85-1.74 (m, 2H), 1.36 (t, J=7.2 Hz, 3H), 0.91-0.83 (m, 2H), 0.61-0.52 (m, 2H).

Step 7: 8-((5-cyclopropyl-2-ethoxy-6-(4-fluorophenyl)pyridin-3-yl)methyl)-3-(3-(iodomethyl)bicyclo[1.1.1]pentan-1-yl)-1-oxa-3,8-diazaspiro[4.5]decan-2-one (7): To a solution of 6 (0.19 g, 0.32 mmol, 1.0 eq) in acetone (8.0 mL) was added NaI (0.24 g, 1.6 mmol, 5.0 eq). The mixture was stirred at 65° C. for 4 hours. The mixture was filtered through a Celite pad, and the filtrate was concentrated to give crude product. H₂O (30 mL) was added, and the mixture was extracted with ethyl acetate (30 mL×2). The combined organic phase was dried with anhydrous Na₂SO₄, filtered and concentrated in vacuum. The residue was purified by prep-TLC (SiO₂, Ethyl acetate:Methanol=10:1, R^{f}=0.5) to give 7 (0.20 g, 100% yield) as a white solid. H NMR (400 MHz, CDCl₃-d) δ 7.78-7.70 (m, 2H), 7.26 (br s, 1H), 7.17-7.08 (m, 2H), 4.39 (q, J=7.2 Hz, 2H), 3.56 (s, 2H), 3.40 (s, 2H), 3.26 (s, 2H), 2.70-2.55 (m, 4H), 2.05 (s, 6H), 2.00-1.90 (m, 3H), 1.84-1.73 (m, 2H), 1.36 (t, J=7.2 Hz, 3H), 0.91-0.83 (m, 2H), 0.58 (q, J=5.2 Hz, 2H).

Step 8: dimethyl ((3-(8-((5-cyclopropyl-2-ethoxy-6-(4-fluorophenyl)pyridin-3-yl)methyl)-2-oxo-1-oxa-3,8-diazaspiro[4.5]decan-3-yl)bicyclo[1.1.1]pentan-1-yl)methyl)phosphonate (8): To a solution of 7 (0.15 g, 0.24 mmol, 1.0 eq) in trimethyl phosphite (3.2 g, 25 mmol, 3.0 mL, 107 eq) was stirred at 120° C. for 3 hours. The reaction mixture was concentrated under reduced pressure to remove solvent. The residue was purified by prep-TLC (SiO₂, Ethyl acetate:Methanol=5:1, R^{f}=0.3) to give 8 (0.13 g, 89% yield) as a white solid. ¹H NMR (400 MHz, CDCl₃-d) δ 7.74 (dd, J=5.6, 8.8 Hz, 2H), 7.27-7.26 (m, 1H), 7.16-7.08 (m, 2H), 4.40 (q, J=7.2 Hz, 2H), 3.74 (d, J=10.8 Hz, 6H), 3.56 (s, 2H), 3.25 (s, 2H), 2.62 (br d, J=10.8 Hz, 4H), 2.16-2.13 (m, 7H), 2.11 (s, 1H), 2.01-1.91 (m, 3H), 1.78 (br s, 2H), 1.36 (t, J=7.2 Hz, 3H), 0.91-0.83 (m, 2H), 0.58 (br d, J=5.2 Hz, 2H).

Step 9: ((3-(8-((5-cyclopropyl-2-ethoxy-6-(4-fluorophenyl)pyridin-3-yl)methyl)-2-oxo-1-oxa-3,8-diazaspiro[4.5]decan-3-yl)bicyclo[1.1.1]pentan-1-yl)methyl)phosphonic acid (Compound 11): To a solution of 8 (0.11 g, 0.18 mmol, 1.0 eq) in DCM (6.0 mL) was added dropwise via syringe TMSBr (0.55 g, 3.6 mmol, 0.47 mL, 20 eq) in DCM (1.0 mL). The mixture was stirred at 20° C. for 1 hour. The reaction mixture was concentrated under reduced pressure to remove solvent. The mixture was quenched by addition of H₂O (0.1 mL). The residue was purified by prep-HPLC (column: Waters Xbridge Prep OBD C18 150×40 mm×10 μm; mobile phase: [A; water (0.05% NH₃.H₂O+10 mM NH₄HCO₃), B: ACN]; B %: 20%-70%, 8 min) to give Compound 11 (71 mg, 66% yield, 100% purity, ammonium salt) as a white solid. LCMS: (ES⁺) m/z (M+H)⁺=586.3. ¹H NMR (400 MHz, DMSO-d₆) δ 7.74 (dd, J=6.0, 8.6 Hz, 2H), 7.32 (s, 1H), 7.27 (t, J=8.8 Hz, 2H), 4.34-4.25 (m, 2H), 3.45 (s, 2H), 3.26 (s, 2H), 2.42 (br s, 4H), 1.97-1.89 (m, 7H), 1.75 (br s, 4H), 1.67 (br d, J=18.0 Hz, 2H), 1.29 (t, J=7.2 Hz, 3H), 0.89-0.81 (m, 2H), 0.53 (q, J=5.2 Hz, 2H).

The following compounds were prepared according to the procedures described above using the appropriate intermediates.

| Cpd | Characterization Data |
|---|---|
| 12 | LCMS: (ES⁺) m/z (M + H)⁺ = 585.3. ¹H NMR (400 MHz, DMSO-d₆) δ 7.46 (dd, J = 5.6, |
| | 8.8 Hz, 2H), 7.28-7.20 (m, 2H), 6.92 (s, 1H), 6.72 (s, 1H), 3.99 (q, J = 7.2 Hz, 2H), |
| | 3.54-3.44 (m, 2H), 3.25 (s, 2H), 2.43 (br s, 4H), 1.94 (s, 6H), 1.79-1.66 (m, 7H), 1.28 |
| | (t, J = 6.8 Hz, 3H), 0.78-0.66 (m, 2H), 0.54-0.45 (m, 2H). |

### Example 7: (4-(8-((2-cyclopropyl-5-ethoxy-4′-fluoro-[1,1′-biphenyl]-4-yl)methyl)-2-oxo-1-oxa-3,8-diazaspiro[4.5]decan-3-yl)phenyl)boronic acid (Compound 13)

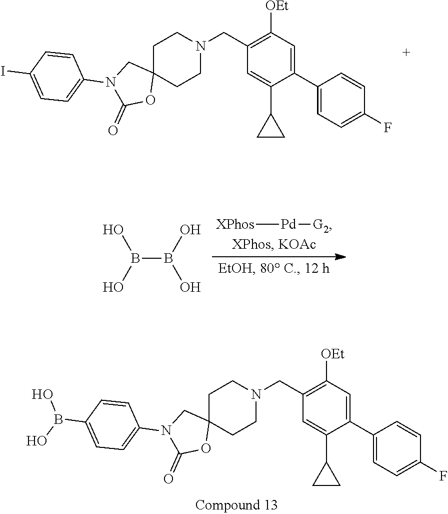

A mixture of 8-[[5-cyclopropyl-2-ethoxy-4-(4-fluorophenyl)phenyl]methyl]-3-(4-iodophenyl)-1-oxa-3,8-diazaspiro[4.5]decan-2-one (100 mg, 0.16 mmol, 1 eq), hypoboric acid (43 mg, 0.48 mmol, 3 eq), XPhos-Pd-G₂ (6.3 mg, 8.0 μmol, 0.05 eq), XPhos (7.6 mg, 16 μmol, 0.1 eq) and KOAc (47 mg, 0.48 mmol, 3 eq) in EtOH (1 mL) was degassed and purged with N_{2 3} times, and then the mixture was stirred at 80° C. for 12 hours under N₂ atmosphere. On completion, the reaction mixture was filtered and concentrated under reduced pressure to give a residue. The residue was purified by prep-HPLC (column: UniSil 3-100 C18 Ultra (150×25 mm×3 μm); mobile phase: A: water (0.225% FA), B: ACN; B %: 25%-55% gradient over 10 min) to give Compound 13 (41.88 mg, 75.96 μmol, 47.59% yield, 98.75% purity) as a white solid. LCMS: (ES⁺) m/z (M+H)⁺=545.2. ¹H NMR (400 MHz, CD₃OD) δ 7.84-7.67 (m, 2H), 7.58-7.51 (m, 2H), 7.48-7.40 (m, 2H), 7.20-7.12 (m, 2H), 7.04 (m, 1H), 6.83 (s, 1H), 4.13-4.05 (m, 2H), 3.98-3.89 (m, 4H), 3.10-2.89 (m, 4H), 2.20-2.00 (m, 4H), 1.84-1.73 (m, 1H), 1.47-1.39 (m, 3H), 0.82-0.75 (m, 2H), 0.65-0.58 (m, 2H).

### Example 8: 1-carbamimidoyl-3-[2-[4-[8-[[5-cyclopropyl-2-ethoxy-4-(4-fluorophenyl)phenyl]methyl]-2-oxo-1-oxa-3,8-diazaspiro[4.5]decan-3-yl]phenyl]ethyl]guanidine (Compound 14)

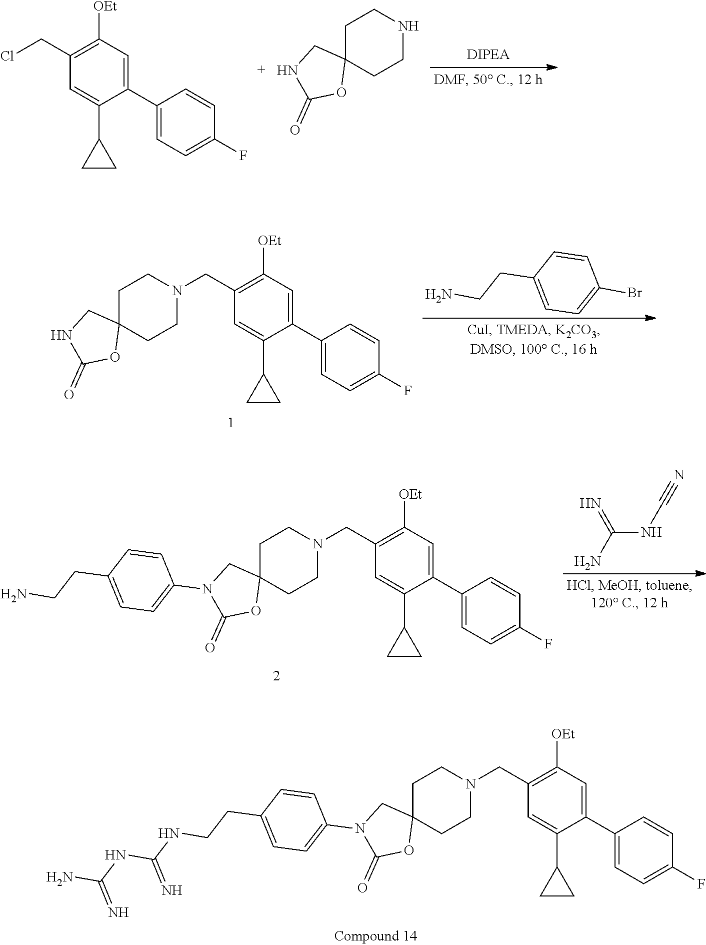

Step 1: 8-((2-cyclopropyl-5-ethoxy-4′-fluoro-[1,1′-biphenyl]-4-yl)methyl)-1-oxa-3,8-diazaspiro[4.5]decan-2-one (1): To a solution of 4-(chloromethyl)-2-cyclopropyl-5-ethoxy-4′-fluoro-1,1′-biphenyl (1.2 g, 3.9 mmol, 1 eq) and 1-oxa-3,8-diazaspiro[4.5]decan-2-one (0.91 g, 4.7 mmol, 1.2 eq, HCl salt) in DMF (10 mL) was added DIPEA (1.5 g, 11.8 mmol, 2.1 mL, 3 eq), then the mixture was stirred at 50° C. for 12 hours. The reaction mixture was diluted with H₂O (80 mL) and extracted with EA (70 mL×2). The combined organic layers were washed with saturated brine (60 mL×2), dried over Na₂SO₄, filtered and concentrated under reduced pressure to give a residue. The residue was purified by column chromatography (SiO₂, Petroleum ether/Ethyl acetate=20/1 to 0/1) to give 1 (1.67 g, 55% yield, 55% purity) as a yellow solid.

Step 2: 3-(4-(2-aminoethyl)phenyl)-8-((2-cyclopropyl-5-ethoxy-4′-fluoro-[1,1′-biphenyl]-4-yl)methyl)-1-oxa-3,8-diazaspiro[4.5]decan-2-one (2): A mixture of 1 (150 mg, 353 μmol, 1 eq), 2-(4-bromophenyl)ethanamine (283 mg, 1.4 mmol, 219 μL, 4 eq), CuI (27 mg, 141 μmol, 0.4 eq), K₂CO₃ (122 mg, 883 μmol, 2.5 eq) and TMEDA (33 mg, 283 μmol, 43 μL, 0.8 eq) in DMSO (1 mL) was degassed and purged with N₂ 3 times, and then the mixture was stirred at 100° C. for 16 hours under N₂ atmosphere. The mixture was diluted with H₂O (5 mL) and extracted with ethyl acetate (10 mL). The combined organic layer was washed with saturated brine (5 mL), dried over anhydrous Na₂SO₄, filtered and concentrated in vacuum to give a residue. The residue was purified by prep-TLC (SiO₂, DCM:MeOH=10:1) to give 2 (120 mg, crude) as a yellow oil. LCMS: (ES⁺) m/z (M+H)⁺=544.5.

Step 3: 1-carbamimidoyl-3-[2-[4-[8-[[5-cyclopropyl-2-ethoxy-4-(4-fluorophenyl)phenyl]methyl]-2-oxo-1-oxa-3,8-diazaspiro[4.5]decan-3-yl]phenyl]ethyl]guanidine (Compound 14): To a solution of 2 (80 mg, 147 μmol, 1 eq) in MeOH (1 mL) was added concentrated aqueous HCl (5 mg, 147 μmol, 5.3 μL, 1 eq), and the mixture was concentrated. To the residue was added toluene (1 mL), and the mixture was concentrated once more. The 1-cyanoguanidine (87 mg, 1.03 mmol, 7 eq) was added, and the reaction was stirred at 120° C. for 12 hours. The mixture was purified by prep-HPLC (column: Welch Xtimate C18 150×25 mm×5 μm; mobile phase: [A: water (0.225% FA), B: ACN]; B %: 25%-55%, 11 min) to give Compound 14 (5.76 mg, 6% yield) as a yellow solid. LCMS: (ES⁺) m/z (M+H)⁺=628.6. ¹H-NMR (400 MHz, CD₃OD) δ=8.47 (brs, 2H), 7.52 (d, J=8.4 Hz, 2H), 7.47-7.42 (m, 2H), 7.29 (d, J=8.4 Hz, 2H), 7.21-7.15 (m, 2H), 7.08 (s, 1H), 6.86 (s, 1H), 4.12 (q, J=7.2 Hz, 2H), 4.04 (s, 2H), 3.93 (s, 2H), 3.48 (t, J=7.2 Hz, 2H), 3.22-3.11 (m, 2H), 3.09-2.97 (m, 2H), 2.86 (t, J=7.2 Hz, 2H), 2.22-2.04 (m, 4H), 1.83-1.76 (m, 1H), 1.46 (t, J=7.2 Hz, 3H), 0.84-0.79 (m, 2H), 0.66-0.62 (m, 2H).

### Example 9: 1-carbamimidoyl-3-[[4-[8-[[5-cyclopropyl-2-ethoxy-4-(4-fluorophenyl)phenyl]methyl]-2-oxo-1-oxa-3,8-diazaspiro[4.5]decan-3-yl]phenyl]methyl]guanidine (Compound 15)

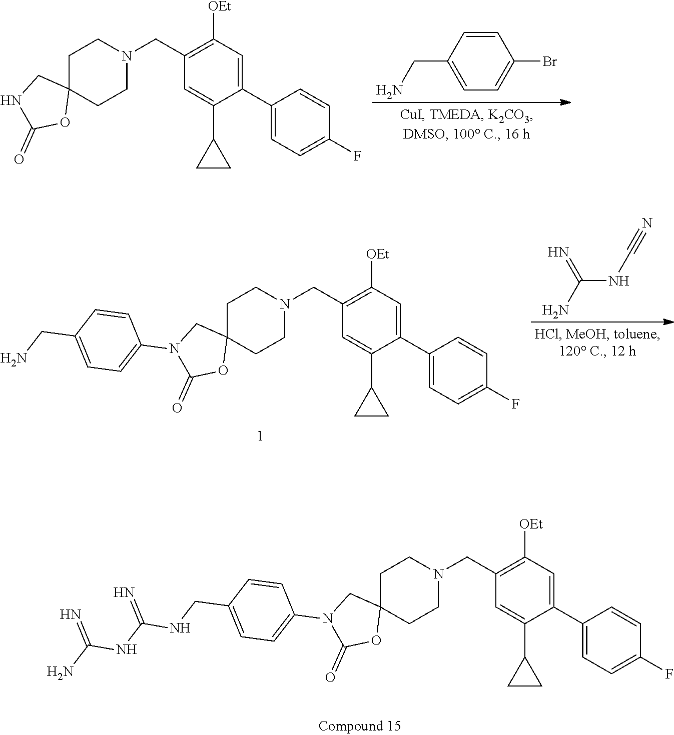 [SMILES: CCOc1cc(-c2ccc(F)cc2)c(C2CC2)cc1CN1CCC2(CC1)CNC(=O)O2 ; NCc1ccc(Br)cc1 ; CCOc1cc(-c2ccc(F)cc2)c(C2CC2)cc1CN1CCC2(CC1)CN(c1ccc(CN)cc1)C(=O)O2 ; N#CNC(=N)N ; CCOc1cc(-c2ccc(F)cc2)c(C2CC2)cc1CN1CCC2(CC1)CN(c1ccc(CNC(=N)NC(=N)N)cc1)C(=O)O2]

Step 1: 3-[4-(aminomethyl)phenyl]-8-[[5-cyclopropyl-2-ethoxy-4-(4-fluorophenyl)phenyl]methyl]-1-oxa-3,8-diazaspiro[4.5]decan-2-one (1): A mixture of (4-bromophenyl)methanamine (263 mg, 1.4 mmol, 179 μL, 4 eq), 8-[[5-cyclopropyl-2-ethoxy-4-(4-fluorophenyl)phenyl]methyl]-1-oxa-3,8-diazaspiro[4.5]decan-2-one (150 mg, 353 μmol, 1 eq), CuI (27 mg, 141 μmol, 0.4 eq), K₂CO₃ (122 mg, 883 μmol, 2.5 eq) and TMEDA (33 mg, 283 μmol, 43 μL, 0.8 eq) in DMSO (1 mL) was degassed and purged with N₂ 3 times, and then the mixture was stirred at 100° C. for 16 hours under N₂ atmosphere. The mixture was diluted with H₂O (5 mL) and extracted with ethyl acetate (10 mL). The organic phase was washed with saturated brine (5 mL), then concentrated in vacuum to give a residue. The residue was purified by prep-TLC (SiO₂, DCM:MeOH=10:1) to give 1 (80 mg, crude). LCMS: (ES⁺) m/z (M+H)⁺=530.3.

Step 2: 1-carbamimidoyl-3-[[4-[8-[[5-cyclopropyl-2-ethoxy-4-(4-fluorophenyl)phenyl]methyl]-2-oxo-1-oxa-3,8-diazaspiro[4.5]decan-3-yl]phenyl]methyl]guanidine (Compound 15): To a solution of 1 (50 mg, 94 μmol, 16 μL, 1 eq) in MeOH (1 mL) was added concentrated aqueous HCl (3.3 mg, 94 μmol, 3.4 μL, 1 eq), and the mixture was concentrated to give a residue. To the residue toluene (1 mL) was added, and the mixture was concentrated once more. Then 1-cyanoguanidine (40 mg, 472 μmol, 5 eq) was added, and the reaction was stirred at 120° C. for 12 hours. The mixture was purified by prep-HPLC (column: Welch Xtimate C18 150×25 mm×5 μm; mobile phase: [A: water (0.225% FA), B: ACN]; B %: 12%-42%, 11 min) to give Compound 15 as a yellow solid. LCMS: (ES⁺) m/z (M+H)⁺=614.4. ¹H NMR (400 MHz, CD₃OD) δ 8.57-8.46 (m, 1H), 7.58 (d, J=8.4 Hz, 2H), 7.48-7.44 (m, 2H), 7.37 (d, J=8.8 Hz, 2H), 7.18 (t, J=8.8 Hz, 2H), 7.05-7.03 (m, 1H), 6.83 (s, 1H), 4.43 (s, 2H), 4.13-4.06 (m, 2H), 3.97-3.84 (m, 4H), 3.07-2.77 (m, 4H), 2.17-1.99 (m, 4H), 1.82-1.77 (m, 1H), 1.44 (t, J=6.8 Hz, 3H), 0.83-0.77 (m, 2H), 0.65-0.60 (m, 2H).

### Example 10: methyl 2-cyclopropyl-4-((2-(4-(4-(3-(1,3-dihydroxy-2-(hydroxymethyl)propan-2-yl)ureido)butyl)phenyl)-3-oxo-2,8-diazaspiro[4.5]decan-8-yl)methyl)-5-ethoxybenzoate (Compound 16)

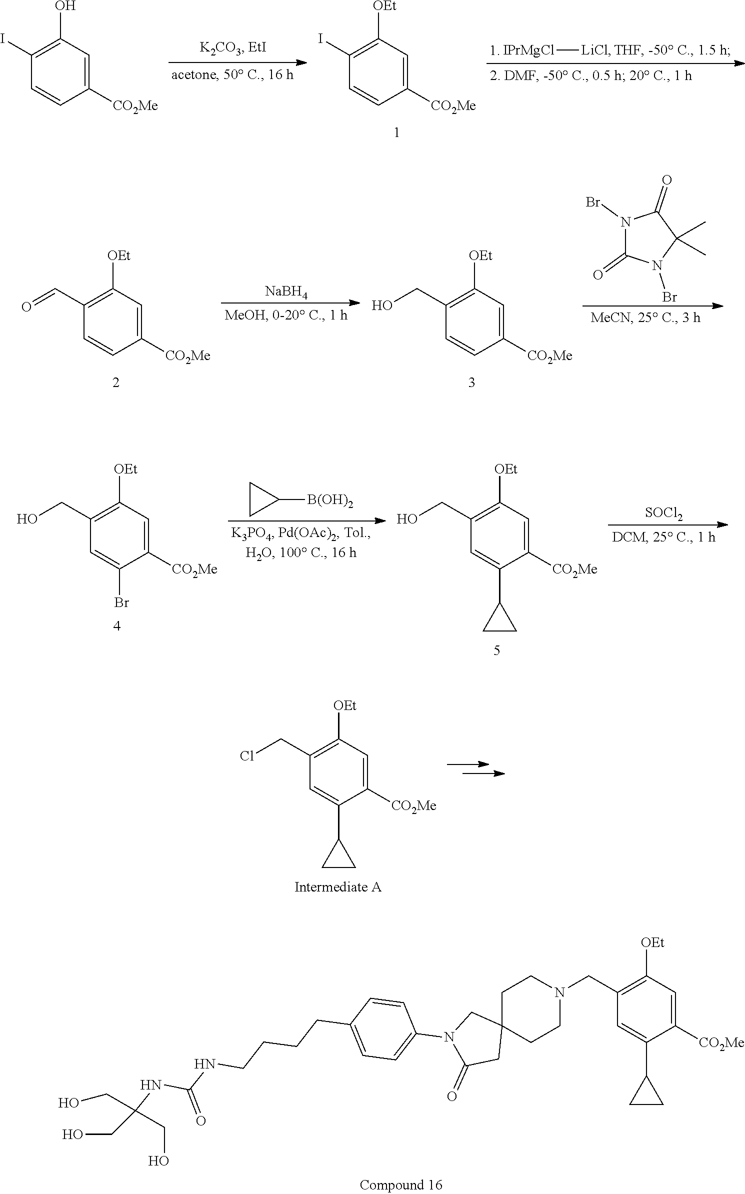

Step 1: methyl 3-ethoxy-4-iodobenzoate (1): To the suspension of methyl 3-hydroxy-4-iodobenzoate (25 g, 90 mmol, 1 eq) and K₂CO₃ (18.6 g, 135 mmol, 1.5 eq) in acetone (250 mL) was added EtI (18.2 g, 117 mmol, 9.35 mL, 1.3 eq), and the mixture was stirred at 50° C. for 16 hours. The reaction suspension was filtered, and the filtrate was concentrated at vacuum (50° C.). The residue was dissolved in EA (400 mL), washed with brine (100 mL×3), dried over Na₂SO₄, filtered and concentrated under vacuum (50° C.) to give 1 (26.4 g, 96% yield) as a light yellow solid. ¹H NMR (400 MHz, CDCl₃) δ 7.85 (d, J=8.0 Hz, 1H), 7.42 (d, J=1.6 Hz, 1H), 7.36 (dd, J₁=8.0 Hz, J₂=1.6 Hz, 1H), 4.16 (q, J=7.2 Hz, 2H), 3.92 (s, 3H), 1.51 (t, J=7.2 Hz, 3H).

Step 2: methyl 3-ethoxy-4-formylbenzoate (2): To the solution of 1 (20 g, 65 mmol, 1 eq) in THF (80 mL) was added i-PrMgCl—LiCl (1.3 M, 101 mL, 2 eq) dropwise at −50° C. under N₂ atmosphere, and the resulting reaction mixture was stirred for 1.5 hours. Then DMF (95.5 g, 1.31 mol, 101 mL, 20 eq) was added dropwise and the reaction mixture was stirred for 0.5 hour at −50° C. and for 1 hour at 20° C. The reaction suspension was quenched with citric acid (300 mL) and extracted with EA (100 mL×3). The combined organic layers were washed with brine (100 mL×3), dried over Na₂SO₄, filtered and concentrated under vacuum (50° C.) to give 2 (14.6 g, crude) as a white solid. ¹H NMR (400 MHz, CDCl₃) δ 10.53 (s, 1H), 7.83-7.90 (m, 1H), 7.59-7.75 (m, 2H), 4.22 (q, J=7.2 Hz, 2H), 3.93 (s, 3H), 1.50 (t, J=7.2 Hz, 3H).

Step 3: methyl 3-ethoxy-4-(hydroxymethyl)benzoate (3): To the solution of 2 (14.6 g, 70 mmol, 1 eq) in MeOH (150 mL) was added NaBH₄ (3.2 g, 84 mmol, 1.2 eq) portionwise at 0° C., and the resulting reaction mixture was stirred for 1 hour at 20° C. under N₂ atmosphere. The solvent was removed under vacuum (50° C.), and the residue was quenched with citric acid (200 mL) and extracted with EA (100 mL×2). The combined organic layers were dried over Na₂SO₄, filtered and concentrated under vacuum (50° C.). The residue was purified by flash silica gel chromatography (80 g Silica Flash Column, eluent of 0 to 30% ethyl acetate/petroleum ether gradient) to give 3 (9.5 g, 59% yield, 92% purity) as a white solid. LCMS: (ES⁺) m/z (M−17)⁺=193.7.

Step 4: methyl 2-bromo-5-ethoxy-4-(hydroxymethyl)benzoate (4): To the solution of 3 (9 g, 43 mmol, 1 eq) in MeCN (100 mL) was added 1,3-dibromo-5,5-dimethylimidazolidine-2,4-dione (6.7 g, 24 mmol, 0.55 eq), and the resulting mixture was stirred at 25° C. for 3 hours. The solvent was removed under vacuum (40° C.). The residue was dissolved in EA (300 mL), washed with brine (100 mL×3), dried over Na₂SO₄, filtered and concentrated under vacuum (50° C.) to give 4 (12.7 g, crude) as a yellow solid. ¹H NMR (400 MHz, CDCl₃) δ 7.54 (s, 1H), 7.21 (s, 1H), 4.62 (s, 2H), 3.99-4.05 (m, 2H), 3.86 (s, 3H), 2.10 (s, 1H), 1.37 (t, J=7.2 Hz, 3H).

Step 5: methyl 2-cyclopropyl-5-ethoxy-4-(hydroxymethyl)benzoate (5): A mixture of 4 (11.5 g, 40 mmol, 1 eq), cyclopropylboronic acid (10 g, 119 mmol, 3 eq), K₃PO₄ (25 g, 119 mmol, 3 eq), PCy₃ (558 mg, 1.99 mmol, 0.05 eq) and Pd(OAc)₂ (447 mg, 2.0 mmol, 0.05 eq) in toluene (120 mL) and H₂O (12 mL) was stirred at 100° C. for 16 hours under N₂ atmosphere. The reaction suspension was filtered, and the filtrate was concentrated under vacuum (50° C.). The residue was dissolved in EA (200 mL) and concentrated under vacuum (50° C.). The residue was purified by flash silica gel chromatography (80 g Silica Flash Column, Eluent of 0 to 30% ethyl acetate/petroleum ether gradient) to give 5 (6.2 g, 56% yield, 90% purity) as a light yellow oil. ¹H NMR (400 MHz, CDCl₃) δ 7.31 (s, 1H), 6.98 (s, 1H), 4.68 (d, J=6.4 Hz, 2H), 4.12 (q, J=6.4 Hz, 2H), 3.92 (s, 3H), 2.49-2.60 (m, 1H), 1.44 (t, J=7.2 Hz, 3H), 0.91-0.97 (m, 2H), 0.64 (m, 2H).

Step 6: methyl 4-(chloromethyl)-2-cyclopropyl-5-ethoxybenzoate (Intermediate A): To a solution of 5 (5.9 g, 21 mmol, 1 eq) in DCM (60 mL) was added SOCl₂ (5.1 g, 42 mmol, 3.1 mL, 2 eq), and the resulting reaction mixture was stirred for 1 hour at 25° C. under N₂ atmosphere. The solvent was removed in vacuo (50° C.) to give the crude product Intermediate A (6.5 g, crude) as a brown oil. LCMS: (ES⁺) m/z (M+H)⁺=269.5. ¹H NMR (400 MHz, CDCl₃) δ 7.31 (s, 1H), 7.07 (s, 1H), 4.62 (s, 2H), 4.12 (q, J=6.8 Hz, 2H), 3.92 (s, 3H), 2.51 (tt, J₁=8.4 Hz, J₂=5.6 Hz, 1H), 1.45 (t, J=7.2 Hz, 3H), 0.92-0.98 (m, 2H), 0.61-0.66 (m, 2H).

Step 7: methyl 2-cyclopropyl-4-((2-(4-(4-(3-(1,3-dihydroxy-2-(hydroxymethyl)propan-2-yl)ureido)butyl)phenyl)-3-oxo-2,8-diazaspiro[4.5]decan-8-yl)methyl)-5-ethoxybenzoate (Compound 16): Following procedures outlined in Example 8, Compound 16 was obtained from Intermediate A. (ES⁺) m/z (M+H)⁺=681.5. ¹H NMR (400 MHz, CD₃OD) δ 8.42 (d, J=4.8 Hz, 1H), 7.47 (d, J=8.0 Hz, 2H), 7.37 (s, 1H), 7.22 (d, J=8.4 Hz, 2H), 7.17 (s, 1H), 4.14 (q, J=6.8 Hz, 2H), 4.05 (s, 2H), 3.91 (s, 3H), 3.77 (s, 2H), 3.64 (s, 6H), 3.13-3.08 (m, 2H), 3.08-2.92 (m, 4H), 2.63 (t, J=7.6 Hz, 2H), 2.57 (s, 2H), 2.53-2.43 (m, 1H), 1.98-1.83 (m, 4H), 1.68-1.60 (m, 2H), 1.53-1.47 (m, 2H), 1.45 (t, J=6.8 Hz, 3H), 0.99-0.91 (m, 2H), 0.69-0.59 (m, 2H).

The following compounds were prepared according to the procedures described above using the appropriate intermediates.

| Cpd | Characterization Data |
|---|---|
| 17 | (ES⁺) m/z (M + H)⁺ = 562.5 |
| 18 | (ES⁺) m/z (M + H)⁺ = 612.5 |
| 19 | (ES⁺) m/z (M + H)⁺ = 592.5 |
| 20 | (ES⁺) m/z (M + H)⁺ = 577.5 |

### Example 11: (4-(8-(5-cyclopropyl-2-ethoxy-4-(methoxycarbonyl)benzyl)-2-oxo-1,3,8-triazaspiro[4.5]decan-3-yl)phenyl)phosphonic acid (Compound 21)

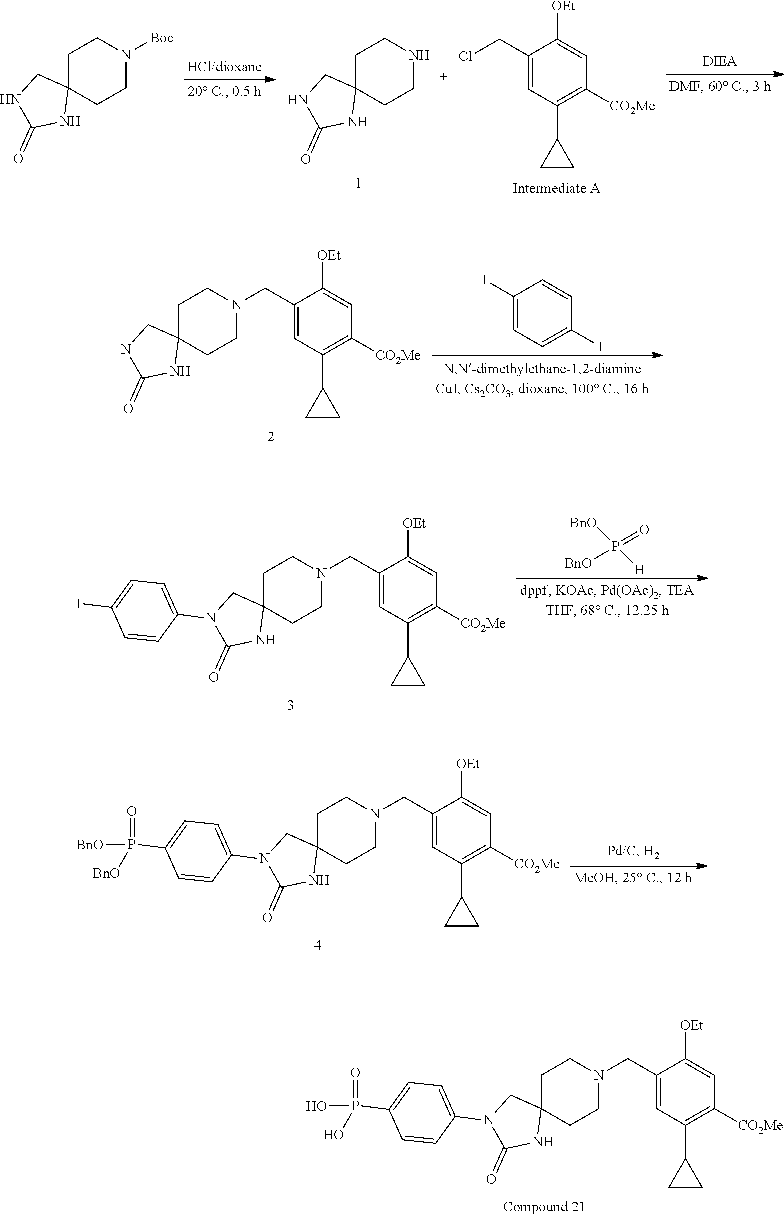

Step 1: 1,3,8-triazaspiro[4.5]decan-2-one (1): To a solution of tert-butyl 2-oxo-1,3,8-triazaspiro[4.5]decane-8-carboxylate (4 g, 16 mmol) in HCl/dioxane (4 M, 10 mL). The mixture was stirred at 20° C. for 0.5 hour. The reaction mixture was concentrated under reduced pressure to give crude 1 (4 g, HCl salt) as a white solid.

Step 2: methyl 2-cyclopropyl-5-ethoxy-4-((2-oxo-1,3,8-triazaspiro[4.5]decan-8-yl)methyl)benzoate (2): To a mixture of 1 (150 mg, 787 umol, 1 eq, HCl salt) and Intermediate A (211 mg, 787 umol, 1 eq) in DMF (3 mL) was added DIEA (408 mg, 3.2 mmol, 550 uL, 5 eq). The resulting reaction mixture was stirred at 60° C. for 3 hours. The reaction mixture was poured into water (10 mL) and extracted with EA (20 mL). The organic layer was separated, washed with saturated brine (10 mL), and concentrated to give 2 (350 mg, crude) as a yellow oil that was used in the next step without purification. LCMS: (ES⁺) m/z (M+H)⁺=388.1.

Step 3: methyl 2-cyclopropyl-5-ethoxy-4-((3-(4-iodophenyl)-2-oxo-1,3,8-triazaspiro[4.5]decan-8-yl)methyl)benzoate (3): To a solution of 1,4-diiodobenzene (596 mg, 1.8 mmol, 2 eq), 2 (350 mg, 903 umol, 1 eq), CuI (172 mg, 903 umol, 1 eq), Cs₂CO₃ (1.18 g, 3.6 mmol, 4 eq) and N,N′-dimethylethane-1,2-diamine (80 mg, 903 umol, 97 uL, 1 eq) in dioxane (2 mL). The mixture was stirred at 110° C. for 16 hours. The mixture was diluted with EA (20 mL) and then filtered. The residue was dissolved in water (20 mL) and concentrated NH₃.H₂O (20 mL) and extracted with EA (30 mL×4). The combined organic layers were washed with saturated brine (20 mL), dried over Na₂SO₄, filtered and concentrated under reduced pressure to give a residue. The crude product was purified by reversed-phase HPLC (column: Phenomenex luna C18 250×50 mm×10 um; mobile phase: [A: water (0.1% FA), B:ACN]; B %: 30%-40% over 15 min) to give 3 (200 mg, 38% yield. LCMS: (ES⁺) m/z (M+H)⁺=590.2.

Step 4: methyl 4-((3-(4-(bis(benzyloxy)phosphoryl)phenyl)-2-oxo-1,3,8-triazaspiro[4.5]decan-8-yl)methyl)-2-cyclopropyl-5-ethoxybenzoate (4): DPPF (9.4 mg, 17 umol, 0.05 eq), Pd(OAc)₂ (1.9 mg, 8.5 umol, 0.025 eq) and KOAc (3.3 mg, 34 umol, 0.1 eq) were placed in a round-bottom flask. The mixture was degassed and purged with N₂ 3 times. Then TEA (41 mg, 407 umol, 57 uL, 1.2 eq) in THE (3 mL) was added, and the mixture was stirred and heated at 68° C. After 15 min, dibenzyl phosphonate (89 mg, 339 umol, 1 eq) and 3 (200 mg, 339 umol, 1 eq) were added. The mixture was stirred at 68° C. for 12 hours. The mixture was concentrated in vacuum (50° C.) to give a residue. The residue was purified by prep-HPLC (column: Phenomenex Synergi C18 150×25×10 um; mobile phase: [A: water (0.225% FA), B: ACN]; B %: 40%-60% over 9 min) to give 4 (50 mg, 20% yield) as a yellow oil. LCMS: (ES⁺) m/z (M+H)⁺=724.5.

Step 5: (4-(8-(5-cyclopropyl-2-ethoxy-4-(methoxycarbonyl)benzyl)-2-oxo-1,3,8-triazaspiro[4.5]decan-3-yl)phenyl)phosphonic acid (Compound 21): To a solution of 4 (40 mg, 55 umol, 1 eq) in MeOH (5 mL) was added 5% Pd/C (29 mg, 14 umol, 0.25 eq) under N₂. The suspension was degassed under vacuum and purged with H₂ several times. The mixture was stirred under H₂ (50 psi) at 25° C. for 12 hours. The mixture was filtered, and the filtrate was concentrated in vacuo. The crude product was purified by prep-HPLC (column: Phenomenex Synergi C18 150×25×10 um; mobile phase: [A: water (0.225% FA), B: ACN]; B %: 6%-36% over 10 min). The solution was lyophilized to give Compound 21 (1.85 mg, 6% yield, 95% purity) as a white solid. LCMS: (ES⁺) m/z (M+H)⁺=544.2. ¹H NMR (CD₃OD, 400 MHz): δ 8.34-8.43 (m, 1H), 7.67-7.88 (m, 2H), 7.47-7.63 (m, 2H), 7.35-7.41 (m, 1H), 7.14-7.23 (m, 1H), 4.14 (br s, 2H), 3.98-4.08 (m, 2H), 3.82-3.97 (m, 5H), 3.74-3.82 (m, 2H), 2.98-3.11 (m, 2H), 2.45-2.53 (m, 1H), 1.83-2.08 (m, 4H), 1.41-1.49 (m, 3H), 0.90-0.98 (m, 2H), 0.61-0.71 (m, 2H).

The following compounds were prepared according to the procedures described above using the appropriate intermediates.

| Cpd | Characterization Data |
|---|---|
| 22 | LCMS: (ES⁺) m/z (M + H)⁺ = 580.4. ¹H NMR (400 MHz, CD₃OD) δ 7.82 (dd, J₁ = 11.2, |
| | J₂ = 8.8 Hz, 2H), 7.48-7.40 (m, 4 H), 7.14 (t, J = 8.8 Hz, 2H), 6.99 (s, 1H), 6.76 (s, 1H), |
| | 4.04 (q, J = 6.8 Hz, 2H), 3.74 (s, 2H), 3.63 (s, 2H), 2.73 (br s, 2H), 2.52 (br s, 2H), 1.84 |
| | (br t, J = 5.4 Hz, 4H), 1.80-1.72 (m, 1H), 1.40 (t, J = 6.8 Hz, 3H), 0.79-0.73 (m, 2H), |
| | 0.62-0.57 (m, 2H). |
| 23 | LCMS: (ES⁺) m/z (M + H)⁺ = 543.0. ¹H NMR (400 MHz, DMSO-d₆) δ 7.65-7.60 (m, |
| | 4H), 7.25 (s, 1H), 7.12 (s, 1H), 4.05 (q, J = 6.8 Hz, 2H), 3.84 (s, 3H), 3.72 (s, 2H), 3.65 |
| | (s, 2H), 2.70 (s, 4H), 2.67 (s, 2H), 2.46-2.40 (m, 1H), 1.70 (s, 4H), 1.34 (t, J = 6.8 Hz, |
| | 3H), 0.91-0.89 (m, 2H), 0.61-0.60 (m, 2H). |
| 24 | LCMS: (ES⁺) m/z (M + H)⁺ = 571.1. ¹H NMR (400 MHz, DMSO-d₆) δ 7.65-7.60 (m, |
| | 4H), 7.19 (s, 1H), 7.11 (s, 1H), 5.17-5.10 (m, 1H), 4.04 (q, J = 6.8 Hz, 2H), 3.66 (s, |
| | 4H), 2.67 (t, J = 2.0 Hz, 4H), 2.48 (s, 2H), 2.39 (s, 1H), 1.69 (s, 4H), 1.31-1.35 (m, |
| | 9H), 0.89 (d, J = 8.4 Hz, 2H), 0.59 (s, 2H). |
| 25 | LCMS: (ES⁺) m/z (M + H)⁺ = 608.2. ¹H NMR (400 MHz, CD₃OD) δ 7.81-7.70 (m, |
| | 2H), 7.57 (d, J = 6.8 Hz, 2H), 7.49-7.41 (m, 2H), 7.25-7.14 (m, 3H), 6.91 (s, 1H), 4.32 |
| | (s, 2H), 4.16 (q, J = 6.8 Hz, 2H), 3.84-3.75 (m, 2H), 3.75-3.34 (m, 4H), 3.29-2.98 |
| | (m, 2H), 2.18-1.85 (m, 4H), 1.84-1.74 (m, 1H), 1.46 (t, J = 6.8 Hz, 3H), 1.17 (t, J = 6.8 |
| | Hz, 3H), 0.87-0.78 (m, 2H), 0.77-0.61 (m, 2H). |

### Example 12: (4-(8-(5-cyclopropyl-2-ethoxy-4-(3-methyl-1,2,4-oxadiazol-5-yl)benzyl)-2-oxo-1-oxa-3,8-diazaspiro[4.5]decan-3-yl)phenyl)phosphonic acid (Compound 26)

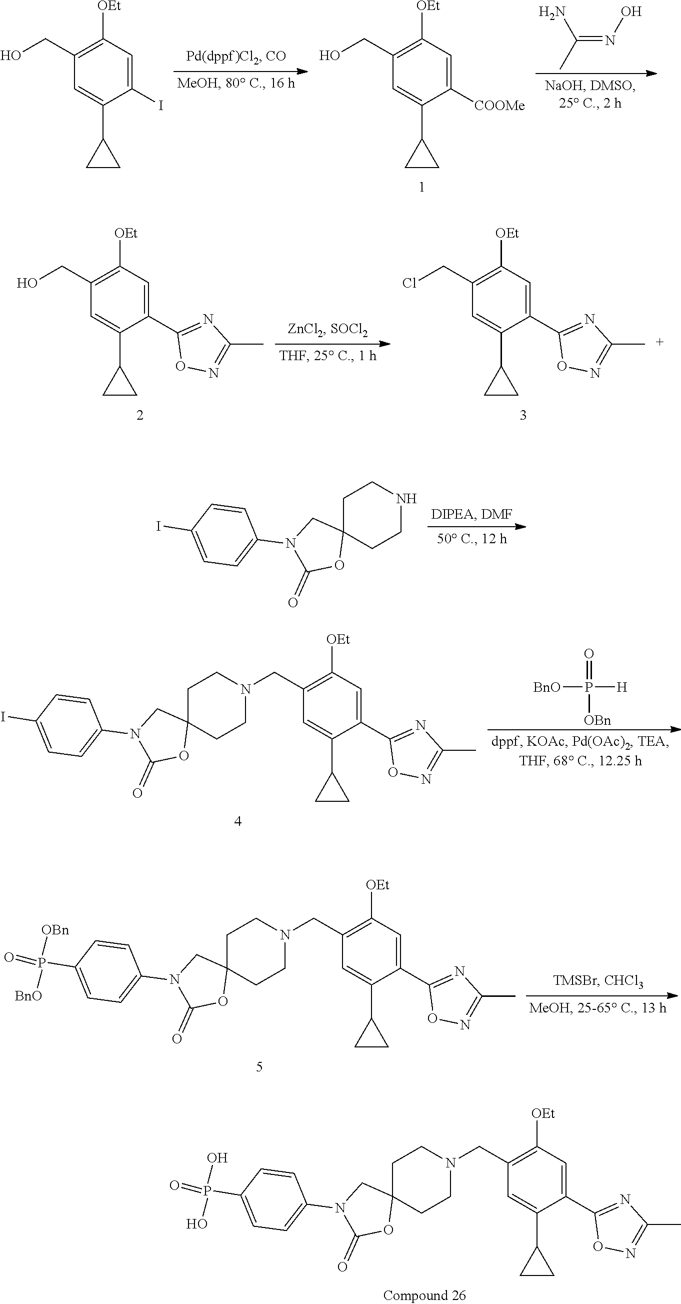

Step 1: methyl 2-cyclopropyl-5-ethoxy-4-(hydroxymethyl)benzoate (1): To a solution of (5-cyclopropyl-2-ethoxy-4-iodophenyl)methanol (1.0 g, 3.1 mmol, 1 eq) in MeOH (25 mL) was added Pd(dppf)Cl₂ (0.23 g, 0.31 mmol, 0.1 eq) and TEA (1.3 g, 13 mmol, 4 eq) under N₂. The suspension was degassed under vacuum and purged with CO several times. The mixture was stirred under CO (50 psi) at 80° C. for 16 hours. The mixture was concentrated in vacuo. The residue was purified by column chromatography (SiO₂, petroleum ether:ethyl acetate, 20:1 to 8:1) to give 1 (0.68 g, 86% yield) as a yellow oil. LCMS: (ES⁺) m/z (M+H)⁺=250.9. ¹H NMR (400 MHz, CDCl₃) δ 7.30 (s, 1H), 6.98 (s, 1H), 4.67 (d, J=6.0 Hz, 2H), 4.11 (t, J=6.8 Hz, 2H), 3.92 (s, 3H), 2.53-2.56 (m, 1H), 2.40 (d, J=6.0 Hz, 1H), 1.44 (t, J=6.8 Hz, 3H), 0.93-0.97 (m, 2H), 0.63-0.64 (m, 2H).

Step 2: (5-cyclopropyl-2-ethoxy-4-(3-methyl-1,2,4-oxadiazol-5-yl)phenyl)methanol (2): To a solution of 1 (0.68 g, 2.7 mmol, 1 eq) and N′-hydroxyacetamidine (0.45 g, 4.1 mmol, 1.5 eq, HCl salt) in DMSO (10 mL) was added NaOH (0.16 g, 4.1 mmol, 1.5 eq), and the mixture was stirred at 25° C. for 2 hours. The mixture was poured into water (30 mL) slowly and adjusted to pH 6 with aqueous HCl (2 M), then extracted with EA (30 mL×3). The combined organic layer was washed with water (20 mL×3) and brine (20 mL×3), dried over Na₂SO₄, filtered and concentrated in vacuo to give 2 (0.75 g, crude) as a yellow oil. LCMS: (ES⁺) m/z (M+H)⁺=274.9.

Step 3: 5-(4-(chloromethyl)-2-cyclopropyl-5-ethoxyphenyl)-3-methyl-1,2,4-oxadiazole (3): To a solution of 2 (0.75 g, 2.7 mmol, 1 eq) in THF (10 mL) was added SOCl₂ (0.49 g, 4.1 mmol, 1.5 eq) and ZnCl₂ (37 mg, 0.27 mmol, 0.1 eq) at 0° C., and the mixture was stirred at 25° C. for 1 hour. The mixture was poured into water (30 mL) slowly and then extracted with EA (40 mL×3). The combined organic layer was washed with water (40 mL×2) and brine (40 mL×2), dried over Na₂SO₄, filtered and concentrated in vacuo. The residue was purified by column chromatography (SiO₂, petroleum ether:ethyl acetate, 10:1 to 1:1) to give 3 (0.40 g, 50% yield) as a white solid. LCMS: (ES⁺) m/z (M+H)⁺=292.8. ¹H NMR (400 MHz, CDCl₃) δ 7.49 (s, 1H), 7.15 (s, 1H), 4.65 (s, 2H), 4.16 (t, J=6.8 Hz, 2H), 2.64-2.60 (m, 1H), 2.50 (s, 3H), 1.47 (t, J=6.8 Hz, 3H), 1.01-1.04 (m, 2H), 0.67-0.68 (m, 2H).

Step 4: 8-(5-cyclopropyl-2-ethoxy-4-(3-methyl-1,2,4-oxadiazol-5-yl)benzyl)-3-(4-iodophenyl)-1-oxa-3,8-diazaspiro[4.5]decan-2-one (4): To a solution of 3-(4-iodophenyl)-1-oxa-3,8-diazaspiro[4.5]decan-2-one (0.2 g, 0.51 mmol, 1 eq, HCl salt) and 3 (0.12 g, 0.41 mmol, 0.8 eq) in DMF (3 mL) was added DIEA (0.26 g, 2.0 mmol, 4 eq), and the mixture was stirred at 50° C. for 12 hour. The mixture was poured into H₂O (30 mL), then extracted with EA (40 mL×3). The combined organic layer was washed with water (30 mL×3) and brine (30 mL×3), dried over Na₂SO₄, filtered and concentrated in vacuo. The residue was purified by column chromatography (SiO₂, Petroleum ether:Ethyl acetate=1:2) to give 4 (0.30 g, 93% yield) as a yellow solid. LCMS: (ES⁺) m/z (M+H)⁺=615.0.

Step 5: dibenzyl (4-(8-(5-cyclopropyl-2-ethoxy-4-(3-methyl-1,2,4-oxadiazol-5-yl)benzyl)-2-oxo-1-oxa-3,8-diazaspiro[4.5]decan-3-yl)phenyl)phosphonate (5): To a solution of DPPF (27 mg, 49 μmol, 0.1 eq), KOAc (9.6 mg, 98 μmol, 0.2 eq), and Pd(OAc)₂ (5.5 mg, 24 μmol, 0.05 eq) in THE (3 mL) was added TEA (74 mg, 0.73 mmol, 1.5 eq) under N₂, and the mixture was stirred and heated at 68° C. After 15 min, 4 (0.3 g, 0.49 mmol, 1 eq) and benzyloxyphosphonoyloxymethylbenzene (0.38 g, 1.5 mmol, 3 eq) in THE (3 mL) were added to the solution, and the mixture was stirred at 68° C. for 12 hour. The mixture was concentrated in vacuo. The residue was purified by prep-HPLC (column: Phenomenex luna C18 150×40 mm×15 μm; mobile phase: A: water (0.225% FA), B: ACN; B %: 23%-53% gradient over 9 min) and lyophilized to give 5 (80 mg, 20% yield, FA salt) as a yellow oil. LCMS: (ES⁺) m/z (M+H)⁺=749.1.

Step 6: (4-(8-(5-cyclopropyl-2-ethoxy-4-(3-methyl-1,2,4-oxadiazol-5-yl)benzyl)-2-oxo-1-oxa-3,8-diazaspiro[4.5]decan-3-yl)phenyl)phosphonic acid (Compound 26): To a solution of 5 (80 mg, 0.10 mmol, 1 eq, FA salt) in CHCl₃ (1.5 mL) was added TMSBr (46 mg, 0.30 mmol, 3 eq), and the mixture was stirred at 25° C. for 1 hour. The mixture was concentrated in vacuo. The residue was dissolved in 4 mL MeOH and stirred at 65° C. for 12 hours, then concentrated in vacuo. The residue was purified by prep-HPLC (column: Phenomenex Synergi C18 150×25 mm×110 μm; mobile phase: A: water (0.225% FA), B: ACN; B %: 13%-43% gradient over 10 min) to give Compound 26 (28 mg, 43% yield, FA) as a white solid. LCMS: (ES⁺) m/z (M−H)⁺=569.0. ¹H NMR (400 MHz, DMSO-d₆) δ 8.14 (s, 1H), 7.57-7.66 (m, 4H), 7.43 (s, 1H), 7.15 (s, 1H), 4.08 (q, J=6.8 Hz, 2H), 3.88 (s, 2H), 3.57 (s, 2H), 2.67 (s, 2H), 2.63-2.61 (m, 1H), 2.43 (s, 3H), 2.33 (s, 2H), 1.91 (s, 4H), 1.35 (t, J=6.8 Hz, 3H), 0.96-0.98 (m, 2H), 0.62-0.64 (m, 2H).

The following compounds were prepared according to the procedures described above using the appropriate intermediates.

| Cpd | Characterization Data |
|---|---|
| 27 | (ES⁺) m/z (M + H)⁺ = 568.0. ¹H NMR (400 MHz, CD₃OD) δ 7.79-7.66 (m, 4H), 7.60 (d, |
| | J = 1.6 Hz, 1H), 7.49 (d, J = 3.6 Hz, 1H), 4.47 (s, 1H), 4.44 (s, 1H), 4.24 (q, J = 6.8 Hz, |
| | 2H), 4.04 (s, 1H), 3.84 (s, 1H), 3.58 (d, J = 12.8 Hz, 2H), 3.46-3.40 (m, 2H), 2.59-2.54 |
| | (m, 1H), 2.47 (s, 3H), 2.32-2.21 (m, 2H), 2.20-2.09 (m, 2H), 1.51 (t, J = 6.8 Hz, |
| | 3H), 1.03-0.98 (m, 2H), 0.83-0.80 (m, 2H). |
| 28 | (ES+) m/z (M + H)+ = 578.3. 1H NMR (400 MHz, CD3OD) δ 7.75 (br t, J = 9.8 Hz, 2H), |
| | 7.58 (br d, J = 6.8 Hz, 2H), 7.50-7.42 (m, 2H), 7.25-7.14 (m, 3H), 6.90 (s, 1H), 4.26 (br |
| | d, J = 15.6 Hz, 2H), 4.14 (br d, J = 7.2 Hz, 2H), 3.66 (br s, 2H), 3.44-3.34 (m, 1H), 3.19 |
| | (br s, 3H), 1.92 (br s, 4H), 1.80 (br s, 1H), 1.50-1.37 (m, 6H), 0.83 (br d, J = 7.2 Hz, |
| | 2H), 0.72 (br s, 2H). |
| 29 | (ES+) m/z (M + H)+ = 579.2. 1H NMR (400 MHz, CD3OD) δ 7.87 (dd, J = 11.2, 8.4 Hz, |
| | 2 H), 7.49 (dd, J = 8.4, 2.4 Hz, 2 H), 7.45-7.38 (m, 2 H), 7.18-7.11 (m, 2 H), 7.00- |
| | 6.94 (m, 1 H), 6.75 (s, 1 H), 4.07-4.00 (m, 2 H), 3.72 (s, 2 H), 3.61 (s, 2 H), 2.71-2.47 |
| | (m, 6 H), 1.84-1.71 (m, 5 H), 1.40 (t, J = 6.8 Hz, 3 H), 0.79-0.73 (m, 2 H), 0.62- |
| | 0.56 (m, 2 H). |
| 30 | (ES⁺) m/z (M + H)⁺ = 595.5. ¹H NMR (400 MHz, CD₃OD) δ 7.51-7.43 (m, 4H), 7.37- |
| | 7.33 (m, 2H), 7.20-7.15 (m, 3H), 6.87 (s, 1H), 4.18 (s, 2H), 4.10 (q, J = 7.2 Hz, 2H), |
| | 3.63 (s, 2H), 3.24-3.10 (m, 2H), 3.02-2.91 (m, 4H), 1.85-1.67 (m, 5H), 1.42 (t, |
| | J = 6.8 Hz, 3H), 0.83-0.77 (m, 2H), 0.74-0.72 (m, 2H). |
| 31 | (ES⁺) m/z (M + H)⁺ = 702.2. ¹H NMR (400 MHz, CD₃OD) δ 7.61 (s, 1H), 7.38 (s, 1H), |
| | 7.34-7.29 (m, 1H), 7.24 (s, 1H), 7.24-7.18 (m, 1H), 6.90 (d, J = 7.6 Hz, 1H), 4.18 (q, |
| | J = 6.8 Hz, 2H), 4.04 (s, 2H), 3.80 (s, 2H), 3.64 (s, 6H), 3.26 (s, 3H), 3.14-2.95 (m, 6H), |
| | 2.78-2.70 (m, 1H), 2.62 (t, J = 7.6 Hz, 2H), 2.06-1.90 (m, 4H), 1.71-1.59 (m, 2H), 1.54- |
| | 1.43 (m, 5H), 1.16-1.09 (m, 2H), 0.93-0.86 (m, 2H). |

### Example 13: 8-((2-cyclopropyl-5-ethoxy-4′-fluoro-[1,1′-biphenyl]-4-yl)methyl)-3-(4-((((2S,3R,4R,5R)-2,3,4,5,6-pentahydroxyhexyl)amino)methyl)phenyl)-1-oxa-3,8-diazaspiro[4.5]decan-2-one (Compound 32)

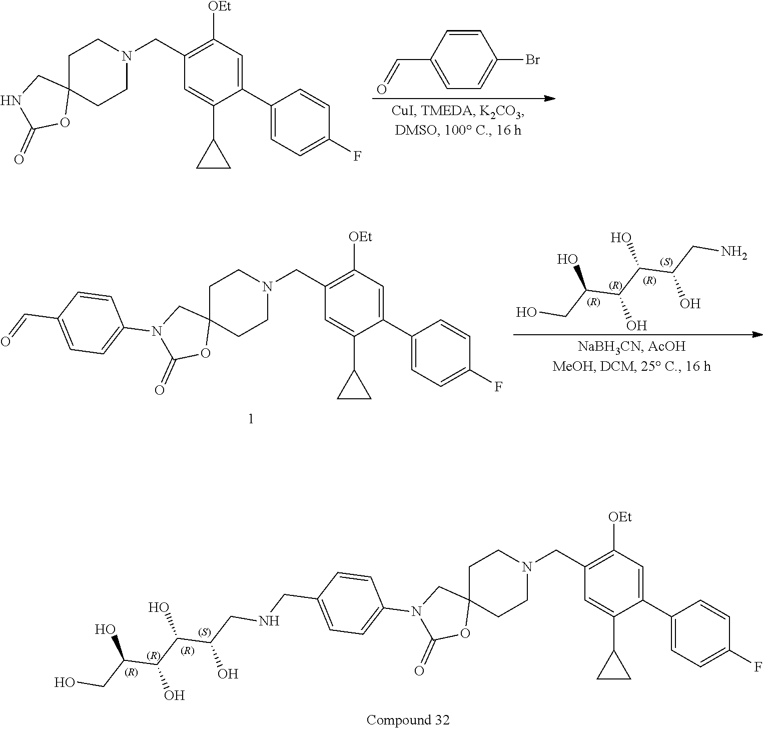

Step 1: 4-(8-((2-cyclopropyl-5-ethoxy-4′-fluoro-[1,1′-biphenyl]-4-yl)methyl)-2-oxo-1-oxa-3,8-diazaspiro[4.5]decan-3-yl)benzaldehyde (1): A mixture of 4-bromobenzaldehyde (131 mg, 707 μmol, 2 eq), 8-[[5-cyclopropyl-2-ethoxy-4-(4-fluorophenyl)phenyl]methyl]-1-oxa-3,8-diazaspiro[4.5]decan-2-one (150 mg, 353 μmol, 1 eq), CuI (27 mg, 141 μmol, 0.4 eq), K₂CO₃ (122 mg, 883 μmol, 2.5 eq) and TMEDA (33 mg, 283 μmol, 43 μL, 0.8 eq) in DMSO (1.5 mL) was degassed and purged with N₂ 3 times, and then the mixture was stirred at 100° C. for 12 hours under N₂ atmosphere. The reaction mixture was concentrated under reduced pressure to give a residue. The residue was purified by prep-TLC (SiO₂, Dichloromethane:Methanol=10:1) to give 1 as a yellow solid. LCMS: (ES⁺) m/z (M+H)⁺=529.4.

Step 2: 8-((2-cyclopropyl-5-ethoxy-4′-fluoro-[1,1′-biphenyl]-4-yl)methyl)-3-(4-((((2S,3R,4R,5R)-2,3,4,5,6-pentahydroxyhexyl)amino)methyl)phenyl)-1-oxa-3,8-diazaspiro [4.5]decan-2-one (Compound 32): To a solution of 1 (60 mg, 114 μmol, 1 eq), (2R,3R,4R,5S)-6-aminohexane-1,2,3,4,5-pentol (21 mg, 114 μmol, 1 eq) in MeOH (1 mL) and DCM (0.5 mL) were added NaBH3CN (7 mg, 114 μmol, 1 eq) and AcOH (7 mg, 114 μmol, 6.49 μL, 1 eq). The mixture was stirred at 25° C. for 16 hours. The reaction mixture was concentrated under reduced pressure to give a residue. The residue was purified by prep-HPLC [column: Welch Xtimate C18 150×25 mm×5 μm; mobile phase: [A: water (0.225% FA), B: ACN]; B %: 12%-42%, 11 min] to give Compound 32 (30.44 mg, 37 yield, 97% purity, FA salt) as a white solid. LCMS: (ES⁺) m/z (M+H)⁺=694.3. ¹H-NMR (CD₃OD, 400 MHz): δ 8.42-8.38 (m, 2H), 7.72-7.69 (m, 2H), 7.55-7.52 (m, 2H), 7.48-7.41 (m, 2H), 7.22-7.16 (m, 2H), 7.10-7.05 (m, 1H), 6.89-6.84 (m, 1H), 4.23-4.22 (m, 2H), 4.14-4.04 (m, 5H), 3.97-3.92 (m, 3H), 3.84-3.81 (m, 1H), 3.78-3.73 (m, 1H), 3.70-3.59 (m, 4H), 3.18-3.12 (m, 3H), 2.22-2.04 (m, 5H), 1.82-1.72 (m, 1H), 1.45-1.40 (m, 3H), 0.81-0.76 (m, 2H), 0.61-0.60 (m, 2H).

### Example 14: ((1s,4s)-4-(8-((2-cyclopropyl-5-ethoxy-4′-fluoro-[1,1′-biphenyl]-4-yl)methyl)-2-oxo-1-oxa-3,8-diazaspiro[4.5]decan-3-yl)cyclohexyl)phosphonic acid (Compound 33) and ((1r,4r)-4-(8-((2-cyclopropyl-5-ethoxy-4′-fluoro-[1,1′-biphenyl]-4-yl)methyl)-2-oxo-1-oxa-3,8-diazaspiro[4.5]decan-3-yl)cyclohexyl)phosphonic acid) (Compound 34)

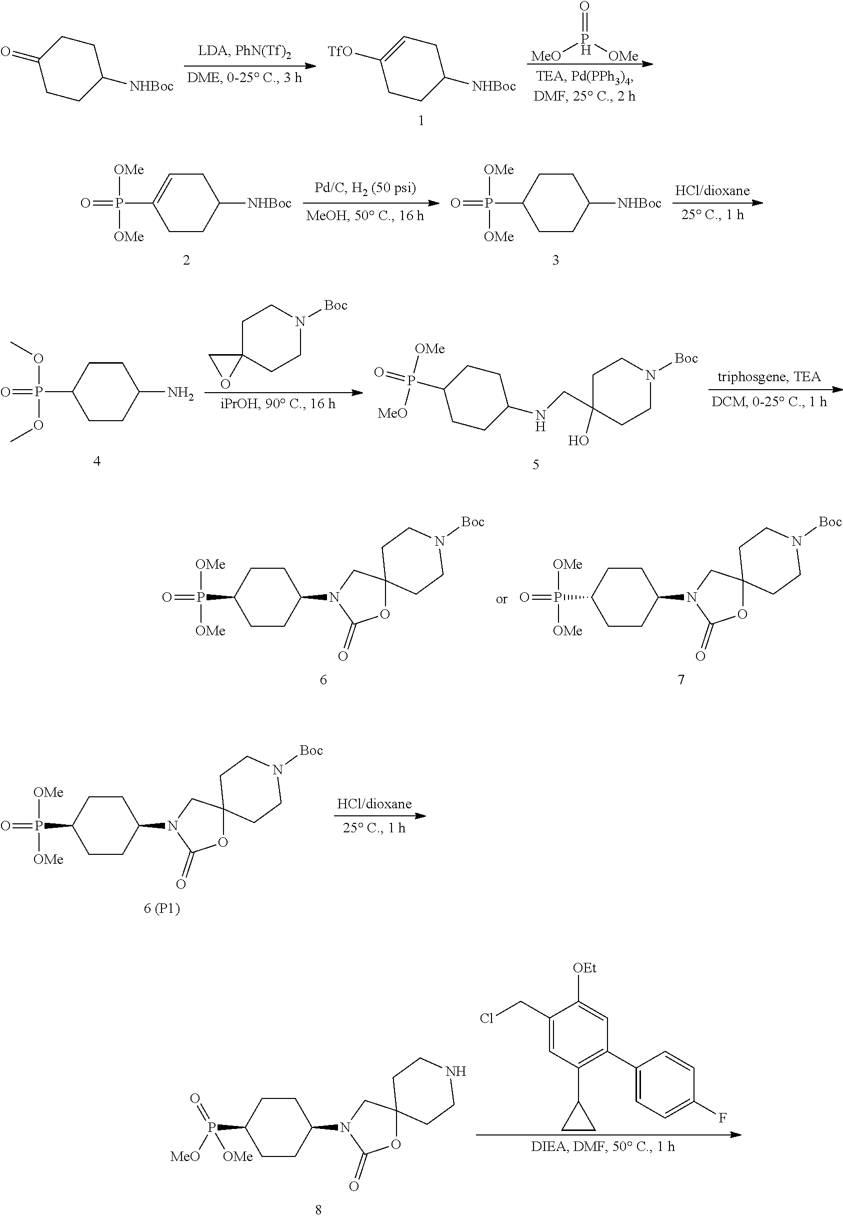

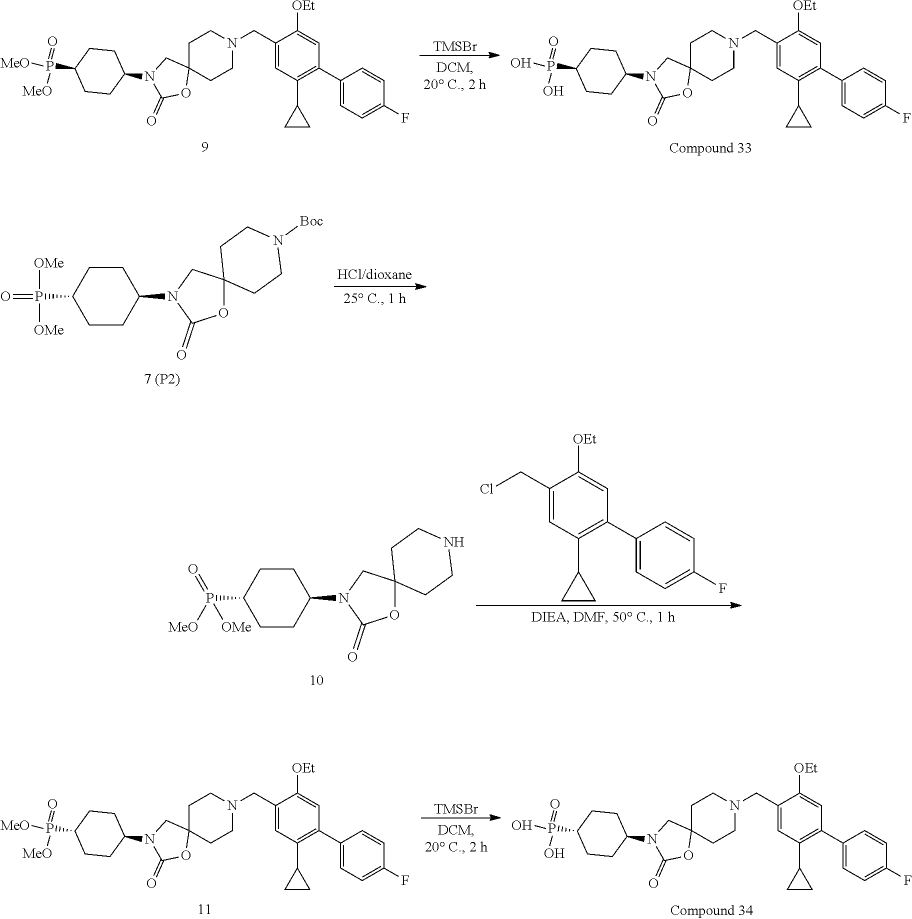

Step 1: 4-((tert-butoxycarbonyl)amino)cyclohex-1-en-1-yl trifluoromethanesulfonate (1): To a mixture of tert-butyl N-(4-oxocyclohexyl)carbamate (10 g, 47 mmol, 10.00 mL, 1 eq) in DME (100 mL) was added LDA (2 M, 46.89 mL, 2 eq) at 0° C. The mixture was stirred at 25° C. for 1 hour, then 1,1,1-trifluoro-N-phenyl-N-((trifluoromethyl)sulfonyl) methanesulfonamide (33.5 g, 94 mmol, 2 eq) was added, and the mixture was stirred at 25° C. for 2 hours. After completion, the mixture was added to H₂O (100 mL). The aqueous phase was extracted with EA (100 mL×2). The combined organic phase was washed with brine (100 mL×2), dried with anhydrous Na₂SO₄, filtered and concentrated in vacuum. The residue was purified by silica gel chromatography (Petroleum ether/Ethyl acetate=100/1 to 20/1) to give product 1 (24 g, crude) as a yellow oil. ¹H NMR (400 MHz, CDCl-d) δ 5.68 (t, J=3.2 Hz, 1H), 5.69-5.66 (m, 1H), 3.85-3.81 (m, 1H), 2.55-2.38 (m, 3H), 2.13-1.93 (m, 2H), 1.81-1.71 (m, 1H), 1.44 (s, 9H).

Step 2: tert-butyl (4-(dimethoxyphosphoryl)cyclohex-3-en-1-yl)carbamate (2): To a mixture of 1 (21 g, 61 mmol, 1 eq), methoxyphosphonoyloxymethane (8.0 g, 73 mmol, 6.7 mL, 1.2 eq) in DMF (200 mL) was added Pd(PPh₃)₄ (7.0 g, 6.1 mmol, 0.1 eq) and TEA (18.5 g, 182 mmol, 25.4 mL, 3 eq). The mixture was stirred at 25° C. for 2 hours under N₂. After completion, the mixture was added to H₂O (600 mL) and the aqueous phase was extracted with EA (500 mL×2). The combined organic phase was washed with brine (200 mL×2), dried with anhydrous Na₂SO₄, filtered and concentrated under reduced pressure to give a residue. The residue was purified by column chromatography (SiO₂, Petroleum ether/Ethyl acetate=100/1 to 0/1) to give 2 (7.5 g, 40% yield) as a yellow solid. ¹H NMR (400 MHz, CDCl₃-d) δ 6.74-6.64 (m, 1H), 4.55 (br s, 1H), 3.86-3.74 (m, 1H), 3.72 (d, J=2.6 Hz, 3H), 3.69 (d, J=2.6 Hz, 3H), 2.58 (br d, J=18.0 Hz, 1H), 2.38-2.20 (m, 2H), 2.09-1.88 (m, 2H), 1.62-1.53 (m, 1H), 1.44 (s, 9H).

Step 3: tert-butyl (4-(dimethoxyphosphoryl)cyclohexyl)carbamate (3): To a mixture of 2 (7.5 g, 24.57 mmol, 1 eq) in MeOH (75 mL) was added 10% Pd/C (750 mg), and the mixture was stirred at 50° C. for 16 hours under H2 (50 psi). The reaction mixture was filtrated, and the filtrate was concentrated to give a residue. The residue was purified by column chromatography (SiO₂, Petroleum ether/Ethyl acetate=50/1 to 0/1) to give 3 (4 g, 53% yield) as a white solid. ¹H NMR (400 MHz, CDCl₃-d) δ 4.38 (br s, 1H), 3.77 (s, 1H), 3.76 (s, 3H), 3.74 (s, 1H), 3.73 (s, 3H), 3.40 (br d, J=3.2 Hz, 1H), 2.15-1.98 (m, 4H), 1.74-1.58 (m, 2H), 1.48 (br dd, J=3.6, 5.6 Hz, 1H), 1.44 (s, 9H), 1.06 (dq, J=3.2, 12.4 Hz, 2H).

Step 4: dimethyl (4-aminocyclohexyl)phosphonate (4): A solution of 3 (4 g, 13 mmol, 1 eq) in HCl/dioxane (4 M, 60 mL) was stirred at 25° C. for 1 hour. The mixture was adjust to pH 9 with aqueous NaHCO₃, and the aqueous phase was concentrated under reduced pressure to give a residue. The residue was extracted with THF (40 mL) twice. The combined organic layers were concentrated under reduced pressure to give a residue. The residue was purified by prep-TLC (SiO₂, Ethyl acetate:Methanol=5:1) to give 4 as a mixture of cis and trans isomers (1.3 g, 48% yield) as a white solid. 1H NMR (400 MHz, CDCl₃-d) δ 4.91 (br s, 2H), 3.75-3.70 (m, 6H), 3.62 (br s, 1H), 2.70-2.57 (m, 1H), 2.04-1.89 (m, 4H), 1.53-1.38 (m, 2H), 1.14-0.97 (m, 2H).

Step 5: tert-butyl 4-(((4-(dimethoxyphosphoryl)cyclohexyl)amino)methyl)-4-hydroxypiperidine-1-carboxylate (5): A mixture of 4 (0.7 g, 3.4 mmol, 1 eq) and tert-butyl 1-oxa-6-azaspiro[2.5]octane-6-carboxylate (793 mg, 3.7 mmol, 1.1 eq) in i-PrOH (18 mL) was degassed and purged with N₂ 3 times, and then the mixture was stirred at 90° C. for 16 hours under N₂ atmosphere. The reaction mixture was concentrated under reduced pressure to give a residue. The residue was purified by column chromatography (SiO₂, Ethyl acetate:Methanol=30/1 to 1/1) to give 5 (1.05 g, 74% yield) as a yellow oil. LCMS: (ES⁺) m/z (M+H)⁺=421.2.

Step 6: tert-butyl 3-((1s,4s)-4-(dimethoxyphosphoryl)cyclohexyl)-2-oxo-1-oxa-3,8-diazaspiro[4.5]decane-8-carboxylate (6) (P1) and tert-butyl 3-((1r,4r)-4-(dimethoxyphosphoryl)cyclohexyl)-2-oxo-1-oxa-3,8-diazaspiro[4.5]decane-8-carboxylate (7) (P2): To a mixture of 5 (550 mg, 1.31 mmol, 1 eq) and TEA (662 mg, 6.5 mmol, 910 μL, 5 eq) in DCM (5 mL) was added triphosgene (466 mg, 1.6 mmol, 1.2 eq) at 0° C. under N₂. The mixture was stirred at 25° C. for 1 hour. The mixture was added to H₂O (20 mL), and the aqueous phase was extracted with DCM (20 mL×2). The combined organic phase was washed with brine (10 mL×2), dried with anhydrous Na₂SO₄, filtered and concentrated under reduced pressure to give a residue. The residue was purified by column chromatography (SiO₂, Ethyl acetate:Methanol=I/O to 10/1) to give 6 (P1) (360 mg, 62% yield) as yellow oil and 7 (P2) (160 mg, 27% yield) as yellow oil. Absolute stereochemistry (cis or trans) has not been confirmed.

Step 7: dimethyl ((1s,4s)-4-(2-oxo-1-oxa-3,8-diazaspiro[4.5]decan-3-yl)cyclohexyl)phosphonate (8): A solution of 6 (P1) (350 mg, 784 μmol, 1 eq) in HCl/dioxane (5 mL) was stirred at 25° C. for 1 hour. The reaction solution was concentrated to dryness. The crude product 8 (296 mg, crude, HCl salt) as a white solid was used in next step directly without purification. LCMS: (ES⁺) m/z (M+H)⁺=347.2.

Step 8: dimethyl ((1s,4s)-4-(8-((2-cyclopropyl-5-ethoxy-4′-fluoro-[1,1′-biphenyl]-4-yl)methyl)-2-oxo-1-oxa-3,8-diazaspiro[4.5]decan-3-yl)cyclohexyl)phosphonate (9): To a solution of 8 (250 mg, 653 μmol, 1 eq, HCl salt), 1-(chloromethyl)-5-cyclopropyl-2-ethoxy-4-(4-fluorophenyl)benzene (239 mg, 784 μmol, 1.2 eq) in DMF (8 mL) was added DIEA (618 mg, 4.8 mmol, 833 μL, 7.3 eq) and NaI (10 mg, 65 μmol, 0.1 eq). The mixture was stirred at 50° C. for 1 hour. The reaction mixture was quenched by addition water (30 mL) at 25° C. and extracted with EA (30 mL×3). The combined organic layers were washed with brine (30 mL), dried over Na₂SO₄, filtered and concentrated under reduced pressure to give a residue. The residue was purified by column chromatography (SiO₂, Ethyl acetate:Methanol=1:0 to 1:1) to give 9 (200 mg, crude) as yellow oil. ¹H NMR (400 MHz, CDCl₃-d) δ 7.41 (dd, J=5.2, 8.4 Hz, 2H), 7.15-7.08 (m, 2H), 6.92 (s, 1H), 6.70 (s, 1H), 4.01 (q, J=7.2 Hz, 2H), 3.76 (d, J=10.6 Hz, 6H), 3.61 (s, 2H), 3.20 (s, 2H), 2.70-2.52 (m, 3H), 2.18-2.05 (m, 2H), 2.00-1.86 (m, 4H), 1.84-1.72 (m, 5H), 1.59-1.51 (m, 2H), 1.44-1.34 (m, 5H), 0.92-0.83 (m, 1H), 0.80-0.74 (m, 2H), 0.61-0.55 (m, 2H).

Step 9: ((1s,4s)-4-(8-((2-cyclopropyl-5-ethoxy-4′-fluoro-[1,1′-biphenyl]-4-yl)methyl)-2-oxo-1-oxa-3,8-diazaspiro[4.5]decan-3-yl)cyclohexyl)phosphonic acid (Compound 33): To a solution of 9 (180 mg, 293 μmol, 1 eq) in DCM (2 mL) was added TMSBr (1.34 g, 8.8 mmol, 1.14 mL, 30 eq). The mixture was stirred at 20° C. for 2 hours. The reaction mixture was quenched by addition water (1 mL) at 20° C. and concentrated under reduced pressure to give a residue. The residue was purified by prep-HPLC (column: Waters Xbridge Prep OBD C18 150×40 mm×10 μm; mobile phase: [A: water (0.05% NH₃.H₂O+10 mM NH₄HCO₃), B: ACN]; B %: 25%-55%, 8 min) to give Compound 33 (designated 1s,4s but of unknown stereochemistry, 30 mg, 17% yield, 99% purity, ammonium salt) as a white solid. LCMS (ES⁺) m/z (M+H)⁺=587.3. ¹H NMR (400 MHz, CD₃OD-d₄) δ 7.45 (dd, J=5.6, 8.4 Hz, 2H), 7.17 (t, J=8.8 Hz, 2H), 7.10 (s, 1H), 6.88 (s, 1H), 4.20-4.10 (m, 4H), 3.61-3.48 (m, 1H), 3.42 (s, 2H), 3.16-3.02 (m, 3H), 2.25-2.01 (m, 6H), 1.93-1.69 (m, 4H), 1.61-1.11 (m, 9H), 0.81 (br d, J=6.8 Hz, 2H), 0.64 (br d, J=4.4 Hz, 2H).

Step 10: dimethyl ((1r,4r)-4-(2-oxo-1-oxa-3,8-diazaspiro[4.5]decan-3-yl)cyclohexyl)phosphonate (10): A solution of 7 (P2) (0.14 g, 0.3 mmol, 1.0 eq) in HCl/dioxane (4 M, 5.0 mL, 64 eq) was stirred at 20° C. for 1 hour. The reaction mixture was concentrated under reduced pressure to remove solvent. The crude product 10 (0.15 g, crude, HCl salt) as a yellow oil was used in the next step directly without purification. ¹H NMR (400 MHz, CD₃OD-d₄) δ 3.78 (d, J=10.8 Hz, 6H), 3.71 (s, 2H), 3.48 (s, 2H), 3.41-3.35 (m, 3H), 3.29-3.23 (m, 2H), 2.21-1.95 (m, 13H).

Step 11: dimethyl ((1r,4r)-4-(8-((2-cyclopropyl-5-ethoxy-4′-fluoro-[1,1′-biphenyl]-4-yl)methyl)-2-oxo-1-oxa-3,8-diazaspiro[4.5]decan-3-yl)cyclohexyl)phosphonate (11): To a solution of 10 (0.13 g, 0.34 mmol, 1.0 eq, HCl salt) and 1-(chloromethyl)-5-cyclopropyl-2-ethoxy-4-(4-fluorophenyl)benzene (81 mg, 0.27 mmol, 0.8 eq) in DMF (3.0 mL) was added DIEA (0.32 g, 2.5 mmol, 0.43 mL, 7.3 eq) and NaI (10 mg, 68 μmol, 0.20 eq). The mixture was stirred at 50° C. for 1 hour. The mixture was quenched with H₂O (20 mL) and extracted with 5:1 v/v DCM/i-PrOH (20 mL×3). The combined organic phase was dried with anhydrous Na₂SO₄, filtered and concentrated in vacuum. The residue was purified by prep-TLC (SiO₂, Ethyl acetate:Methanol=3:1, R^{f}=0.26) to give 11 (85 mg, 41% yield) as a white solid. ¹H NMR (400 MHz, CDCl₃-d) δ 7.42 (dd, J=5.6, 8.0 Hz, 2H), 7.17-7.06 (m, 2H), 6.93 (br s, 1H), 6.70 (s, 1H), 4.01 (q, J=6.8 Hz, 2H), 3.77 (d, J=10.4 Hz, 6H), 3.63 (br s, 2H), 3.50 (s, 1H), 3.28 (s, 2H), 2.64 (br s, 3H), 2.19-1.90 (m, 6H), 1.86-1.73 (m, 4H), 1.71-1.54 (m, 7H), 1.39 (t, J=6.8 Hz, 3H), 0.81-0.73 (m, 2H), 0.60 (br d, J=4.4 Hz, 2H).

Step 12: ((1r,4r)-4-(8-((2-cyclopropyl-5-ethoxy-4′-fluoro-[1,1′-biphenyl]-4-yl)methyl)-2-oxo-1-oxa-3,8-diazaspiro[4.5]decan-3-yl)cyclohexyl)phosphonic acid (Compound 34): To a solution of 11 (65 mg, 0.11 mmol, 1.0 eq) in DCM (6.0 mL) was added drop-wise via syringe TMSBr (0.32 g, 2.1 mmol, 0.27 mL, 20 eq) in DCM (1.0 mL). The mixture was stirred at 20° C. for 3 hours. Then bromo(trimethyl)silane (0.16 g, 1.1 mmol, 0.14 mL, 10 eq) in DCM (0.50 mL) was added drop-wise via syringe. The mixture was stirred at 20° C. for 1 hour. The reaction mixture was concentrated under N₂. The mixture was quenched with H₂O (0.50 mL). The residue was purified by prep-HPLC (column: Waters Xbridge Prep OBD C18 150×40 mm×10 μm; mobile phase: [A: water (10 mM NH₄HCO₃), B: ACN]; B %: 25%-55%, 8 min) to give a white solid that was lyophilized from water. Then H₂O (5.0 mL) and NH₃.H₂O (0.3 mL) was added. The mixture was lyophilized to give Compound 34 (designated 1r,4r but of unknown stereochemistry, 31 mg, 49% yield, 99% purity, ammonium salt) as a white solid. LCMS (ES⁺) m/z (M+H)⁺=587.2. ¹H NMR (400 MHz, DMSO-d₆) δ 7.48 (dd, J=6.0, 8.4 Hz, 2H), 7.25 (t, J=8.4 Hz, 2H), 6.94 (s, 1H), 6.74 (s, 1H), 4.01 (q, J=6.8 Hz, 2H), 3.55-3.46 (m, 6H), 3.23 (s, 2H), 2.47-2.41 (m, 2H), 2.01 (br d, J=9.6 Hz, 4H), 1.79-1.71 (m, 4H), 1.69-1.46 (m, 3H), 1.37 (br d, J=8.4 Hz, 2H), 1.30 (t, J=6.8 Hz, 3H), 0.73 (br d, J=8.8 Hz, 2H), 0.51 (br d, J=4.4 Hz, 2H).

### Example 15: (4-(8-(5-cyclobutyl-2-ethoxy-4-(5-fluoropyridin-2-yl)benzyl)-2-oxo-1-oxa-3,8-diazaspiro[4.5]decan-3-yl)phenyl)phosphonic acid (Compound 35)

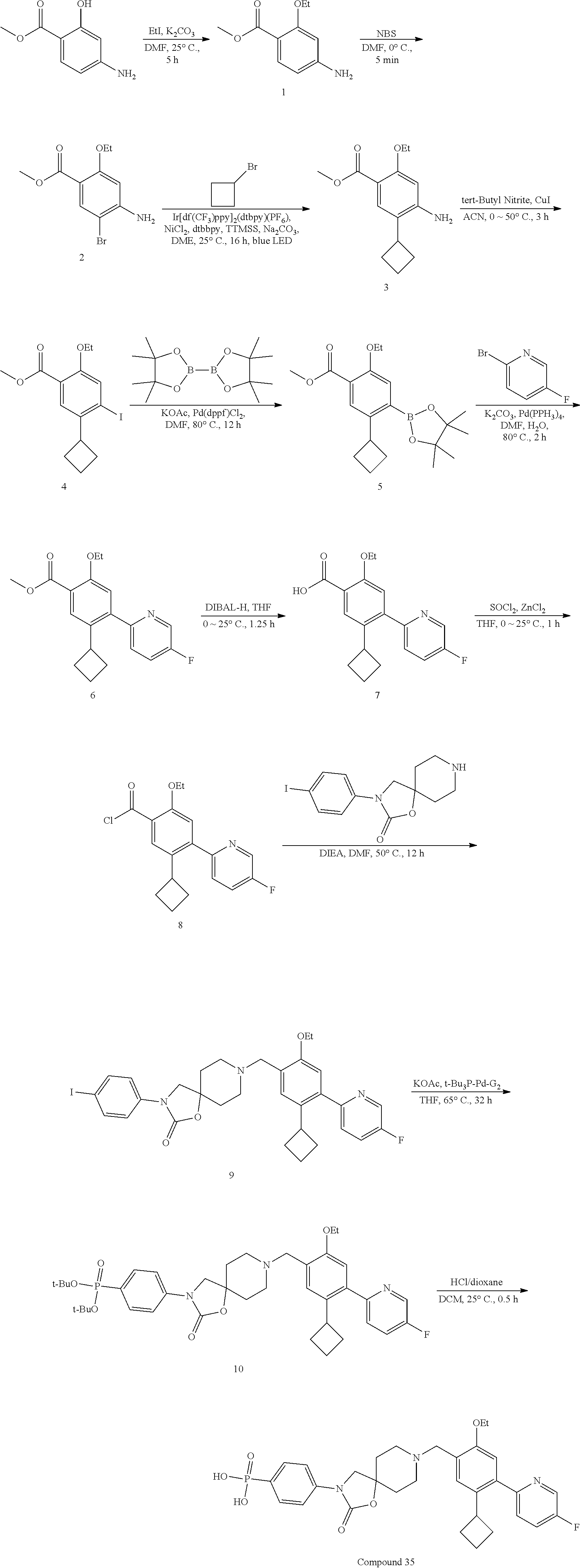

Step 1: methyl 4-amino-2-ethoxybenzoate (1): To a solution of methyl 4-amino-2-hydroxy-benzoate (15 g, 90 mmol, 1 eq) in DMF (90 mL) was added Cs₂CO₃ (29 g, 90 mmol, 1 eq) and iodoethane (14 g, 90 mmol, 7.2 mL, 1 eq). The mixture was stirred at 25° C. for 5 hours. The mixture was poured into 50 mL of H₂O and extracted with EA (40 mL×3). The combined organic layer was washed with water (50 mL×2) and brine (50 mL×2), dried over Na₂SO₄, filtered and concentrated in vacuo. The residue was purified by flash silica gel chromatography (ISCO®; 80 g SepaFlash® Silica Flash Column, Eluent of 0 to 25% Ethyl acetate/Petroleum ether gradient) to give 1 (11.8 g, 67% yield) as a yellow solid. LCMS: (ES⁺) m/z (M+H)⁺=195.9.

Step 2: methyl 4-amino-5-bromo-2-ethoxybenzoate (2): To a solution of 1 (11.3 g, 57.9 mmol, 1 eq) in DMF (80 mL) was added NBS (10.8 g, 60.8 mmol, 1.05 eq). The mixture was stirred at 0° C. for 5 min. The mixture was poured into 50 mL of H₂O and extracted with EA (40 mL×3). The combined organic layer was washed with water (50 mL×2) and brine (50 mL×2), dried over Na₂SO₄, filtered and concentrated in vacuo. The residue was purified by flash silica gel chromatography (ISCO®; 20 g SepaFlash® Silica Flash Column, Eluent of 0 to 60% Ethyl acetate/Petroleum ether gradient) to give 2 (11.8 g, 72% yield, 97% purity) as a yellow solid. LCMS: (ES⁺) m/z (M−31)⁺=243.9. ¹H NMR (400 MHz, CDCl₃) δ 7.98 (s, 1H), 6.29 (s, 1H), 4.46 (br s, 2H), 4.08-3.99 (m, 2H), 3.83 (s, 3H), 1.47 (t, J=7.2 Hz, 3H).

Step 3: methyl 4-amino-5-cyclobutyl-2-ethoxybenzoate (3): A mixture of 2 (4 g, 15 mmol, 1 eq), bromocyclobutane (3.2 g, 23 mmol, 2.2 mL, 1.6 eq), Ir[dF(CF₃)ppy]2(dtbpy)(PF₆) (164 mg, 146 μmol, 0.01 eq), NiCl₂.dtbbpy (29 mg, 73 μmol, 0.005 eq), TTMSS (4.4 g, 18 mmol, 5.4 mL, 1.2 eq), Na₂CO₃ (3.1 g, 29 mmol, 2 eq) in DME (40 mL) under nitrogen was stirred and irradiated with a 34 W blue LED lamp (7 cm away), with a cooling fan to keep the reaction temperature at 25° C. for 16 hours. The mixture was poured into 50 mL of H₂O and extracted with EA (40 mL×3). The combined organic layer was washed with water (50 mL×2) and brine (50 mL×2), dried over Na₂SO₄, filtered and concentrated in vacuo to give a residue. The residue was purified by flash silica gel chromatography (ISCO®; 4 g SepaFlash® Silica Flash Column, Eluent of 0 to 40% Ethyl acetate/Petroleum ether gradient) to give 3 (1.3 g, 30% yield, 84% purity) as a yellow solid.

Step 4: methyl 5-cyclobutyl-2-ethoxy-4-iodobenzoate (4): To a solution of 3 (1.3 g, 5.2 mmol, 1 eq) in ACN (30 mL) was added CuI (1.5 g, 7.8 mmol, 1.5 eq) and tert-butyl nitrite (1.1 g, 10 mmol, 1.2 mL, 2 eq) dropwise at 0° C. The mixture was stirred at 25° C. for 1 hour, then heated to 50° C. for 2 hours. The mixture was poured into 40 mL of H₂O and extracted with EA (20 mL×3). The combined organic layer was washed with water (40 mL×2) and brine (40 mL×2), dried over Na₂SO₄, filtered and concentrated in vacuo. The residue was purified by flash silica gel chromatography (ISCO®; 12 g SepaFlash® Silica Flash Column, Eluent of 0 to 30% Ethyl acetate/Petroleum ether gradient) to give 4 (0.95 g, 51% yield) as a yellow oil. LCMS: (ES⁺) m/z (M+H)⁺=360.8. ¹H NMR (400 MHz, CDCl₃) δ 7.66 (s, 1H), 7.41 (s, 1H), 4.12-4.04 (m, 2H), 3.90 (s, 3H), 3.63-3.53 (m, 1H), 2.48-2.39 (m, 2H), 2.14-1.96 (m, 3H), 1.85-1.75 (m, 1H), 1.45 (t, J=6.8 Hz, 3H).

Step 5: methyl 5-cyclobutyl-2-ethoxy-4-(4,4,5,5-tetramethyl-1,3,2-dioxaborolan-2-yl)benzoate (5): A solution of 4 (0.65 g, 1.8 mmol, 1 eq), 4,4,5,5-tetramethyl-2-(4,4,5,5-tetramethyl-1,3,2-dioxaborolan-2-yl)-1,3,2-dioxaborolane (458 mg, 1.8 mmol, 1 eq), KOAc (531 mg, 5.4 mmol, 3 eq) and Pd(dppf)Cl₂ (66 mg, 90 μmol, 0.05 eq) in DMF (6 mL) was prepared in a glove box. Then the mixture was stirred at 80° C. for 12 hours under N₂ atmosphere. The mixture was poured into 30 mL of H₂O and extracted with EA (50 mL×3). The combined organic layer was washed with water (30 mL×2), saturated sodium sulfite and brine (30 mL×2), dried over Na₂SO₄, filtered and concentrated in vacuo. The crude product was purified by reversed-phase HPLC (column: Phenomenex luna C18 250×50 mm×10 μm; mobile phase: [A: water (0.1% FA, v/v), B: ACN]; B %: 50%-60% gradient over 30 min) to give 5 (350 mg, 54% yield) as a white solid. LCMS: (ES⁺) m/z (M+H)⁺=361.3. ¹H NMR (400 MHz, CDCl₃) δ 7.52 (s, 1H), 7.27 (s, 1H), 3.98-3.90 (m, 2H), 3.76 (s, 3H), 3.49-3.36 (m, 2H), 2.34-2.24 (m, 2H), 1.98-1.78 (m, 1H), 1.71-1.62 (m, 1H), 1.43 (t, J=6.8 Hz, 3H), 1.36 (s, 12H).

Step 6: methyl 5-cyclobutyl-2-ethoxy-4-(5-fluoropyridin-2-yl)benzoate (6): A solution of 5 (250 mg, 694 μmol, 1 eq), 2-bromo-5-fluoropyridine (122 mg, 694 μmol, 1 eq), K₂CO₃ (288 mg, 2.1 mmol, 3 eq) and Pd(PPh₃)₄ (40 mg, 35 μmol, 0.05 eq) in DMF (4 mL) and H₂O (1 mL) was de-gassed and then heated to 80° C. for 2 hours under N₂. The mixture was poured into 40 mL of H₂O and extracted with EA (20 mL×3). The combined organic layer was washed with water (40 mL×2) and brine (40 mL×2), dried over Na₂SO₄, filtered and concentrated in vacuo. The residue was purified by flash silica gel chromatography (ISCO®; 4 g SepaFlash® Silica Flash Column, Eluent of 0 to 25% Ethyl acetate/Petroleum ether gradient) to give 6 (160 mg, 68% yield, 98% purity) as a colorless oil. LCMS: (ES⁺) m/z (M+H)⁺=330.2.

Step 7: (5-cyclobutyl-2-ethoxy-4-(5-fluoropyridin-2-yl)phenyl)methanol (7): To a solution of 6 (210 mg, 638 μmol, 1 eq) in THE (6 mL) was added dropwise DIBAL-H (1 M, 1.9 mL, 3 eq) at 0° C. over 15 min. After addition, the resulting mixture was stirred at 25° C. for 1 hour. The reaction mixture was quenched by addition of H₂O at 0° C., then adjusted to pH 4 with 3M aqueous HCl, diluted with water (40 mL) and extracted with EtOAc (30 mL×2). The combined organic layers were washed with saturated brine (40 mL×2), dried over anhydrous Na₂SO₄, filtered and concentrated under reduced pressure to give 7 (200 mg, crude) as a white solid.

Step 8: 2-(4-(chloromethyl)-2-cyclobutyl-5-ethoxyphenyl)-5-fluoropyridine (8): To a mixture of 7 (0.2 g, 664 μmol, 1 eq) in THE (4 mL) was added SOCl₂ (118 mg, 996 μmol, 72 μL, 1.5 eq) and ZnCl₂ (9.1 mg, 66 μmol, 3.1 μL, 0.1 eq) at 0° C. The mixture was stirred at 25° C. for 1 hour. The mixture was quenched by slow addition of saturated aqueous NaHCO₃ (10 mL) and extracted with EA (40 mL×3). The combined organic layer was washed with water (20 mL×2) and brine (20 mL×2), dried over Na₂SO₄, filtered and concentrated in vacuo to give 8 (180 mg, 85% yield) as a yellow oil.

Step 9: 8-(5-cyclobutyl-2-ethoxy-4-(5-fluoropyridin-2-yl)benzyl)-3-(4-iodophenyl)-1-oxa-3,8-diazaspiro[4.5]decan-2-one (9): To a solution of 8 (90 mg, 281 μmol, 1 eq) and 3-(4-iodophenyl)-1-oxa-3,8-diazaspiro[4.5]decan-2-one (121 mg, 338 μmol, 1.2 eq, HCl salt) in DMF (4 mL) was added DIEA (145 mg, 1.1 mmol, 196 μL, 4 eq). The mixture was stirred at 50° C. for 12 hours. The mixture was poured into 40 mL of H₂O and extracted with EA (30 mL×3). The combined organic layer was washed with water (40 mL×2) and brine (40 mL×2), dried over Na₂SO₄, filtered and concentrated in vacuo. The crude product was purified by reversed-phase HPLC (column: Phenomenex luna C18 250×50 mm×10 μm; mobile phase: [A: water (0.1% FA, v/v), B: ACN]; B %: 40%-50% gradient over 30 min) to give 9 (150 mg, 79% yield, 95% purity) as a colorless oil. LCMS: (ES⁺) m/z (M+H)⁺=642.0.

Step 10: di-tert-butyl (4-(8-(5-cyclobutyl-2-ethoxy-4-(5-fluoropyridin-2-yl)benzyl)-2-oxo-1-oxa-3,8-diazaspiro[4.5]decan-3-yl)phenyl)phosphonate (10): A mixture of 9 (150 mg, 234 μmol, 1 eq), 2-tert-butoxyphosphonoyloxy-2-methylpropane (227 mg, 1.2 mmol, 5 eq), KOAc (69 mg, 701 μmol, 3 eq), and tBu₃P—Pd-G2 (10 mg, 20 μmol, 0.087 eq) in THE (2 mL) was prepared in a glove box. Then the mixture was stirred at 65° C. for 32 hours. The mixture was poured into 40 mL of H₂O and extracted with EA (40 mL×3). The combined organic layer was washed with water (30 mL×2) and brine (30 mL×2), dried over Na₂SO₄, filtered and concentrated in vacuo. The crude product was purified by reversed-phase HPLC (column: Phenomenex luna C18 250×50 mm×10 μm; mobile phase: [A: water (0.1% FA, v/v), B: ACN]; B %: 30%-45% gradient over 30 min) to give 10 (170 mg, 225.51 μmol, 96.45% yield, FA salt) as a yellow oil. LCMS: (ES⁺) m/z (M+H)⁺=708.3.

Step 11: (4-(8-(5-cyclobutyl-2-ethoxy-4-(5-fluoropyridin-2-yl)benzyl)-2-oxo-1-oxa-3,8-diazaspiro[4.5]decan-3-yl)phenyl)phosphonic acid (Compound 35): To a solution of 10 (170 mg, 240 μmol, 1 eq) in DCM (1 mL) was added HCl/dioxane (4 M, 1.80 mL, 30 eq). The mixture was stirred at 25° C. for 30 minutes. The reaction mixture was concentrated under reduced pressure. The residue was purified by HPLC (column: Phenomenex Gemini NX-C₁₈ (75×30 mm×3 μm); mobile phase: [A: water (0.05% ammonia hydroxide v/v), B: ACN]; B %: 10%-40%, 8 min) to give Compound 35 (43.17 mg, 29% yield, 100% purity, ammonium salt) as a white solid. LCMS: (ES⁺) m/z (M+H)⁺=596.3. ¹H NMR (400 MHz, CD₃OD) δ 8.53 (d, J=2.8 Hz, 1H), 7.83 (m, 2H), 7.72 (m, 1H), 7.61-7.54 (m, 3H), 7.52 (m, 1H), 6.98 (s, 1H), 4.31 (br s, 2H), 4.15 (m, 2H), 3.85 (br d, J=8.8 Hz, 2H), 3.70 (m, 1H), 3.44-3.33 (m, 2H), 3.27-3.14 (m, 2H), 2.24-2.08 (m, 4H), 2.08-1.92 (m, 4H), 1.92-1.81 (m, 1H), 1.80-1.68 (m, 1H), 1.46 (t, J=6.8 Hz, 3H).

The following compounds were prepared according to the procedures described above using the appropriate intermediates.

| Cpd | Characterization Data |
|---|---|
| 36 | LCMS: (ES+) m/z (M + H)⁺ = 595.1. ¹H NMR (400 MHz, CD₃OD) δ ppm 7.82 (m, |
| | 2H), 7.69 (br d, J = 6.4 Hz, 2H), 7.63 (br d, J = 3.6 Hz, 1H), 7.33-7.25 (m, 2H), 7.21-7.12 |
| | (m, 2H), 6.87 (s, 1H), 4.49 (br s, 2H), 4.17 (m, 2H), 4.00 (br s, 2H), 3.69-3.52 (m, 3H), |
| | 3.52-3.34 (m, 2H), 2.33 (br s, 4H), 2.15-1.99 (m, 4H), 1.92-1.70 (m, 2H), 1.47 (t, .J = 7.2 |
| | Hz, 3H). |
| 37 | LCMS: (ES⁺) m/z (M + H)⁺ = 583.3. ¹H NMR (400 MHz, DMSO-d6) δ ppm 7.67-7.50 |
| | (m, 4H), 7.37-7.30 (m, 3H), 7.29-7.21 (m, 2H), 6.67 (s, 1H), 4.00 (d, J = 7.2 Hz, 2H), |
| | 3.89 (s, 2H), 3.60-3.57 (m, 2H), 2.87 (s, 1H), 2.60 (d, J = 5.2 Hz, 2H), 2.52 (s, 2H), |
| | 1.94 (s, 4H), 1.31 (t, J = 7.2 Hz, 3H), 1.09 (d, J = 6.8 Hz, 6H). |

### Example 16: (4-(((4-((2-cyclopropyl-5-ethoxy-4′-fluoro-[1,1′-biphenyl]-4-yl)methyl)piperazin-1-yl)sulfonyl)methyl)phenyl)phosphonic acid (Compound 38)

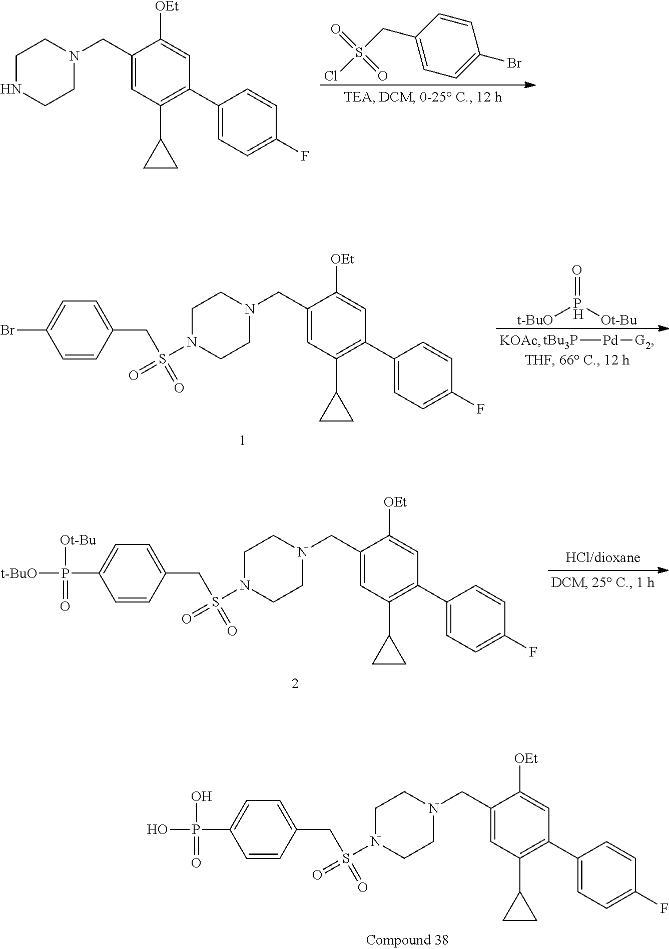

Step 1: 1-((4-bromobenzyl)sulfonyl)-4-((2-cyclopropyl-5-ethoxy-4′-fluoro-[1,1′-biphenyl]-4-yl)methyl)piperazine (1): To a solution of 1-[[5-cyclopropyl-2-ethoxy-4-(4-fluorophenyl)phenyl]methyl]piperazine (0.50 g, 1.2 mmol, 1 eq, 2 HCl salt) and TEA (0.47 g, 4.7 mmol, 4 eq) in DCM (10 mL) was added (4-bromophenyl)methanesulfonyl chloride (0.47 g, 1.8 mmol, 1.5 eq) at 0° C., and the mixture was stirred at 25° C. for 12 hours. The reaction mixture was diluted with H₂O (20 mL) and extracted with ethyl acetate (20 mL×3). The combined organic layers were washed with brine (20 mL×3), dried over Na₂SO₄, filtered and concentrated under reduced pressure. The residue was purified by column chromatography (SiO₂, petroleum ether/ethyl acetate, 3:1) to give 1 (0.36 g, 51% yield) as a white solid. LCMS: (ES⁺) m/z (M+H)⁺=589.2.

Step 2: di-tert-butyl (4-(((4-((2-cyclopropyl-5-ethoxy-4′-fluoro-[1,1′-biphenyl]-4-yl)methyl)piperazin-1-yl)sulfonyl)methyl)phenyl)phosphonate (2): To a solution of 1 (0.25 g, 0.43 mmol, 1 eq) and di-tert-butyl hydrogen phosphite (0.41 g, 2.1 mmol, 5 eq) in THF (6 mL) was added KOAc (0.13 g, 1.3 mmol, 3 eq) and tBu₃P—Pd-G2 (17 mg, 34 μmol, 0.10 eq) under N₂, and the mixture was stirred at 66° C. for 12 hours. The reaction mixture was diluted with H₂O (20 mL) and extracted with ethyl acetate (20 mL×3). The combined organic layers were washed with brine (20 mL×3), dried over Na₂SO₄, filtered and concentrated under reduced pressure to give 2 (0.25 g, crude) as a yellow oil. LCMS: (ES⁺) m/z (M+H)⁺=701.1.

Step 3: (4-(((4-((2-cyclopropyl-5-ethoxy-4′-fluoro-[1,1′-biphenyl]-4-yl)methyl)piperazin-1-yl)sulfonyl)methyl)phenyl)phosphonic acid (Compound 38): To a solution of 2 (0.13 g, 0.18 mmol, 1 eq) in DCM (2 mL) was added HCl/dioxane (4 M, 4 mL, 90 eq), and the mixture was stirred at 25° C. for 1 hour. The mixture was concentrated in vacuo. The residue was purified by prep-HPLC (column: Xtimate C18 150×40 mm×10 μm; mobile phase: A: water (0.05% ammonium hydroxide, v/v), B: ACN; B %:20%-50% gradient over 10 min) to give Compound 38 (64 mg, 61% yield, 99% purity) as a white solid. LCMS: (ES⁺) m/z (M+H)⁺=589.3. ¹H NMR (400 MHz, CD₃OD) δ 7.83 (dd, J₁=12.0 Hz, J₂=8.0 Hz, 2H), 7.46-7.40 (m, 4H), 7.14 (t, J=8.8 Hz, 2H), 6.94 (s, 1H), 6.76 (s, 1H), 4.38 (s, 2H), 4.03 (q, J=7.2 Hz, 2H), 3.64 (s, 2H), 3.19 (s, 4H), 2.57 (s, 4H), 1.79-1.72 (m, 1H), 1.39 (t, J=7.2 Hz, 3H), 0.79-0.74 (m, 2H), 0.59-0.57 (m, 2H)

The following compound was prepared according to the procedures described above using the appropriate intermediates.

| Cpd | Characterization Data |
|---|---|
| 39 | (ES⁺) m/z (M + H)⁺ = 617.3. ¹H NMR (400 MHz, CD₃OD) δ 7.79 (dd, J₁ = 12.0 Hz, J₂ = |
| | 8.0 Hz, 2H), 7.48-7.40 (m, 4H), 7.17-7.13 (m, 2H), 6.98 (s, 1H), 6.79 (s, 1H), 4.37 (s, |
| | 2H), 4.05 (q, J = 6.8 Hz, 2H), 3.79 (s, 4H), 3.22 (s, 4H), 2.71 (s, 4H), 1.79-1.72 (m, |
| | 1H), 1.40 (t, J = 6.8 Hz, 3H), 0.80-0.76 (m, 2H), 0.60-0.58 (m, 2H). |

### Example 17: 4-(8-(5-cyclopropyl-4-(1,2-dihydroxyethyl)-2-ethoxybenzyl)-2-oxo-1,3,8-triazaspiro[4.5]decan-3-yl)benzoic acid (Compound 40)

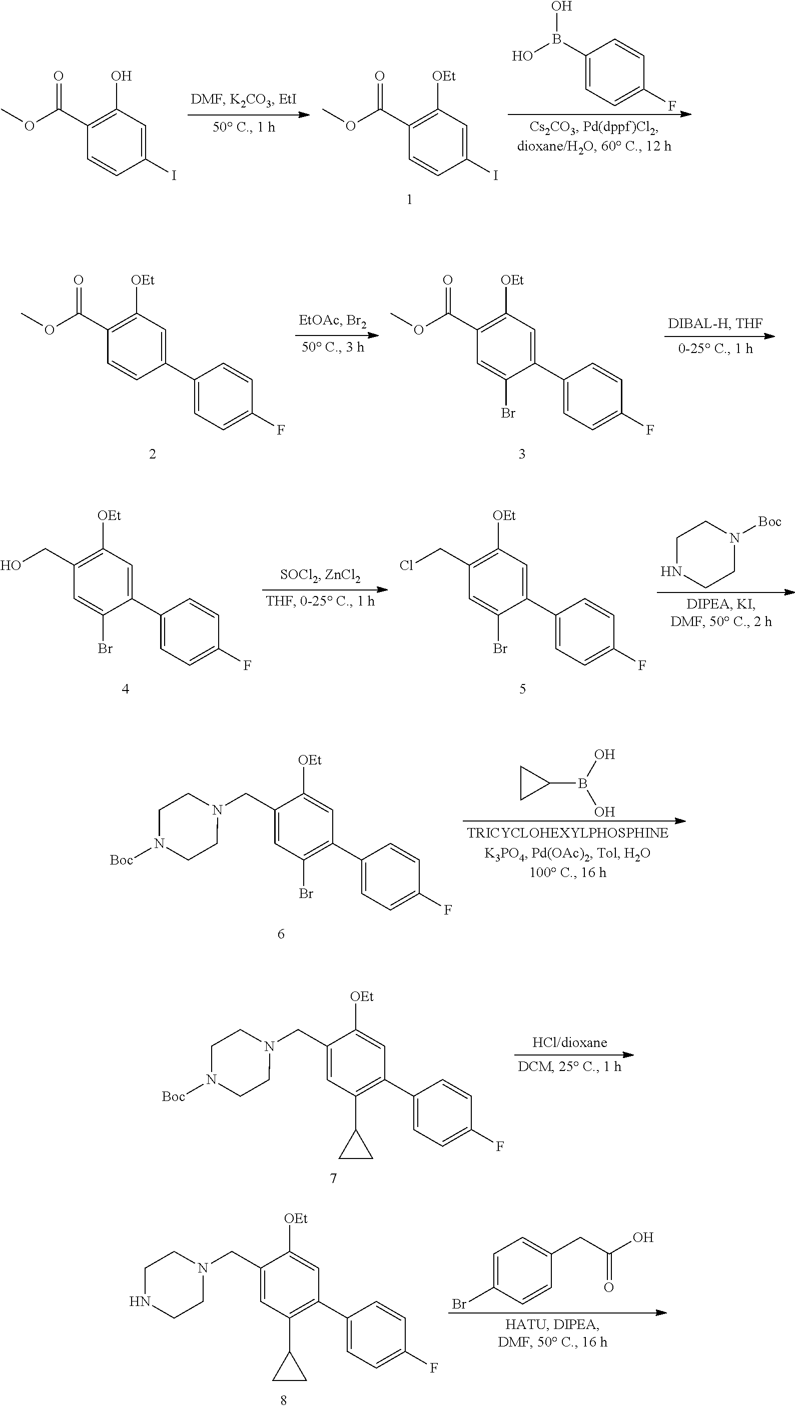

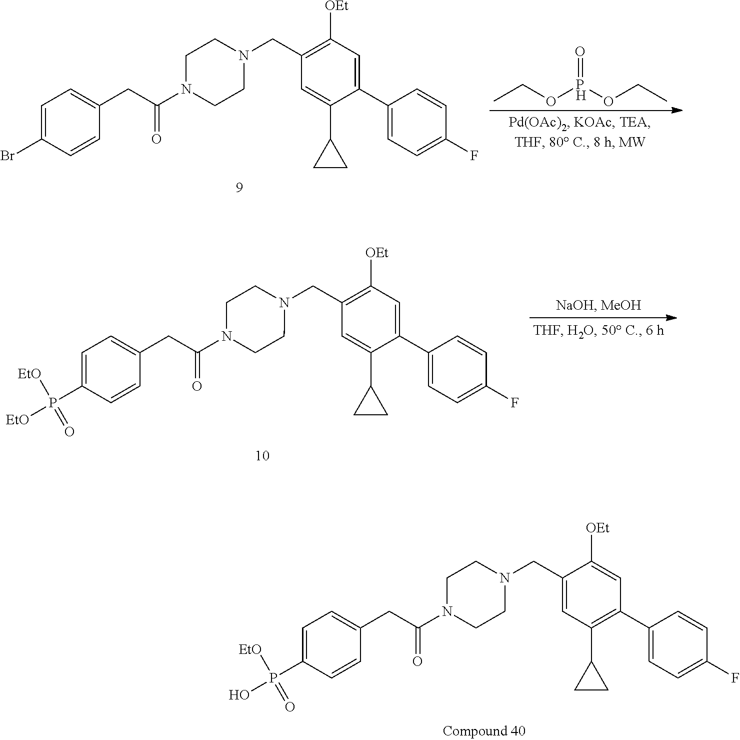

Step 1: methyl 2-ethoxy-4-iodo-benzoate (1): To a solution of methyl 2-hydroxy-4-iodo-benzoate (13 g, 47 mmol, 1 eq) in DMF (130 mL) was added K₂CO₃ (13 g, 94 mmol, 2 eq) and EtI (14.6 g, 94 mmol, 7.5 mL, 2 eq). The mixture was stirred at 50° C. for 1 hour. The reaction mixture was poured into H₂O (50 mL) and extracted with EA (50 mL×3). The combined organic layer was washed with water (50 mL×2) and brine (50 mL×2), dried over Na₂SO₄, filtered and concentrated in vacuo to give 1 (14.2 g, crude) as a yellow oil.

Step 2: methyl 2-ethoxy-4-(4-fluorophenyl)benzoate (2): To a mixture of 1 (7.5 g, 25 mmol, 1 eq), (4-fluorophenyl)boronic acid (3.8 g, 27 mmol, 1.1 eq), Cs₂CO₃ (16 g, 49 mmol, 2 eq) and Pd(dppf)Cl₂ (896 mg, 1.2 mmol, 0.05 eq) was added H₂O (20 mL) and dioxane (60 mL). Then the resulting mixture was stirred at 60° C. for 12 hours. The reaction mixture was diluted with H₂O (100 mL) and extracted with EA (90 mL×2). The combined organic layers were washed with saturated brine (50 mL×2), dried over Na₂SO₄, filtered, and concentrated under reduced pressure to give a residue. The residue was purified by column chromatography (SiO₂, petroleum ether/ethyl acetate, 100/1 to 10/1) to give 2 (6.7 g, 99% yield) as a yellow solid.

Step 3: methyl 5-bromo-2-ethoxy-4-(4-fluorophenyl)benzoate (3): To a solution of 2 (7.2 g, 26 mmol, 1 eq) in EtOAc (72 mL) was added Br₂ (5.0 g, 32 mmol, 1.6 mL, 1.2 eq). Then the mixture was stirred at 50° C. for 3 hours. The reaction mixture was diluted with H₂O (120 mL) and extracted with EA (75 mL×2). The combined organic layers were washed with saturated brine (50 mL×2), dried over Na₂SO₄, filtered, and concentrated under reduced pressure to give a residue. The residue was purified by column chromatography (SiO₂, petroleum ether/ethyl acetate, 50/1 to 5/1) to give 3 (5.5 g, 59% yield) as a white solid. ¹H NMR (400 MHz, CD₃OD) δ 8.07 (s, 1H), 7.35-7.42 (m, 2H), 7.17-7.10 (m, 2H), 6.90 (s, 1H), 4.11 (q, J=7.2 Hz, 2H), 3.91 (s, 3H), 1.47 (t, J=6.8 Hz, 3H).

Step 4: (2-bromo-5-ethoxy-4′-fluoro-[1,1′-biphenyl]-4-yl)methanol (4): To a solution of 3 (5.0 g, 14 mmol, 1 eq) in THE (50 mL) was added DIBAL-H (1 M, 35 mL, 2.5 eq) at 0° C. Then the mixture was stirred at 25° C. for 1 hour. The mixture was quenched with saturated aqueous NH₄Cl and adjusted to pH 3 with 1 N aqueous HCl. The reaction mixture was extracted with EA (50 mL×2). The combined organic layers were washed with saturated brine (40 mL×2), dried over Na₂SO₄, filtered, and concentrated under reduced pressure to give 4 (4.5 g, 98% yield) as a colorless oil.

Step 5: 2-bromo-4-(chloromethyl)-5-ethoxy-4′-fluoro-1,1′-biphenyl (5): To a solution of 4 (4.5 g, 14 mmol, 1 eq) and ZnCl₂ (189 mg, 1.4 mmol, 65 μL, 0.1 eq) in THF (50 mL) was added SOCl₂ (2.5 g, 21 mmol, 1.5 mL, 1.5 eq) at 0° C. Then the mixture was stirred at 25° C. for 1 hour. The solution mixture was quenched with slow addition of saturated aqueous NaHCO₃ (40 mL) and extracted with ethyl acetate (60 mL×3). The combined organic layers were washed with saturated brine (30 mL×2), dried over Na₂SO₄, filtered, and concentrated under reduced pressure to give 5 (4 g, 84% yield) as an off-white solid.

Step 6: tert-butyl 4-((2-bromo-5-ethoxy-4′-fluoro-[1,1′-biphenyl]-4-yl)methyl)piperazine-1-carboxylate (6): To a solution of 5 (3.0 g, 8.7 mmol, 1 eq), tert-butyl piperazine-1-carboxylate (1.6 g, 8.7 mmol, 1 eq) and KI (140 mg, 870 μmol, 0.1 eq) in DMF (20 mL) was added DIEA (2.3 g, 17 mmol, 3.0 mL, 2 eq). The mixture was stirred at 50° C. for 2 hours. The mixture was poured into H₂O (100 mL) and extracted with EA (60 mL×3). The combined organic layer was washed with water (60 mL×2) and brine (60 mL×2), dried over Na₂SO₄, filtered and concentrated in vacuo to give 6 (4.4 g, crude) as a yellow oil. LCMS: (ES⁺) m/z (M+H)⁺=495.2.

Step 7: tert-butyl 4-((2-cyclopropyl-5-ethoxy-4′-fluoro-[1,1′-biphenyl]-4-yl)methyl)piperazine-1-carboxylate (7): To a solution of 6 (4.4 g, 8.9 mmol, 1 eq), cyclopropylboronic acid (2.3 g, 27 mmol, 3 eq), K₃PO₄ (5.7 g, 27 mmol, 3 eq), and tricyclohexylphosphine (250 mg, 0.89 mmol, 0.29 mL, 0.1 eq) in toluene (40 mL) and H₂O (4 mL) was added Pd(OAc)₂ (100 mg, 0.45 mmol, 0.05 eq) under N₂, and the mixture was stirred at 100° C. for 12 hours. The reaction mixture was diluted with H₂O (50 mL) and extracted with EA (40 mL×2). The combined organic layers were washed with saturated brine (40 mL×2), dried over Na₂SO₄, filtered, and concentrated under reduced pressure to give a residue. The residue was purified by flash silica gel chromatography (20 g SepaFlash® Silica Flash Column, eluent of 0 to 20% ethyl acetate/petroleum ether gradient) to give 7 (3.9 g, 96% yield) as a yellow oil. LCMS: (ES⁺) m/z (M+H)⁺=455.2. ¹H NMR (400 MHz, CDCl₃) δ ppm 7.56-7.36 (m, 2H), 7.11 (t, J=8.8 Hz, 2H), 6.95 (s, 1H), 6.70 (s, 1H), 4.02 (m, 2H), 3.59 (s, 2H), 3.50-3.38 (m, 4H), 2.47 (t, J=4.8 Hz, 4H), 1.81-1.73 (m, 1H), 1.47 (s, 9H), 1.40 (t, J=7.2 Hz, 3H), 0.81-0.73 (m, 2H), 0.62-0.59 (m, 2H).

Step 8: 1-((2-cyclopropyl-5-ethoxy-4′-fluoro-[1,1′-biphenyl]-4-yl)methyl)piperazine (8): To a solution of 7 (3.9 g, 8.6 mmol, 1 eq) in DCM (10 mL) was added HCl/dioxane (4 M, 54 mL, 25 eq). The mixture was stirred at 25° C. for 1 hour. The reaction mixture was concentrated in vacuo to give 8 (3.5 g, crude, HCl salt) as a white solid. LCMS: (ES⁺) m/z (M+H)⁺=355.4.

Step 9: 2-(4-bromophenyl)-1-(4-((2-cyclopropyl-5-ethoxy-4′-fluoro-[1,1′-biphenyl]-4-yl)methyl)piperazin-1-yl)ethanone (9): To a solution of 8 (1 g, 2.6 mmol, 1 eq, HCl salt) and 2-(4-bromophenyl)acetic acid (1.1 g, 5.1 mmol, 2 eq) in DMF (10 mL) was added HATU (1.2 g, 3.1 mmol, 1.2 eq) and DIEA (0.99 g, 7.7 mmol, 1.3 mL, 3 eq). The mixture was stirred at 50° C. for 16 hours. The mixture was poured into H₂O (50 mL) and extracted with EA (40 mL×3). The combined organic layer was washed with water (50 mL×2) and brine (50 mL×2), dried over Na₂SO₄, filtered and concentrated in vacuo. The residue was purified by flash silica gel chromatography (ISCO®; 20 g SepaFlash® Silica Flash Column, eluent of 0-50% ethyl acetate/petroleum ether gradient, 30 mL/min) to give 9 (1.2 g, 85% yield) as a yellow oil. LCMS: (ES⁺) m/z (M+H)⁺=531.1.

Step 10: diethyl (4-(2-(4-((2-cyclopropyl-5-ethoxy-4′-fluoro-[1,1′-biphenyl]-4-yl)methyl)piperazin-1-yl)-2-oxoethyl)phenyl)phosphonate (10): A mixture of 9 (200 mg, 0.36 mmol, 1 eq), 1-ethoxyphosphonoyloxyethane (250 mg, 1.8 mmol, 0.23 mL, 5 eq), Pd(OAc)₂ (4.1 mg, 18 μmol, 0.05 eq), KOAc (3.6 mg, 36 μmol, 0.1 eq), TEA (44 mg, 0.44 mmol, 61 μL, 1.2 eq) and DPPF (20 mg, 36 μmol, 0.1 eq) were taken up into a microwave tube in THF (3 mL). The sealed tube was heated at 80° C. for 8 hours under microwave. The mixture was concentrated to give 10 (220 mg, crude) as a yellow oil. LCMS: (ES⁺) m/z (M+H)⁺=609.1.

Step 11: ethyl hydrogen (4-(2-(4-((2-cyclopropyl-5-ethoxy-4′-fluoro-[1,1′-biphenyl]-4-yl)methyl)piperazin-1-yl)-2-oxoethyl)phenyl)phosphonate (Compound 40): To a solution of 10 (220 mg, 0.36 mmol, 1 eq) in THE (2 mL), EtOH (2 mL) and H₂O (1 mL) was added NaOH (140 mg, 3.6 mmol, 10 eq). The mixture was stirred at 40° C. for 6 hours. The reaction mixture was concentrated in vacuo. The residue was purified by prep-HPLC (column: Xtimate C18 150×40 mm×10 μm; mobile phase: A: water (0.05% ammonium hydroxide v/v), B: ACN; B %: 28%-58% gradient over 10 min) to give Compound 40 (38.81 mg, 65 μmol, 18% yield, 100% purity, NH₄⁺ salt) was obtained as a brown solid. LCMS: (ES⁺) m/z (M+H)⁺=581.2. ¹H NMR (400 MHz, CD₃OD) δ 7.78-7.30 (m, 2H), 7.46-7.38 (m, 2H), 7.31-7.28 (m, 2H), 7.18-7.10 (m, 2H), 6.94 (s, 1H), 6.74 (s, 1H), 4.04-3.98 (m, 2H), 3.83-3.74 (m, 4H), 3.66-3.59 (m, 2H), 3.58-3.50 (m, 4H), 2.47 (t, J=5.2 Hz, 2H), 2.39-2.36 (m, 2H), 1.79-1.71 (m, 1H), 1.37 (t, J=6.8 Hz, 3H), 1.16 (t, J=7.2 Hz, 3H), 0.79-0.71 (m, 2H), 0.60-0.52 (m, 2H).

### Example 18: 4-(8-(5-cyclopropyl-4-(1,2-dihydroxyethyl)-2-ethoxybenzyl)-2-oxo-1,3,8-triazaspiro[4.5]decan-3-yl)benzoic acid (Compound 41)

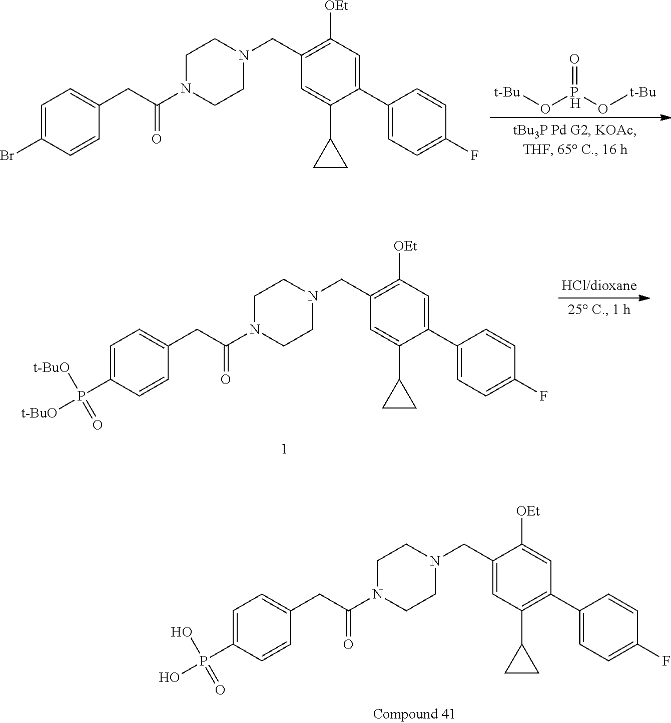

Step 1: di-tert-butyl (4-(2-(4-((2-cyclopropyl-5-ethoxy-4′-fluoro-[1,1′-biphenyl]-4-yl)methyl)piperazin-1-yl)-2-oxoethyl)phenyl)phosphonate (1): To a mixture of 2-(4-bromophenyl)-1-[4-[[5-cyclopropyl-2-ethoxy-4-(4-fluorophenyl)phenyl]methyl]piperazin-1-yl]ethanone (300 mg, 0.54 mmol, 1 eq), 2-tert-butoxyphosphonoyloxy-2-methylpropane (530 mg, 2.7 mmol, 5 eq), and KOAc (160 mg, 1.6 mmol, 3 eq) in THE (6 mL) in a glove box was added tBu₃P—Pd-G2 (22 mg, 44 μmol, 0.08 eq). The mixture was stirred at 65° C. for 16 hours under N₂ atmosphere. The mixture was poured into H₂O (20 mL) and extracted with EA (10 mL×3). The combined organic layer was washed with water (20 mL×2) and brine (20 mL×2), dried over Na₂SO₄, and concentrated in vacuo. The residue was purified by flash silica gel chromatography (ISCO®; 4 g SepaFlash® Silica Flash Column, eluent of 0 to 20% MeOH/ethyl acetate) to give 1 (240 mg, 360 μmol, 66% yield) as a yellow oil. LCMS: (ES⁺) m/z (M−31)⁺=665.2.

Step 2: 4-(8-(5-cyclopropyl-4-(dimethylphosphoryl)-2-ethoxybenzyl)-2-oxo-1,3,8-triazaspiro[4.5]decan-3-yl)benzoic acid (Compound 41): A solution of 1 (240 mg, 0.36 mmol, 1 eq) and HCl/dioxane (4 M, 1.8 mL, 20 eq) was stirred at 25° C. for 1 hour. The reaction mixture was concentrated in vacuo. The residue was purified by prep-HPLC (column: Phenomenex Gemini NX-C18 75×30 mm×3 μm; mobile phase: A: water (0.05% ammonium hydroxide v/v), B: ACN; B %: 2%-32% gradient over 7 min) to give Compound 41 (23.75 mg, 11% yield, 100% purity, ammonium salt) as an off-white solid. LCMS: (ES⁺) m/z (M+H)⁺=553.1. ¹H NMR (400 MHz, CD₃OD) δ ppm 7.82-7.76 (m, 2H), 7.46-7.38 (m, 2H), 7.30-7.25 (m, 2H), 7.17 (t, J=8.8 Hz, 2H), 6.94 (s, 1H), 6.74 (s, 1H), 4.11-4.06 (m, 2H), 3.95 (s, 2H), 3.83 (s, 2H), 3.75-3.68 (m, 4H), 2.87-2.78 (m, 2H), 1.80-1.74 (m, 1H), 1.42 (t, J=6.8 Hz, 3H), 0.80-0.75 (m, 2H), 0.64-0.61 (m, 2H).

The following compound was prepared according to the procedures described above using the appropriate intermediates.

| Cpd | Characterization Data |
|---|---|
| 42 | (ES⁺) m/z (M + H)⁺ = 632.3. ¹H NMR (400 MHz, CDCl₃) δ 8.37 (s, 1H), 7.47 (s, 2H), |
| | 7.27 (s, 1H), 7.06 (s, 3H), 4.10-3.99 (m, 5H), 3.89 (s, 3H), 3.74-3.48 (m, 11H), 3.07 |
| | (s, 2H), 2.51 (m, 5H), 1.37 (t, J = 6.8, 3H), 0.91 (m, 2H), 0.60 (m, 2H). |

### II. Biological Evaluation

### Example A-1: In Vitro Activity Assay

Inositol Phosphate Accumulation Assay

CHO-K₁ cells stably co-expressing human SSTR5 with Gqi5 were developed using Jump-In technology from Thermo-Fisher. Gqi5 is the mouse G alpha q protein, that was modified to interact with Gi-coupled GPCRs as described previously (Coward, P.; Chan, S. D.; Wada, H. G.; Humphries, G. M.; Conklin, B. R. Chimeric G proteins Allow a High-Throughput Signaling Assay of Gi-Coupled Receptors. Anal Biochem. 1999, 270(2), 242-248).

Co-expression of Gqi5 with SSTR5 allowed monitoring of SSTR5 activity by following IP1 accumulation. The assay was performed in a 384-well plate format using the IP1 assay kit from Cis-Bio in an antagonist mode, i.e., pre-incubation with antagonist following by receptor activation by agonist at a concentration generating 90% of full activation. Frozen cells expressing human SSTR5 were thawed, washed, and then plated in DMEM supplemented with 10% FBS and non-essential amino acids. 40 μL of 2.5×105 cells/mL were plated on a Poly D-Lysine coated 384-well white plate. The cells were then incubated for 16 hr. at 37° C./5% CO₂. After 16 hour the medium was removed, and 10 μL of stimulation buffer was added to the cells. Test compounds were dissolved in DMSO at concentrations 2000-fold that of the final assay concentrations. 7.5 nL compound solutions were transferred to the cell plates using a Labcyte Echo® acoustic liquid handler. The plates were then incubated for 15 minutes at 37° C./5% CO₂. After the first incubation, 5 μL of 30 nM SST28 were added to the cells, and the cells were incubated for 90 minutes at 37° C./5% CO₂. 5 μL of detection buffer (prepared as described in the IP-1 kit) was added to each well, and the plates were incubated at RT for 1 hour.

TR-FRET was measured using a ClarioSTAR plate reader, calculating the ratio between emissions at 665 nm and 620 nm (HTRF ratio). The HTRF ratio for positive (Max) and negative (Min) controls were used to normalize HTRF data and generate values for % inhibition. IC₅₀ and maximal inhibition values were determined using a standard 4-parameter fit.

The table below summarizes the assay data obtained for representative compounds.

| Cpd. | SSTR5 IC₅₀ ^{a} |
|---|---|
| 1 | +++ |
| 2 | +++ |
| 3 | +++ |
| 4 | +++ |
| 5 | +++ |
| 6 | +++ |
| 7 | +++ |
| 8 | +++ |
| 9 | +++ |
| 10 | +++ |
| 11 | +++ |
| 12 | +++ |
| 13 | +++ |
| 14 | +++ |
| 16 | − |
| 17 | ++ |
| 18 | + |
| 19 | + |
| 20 | + |
| 21 | +++ |
| 22 | +++ |
| 23 | − |
| 24 | ++ |
| 25 | +++ |
| 26 | +++ |
| 27 | +++ |
| 28 | +++ |
| 29 | +++ |
| 30 | +++ |
| 31 | ++ |
| 32 | +++ |
| 33 | +++ |
| 34 | +++ |
| 35 | +++ |
| 36 | +++ |
| 37 | +++ |
| 38 | +++ |
| 39 | +++ |
| 40 | +++ |
| 41 | +++ |
| 42 | − |

^{a} +++ ≤ 100 nM < ++ ≤ 1000 nM < + ≤ 5000 nM < −.

### Example A-2: Oral Bioavailability of the Compounds after Oral Dosing in Rat

Oral bioavailability of the compounds was determined in Sprague Dawley rats. The table below summarizes the results. Each compound was dosed intravenously (IV) at 1 mg/kg and orally (PO) 5 mg/kg using the respective vehicles listed below. The compounds display low (<10%) oral bioavailability (F %).

| Cpd | F % | IV vehicle | PO vehicle |
|---|---|---|---|
| 1 | 0.9% | 5% DMSO + | 0.5% methyl |
| | | 30% PEG400 + | cellulose in |
| | | 65% water | water |
| 2 | 8.7% | 5% DMSO + | 0.5% methyl |
| | | 30% PEG400 + | cellulose in |
| | | 65% water | water |
| 3 | 1.9% | 5% DMSO + | 5% DMSO + |
| | | 30% PEG400 + | 30% PEG400 + |
| | | 65% water | 65% wate |
| 9 | 0.2% | 5% DMSO + | 0.5% methyl |
| | | 30% PEG400 + | cellulose in |
| | | 65% water | water |

We claim:

1. A compound of Formula (I): 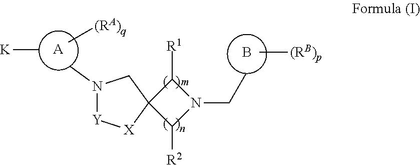
or a pharmaceutically acceptable salt, solvate, stereoisomer, or prodrug thereof, wherein:
X is —O—, —NR³—, or —C(R⁴)₂—;
Y is —C(═O)—, or —S(═O)₂—;
Ring A is aryl, heteroaryl, cycloalkyl, or heterocycloalkyl;
Ring B is aryl or heteroaryl;
K is —(CH₂)ⱼ-G;
G is —P(═O)(OH)₂, —P(═O)(OH)(R^{D}), —P(═O)(OH)(H), —P(═O)(OH)(OR^{D}), —B(OH)₂, —B(OR^{D})(OH), —NHC(═O)H, —NHC(═O)(R^{D}), —NHS(═O)₂(R^{D}), —NHC(═O)NHS(═O)₂(R^{D}), —N(R^{D})C(═O)NHS(═O)₂(R^{D}), —C(═O)NHS(═O)₂(R^{D}), —S(═O)₂NHC(═O)(R^{D}), —NHC(═O)NH₂, —NHC(═O)NH(R^{D}), —NHC(═NH)NH₂, —NHC(═NH)NH(R^{D}), —NHC(═NH)N(R^{D})₂, —N(R^{D})C(═NH)NH₂, —N(R^{D})C(═NH)NH(R^{D}), —N(R^{D})C(═NH)N(R^{D})₂, —NHC(═N(R^{D}))NH₂, —NHC(═N(R^{D}))NH(R^{D}), —NHC(═N(R^{D}))N(R^{D})₂, N(R^{D})C(═N(R^{D}))NH₂, —N(R^{D})C(═N(R^{D}))NH(R^{D}), —N(R^{D})C(═N(R^{D}))N(R^{D})₂, —NHC(═NH)NHC(═NH)NH₂, —N(R^{D})C(═NH)NHC(═NH)NH₂, 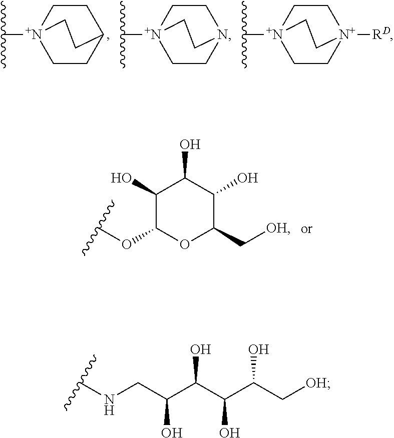
j is 0-4;
each R¹ and R² is independently hydrogen, C₁₋₆ alkyl, or C₁₋₆ fluoroalkyl;
or one R¹ and one R² are taken together to form a ring;
R³ is hydrogen, C₁₋₆ alkyl, C₁₋₆ fluoroalkyl, or C₃₋₆ cycloalkyl;
each R⁴ is independently hydrogen, C₁₋₆ alkyl, C₁₋₆ fluoroalkyl, or C₃₋₆ cycloalkyl;
each R^{D} is independently C₁₋₆ alkyl or C₃₋₆ cycloalkyl; wherein the alkyl and cycloalkyl are unsubstituted or substituted by 1-3 halogen or —OH groups;
each R^{A} is independently halogen, —OH, —O—(C₁-C₆ alkyl), C₁-C₆ alkyl, C₃-C₆ cycloalkyl, 3- to 8-membered heterocycloalkyl, wherein each alkyl, cycloalkyl, and heterocycloalkyl is unsubstituted or substituted with 1, 2, or 3 substituents selected from halogen, —CN, —OH, —O—(C₁-C₆ alkyl), C₁-C₆ alkyl, C₁-C₆ fluoroalkyl, C₁-C₆ hydroxyalkyl, —O—(C₁-C₆ fluoroalkyl), C₃-C₆ cycloalkyl, and 3- to 6-membered heterocycloalkyl;
each R^{B} is independently halogen, C₁-C₆ alkyl, C₃-C₆ cycloalkyl, C₃-C₆ cycloalkenyl, 3- to 8-membered heterocycloalkyl, 3- to 8-membered heterocycloalkenyl, aryl, heteroaryl, —CN, —OR⁹, —OCH₂R⁹, —CO₂R⁹, —CH₂CO₂R⁹, —OC(═O)R⁹, —C(═O)N(R⁹)₂, —N(R⁹)₂, —NR⁹C(═O)R⁹, —NR⁹C(═O)OR¹⁰, —OC(═O)NR⁹, —NR⁹C(═O)N(R⁹)₂, —C(R⁹)═N—OR⁹, —SR⁹, —S(═O)R¹⁰, —S(═O)₂R¹⁰, —S(═O)₂N(R⁹)₂, —P(═O)(OR⁹)₂, —P(═O)(OR⁹)R¹⁰ or —P(═O)(R¹⁰)₂, wherein each alkyl, aryl, and heteroaryl is unsubstituted or substituted with 1, 2, or 3 substituents selected from halogen, —CN, —OH, —O—(C₁-C₆ alkyl), —CO₂—(C₁-C₆ alkyl), C₁-C₆ alkyl, C₁-C₆ fluoroalkyl, C₁-C₆ hydroxyalkyl, —O—(C₁-C₆ fluoroalkyl), C₃-C₆ cycloalkyl, and 3- to 6-membered heterocycloalkyl; and wherein each cycloalkyl, cycloalkenyl, heterocycloalkyl, and heterocycloalkenyl is unsubstituted or substituted with 1, 2, or 3 substituents selected from halogen, —CN, —OH, ═O, —O—(C₁-C₆ alkyl), C₁-C₆ alkyl, C₁-C₆ fluoroalkyl, C₁-C₆ hydroxyalkyl, —O—(C₁-C₆ fluoroalkyl), C₃-C₆ cycloalkyl, and 3- to 6-membered heterocycloalkyl;
each R⁹ is independently selected from hydrogen, C₁-C₆ alkyl, C₁-C₆ fluoroalkyl, C₃-C₆ cycloalkyl, 3- to 8-membered heterocycloalkyl, phenyl, and monocyclic heteroaryl, wherein each alkyl, fluoroalkyl, cycloalkyl, heterocycloalkyl, phenyl, and heteroaryl is unsubstituted or substituted with 1, 2, or 3 substituents selected from halogen, —CN, —OH, —O—(C₁-C₆ alkyl), —NH₂, —NH(C₁-C₆ alkyl), —N(C₁-C₆ alkyl)₂, C₁-C₆ alkyl, C₁-C₆ fluoroalkyl, C₁-C₆ hydroxyalkyl, —O—(C₁-C₆ fluoroalkyl), C₃-C₆ cycloalkyl, 3- to 6-membered heterocycloalkyl, and 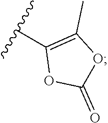 [SMILES: *c1oc(=O)oc1C]
or two R⁹ on the same N atom are taken together with the N atom to which they are attached to form a N-containing heterocycle, which is unsubstituted or substituted with 1, 2, or 3 substituents selected from halogen, —CN, —OH, —O—(C₁-C₆ alkyl), —NH₂, —NH(C₁-C₆ alkyl), —N(C₁-C₆ alkyl)₂, C₁-C₆ alkyl, C₁-C₆ fluoroalkyl, C₁-C₆ hydroxyalkyl, —O—(C₁-C₆ fluoroalkyl), C₃-C₆ cycloalkyl, and 3- to 6-membered heterocycloalkyl;
each R¹⁰ is independently selected from C₁-C₆ alkyl, C₁-C₆ fluoroalkyl, C₃-C₆ cycloalkyl, 3- to 8-membered heterocycloalkyl, phenyl, and monocyclic heteroaryl, wherein each alkyl, fluoroalkyl, cycloalkyl, heterocycloalkyl, phenyl, and heteroaryl is unsubstituted or substituted with 1, 2, or 3 substituents selected from halogen, —CN, —OH, —O—(C₁-C₆ alkyl), —NH₂, —NH(C₁-C₆ alkyl), —N(C₁-C₆ alkyl)₂, C₁-C₆ alkyl, C₁-C₆ fluoroalkyl, C₁-C₆ hydroxyalkyl, —O—(C₁-C₆ fluoroalkyl), C₃-C₆ cycloalkyl, 3- to 6-membered heterocycloalkyl, and 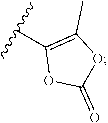 [SMILES: *c1oc(=O)oc1C]
m is 1 or 2;
n is 1 or 2;
p is 0-4; and
q is 0-4.

2. The compound of claim 1, or a pharmaceutically acceptable salt, solvate, stereoisomer, or prodrug thereof, wherein:
Ring B is phenyl or 6-membered heteroaryl;
each R¹ and R² is independently hydrogen or C₁₋₆ alkyl;
m is 2; and
n is 2.

3. The compound of claim 1, or a pharmaceutically acceptable salt, solvate, stereoisomer, or prodrug thereof, wherein the compound has the structure of Formula (Ia-1), or a pharmaceutically acceptable salt, solvate, stereoisomer, or prodrug thereof:

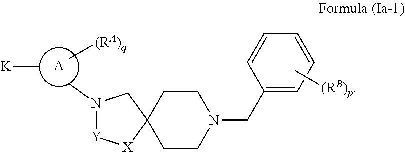

4. The compound of any one of claims 1-3, or a pharmaceutically acceptable salt, solvate, stereoisomer, or prodrug thereof, wherein:
X is —O—, and Y is —C(═O)—;
or X is —NR³—, and Y is —C(═O)—;
or X is —C(R⁴)₂—; and Y is —C(═O)—;
or X is —O—, and Y is —S(═O)₂—;
or X is —NR³—, and Y is —S(═O)₂—;
or X is —C(R⁴)₂—; and Y is —S(═O)₂—.

5. The compound of any one of claims 1-4, or a pharmaceutically acceptable salt, solvate, stereoisomer, or prodrug thereof, wherein:
X is —O—, and Y is —C(═O)—;
or X is —NP³—, and Y is —C(═O)—;
or X is —C(R⁴)₂—; and Y is —C(═O)—;
or X is —NR³—, and Y is —S(═O)₂—.

6. The compound of claim 1, or a pharmaceutically acceptable salt, solvate, stereoisomer, or prodrug thereof, wherein the compound has the structure of Formula (Ib), Formula (Ic), Formula (Id), or Formula (Ie), or a pharmaceutically acceptable salt, solvate, stereoisomer, or prodrug thereof:

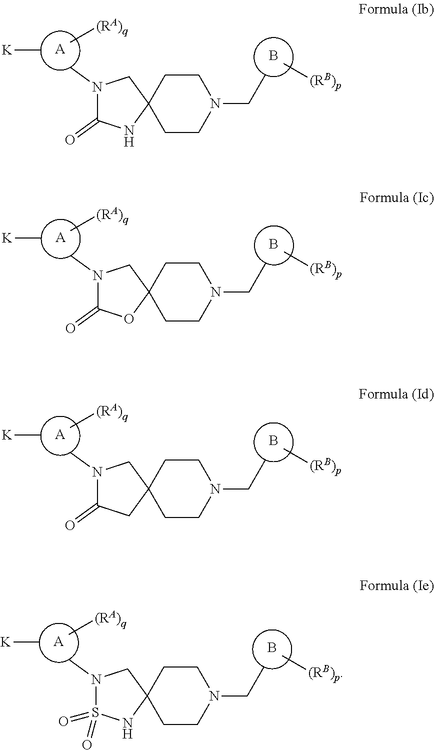

7. The compound of any one of claims 1-6, or a pharmaceutically acceptable salt, solvate, stereoisomer, or prodrug thereof, wherein:
each R^{B} is independently halogen, C₁-C₆ alkyl, C₃-C₆ cycloalkyl, C₃-C₆ cycloalkenyl, 3- to 8-membered heterocycloalkyl, 3- to 8-membered heterocycloalkenyl, aryl, heteroaryl, —CN, —OR⁹, —OCH₂R⁹, —CO₂R⁹, —CH₂CO₂R⁹, —OC(═O)R⁹, —C(═O)N(R⁹)₂, —N(R⁹)₂, —NR⁹C(═O)R⁹, —NR⁹C(═O)OR¹⁰, —OC(═O)NR⁹, —NR⁹C(═O)N(R⁹)₂, —C(R⁹)═N—OR⁹, —SR⁹, —S(═O)R¹⁰, —S(═O)₂R¹⁰, —S(═O)₂N(R⁹)₂, —P(═O)(OR⁹)₂, —P(═O)(OR⁹)R¹⁰ or —P(═O)(R¹⁰)₂, wherein each alkyl, aryl, and heteroaryl is unsubstituted or substituted with 1, 2, or 3 substituents selected from halogen, —CN, —OH, —O—(C₁-C₆ alkyl), —CO₂—(C₁-C₆ alkyl), C₁-C₆ alkyl, C₁-C₆ fluoroalkyl, C₁-C₆ hydroxyalkyl, —O—(C₁-C₆ fluoroalkyl), C₃-C₆ cycloalkyl, and 3- to 6-membered heterocycloalkyl; and
wherein each cycloalkyl, cycloalkenyl, heterocycloalkyl, and heterocycloalkenyl is unsubstituted or substituted with 1, 2, or 3 substituents selected from halogen, —CN, —OH, ═O, —O—(C₁-C₆ alkyl), C₁-C₆ alkyl, C₁-C₆ fluoroalkyl, C₁-C₆ hydroxyalkyl, —O—(C₁-C₆ fluoroalkyl), C₃-C₆ cycloalkyl, and 3- to 6-membered heterocycloalkyl; and
p is 1-4.

8. The compound of any one of claims 1-7, or a pharmaceutically acceptable salt, solvate, stereoisomer, or prodrug thereof, wherein:
each R^{B} is independently halogen, C₁-C₆ alkyl, phenyl, C₃-C₆ cycloalkyl, 3- to 6-membered heterocycloalkyl, 3- to 6-membered heterocycloalkenyl, 5-membered heteroaryl, 6-membered heteroaryl, —CN, —OR⁹, —CH₂CO₂R⁹, —CO₂R⁹, —C(═O)N(R⁹)₂, —N(R⁹)₂, —S(═O)₂R¹⁰, —S(═O)₂N(R⁹)₂, or —P(═O)(R¹⁰)₂, wherein each alkyl, phenyl, and heteroaryl is unsubstituted or substituted with 1, 2, or 3 substituents selected from halogen, —CN, —OH, —O—(C₁-C₆ alkyl), C₁-C₆ alkyl, C₁-C₆ fluoroalkyl, C₁-C₆ hydroxyalkyl, —O—(C₁-C₆ fluoroalkyl), C₃-C₆ cycloalkyl, and 3- to 6-membered heterocycloalkyl; and wherein each cycloalkyl, heterocycloalkyl, and heterocycloalkenyl is unsubstituted or substituted with 1, 2, or 3 substituents selected from halogen, —CN, —OH, ═O, —O—(C₁-C₆ alkyl), C₁-C₆ alkyl, C₁-C₆ fluoroalkyl, C₁-C₆ hydroxyalkyl, —O—(C₁-C₆ fluoroalkyl), C₃-C₆ cycloalkyl, and 3- to 6-membered heterocycloalkyl.

9. The compound of any one of claims 1-8, or a pharmaceutically acceptable salt, solvate, stereoisomer, or prodrug thereof, wherein:
each R^{B} is independently halogen, C₁-C₆ alkyl, phenyl, C₃-C₆ cycloalkyl, 5-membered heteroaryl, 6-membered heteroaryl, —CN, —OR⁹, —CH₂CO₂R⁹, —CO₂R⁹, —C(═O)N(R⁹)₂, or —S(═O)₂R¹⁰, wherein each alkyl, cycloalkyl, phenyl, and heteroaryl is unsubstituted or substituted with 1, 2, or 3 substituents selected from —F, —Cl, —Br, —CN, —OH, —CH₂OH, —O—(C₁-C₆ alkyl), C₁-C₆ alkyl, and C₁-C₆ fluoroalkyl.

10. The compound of claim 1, or a pharmaceutically acceptable salt, solvate, stereoisomer, or prodrug thereof, wherein the compound has the structure of Formula (If), or a pharmaceutically acceptable salt, solvate, stereoisomer, or prodrug thereof:

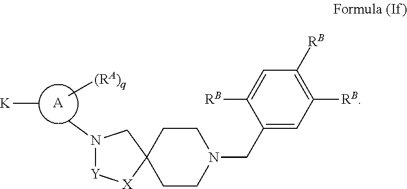

11. The compound of claim 1, or a pharmaceutically acceptable salt, solvate, stereoisomer, or prodrug thereof, wherein the compound has the structure of Formula (Ig), or a pharmaceutically acceptable salt, solvate, stereoisomer, or prodrug thereof:

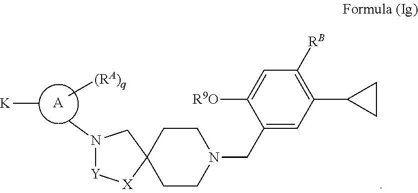

12. The compound of claim 11, or a pharmaceutically acceptable salt, solvate, stereoisomer, or prodrug thereof, wherein:
R^{B} is phenyl, oxadiazolyl, pyridinyl, —CN, —CH₂CO₂R⁹, —CO₂R⁹, or —S(═O)₂R¹⁰ wherein the phenyl, oxadiazolyl, or pyridinyl is unsubstituted or substituted with 1, 2, or 3 substituents selected from —F, —Cl, —Br, —CN, —OH, —CH₂OH, —O—(C₁-C₆ alkyl), C₁-C₆ alkyl, C₁-C₆ fluoroalkyl.

13. The compound of any one of claims 1-12, or a pharmaceutically acceptable salt, solvate, stereoisomer, or prodrug thereof, wherein:
Ring A is phenyl, monocyclic heteroaryl, monocyclic cycloalkyl, spirocyclic cycloalkyl, bridged cycloalkyl, monocyclic heterocycloalkyl, spirocyclic heterocycloalkyl, or bridged heterocycloalkyl;
each R^{A} is independently halogen, —OH, —O—(C₁-C₆ alkyl), C₁-C₆ alkyl, C₃-C₆ cycloalkyl, wherein each alkyl and cycloalkyl is unsubstituted or substituted with 1, 2, or 3 substituents selected from halogen, —CN, —OH, —O—(C₁-C₆ alkyl), C₁-C₆ alkyl, and C₁-C₆ fluoroalkyl; and
q is 0-2.

14. The compound of any one of claims 1-12, or a pharmaceutically acceptable salt, solvate, stereoisomer, or prodrug thereof, wherein:
Ring A is phenyl, monocyclic C₃-C₆ cycloalkyl, or bridged cycloalkyl;
each R^{A} is independently halogen, —OH, —O—(C₁-C₆ alkyl), or C₁-C₆ alkyl; and
q is 0-2.

15. The compound of any one of claims 1-12, or a pharmaceutically acceptable salt, solvate, stereoisomer, or prodrug thereof, wherein:
Ring A is phenyl, cyclohexyl, or 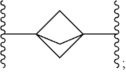 [SMILES: *C12CC(*)(C1)C2]
each R^{A} is independently halogen, —OH, —O—(C₁-C₆ alkyl), or C₁-C₆ alkyl; and
q is 0-2.

16. The compound of any one of claims 1-15, or a pharmaceutically acceptable salt, solvate, stereoisomer, or prodrug thereof, wherein:
Ring A is phenyl; and
q is 0.

17. The compound of any one of claims 1-12, or a pharmaceutically acceptable salt, solvate, stereoisomer, or prodrug thereof, wherein:
X is —O—, and Y is —C(═O)—.

18. The compound of claim 17, or a pharmaceutically acceptable salt, solvate, stereoisomer, or prodrug thereof, wherein:
Ring A is phenyl or heteroaryl.

19. The compound of claim 18, or a pharmaceutically acceptable salt, solvate, stereoisomer, or prodrug thereof, wherein:
Ring A is phenyl.

20. The compound of claim 17, or a pharmaceutically acceptable salt, solvate, stereoisomer, or prodrug thereof, wherein:
Ring A is monocyclic cycloalkyl, spirocyclic cycloalkyl, bridged cycloalkyl, monocyclic heterocycloalkyl, spirocyclic heterocycloalkyl, or bridged heterocycloalkyl.

21. The compound of claim 20, or a pharmaceutically acceptable salt, solvate, stereoisomer, or prodrug thereof, wherein:
Ring A is monocyclic C₃-C₆ cycloalkyl, or bridged cycloalkyl.

22. The compound of claim 21, or a pharmaceutically acceptable salt, solvate, stereoisomer, or prodrug thereof, wherein:
Ring A is cyclohexyl or

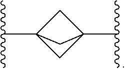 [SMILES: *C12CC(*)(C1)C2]

23. The compound of any one of claims 17-22, or a pharmaceutically acceptable salt, solvate, stereoisomer, or prodrug thereof, wherein:
each R^{A} is independently halogen, —OH, —O—(C₁-C₆ alkyl), C₁-C₆ alkyl, C₃-C₆ cycloalkyl, wherein each alkyl and cycloalkyl is unsubstituted or substituted with 1, 2, or 3 substituents selected from halogen, —CN, —OH, —O—(C₁-C₆ alkyl), C₁-C₆ alkyl, and C₁-C₆ fluoroalkyl; and
q is 0-2.

24. The compound of any claim 23, or a pharmaceutically acceptable salt, solvate, stereoisomer, or prodrug thereof, wherein:
each R^{A} is independently halogen, —OH, —O—(C₁-C₆ alkyl), or C₁-C₆ alkyl.

25. The compound of claim 24, or a pharmaceutically acceptable salt, solvate, stereoisomer, or prodrug thereof, wherein:
each R^{A} is independently C₁-C₆ alkyl.

26. The compound of any one of claims 17-22, or a pharmaceutically acceptable salt, solvate, stereoisomer, or prodrug thereof, wherein:
q is 0.

27. The compound of any one of claims 1-16, or a pharmaceutically acceptable salt, solvate, stereoisomer, or prodrug thereof, wherein:
X is —NR³—, and Y is —C(═O)—;
or X is —C(R⁴)₂—; and Y is —C(═O)—;
or X is —O—, and Y is —S(═O)₂—;
or X is —NR³—, and Y is —S(═O)₂—;
or X is —C(R⁴)₂—; and Y is —S(═O)₂—.

28. The compound of claim 1, or a pharmaceutically acceptable salt, solvate, stereoisomer, or prodrug thereof, wherein the compound has the structure of Formula (Ih-1), or a pharmaceutically acceptable salt, solvate, stereoisomer, or prodrug thereof:

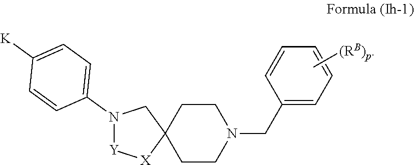

29. The compound of claim 1, or a pharmaceutically acceptable salt, solvate, stereoisomer, or prodrug thereof, wherein the compound has the structure of Formula (Ii), Formula (Ij), Formula (Ik), or Formula (Il), or a pharmaceutically acceptable salt, solvate, stereoisomer, or prodrug thereof:

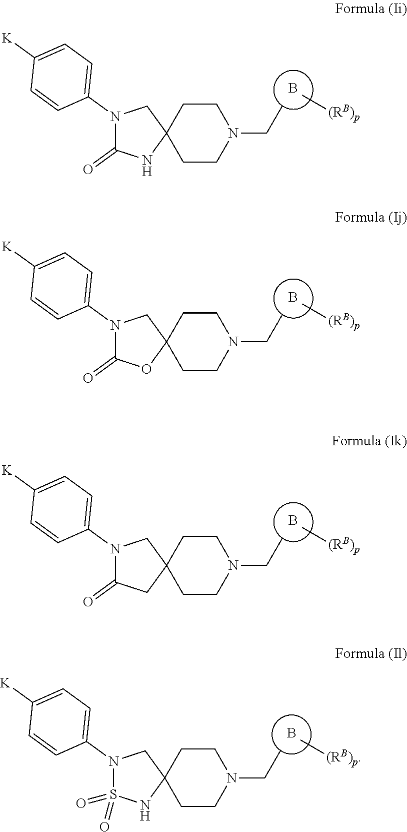

30. The compound of any one of claims 1-29, or a pharmaceutically acceptable salt, solvate, stereoisomer, or prodrug thereof, wherein:
K is —(CH₂)ⱼ-G; and
j is 0 or 1.

31. The compound of any one of claims 1-30, or a pharmaceutically acceptable salt, solvate, stereoisomer, or prodrug thereof, wherein:
G is —P(═O)(OH)₂, —P(═O)(OH)(R^{D}), —P(═O)(OH)(OR^{D}), —B(OH)₂, —B(OR^{D})(OH), —NHC(═O)H, —NHC(═O)(R^{D}), —NHS(═O)₂(R^{D}), —NHC(═O)NH₂, —NHC(═O)NH(R^{D}), —N(R^{D})C(═O)NHS(═O)₂(R^{D}), or —C(═O)NHS(═O)₂(R^{D}); and
R^{D} is alkyl which is unsubstituted or substituted with 1, 2, or 3 —OH groups.

32. The compound of any one of claims 1-31, or a pharmaceutically acceptable salt, solvate, stereoisomer, or prodrug thereof, wherein:
G is —P(═O)(OH)₂, —P(═O)(OH)(R^{D}), or —P(═O)(OH)(OR^{D});
R^{D} is C₁₋₆ alkyl; and
j is 0 or 1.

33. The compound of any one of claims 1-32, or a pharmaceutically acceptable salt, solvate, stereoisomer, or prodrug thereof, wherein:
K is —(CH₂)ⱼP(═O)(OH)₂, —(CH₂)ⱼP(═O)(OMe)(OH), —(CH₂)ⱼP(═O)(OEt)(OH), —(CH₂)ⱼP(═O)(Me)(OH), or —(CH₂)ⱼP(═O)(Et)(OH); and
j is 0 or 1.

34. The compound of any one of claims 1-33, or a pharmaceutically acceptable salt, solvate, stereoisomer, or prodrug thereof, wherein:
K is —P(═O)(OH)₂, —P(═O)(OEt)(OH), or —P(═O)(Me)(OH).

35. The compound of claim 34, or a pharmaceutically acceptable salt, solvate, stereoisomer, or prodrug thereof, wherein the compound has the structure of Formula (Ij-a) or Formula (Ij-b), or a pharmaceutically acceptable salt, solvate, stereoisomer, or prodrug thereof:

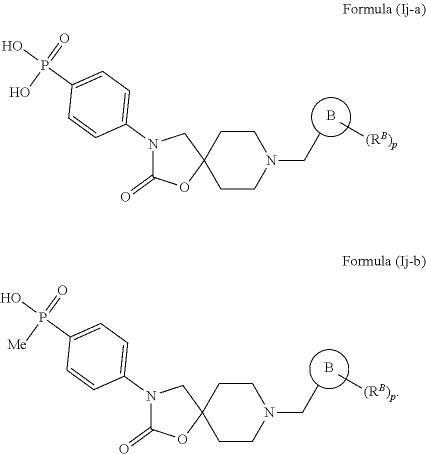

36. The compound of claim 1, wherein the compound is:
(4-(8-((2-cyclopropyl-5-ethoxy-4′-fluoro-[1,1′-biphenyl]-4-yl)methyl)-2-oxo-1-oxa-3,8-diazaspiro[4.5]decan-3-yl)phenyl)phosphonic acid;
(4-(8-((2-cyclopropyl-5-ethoxy-4′-fluoro-[1,1′-biphenyl]-4-yl)methyl)-2-oxo-1-oxa-3,8-diazaspiro[4.5]decan-3-yl)phenyl)(methyl)phosphinic acid;
(4-(8-((2-cyclopropyl-5-ethoxy-4′-fluoro-[1,1′-biphenyl]-4-yl)methyl)-2-oxo-1-oxa-3,8-diazaspiro[4.5]decan-3-yl)phenyl)phosphinic acid;
(4-(8-((5-cyclopropyl-2-ethoxy-6-(4-fluorophenyl)pyridin-3-yl)methyl)-2-oxo-1-oxa-3,8-diazaspiro[4.5]decan-3-yl)phenyl)(methyl)phosphinic acid;
(6-(8-((2-cyclopropyl-5-ethoxy-4′-fluoro-[1,1′-biphenyl]-4-yl)methyl)-2-oxo-1-oxa-3,8-diazaspiro[4.5]decan-3-yl)pyridin-3-yl)phosphonic acid;
(5-(8-((2-cyclopropyl-5-ethoxy-4′-fluoro-[1,1′-biphenyl]-4-yl)methyl)-2-oxo-1-oxa-3,8-diazaspiro[4.5]decan-3-yl)pyridin-2-yl)phosphonic acid;
(4-(8-((2-cyclopropyl-5-ethoxy-4′-fluoro-[1,1′-biphenyl]-4-yl)methyl)-3-oxo-2,8-diazaspiro[4.5]decan-2-yl)phenyl)(methyl)phosphinic acid;
(4-(8-(4-cyano-5-cyclopropyl-2-ethoxybenzyl)-2-oxo-1-oxa-3,8-diazaspiro[4.5]decan-3-yl)phenyl)(methyl)phosphinic acid;
(4-(8-(5-cyclopropyl-2-ethoxy-4-(5-fluoropyridin-2-yl)benzyl)-2-oxo-1-oxa-3,8-diazaspiro[4.5]decan-3-yl)phenyl)phosphonic acid;
(4-(8-((5-cyclopropyl-2-ethoxy-6-(4-fluorophenyl)pyridin-3-yl)methyl)-2-oxo-1-oxa-3,8-diazaspiro[4.5]decan-3-yl)phenyl)phosphonic acid;
((3-(8-((5-cyclopropyl-2-ethoxy-6-(4-fluorophenyl)pyridin-3-yl)methyl)-2-oxo-1-oxa-3,8-diazaspiro[4.5]decan-3-yl)bicyclo[1.1.1]pentan-1-yl)methyl)phosphonic acid;
((3-(8-((2-cyclopropyl-5-ethoxy-4′-fluoro-[1,1′-biphenyl]-4-yl)methyl)-2-oxo-1-oxa-3,8-diazaspiro[4.5]decan-3-yl)bicyclo[1.1.1]pentan-1-yl)methyl)phosphonic acid;
(4-(8-((2-cyclopropyl-5-ethoxy-4′-fluoro-[1,1′-biphenyl]-4-yl)methyl)-2-oxo-1-oxa-3,8-diazaspiro[4.5]decan-3-yl)phenyl)boronic acid;
1-carbamimidoyl-3-[2-[4-[8-[[5-cyclopropyl-2-ethoxy-4-(4-fluorophenyl)phenyl]methyl]-2-oxo-1-oxa-3,8-diazaspiro[4.5]decan-3-yl]phenyl]ethyl]guanidine;
methyl 2-cyclopropyl-4-((2-(4-(4-(3-(1,3-dihydroxy-2-(hydroxymethyl)propan-2-yl)ureido)butyl)phenyl)-3-oxo-2,8-diazaspiro[4.5]decan-8-yl)methyl)-5-ethoxybenzoate;
methyl 2-cyclopropyl-5-ethoxy-4-((2-(4-(4-formamidobutyl)phenyl)-3-oxo-2,8-diazaspiro[4.5]decan-8-yl)methyl)benzoate;
methyl 2-cyclopropyl-5-ethoxy-4-((2-(4-(4-(methylsulfonamido)butyl)phenyl)-3-oxo-2,8-diazaspiro[4.5]decan-8-yl)methyl)benzoate;
methyl 2-cyclopropyl-5-ethoxy-4-((2-(4-(4-(2-hydroxyacetamido)butyl)phenyl)-3-oxo-2,8-diazaspiro[4.5]decan-8-yl)methyl)benzoate;
methyl 2-cyclopropyl-5-ethoxy-4-((3-oxo-2-(4-(4-ureidobutyl)phenyl)-2,8-diazaspiro[4.5]decan-8-yl)methyl)benzoate;
(4-(8-(5-cyclopropyl-2-ethoxy-4-(methoxycarbonyl)benzyl)-2-oxo-1,3,8-triazaspiro[4.5]decan-3-yl)phenyl)phosphonic acid;
(4-(8-((2-cyclopropyl-5-ethoxy-4′-fluoro-[1,1′-biphenyl]-4-yl)methyl)-2-oxo-1,3,8-triazaspiro[4.5]decan-3-yl)phenyl)phosphonic acid;
(4-(8-(5-cyclopropyl-2-ethoxy-4-(methoxycarbonyl)benzyl)-3-oxo-2,8-diazaspiro[4.5]decan-2-yl)phenyl)phosphonic acid;
(4-(8-(5-cyclopropyl-2-ethoxy-4-(isopropoxycarbonyl)benzyl)-3-oxo-2,8-diazaspiro[4.5]decan-2-yl)phenyl)phosphonic acid;
ethyl hydrogen (4-(8-((2-cyclopropyl-5-ethoxy-4′-fluoro-[1,1′-biphenyl]-4-yl)methyl)-2-oxo-1,3,8-triazaspiro[4.5]decan-3-yl)phenyl)phosphonate;
(4-(8-(5-cyclopropyl-2-ethoxy-4-(3-methyl-1,2,4-oxadiazol-5-yl)benzyl)-2-oxo-1-oxa-3,8-diazaspiro[4.5]decan-3-yl)phenyl)phosphonic acid;
(4-(8-(5-cyclopropyl-2-ethoxy-4-(3-methyl-1,2,4-oxadiazol-5-yl)benzyl)-2-oxo-1,3,8-triazaspiro[4.5]decan-3-yl)phenyl)phosphonic acid;
(4-(8-((2-cyclopropyl-5-ethoxy-4′-fluoro-[1,1′-biphenyl]-4-yl)methyl)-2-oxo-1,3,8-triazaspiro[4.5]decan-3-yl)phenyl)(methyl)phosphinic acid;
(4-(8-((2-cyclopropyl-5-ethoxy-4′-fluoro-[1,1′-biphenyl]-4-yl)methyl)-3-oxo-2,8-diazaspiro[4.5]decan-2-yl)phenyl)phosphonic acid;
(4-(8-((2-cyclopropyl-5-ethoxy-4′-fluoro-[1,1′-biphenyl]-4-yl)methyl)-2-oxo-1-oxa-3,8-diazaspiro[4.5]decan-3-yl)benzyl)phosphonic acid;
1-(4-(3-(8-(5-cyclopropyl-2-ethoxy-4-(methylsulfonyl)benzyl)-2-oxo-1,3,8-triazaspiro[4.5]decan-3-yl)phenyl)butyl)-3-(1,3-dihydroxy-2-(hydroxymethyl)propan-2-yl)urea;
8-((2-cyclopropyl-5-ethoxy-4′-fluoro-[1,1′-biphenyl]-4-yl)methyl)-3-(4-((((2S,3R,4R,5R)-2,3,4,5,6-pentahydroxyhexyl)amino)methyl)phenyl)-1-oxa-3,8-diazaspiro[4.5]decan-2-one;
((cis)-4-(8-((2-cyclopropyl-5-ethoxy-4′-fluoro-[1,1′-biphenyl]-4-yl)methyl)-2-oxo-1-oxa-3,8-diazaspiro[4.5]decan-3-yl)cyclohexyl)phosphonic acid;
((trans)-4-(8-((2-cyclopropyl-5-ethoxy-4′-fluoro-[1,1′-biphenyl]-4-yl)methyl)-2-oxo-1-oxa-3,8-diazaspiro[4.5]decan-3-yl)cyclohexyl)phosphonic acid;
(4-(8-(5-cyclobutyl-2-ethoxy-4-(5-fluoropyridin-2-yl)benzyl)-2-oxo-1-oxa-3,8-diazaspiro[4.5]decan-3-yl)phenyl)phosphonic acid;
(4-(8-((2-cyclobutyl-5-ethoxy-4′-fluoro-[1,1′-biphenyl]-4-yl)methyl)-2-oxo-1-oxa-3,8-diazaspiro[4.5]decan-3-yl)phenyl)phosphonic acid;
(4-(8-((5-ethoxy-4′-fluoro-2-isopropyl-[1,1′-biphenyl]-4-yl)methyl)-2-oxo-1-oxa-3,8-diazaspiro[4.5]decan-3-yl)phenyl)phosphonic acid;
or a pharmaceutically acceptable salt, solvate, stereoisomer, or prodrug thereof.

37. A compound of Formula (II): 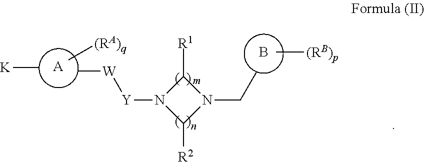
or a pharmaceutically acceptable salt, solvate, stereoisomer, or prodrug thereof, wherein:
W is a bond, —O—, —NR³—, or —C(R⁴)₂—;
Y is —C(═O)—, or —S(═O)₂—;
Ring A is aryl, heteroaryl, cycloalkyl, or heterocycloalkyl;
Ring B is aryl or heteroaryl;
K is —(CH₂)ⱼ-G;
G is —P(═O)(OH)₂, —P(═O)(OH)(R^{D}), —P(═O)(OH)(H), —P(═O)(OH)(OR^{D}), —B(OH)₂, —B(OR^{D})(OH), —NHC(═O)H, —NHC(═O)(R^{D}), —NHS(═O)₂(R^{D}), —NHC(═O)NHS(═O)₂(R^{D}), —N(R^{D})C(═O)NHS(═O)₂(R^{D}), —C(═O)NHS(═O)₂(R^{D}), —S(═O)₂NHC(═O)(R^{D}), —NHC(═O)NH₂, —NHC(═O)NH(R^{D}), —NHC(═NH)NH₂, —NHC(═NH)NH(R^{D}), —NHC(═NH)N(R^{D})₂, —N(R^{D})C(═NH)NH₂, —N(R^{D})C(═NH)NH(R^{D}), —N(R^{D})C(═NH)N(R^{D})₂, —NHC(═N(R^{D}))NIH2, —NHC(═N(R^{D}))NH(R^{D}), —NHC(═N(R^{D}))N(R^{D})₂, N(R^{D})C(═N(R^{D}))NH₂, —N(R^{D})C(═N(R^{D}))NH(R^{D}), —N(R^{D})C(═N(R^{D}))N(R^{D})₂, —NHC(═NH)NHC(═NH)NH₂, —N(R^{D})C(═NH)NHC(═NH)NH₂, 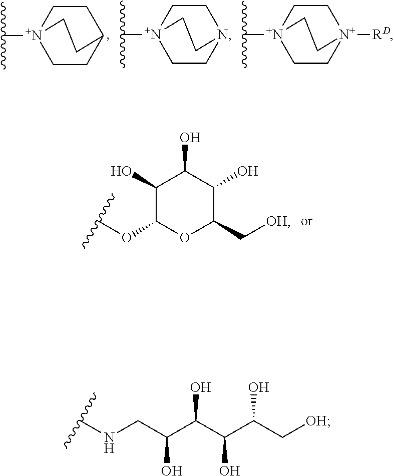
j is 0-4;
wherein when K is —B(OH)₂, W is —O—, —NR³—, or —C(R⁴)₂—;
each R¹ and R² is independently hydrogen, C₁₋₆ alkyl, or C₁₋₆ fluoroalkyl;
or one R¹ and one R² are taken together to form a ring;
R³ is hydrogen, C₁₋₆ alkyl, C₁₋₆ fluoroalkyl, or C₃₋₆ cycloalkyl;
each R⁴ is independently hydrogen, C₁₋₆ alkyl, C₁₋₆ fluoroalkyl, or C₃₋₆ cycloalkyl;
each R^{D} is independently C₁₋₆ alkyl or C₃₋₆ cycloalkyl; wherein the alkyl and cycloalkyl are unsubstituted or substituted by 1-3 halogen or —OH groups;
each R^{A} is independently halogen, —OH, —O—(C₁-C₆ alkyl), C₁-C₆ alkyl, C₃-C₆ cycloalkyl, 3- to 8-membered heterocycloalkyl, wherein each alkyl, cycloalkyl, and heterocycloalkyl is unsubstituted or substituted with 1, 2, or 3 substituents selected from halogen, —CN, —OH, —O—(C₁-C₆ alkyl), C₁-C₆ alkyl, C₁-C₆ fluoroalkyl, C₁-C₆ hydroxyalkyl, —O—(C₁-C₆ fluoroalkyl), C₃-C₆ cycloalkyl, and 3- to 6-membered heterocycloalkyl;
each R^{B} is independently halogen, C₁-C₆ alkyl, C₃-C₆ cycloalkyl, C₃-C₆ cycloalkenyl, 3- to 8-membered heterocycloalkyl, 3- to 8-membered heterocycloalkenyl, aryl, heteroaryl, —CN, —OR⁹, —OCH₂R⁹, —CO₂R⁹, —CH₂CO₂R⁹, —OC(═O)R⁹, —C(═O)N(R⁹)₂, —N(R⁹)₂, —NR⁹C(═O)R⁹, —NR⁹C(═O)OR¹⁰, —OC(═O)NR⁹, —NR⁹C(═O)N(R⁹)₂, —C(R⁹)═N—OR⁹, —SR⁹, —S(═O)R¹⁰, —S(═O)₂R¹⁰, —S(═O)₂N(R⁹)₂, —P(═O)(OR⁹)₂, —P(═O)(OR⁹)R¹⁰ or —P(═O)(R¹⁰)₂, wherein each alkyl, aryl, and heteroaryl is unsubstituted or substituted with 1, 2, or 3 substituents selected from halogen, —CN, —OH, —O—(C₁-C₆ alkyl), —CO₂—(C₁-C₆ alkyl), C₁-C₆ alkyl, C₁-C₆ fluoroalkyl, C₁-C₆ hydroxyalkyl, —O—(C₁-C₆ fluoroalkyl), C₃-C₆ cycloalkyl, and 3- to 6-membered heterocycloalkyl; and
wherein each cycloalkyl, cycloalkenyl, heterocycloalkyl, and heterocycloalkenyl is unsubstituted or substituted with 1, 2, or 3 substituents selected from halogen, —CN, —OH, ═O, —O—(C₁-C₆ alkyl), C₁-C₆ alkyl, C₁-C₆ fluoroalkyl, C₁-C₆ hydroxyalkyl, —O—(C₁-C₆ fluoroalkyl), C₃-C₆ cycloalkyl, and 3- to 6-membered heterocycloalkyl;
each R⁹ is independently selected from hydrogen, C₁-C₆ alkyl, C₁-C₆ fluoroalkyl, C₃-C₆ cycloalkyl, 3- to 8-membered heterocycloalkyl, phenyl, benzyl, and monocyclic heteroaryl, wherein each alkyl, fluoroalkyl, cycloalkyl, heterocycloalkyl, phenyl, benzyl, and heteroaryl is unsubstituted or substituted with 1, 2, or 3 substituents selected from halogen, —CN, —OH, —O—(C₁-C₆ alkyl)₂, —NH₂, —NH(C₁-C₆ alkyl), —N(C₁-C₆ alkyl)₂, C₁-C₆ alkyl, C₁-C₆ fluoroalkyl, C₁-C₆ hydroxyalkyl, —O—(C₁-C₆ fluoroalkl), C₃-C₆ cycloalkyl, 3- to 6-membered heterocycloalkyl, and 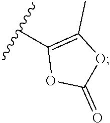 [SMILES: *c1oc(=O)oc1C]
or two R⁹ on the same N atom are taken together with the N atom to which they are attached to form a N-containing heterocycle, which is unsubstituted or substituted with 1, 2, or 3 substituents selected from halogen, —CN, —OH, —O—(C₁-C₆ alkyl), —NH₂, —NH(C₁-C₆ alkyl), —N(C₁-C₆ alkyl)₂, C₁-C₆ alkyl, C₁-C₆ fluoroalkyl, C₁-C₆ hydroxyalkyl, —O—(C₁-C₆ fluoroalkyl), C₃-C₆ cycloalkyl, and 3- to 6-membered heterocycloalkyl;
each R¹⁰ is independently selected from C₁-C₆ alkyl, C₁-C₆ fluoroalkyl, C₃-C₆ cycloalkyl, 3- to 8-membered heterocycloalkyl, phenyl, benzyl, and monocyclic heteroaryl, wherein each alkyl, fluoroalkyl, cycloalkyl, heterocycloalkyl, phenyl, benzyl, and heteroaryl is unsubstituted or substituted with 1, 2, or 3 substituents selected from halogen, —CN, —OH, —O—(C₁-C₆ alkyl), —NH₂, —NH(C₁-C₆ alkyl), —N(C₁-C₆ alkyl)₂, C₁-C₆ alkyl, C₁-C₆ fluoroalkyl, C₁-C₆ hydroxyalkyl, —O—(C₁-C₆ fluoroalkyl), C₃-C₆ cycloalkyl, 3- to 6-membered heterocycloalkyl, and 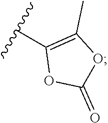 [SMILES: *c1oc(=O)oc1C]
m is 1 or 2;
n is 1 or 2;
p is 1-4; and
q is 0-4.

38. The compound of claim 37, or a pharmaceutically acceptable salt, solvate, stereoisomer, or prodrug thereof, wherein:
Ring B is phenyl or 6-membered heteroaryl;
each R¹ and R² is independently hydrogen or C₁₋₆ alkyl;
m is 2; and
n is 2.

39. The compound of claim 37, or a pharmaceutically acceptable salt, solvate, stereoisomer, or prodrug thereof, wherein the compound has the structure of Formula (IIa-1), or a pharmaceutically acceptable salt, solvate, stereoisomer, or prodrug thereof:

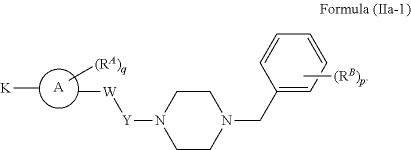

40. The compound of any one of claims 37-39, or a pharmaceutically acceptable salt, solvate, stereoisomer, or prodrug thereof, wherein:
W is a bond, and Y is —C(═O)—;
or W is —O—, and Y is —C(═O)—;
or W is —NR³—, and Y is —C(═O)—;
or W is —C(R⁴)₂—; and Y is —C(═O)—;
or W is a bond, and Y is —S(═O)₂—;
or W is —O—, and Y is —S(═O)₂—;
or W is —NR³—, and Y is —S(═O)₂—;
or W is —C(R⁴)₂—; and Y is —S(═O)₂—.

41. The compound of any one of claims 37-40, or a pharmaceutically acceptable salt, solvate, stereoisomer, or prodrug thereof, wherein:
W is —O—, and Y is —C(═O)—;
or W is —NR³—, and Y is —C(═O)—;
or W is —C(R⁴)₂—; and Y is —C(═O)—;
or W is a bond, and Y is —C(═O)—.

42. The compound of claim 37, or a pharmaceutically acceptable salt, solvate, stereoisomer, or prodrug thereof, wherein the compound has the structure of Formula (IIb), Formula (IIc), Formula (IId), or Formula (IIe), or a pharmaceutically acceptable salt, solvate, stereoisomer, or prodrug thereof:

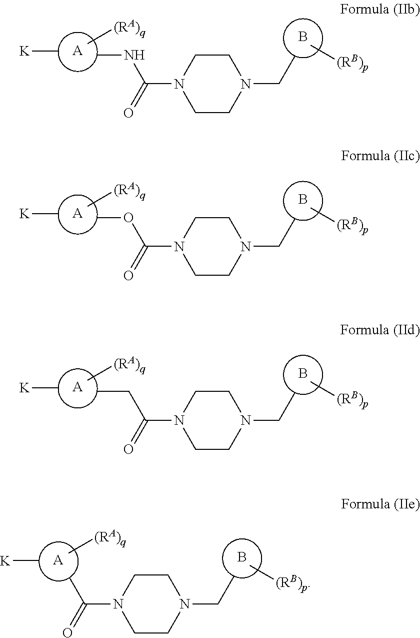

43. The compound of any one of claims 37-42, or a pharmaceutically acceptable salt, solvate, stereoisomer, or prodrug thereof, wherein:
each R^{B} is independently halogen, C₁-C₆ alkyl, phenyl, C₃-C₆ cycloalkyl, 3- to 6-membered heterocycloalkyl, 3- to 6-membered heterocycloalkenyl, 5-membered heteroaryl, 6-membered heteroaryl, —CN, —OR⁹, —CH₂CO₂R⁹, —CO₂R⁹, —C(═O)N(R⁹)₂, —N(R⁹)₂, —S(═O)₂R¹⁰, —S(═O)₂N(R⁹)₂, or —P(═O)(R¹⁰)₂, wherein each alkyl, phenyl, and heteroaryl is unsubstituted or substituted with 1, 2, or 3 substituents selected from halogen, —CN, —OH, —O—(C₁-C₆ alkyl), C₁-C₆ alkyl, C₁-C₆ fluoroalkyl, C₁-C₆ hydroxyalkyl, —O—(C₁-C₆ fluoroalkyl), C₃-C₆ cycloalkyl, and 3- to 6-membered heterocycloalkyl; and wherein each cycloalkyl, heterocycloalkyl, and heterocycloalkenyl is unsubstituted or substituted with 1, 2, or 3 substituents selected from halogen, —CN, —OH, ═O, —O—(C₁-C₆ alkyl), C₁-C₆ alkyl, C₁-C₆ fluoroalkyl, C₁-C₆ hydroxyalkyl, —O—(C₁-C₆ fluoroalkyl), C₃-C₆ cycloalkyl, and 3- to 6-membered heterocycloalkyl.

44. The compound of any one of claims 37-43, or a pharmaceutically acceptable salt, solvate, stereoisomer, or prodrug thereof, wherein:
each R^{B} is independently halogen, C₁-C₆ alkyl, phenyl, C₃-C₆ cycloalkyl, 5-membered heteroaryl, 6-membered heteroaryl, —CN, —OR⁹, —CH₂CO₂R⁹, —CO₂R⁹, —C(═O)N(R⁹)₂, or —S(═O)₂R¹⁰, wherein each alkyl, cycloalkyl, phenyl, and heteroaryl is unsubstituted or substituted with 1, 2, or 3 substituents selected from —F, —Cl, —Br, —CN, —OH, —CH₂OH, —O—(C₁-C₆ alkyl), C₁-C₆ alkyl, C₁-C₆ fluoroalkyl.

45. The compound of claim 37, or a pharmaceutically acceptable salt, solvate, stereoisomer, or prodrug thereof, wherein the compound has the structure of Formula (IIf), or a pharmaceutically acceptable salt, solvate, stereoisomer, or prodrug thereof:

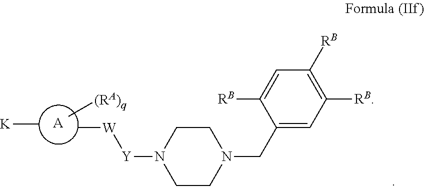

46. The compound of claim 37, or a pharmaceutically acceptable salt, solvate, stereoisomer, or prodrug thereof, wherein the compound has the structure of Formula (JIg), or a pharmaceutically acceptable salt, solvate, stereoisomer, or prodrug thereof:

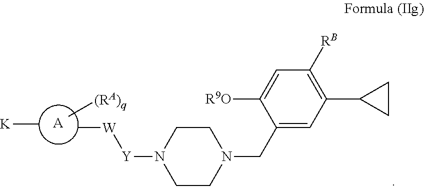

47. The compound of claim 46, or a pharmaceutically acceptable salt, solvate, stereoisomer, or prodrug thereof, wherein:
R^{B} is phenyl, oxadiazolyl, pyridinyl, —CN, —CH₂CO₂R⁹, —CO₂R⁹, or —S(═O)₂R¹⁰ wherein the phenyl, oxadiazolyl, or pyridinyl is unsubstituted or substituted with 1, 2, or 3 substituents selected from —F, —Cl, —Br, —CN, —OH, —CH₂OH, —O—(C₁-C₆ alkyl), C₁-C₆ alkyl, C₁-C₆ fluoroalkyl.

48. The compound of any one of claims 37-47, or a pharmaceutically acceptable salt, solvate, stereoisomer, or prodrug thereof, wherein:
Ring A is phenyl, monocyclic heteroaryl, monocyclic cycloalkyl, spirocyclic cycloalkyl, bridged cycloalkyl, monocyclic heterocycloalkyl, spirocyclic heterocycloalkyl, or bridged heterocycloalkyl;
each R^{A} is independently halogen, —OH, —O—(C₁-C₆ alkyl), C₁-C₆ alkyl, C₃-C₆ cycloalkyl, wherein each alkyl and cycloalkyl is unsubstituted or substituted with 1, 2, or 3 substituents selected from halogen, —CN, —OH, —O—(C₁-C₆ alkyl), C₁-C₆ alkyl, and C₁-C₆ fluoroalkyl; and
q is 0-2.

49. The compound of any one of claims 37-47, or a pharmaceutically acceptable salt, solvate, stereoisomer, or prodrug thereof, wherein:
Ring A is phenyl, monocyclic C₃-C₆ cycloalkyl, or bridged cycloalkyl;
each R^{A} is independently halogen, —OH, —O—(C₁-C₆ alkyl), or C₁-C₆ alkyl; and
q is 0-2.

50. The compound of any one of claims 37-47, or a pharmaceutically acceptable salt, solvate, stereoisomer, or prodrug thereof, wherein:
Ring A is phenyl, cyclohexyl, or 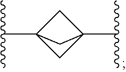 [SMILES: *C12CC(*)(C1)C2]
each R^{A} is independently halogen, —OH, —O—(C₁-C₆ alkyl), or C₁-C₆ alkyl; and
q is 0-2.

51. The compound of any one of claims 37-47, or a pharmaceutically acceptable salt, solvate, stereoisomer, or prodrug thereof, wherein:
Ring A is phenyl; and
q is 0.

52. The compound of claim 37, or a pharmaceutically acceptable salt, solvate, stereoisomer, or prodrug thereof, wherein the compound has the structure of Formula (IIh), or a pharmaceutically acceptable salt, solvate, stereoisomer, or prodrug thereof:

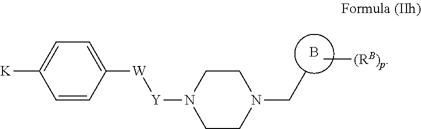

53. The compound of any one of claims 37-52, or a pharmaceutically acceptable salt, solvate, stereoisomer, or prodrug thereof, wherein:
K is —(CH₂)ⱼ-G; and
j is 0 or 1.

54. The compound of any one of claims 37-53, or a pharmaceutically acceptable salt, solvate, stereoisomer, or prodrug thereof, wherein:
G is —P(═O)(OH)₂, —P(═O)(OH)(R^{D}), —P(═O)(OH)(OR^{D}), —B(OH)₂, —B(OR^{D})(OH), —NHC(═O)H, —NHC(═O)(R^{D}), —NHS(═O)₂(R^{D}), —NHC(═O)NH₂, —NHC(═O)NH(R^{D}), —N(R^{D})C(═O)NHS(═O)₂(R^{D}), or —C(═O)NHS(═O)₂(R^{D}); and
R^{D} is alkyl which is unsubstituted or substituted with 1, 2, or 3 —OH groups.

55. The compound of any one of claims 37-54, or a pharmaceutically acceptable salt, solvate, stereoisomer, or prodrug thereof, wherein:
G is —P(═O)(OH)₂, —P(═O)(OH)(R^{D}), or —P(═O)(OH)(OR^{D});
R^{D} is C₁₋₆ alkyl; and
j is 0 or 1.

56. The compound of any one of claims 37-55, or a pharmaceutically acceptable salt, solvate, stereoisomer, or prodrug thereof, wherein:
K is —(CH₂)ⱼP(═O)(OH)₂, —(CH₂)ⱼP(═O)(OMe)(OH), —(CH₂)ⱼP(═O)(OEt)(OH), —(CH₂)ⱼP(═O)(Me)(OH), or —(CH₂)ⱼP(═O)(Et)(OH); and
j is 0 or 1.

57. The compound of any one of claims 37-56, or a pharmaceutically acceptable salt, solvate, stereoisomer, or prodrug thereof, wherein:
K is —P(═O)(OH)₂, —P(═O)(OEt)(OH), or —P(═O)(Me)(OH).

58. The compound of claim 37, wherein the compound is:
(4-(((4-((2-cyclopropyl-5-ethoxy-4′-fluoro-[1,1′-biphenyl]-4-yl)methyl)piperazin-1-yl)sulfonyl)methyl)phenyl)phosphonic acid;
ethyl hydrogen (4-(((4-((2-cyclopropyl-5-ethoxy-4′-fluoro-[1,1′-biphenyl]-4-yl)methyl)piperazin-1-yl)sulfonyl)methyl)phenyl)phosphonate;
ethyl hydrogen (4-(2-(4-((2-cyclopropyl-5-ethoxy-4′-fluoro-[1,1′-biphenyl]-4-yl)methyl)piperazin-1-yl)-2-oxoethyl)phenyl)phosphonate;
(4-(2-(4-((2-cyclopropyl-5-ethoxy-4′-fluoro-[1,1′-biphenyl]-4-yl)methyl)piperazin-1-yl)-2-oxoethyl)phenyl)phosphonic acid;
4-((((2S,3R,4R,5R)-2,3,4,5,6-pentahydroxyhexyl)amino)methyl)phenyl 4-(5-cyclopropyl-2-ethoxy-4-(methoxycarbonyl)benzyl)piperazine-1-carboxylate;
or a pharmaceutically acceptable salt, solvate, stereoisomer, or prodrug thereof.

59. A pharmaceutical composition comprising a compound of any one of claims 1-58, or a pharmaceutically acceptable salt, solvate, stereoisomer, or prodrug thereof, and at least one pharmaceutically acceptable excipient.

60. A method of treating a condition or disorder involving the gut-brain axis in a subject in need thereof, the method comprising administering to the subject a therapeutically effective amount of a compound of any one of claims 1-58, or a pharmaceutically acceptable salt, solvate, stereoisomer, or prodrug thereof.

61. The method of claim 60, wherein the condition or disorder is associated with SSTR5 activity.

62. The method of claim 60 or 61, wherein the condition or disorder is a metabolic disorder.

63. The method of claim 62, wherein the condition or disorder is type 2 diabetes, hyperglycemia, metabolic syndrome, obesity, hypercholesterolemia, nonalcoholic steatohepatitis, or hypertension.

64. The method of claim 60 or 61, wherein the condition or disorder is a nutritional disorder.

65. The method of claim 64, wherein the condition or disorder is short bowel syndrome, intestinal failure, or intestinal insufficiency.

66. A method of augmenting weight loss or preventing weigth gain or weight regain, the method comprising administering to the subject a therapeutically effective amount of a compound of any one of claims 1-58, or a pharmaceutically acceptable salt, solvate, stereoisomer, or prodrug thereof.

67. The method of claim 66, wherein the subject has had bariatric surgery.

68. A method of treating gastrointestinal injury resulting from toxic insults such as radiation or chemotherapy in a subject in need thereof, the method comprising administering to the subject a therapeutically effective amount of a compound of any one of claims 1-58, or a pharmaceutically acceptable salt, solvate, stereoisomer, or prodrug thereof.

69. The method of any one of claims 60-68, wherein the compound is gut-restricted.

70. The method of claim 69, wherein the compound has low systemic exposure.

71. The method of any one of claims 60-70, further comprising administering one or more additional therapeutic agents to the subject.

72. The method of claim 71, wherein the one or more additional therapeutic agents are selected from a TGR5 agonist, a GPR40 agonist, a GPR119 agonist, a CCK1 agonist, a PDE4 inhibitor, a DPP-4 inhibitor, a GLP-1 receptor agonist, metformin, or a combination thereof.

73. The method of claim 72, wherein the TGR5 agonist, GPR40 agonist, GPR119 agonist, or CCK1 agonist is gut-restricted.
